(19) United States
(12) Patent Application Publication
(10) Pub. No.: US 2003/0170630 A1
(43) Pub. Date: Sep. 11, 2003

(54) Proteins and nucleic acids encoding same

(76) Inventors: John P. Alsobrook II, Madison, CT (US); Velizar T. Tchernev, Branford, CT (US); Xiaohong Liu, Canton, MA (US); Kimberly A. Spytek, New Haven, CT (US); Bryan D. Zerhusen, Branford, CT (US); Meera Patturajan, Branford, CT (US); Denise M. Lepley, Branford, CT (US); Catherine E. Burgess, Wethersfield, CT (US); Richard A. Shimkets, Guilford, CT (US); William M. Grosse, Branford, CT (US); Edward S. Szekeres JR., Branford, CT (US); Corine A.M. Vernet, Branford, CT (US); Li Li, Branford, CT (US); Stacie J. Casman, North Haven, CT (US); Ference L. Boldog, North Haven, CT (US); Linda Gorman, Branford, CT (US); Esha A. Gangolli, Madison, CT (US); Elma R. Fernandes, Branford, CT (US); Danier K. Rieger, Branford, CT (US); Shlomit R. Edinger, New Haven, CT (US); Erik Gunther, Branford, CT (US); Isabelle Millet, Milford, CT (US); Paul Sciore, North Haven, CT (US); Karen Ellerman, Branford, CT (US); John R. MacDougall, Hamden, CT (US); Glennda Smithson, Guilford, CT (US)

Correspondence Address: Ivor R. Elrifi, Mintz, Levin, Cohn, Ferris,, Glovsky and Popeo, P.C., One Financial Center, Boston, MA, 02111, US

(21) Appl. No.: 10/032,189

(22) Filed: Dec. 21, 2001

Related U.S. Application Data
(60) Provisional application No. 60/257,495, filed on Dec. 21, 2000.
(60) Provisional application No. 60/258,171, filed on Dec. 22, 2000.
(60) Provisional application No. 60/269,940, filed on Feb. 20, 2001.
(60) Provisional application No. 60/274,192, filed on Mar. 8, 2001.
(60) Provisional application No. 60/277,826, filed on Mar. 22, 2001.
(60) Provisional application No. 60/279,840, filed on Mar. 29, 2001.
(60) Provisional application No. 60/282,981, filed on Apr. 11, 2001.
(60) Provisional application No. 60/283,656, filed on Apr. 13, 2001.
(60) Provisional application No. 60/309,247, filed on Jul. 31, 2001.
(60) Provisional application No. 60/311,754, filed on Aug. 10, 2001.
(60) Provisional application No. 60/313,331, filed on Aug. 17, 2001.

(51) Int. Cl. C12Q 1/68; C07H 21/04; C12N 9/00; C12P 21/02; C12N 5/06

(52) U.S. Cl. 435/6; 435/69.1; 435/183; 435/320.1; 435/325; 536/23.2

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S. Ser. Nos. 60/257,495, filed Dec. 21, 2000; 60/258,171 filed Dec. 22, 2000; 60/269,940, filed Feb. 20, 2001; 60/274,192 filed Mar. 8, 2001; 60/277,826, filed Mar. 22,2001; 60/279,840 filed Mar. 29,2001; 60/282,981, filed Apr. 11, 2001; 60/283,656 filed Apr. 13, 2001; 60/309,247, filed Jul. 31, 2001; 60/311,754, filed Aug. 10, 2001; and 60/313,331, filed Aug. 17, 2001; each of which is incorporated by reference in its entirety.

### FIELD OF THE INVENTION

The invention generally relates to nucleic acids and polypeptides encoded thereby.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, NOV10, NOV11, NOV12, and NOV13 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58. The nucleic acid can be, for example, a genomic DNA fragment or a cDNA molecule that includes the nucleic acid sequence of any of SEQ ID NOS: 1, 3, 5, 7, 9, 11, 13, 15, 17, 19, 21, 23, 25, 27, 29, 31,33, 35, 37, 39, 41,43, 45, 47,49, 51, 53, 55, and 57.

Also included in the invention is an oligonucleotide, e.g., an oligonucleotide which includes at least 6 contiguous nucleotides of a NOVX nucleic acid (e.g., SEQ ID NOS: 3, 5, 7, 9, 11, 13, 15, 17, 19, 21, 23, 25, 27, 29, 31, 33, 35, 37, 39, 41, 43, 45, 47, 49, 51, 53, 55, and 57) or a complement of said oligonucleotide.

Also included in the invention are substantially purified NOVX polypeptides (SEQ ID NOS: 2, 4, 6, 8, 10, 12, 14, 16, 18, 20, 22, 24, 26, 28, 30, 32, 34, 26, 28,40, 42, 44, 46,48, 50, 52, 54, 56, and 58). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., asthma, allergies, emphysema, bronchitis, autoimmune disease, immunodeficiencies, transplantation, graft versus host disease, arthritis, tendonitis, scleroderma, systemic lupus erythematosus, ARDS, lymphedema, allergic encephalomyelitis, experimental allergic encephalomyelitis (EAE), various forms of arthritis, bacterial infections, cystic fibrosis, lung cancer, adrenoleukodystrophy, congenital adrenal hyperplasia, leukodystrophies, cancer such as AML, coronary artery disease, stroke, hypertension, myocardial infarction, atherosclerosis, hemophilia, hypercoagulation, idiopathic thrombocytopenic purpura, aneurysm, hypertension, myocardial infarction, embolism, cardiovascular disorders, bypass surgery, hypertriglyceridemia, hypoalphalipoproteinemia, hyperlipidemia, noninsulin-dependent diabetes mellitus, obesity, diabetes, Diabetes insipidus nephrogenic, autosomal dominant; Diabetes insipidus, nephrogenic, autosomal recessive; Tangier disease, LCAT deficiency, ‘fish-eye’ disease, Von Hippel-Lindau (VHL) syndrome, tuberous sclerosis, hypercalceimia, Lesch-Nyhan syndrome, cirrhosis, inflammatory bowel disease, diverticular disease, Hirschsprung's disease, Crohn's Disease, appendicitis, ulcers, laryngitis, muscular dystrophy, myasthenia gravis, endometriosis, pancreatitis, hyperparathyroidism, hypoparathyroidism, xerostomia, psoriasis, actinic keratosis, acne, hair growth/loss, allopecia, pigmentation disorders, endocrine disorders, tonsillitis, cystitis, incontinence, uveitis, corneal fibroblast proliferation, amyotrophic lateral sclerosis, acute pancreatitis, cerebral cryptococcosis, colitis, thyroiditis, nonsyndromic deafness, keratinization disorders, gap-junction-related neuropathies and other pathological conditions of the nervous system, where dysfunctions of junctional communication are considered to play a casual role, demyelinating neuropathies (including Charcot-Marie-Tooth disease), erythrokeratodermia variabilis (EKV), atrioventricular (AV) conduction defects such as arrhythmia, lens cataract, osteoporosis, osteoarthirtis, Achalasia-addisonianism-alacrimia syndrome; Cataract, polymorphic and lamellar; Cyclic ichthyosis with epidermolytic hyperkeratosis; Enuresis, nocturnal, 2; Epidermolysis bullosa simplex, Koebner, Dowling-Meara, and Weber-Cockayne types; Epidermolytic hyperkeratosis; Fundus albipunctatus; Glioma; Ichthyosis bullosa of Siemens; Keratoderma, palmoplantar, nonepidermolytic; Meesmann corneal dystrophy; Monilethrix; Myopathy, congenital; Pachyonychia congenita, Jackson-Lawler type; Pachyonychia congenita, Jadassohn-Lewandowsky type; Palmoplantar keratoderma, Bothnia type; Persistent Mullerian duct syndrome, type II; Spastic paraplegia-10; White sponge nevus; Liver disease, susceptibility to, from hepatotoxins or viruses; Alzheimer's disease, Parkinson's disease, Huntington's disease, cerebral palsy, epilepsy, multiple sclerosis, ataxia-telangiectasia, behavioral disorders, addiction, anxiety, pain, neuroprotection, fertility, growth and reproductive disorders, renal artery stenosis, interstitial nephritis, glomerulonephritis, polycystic kidney disease, renal tubular acidosis, IgA nephropathy, and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g. a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1a | CG55750-01 | 1 | 2 | Airway Trypsin-Like |
| | | | | Protease-like |
| 1b | 168446573 | 3 | 4 | Airway Trypsin-Like |
| | | | | Protease-like |
| 1c | 168446539 | 5 | 6 | Airway Trypsin-Like |
| | | | | Protease-like |
| 1d | 168446547 | 7 | 8 | Airway Trypsin-Like |
| | | | | Protease-like |
| 2 | CG55782-01 | 9 | 10 | P450-like |
| 3a | CG55771-01 | 11 | 12 | Apolipoprotein A-I |
| | | | | precursor-like |
| 3b | CG55771-02 | 13 | 14 | Apolipoprotein A-I |
| | | | | precursor-like |
| 4a | CG55700-01 | 15 | 16 | HSP90 co-chaperone-like |
| 4b | CG55700-02 | 17 | 18 | HSP90 Co-Chaperone |
| | | | | (Progesterone Receptor |
| | | | | Complex P23) - like |
| 4c | CG55700-03 | 19 | 20 | HSP90 co-chaperone-like |
| 5 | CG55706-01 | 21 | 22 | Type III adenylyl cyclase- |
| | | | | like |
| 6a | CG50389-02 | 23 | 24 | Interleukin 1 receptor |
| | | | | related protein-like |
| 6b | CG50389-03 | 25 | 26 | Interleukin 1 receptor |
| | | | | related protein-like |
| 6c | CG50389-04 | 27 | 28 | Interleukin 1 receptor |
| | | | | related protein-like |
| 7 | CG50389-01 | 29 | 30 | Interleukin 1 receptor |
| | | | | related protein-like |
| 8 | CG50387-02 | 31 | 32 | Connexin GJA3-like |
| 9 | CG50271-01 | 33 | 34 | Olfactory Receptor-like |
| 10 | CG55844-01 | 35 | 36 | P450-like |
| 11a | CG55752-01 | 37 | 38 | Alpha Glucosidase 2, Alpha |
| | | | | Neutral Subunit-like |
| 11b | CG55752-02 | 39 | 40 | Alpha Glucosidase 2-like |
| 11c | CG55752-03 | 41 | 42 | Glucosidase II-like |
| 11d | CG55752-04 | 43 | 44 | Glucosidase II-like |
| 12a | CG55776-01 | 45 | 46 | Mechanical stress induced |
| | | | | protein-like |
| 12b | 174124289 | 47 | 48 | Mechanical stress induced |
| | | | | protein-like |
| 12c | 174124313 | 49 | 50 | Mechanical stress induced |
| | | | | protein-like |
| 12d | 174124322 | 51 | 52 | Mechanical stress induced |
| | | | | protein-like |
| 12e | 174124322 | 53 | 54 | Mechanical stress induced |
| | | | | protein-like |
| 12f | CG55776-03 | 55 | 56 | Mechanical stress induced |
| | | | | protein-like |
| 13 | CG55908-01 | 57 | 58 | Integrin-like FG-GAP domain |
| | | | | containing novel protein- |
| | | | | like |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1 is homologous to a Airway Trypsin-Like Protease-like family of proteins. Thus, the NOV1 nucleic acids, polypeptides, antibodies and related compounds according to the invention will be useful in therapeutic and diagnostic applications implicated in, for example; asthma and cystic fibrosis, allergies, emphysema, bronchitis, lung cancer, or other pathologie or conditions.

NOV2 is homologous to the P450-like family of proteins. Thus NOV2 nucleic acids, polypeptides, antibodies and related compounds according to the invention will be useful in therapeutic and diagnostic applications implicated in various pathologies and disorders.

NOV3 is homologous to a family of Apolipoprotein A-I precursor-like proteins. Thus, the NOV3 nucleic acids and polypeptides, antibodies and related compounds according to the invention will be useful in therapeutic and diagnostic applications implicated in, for example: coronary artery disease, stroke, hypertriglyceridemia, hypoalphalipoproteinemia, hyperlipidemia, Tangier disease, LCAT deficiency, ‘fish-eye’ disease, noninsulin-dependent diabetes mellitus, hypertension, myocardial infarction, atherosclerosis, and/or other pathologies.

NOV4 is homologous to the HSP90 co-chaperone-like family of proteins. Thus, NOV4 nucleic acids, polypeptides, antibodies and related compounds according to the invention will be useful in therapeutic and diagnostic applications implicated in, for example: adrenoleukodystrophy, congenital adrenal hyperplasia, hemophilia, hypercoagulation, idiopathic thrombocytopenic purpura, autoimmune disease, allergies, asthma, immunodeficiencies, transplantation, graft versus host disease, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, arthritis, tendonitis, fertility, atherosclerosis, aneurysm, hypertension, fibromuscular dysplasia, stroke, scleroderma, obesity, myocardial infarction, embolism, cardiovascular disorders, bypass surgery, cirrhosis, inflammatory bowel disease, diverticular disease, Hirschsprung's disease, Crohn's Disease, appendicitis, ulcers, diabetes, renal artery stenosis, interstitial nephritis, glomerulonephritis, polycystic kidney disease, systemic lupus erythematosus, renal tubular acidosis, IgA nephropathy, laryngitis, emphysema, ARDS, lymphedema, muscular dystrophy, myasthenia gravis, endometriosis, pancreatitis, hyperparathyroidism, hypoparathyroidism, growth and reproductive disorders, xerostomia, psoriasis, actinic keratosis, acne, hair growth/loss, allopecia, pigmentation disorders, endocrine disorders, tonsillitis, cystitis, incontinence, and/or other pathologies.

NOV5 is homologous to the Type III adenylyl cyclase-like family of proteins. Thus NOV5 nucleic acids, polypeptides, antibodies and related compounds according to the invention will be useful in therapeutic and diagnostic applications implicated in, diabetes, heart failure, neurological diseases such as epilepsy, sleep disorder, parkinsonism, Huntington's disease, Alzheimer's disease, depression, schizophrenia diseases, disorders and conditions.

NOV6 is homologous to the Interleukin 1 receptor related protein-like family of proteins. Thus NOV6 nucleic acids, polypeptides, antibodies and related compounds according to the invention will be useful in therapeutic and diagnostic applications implicated in, for example: uveitis and corneal fibroblast proliferation, allergic encephalomyelitis, amyotrophic laternal sclerosis, acute pancreatitis, cerebral cryptococcosis, autoimmune disease including Type 1 diabetes mellitus (DM), experimental allergic encephalomyelitis (EAE), systemic lupus erythematosus (SLE), colitis, thyroiditis and various forms of arthritis, cancer such as AML, bacterial infections, and/or other pathologies/disorders.

NOV7 is homologous to members of the Interleukin 1 receptor related protein-like family of proteins. Thus, the NOV7 nucleic acids, polypeptides, antibodies and related compounds according to the invention will be useful in therapeutic and diagnostic applications implicated in, for example; uveitis and corneal fibroblast proliferation, allergic encephalomyelitis, amyotrophic lateral sclerosis, acute pancreatitis, cerebral cryptococcosis, autoimmune disease including Type 1 diabetes mellitus (DM), experimental allergic encephalomyelitis (EAE), systemic lupus erythematosus (SLE), colitis, thyroiditis and various forms of arthritis, cancer such as AML, bacterial infections, and/or other pathologies/disorders.

NOV8 is homologous to the connexin GJA3-like family of proteins. Thus, NOV8 nucleic acids and polypeptides, antibodies and related compounds according to the invention will be useful in therapeutic and diagnostic applications implicated in, for example; ) nonsyndromic deafness, keratinization disorders, gap-junction-related neuropathies and other pathological conditions of the nervous system, where dysfunctions of junctional communication are considered to play a casual role, demyelinating neuropathies (including Charcot-Marie-Tooth disease), erythrokeratodermia variabilis (EKV), atrioventricular (AV) conduction defects such as arrhythmia, lens cataract, and/or other pathologies/disorders.

NOV9 is homologous to the Olfactory Receptor-like family of proteins. Thus, NOV9 nucleic acids and polypeptides, antibodies and related compounds according to the invention will be useful in therapeutic and diagnostic applications implicated in various pathologies or disorders.

NOV10 is homologous to the P450-like family of proteins. Thus, NOV10 nucleic acids and polypeptides, antibodies and related compounds according to the invention will be useful in various pathologies or disorders.

NOV11 is homologous to the Integrin-like FG-GAP domain containing novel protein-like family of proteins. Thus, NOV11 nucleic acids and polypeptides, antibodies and related compounds according to the invention will be useful in therapeutic and diagnostic applications implicated in various pathologies or disorders.

NOV12 is homologous to the Mechanical stress induced protein-like family of proteins. Thus, NOV12 nucleic acids and polypeptides, antibodies and related compounds according to the invention will be useful in therapeutic and diagnostic applications implicated in, for example; osteoporosis, osteoarthritis, cardiac hypertrophy, atherosclerosis, hypertension, restenosis, and/or other pathologies/disorders.

NOV13 is homologous to the Integrin-like FG-GAP domain containing novel protein-like family of proteins. Thus, NOV13 nucleic acids and polypeptides, antibodies and related compounds according to the invention will be useful in therapeutic and diagnostic applications implicated in, for example; Achalasia-addisonianism-alacrimia syndrome; Cataract, polymorphic and lamellar; Cyclic ichthyosis with epidermolytic hyperkeratosis; Diabetes insipidus, nephrogenic, autosomal dominant; Diabetes insipidus, nephrogenic, autosomal recessive; Enuresis, nocturnal, 2; Epidermolysis bullosa simplex, Koebner, Dowling-Meara, and Weber-Cockayne types; Epidermolytic hyperkeratosis; Fundus albipunctatus; Glioma; Ichthyosis bullosa of Siemens; Keratoderma, palmoplantar, nonepidermolytic; Meesmann corneal dystrophy; Monilethrix; Myopathy, congenital; Pachyonychia congenita, Jackson-Lawler type; Pachyonychia congenita, Jadassohn-Lewandowsky type; Palmoplantar keratoderma, Bothnia type; Persistent Mullerian duct syndrome, type II; Spastic paraplegia-10; White sponge nevus; Liver disease, susceptibility to, from hepatotoxins or viruses; Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection; lymphedema, allergies, and/or other pathologies/disorders.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

NOV1 includes three novel Airway Trypsin-Like Protease-like proteins disclosed below. The disclosed sequences have been named NOV1a, NOV1b, and NOV1c.

NOV1a

A disclosed NOV1a nucleic acid of 1386 nucleotides (also referred to as CG55750-01) encoding a Airway Trypsin-Like Protease-like protein is shown in Table 1A. An open reading frame was identified beginning with an ATG initiation codon at nucleotides 64-66 and ending with a TGA codon at nucleotides 1324-1326. A putative untranslated region upstream from the initiation codon and downstream from the termination codon is underlined in Table 1A. The start and stop codons are in bold letters.

TABLE 1A
NOV1a nucleotide sequence.

+TR,1(SEQ ID NO:1)

AAAAGGAACATTTAGTCTTAAAATCCTATTCATTTTTAACACACAATTCTTTCTCAAAAGGCCATGACACTG
GGTAGAAGAGTGAGTTCACTGAAACCATGGATGTTTGCCCTTATTGTCAGAGCTGTTGTGTTGATTCTGGTG
ATACTCATTGGTCTCCTTGTTTATTTTTTGGCATATAAGTTTTACTATTACCAAACCTCCTTCCAGATCCCC
AGTATTGAATATAATTTAGCTATTAATACTTGTGTGACACAAGAGGAGAGAATCTATGACAATAAAATGTGT
AAAATAATGTCTAGGATATTTCGACATTCTTCTGTAGGCGGTCGATTTATCAAATCTCATGTTATCAAATTA
AGGCCAAGTAATGACAATTTGAAAGCAGATGTATTGCTTAAATTTCAGTTTATTCCTAACAATGAGAACGCA
ATAAAAACACAAGCTGATAACATTTTGCATCAGAAGTTGAAATCAAATGAAAGCTCTTTGACCATAAACAAA
CCATCATTTAGACTCACACCTATTGACAGCAAAAAGATGAGGAATCTTCTCAACAGTCGCTGTGGAATAAGG
ATGACATCTTCAAACATGCCATTACCAGCATCCTCTTCTACTCAAAGAATTGTCCAAGGAAGGGAAACAGCT
ATGGAAGGGGAATGGCCATGGCAGGCCAGCCTCCAGCTCATAGGGTCAGGCCATCAGTGTGGAGCCAGCCTC
ATCAGTAACACATGGCTGCTCACAGCAGCTCACTGCTTTTGGAAAAATAAAGACCCAAGTCAATGGATTGCT
ACTTTTGGTGCAACTATAACACCACCCGCAGTGAAACGAAATGTGAGGAAAATTATTCTTCATGAGAATTAC
CATAGAGAAACAAATGAAAATGACATTGCTTTGGTTCAGCTCTCTACTGGAGTTGAGTTTTCAAATATAGTC
CAGAGAGTTTGCCTCCCAGACTCATCTATAAAGTTGCCACCTAAAACAAGTGTGTTCGTCACAGGATTTGGA
TCCATTGTAGATGATGGACCTATACAAAATACACTTCGGCAAGCCAGAGTGGAAACCATAAGCACTGATGTG
TGTAACAGAAAGGATGTGTATGATGGCCTGATAACTCCAGGAATGTTATGTGCTGGATCCATGGAAGGAAAA
ATAGATGCATGTAAGGGAGATTCTGGTGGACCTCTGGTTTATGATAATCATGACATCTGGTACATTGTAGGT
ATAGTAAGTTGGGGACAATCATGTGCACTTCCCAAAAAACCTGGAGTCTACACCAGAGTAACTAAGTATCGA
GATTGGATTGCCTCAAAGACTGGTATGTAGTGTGGATTGTCCATGAGTTATACACATGGCACACAGAGCTGA
TACTCCTGCGTATTTGTA

In a search of public sequence databases, the NOV1a nucleic acid sequence, located on chromsome 4 has 489 of 707 bases (69%) identical to a gb:GENBANK-ID:AF064819|acc:AF064819.1 mRNA from Homo sapiens (Homo sapiens serine protease DESC1 (DESC1) mRNA, complete cds). Public nucleotide databases include all GenBank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., Airway Trypsin-Like Protease mRNA from Homo sapiens, matched the Query NOV1 sequence purely by chance is 1.3e⁻⁴¹. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. (Wootton and Federhen, Methods Enzymol 266:554-571, 1996).

The disclosed NOV1a polypeptide (SEQ ID NO: 2) encoded by SEQ ID NO: 1 has 420 amino acid residues and is presented in Table 1B using the one-letter amino acid code. Signal P, Psort and/or Hydropathy results predict that NOV1a has a signal peptide and is likely to be localized in the plasma membrane with a certainty of 0.6850. In other embodiments, NOV1a may also be localized to the endoplasmic reticulum (membrane) with acertainty of 0.6400, the Golgi body with a certainty of 0.1700 or in the endoplasmic reticulum (lumen) with a certainty of 0.1000. The most likely cleavage site for a NOV1a peptide is between amino acids 38 and 39, at: FLA-YK.

TABLE 1B
Encoded NOV1a protein sequence.

(SEQ ID NO:2)

| | |
|---|---|
| MTLGRRVSSLKPWMFALIVRAVVLILVILIGLLVYFLAYKFYYYQTSFQIPSIEYNLAINTCVTOEERIYDN | +TL,45 |
| KMCKIMSRIFRHSSVGGRFIKSHVIKLRPSNDNLKAnVLLKFQFIPNNENAIKTOADNILHQKIKSNESSLT | |
| INKPSFRLTPIDSKRNLLNSRCGIRMTSSNMPLPASSSTORIVQGRETAJVIEGEWPWQASLQLIGSGHQCG | |
| ASLISNTWLLTAAHCFWKNKDPTQWIATFGATITPPAVKRNVRKIILHENYHRETNENDIALVQLSTGVEFS | |
| NIVQRVCLPDSSIKLPPKTSVFVTGFGSIVDDGPIQNTLRQARVETISTDVCNRKDVYDGLITPGMLCAGFM | |
| EGKIDACKGDSGGPLVYDNHDIWYIVGIVSWGQSCALPKKPGVYTRVTKYRDWIASKTGM+TZ,1/45 | |

A search of sequence databases reveals that the NOV1 a amino acid sequence has 192 of 411 amino acid residues (46%) identical to, and 267 of 411 amino acid residues (64%) similar to, the 418 amino acid residue ptnr:SPTREMBL-ACC:060235 protein from Homo sapiens (Human) (Airway Trypsin-Like Protease) (E=3.1e⁻⁹⁵). Public amino acid databases include the GenBank databases, SwissProt, PDB and PIR.

NOV1b

A disclosed NOV1b nucleic acid of 708 nucleotides (also referred to as 168446573) encoding a novel Airway Trypsin-Like Protease-like protein is shown in Table 1C. An open reading frame was identified beginning with an AGA initiation codon at nucleotides 1-3 and ending at nucleotides 706-708. The start codon is in bold letters in Table 1C. Since the start codon of NOV1b is not a traditional initiation codon, and NOV1b has no termination codon, NOV1b could be a partial open reading frame that could be extended in the 5′ and/or 3′ direction(s).

TABLE 1C
NOV1b nucleotide sequence.

(SEQ ID NO:3)
AGATCTGTCCAAGGAAGGGAAACAGCTATGGAAGGGGAATGGCCATGGCAGGCCAGCCTCCAGCTCATAGGG
TCAGGCCATCAGTGTGGAGCCAGCCTCATCAGTAACACATGGCTGCTCACAGCAGCTCACTGCTTTTGGAAA
AATAAAGACCCAACTCAATGGATTGCTACTTTTGGTGCAACTATAACACCACCCGCAGTGAAACGAAATGTG
AGGAAAATTATTCTTCATGAGAATTACCATAGAGAAACAAATGAAAATGACATTGCTTTGGTTCAGCTCTCT
ACTGGAGTCGGGTTTTCAAATATAGTCCAGAGAGTTTGCCTCCCAGACTCATCTATAAAGTTGCCACCTAAA
ACAAGTGTGTTCGTCACAGGATTTGGATCCATTGTAGATGATGGACCTATACAAAATACACTTCGGCAAGCC
AGAGTGGAAACCATAAGCACTGATGTGTGTAACAGAAAGGATGTGTATGATGGCCTGATAACTCCAGGAATG
TTATGTGCTGGATTCATGGAAGGAAAAATAGATGCATGTAAGGGAGATTCTGGTGGACCTCTGGTTTATGAT
AATCATGACATCTGGTACATTGTAGGTATAGTAAGTTGGGGACAATCATGTGCACTTCCCAAAAAACCTGGA
GTCTACACCAGAGTAACTAAGTATCGAGATTGGATTGCCTCAAAGACTGGTATGCTCGAG

The disclosed NOV1b polypeptide (SEQ ID NO: 4) encoded by SEQ ID NO: 3 has 236 amino acid residues and is presented in Table 1D using the one-letter amino acid code.

TABLE 1D
Encoded NOV1b protein sequence.

(SEQ ID NO:4)

RSVQGRETANEGEWOWQASKQKUGSGHQCGASLISNTWLLTAAHCFWKNKDPTQWIATFGATITPPAVKRNV
RKIILHENYHRETNENDIALVQLSTGVGFSNIVQRVCLPDSSIKLPPKTSVFVTGFGSIVDDGPIQNTLRQA
RVETISTDVCNRKDVYDGLITPGMLCAGFMEGKIDACKGDSGGPLVYDNHDIWYIVGIVSWGQSCALPKKPG
VYTRVTKYRDWIASKTGMLE

NOV1c

A disclosed NOV1c nucleic acid of 708 nucleotides (also referred to as 168446539) encoding a novel Airway Trypsin-Like Protease-like protein is shown in Table 1E. An open reading frame was identified beginning with an AGA initiation codon at nucleotides 1-3 and ending at nucleotides 706-708. The start codon is in bold letters in Table 1E. Since the start codon of NOV1c is not a traditional initiation codon, and NOV1c has no termination codon, NOV1c could be a partial open reading frame that could be extended in the 5′ and/or 3′ direction(s).

TABLE 1E
NOV1c nucleotide sequence.

(SEQ ID NO:5)

AGATCTGTCCAAGGAAGGGAAACAGCTATGGAAGGGGAATGGCCATGGCAGGCCAGCCTCCAGCTCATAGGG
TCAGGCCATCAGTGTGGAGCCAGCCTCATCAGTAACACATGGCTGCTCACAGCAGCTCACTGCTTTTGGAAA
AATAAAGACCCAACTCAATGGATTGCTACTTTTGGTGCAACTATAACACCACCCGCAGTGAAACGAAATGTG
AGGAAAATTATTCTTCATGAGAATTACCATAGAGAAACAAATGAAAATGACATTGCTTTGGTTCAGCTCTCT
ACTGGAGTTGAGTTTTCAAATATAGTCCAGAGAGTTTACCTCCCAGACTCATCTATAAAGTTGCCACCTAAA
ACAAGTGTGTTCGTCACAGGATTTGGATCCATTGTAGATGATGGACCTATACAAAATACACTTCGGCAAGCC
AGAGTGGAAACCATAAGCACTGATGTGTGTAACAGAAAGGATGTGTATGATGGCCTGATAACTCCAGGAATG
TTATGTGCTGGATTCATGGAAGGAAAAATAGATGCATGTAAGGGAGATTCTGGTGGACCTCTGGTTTATGAT
AATCATGACATCTGGTACATTGTAGGTATAGTAAGTTGGGGACAATCATGTGCACTTCCCAAAAAACCTGGA
GTCTACACCAGAGTAACTAAGTATCGAGATTGOATTGCCTCAAAGACTGGTATGCTCGAG

The reverse complement is shown in Table 1F.

TABLE 1F
NOV1c reverse complement nucleotide sequence.

(SEQ ID NO:59)

CTCGAGCATACCAGTCTTTGAGGCAATCCAATCTCGATACTTAGTTACTCTGGTGTAGACTCCAGGTTTTTT
GGGAAGTGCACATGATTGTCCCCAACTTACTATACCTACAATGTACCAGATGTCATGATTATCATAAACCAG
AGGTCCACCAGAATCTCCCTTACATGCATCTATTTTTCCTTCCATGAATCCAGCACATAACATTCCTGGAGT
TATCAGGCCATCATACACATCCTTTCTGTTACACACATCAGTGCTTATGGTTTCCACTCTGGCTTGCCGAAG
TGTATTTTGTATAGGTCCATCATCTACAATGGATCCAAATCCTGTGACGAACACACTTGTTTTAGGTGGCAA
CTTTATAGATGAGTCTGCGAGGTAAACTCTCTGGACTATATTTGAAAACTCAACTCCAGTAGAGAGCTGAAC
CAAAGCAATGTCATTTTCATTTGTTTCTCTATGGTAATTCTCATGAAGAATAATTTTCCTCACATTTCGTTT
CACTGCGGGTGGTGTTATAGTTGCACCAAAAGTAGCAATCCATTGAGTTGGGTCTTTATTTTTCCAAAAGCA
GTGAGCTGCTGTGAGCAGCCATGTGTTACTGATGAGGCTGGCTCCACACTCATGGCCTCACCCTATGAGCTC
GACGCTGGCCTGCCATGGCCATTCCCCTTCCATAGCTGTTTCCCTTCCTTGGACAGATCT

The disclosed NOV1c polypeptide (SEQ ID NO: 6) encoded by SEQ ID NO: 5 has 236 amino acid residues and is presented in Table 1G using the one-letter amino acid code.

TABLE 1G
Encoded NOV1c protein sequence.

| | |
|---|---|
| RSVQGRETAMEGEWPWQASLQLIGSGHQCGASLISNTWLLTAAHCFWKNKDPTQWIATFGATITPPAVKRNV | (SEQ ID NO:6) |
| RKIILHENYHRETNENDIALVQLSTGVEFSNIVQRVYLPDSSIKLPPKTSVFVTGFGSIVDDGPIQNTLRQA | |
| RVETISTDVCNRKDVYDGLITPGMLCAGFMEGKIDACKGDSGGPLVYDNHDIWYIVGIVSWGQSCALPKKPG | |
| VYTRVTKYRDWIASKTGMLE | |

NOV1d

A disclosed NOV1 d nucleic acid of 708 nucleotides (also referred to as 168446547) encoding a novel Airway Trypsin-Like Protease-like protein is shown in Table 1H. An open reading frame was identified beginning with an AGA initiation codon at nucleotides 1-3 and ending at nucleotides 706-708. The start codon is in bold letters in Table 1H. Since the start codon of NOV1d is not a traditional initiation codon, and NOV1d has no termination codon, NOV1d could be a partial open reading frame that could be extended in the 5′ and/or 3′ direction(s).

TABLE 1H
NOV1d nucleotide sequence.

| | |
|---|---|
| AGATCTGTCCAAGGAAGGGAAACAGCTATGGAAGGGGAATGGCCATGGCAGGCCAGCCTCCAGCTCATAGGG | (SEQ ID NO:7) |
| TCACGCCATCAGTGTGGAGCCAGCCTCATCAGTAACACATGGCTGCTCACAGCAGCTCACTGCTTTTGGAAA | |
| AATAAAGACCCAACTCAATGGATTGCTACTTTTGGTGCAACTATAACACCACCCGCAGTGAAACGAAATGTG | |
| AGGAAAATTATTCTTCATGAGAATTACCATAGAGAAACAAATGAAAATGACATTGCTTTGGTTCAGCTCTCT | |
| ACTGGAGTTGAGTTTTCAAATATAGTCCAGAGAGTTTGCCTCCCAGACTCATCTATAAAGTTGCCACCTAAA | |
| ACAAGTGTGCTCGTCACAGGATTTGGATCCATTGTAGATGATGGACCTATACAAAATACACTTCGGCAAGCC | |
| AGAGTGGAAACCATAAGCACTGATGTGTGTAACAGAAAGGATGTGTATGATGGCCTGATAACTCCAGGAATG | |
| TTATGTGCTGGATTCATGGAAGGAAAAATAGATGCATGTAAGGGAGATTCTGGTGGACCTCTGGTTTATGAT | |
| AATCATGACATCTCGTACATTGTAGGTATAGTAAGTTGCGGACAATCATGTGCACTTCCCAAAAAACCTGGA | |
| GTCTACACCAGAGTAACTAAGTATCGAGATTGGATTGCCTCAAAGACTGGTATGCTCGAG | |

The disclosed NOV1d polypeptide (SEQ ID NO: 8) encoded by SEQ ID NO: 7 has 236 amino acid residues and is presented in Table 1I using the one-letter amino acid code.

TABLE 1I
Encoded NOV1d protein seqnence.

| | |
|---|---|
| RSVQGRETANEGEWPWQASLQLIGSCHQCGASLISNTWLLTAAHCFWKNKDPTQWIATFGATITPPAVKRNV | (SEQ ID NO:8) |
| RKIILHENYNRETNENDIALVQLSTGVEFSNIVQRVCLPDSSIKLPPKTSVLVTGFGSIVDDGPIQNTLRQA | |
| RVETISTDVONRKDVYDGLITPGMLCAGFMEGKIDACKGDSGGPLVYDNHDIWYIVOIVSWGQSCALPKKPG | |
| VYTRVTKYRDWIASKTGMLE | |

Homologies to either of the above NOV1 proteins will be shared by the other NOV1 protein insofar as they are homologous to each other as shown below. Any reference to NOV1 is assumed to refer to all three of the NOV1 proteins in general, unless otherwise noted.

The disclosed NOV1a polypeptide has homology to the amino acid sequences shown in the BLASTP data listed in Table 1J.

TABLE 1J
BLAST results for NOV1a

| Gene Index/ | | Length | Identity | Positives | |
|---|---|---|---|---|---|
| Identifier | Protein/ Organism | (aa) | (%) | (%) | Expect |
| gi\|17446381\|ref\|XP_{—} | similar to DESC1 | 246 | 200/247 | 214/247 | e−109 |
| 068225.1\| | protein (H. sapiens) | | (80%) | (85%) | |
| (XM_068225) | [Homo sapiens] | | | | |
| gi\|4758508\|ref\|NP_{—} | airway trypsin- | 418 | 180/390 | 251/390 | 4e−94 |
| 004253.1\| | like protease | | (46%) | (64%) | |
| (NM_004262) | [Homo sapiens] | | | | |
| gi\|17437609\|ref\|XP_{—} | similar to DESC1 protein | 345 | 160/346 | 214/346 | 1e−82 |
| 003340.5\| | (H. sapiens) | | (46%) | (61%) | |
| (XM_003340) | [Homo sapiens] | | | | |
| gi\|7661558\|ref\|NP_{—} | DESC1 protein | 422 | 160/346 | 214/346 | 1e−82 |
| 054777.1\| | [Homo sapiens | | (46%) | (61%) | |
| (NM_014058) | | | | | |
| gi\|17446387\|ref\|XP_{—} | similar to airway | 406 | 139/269 | 179/269 | 6e−75 |
| 068227.1\| | trypsin-like | | (51%) | (65%) | |
| (XM_068227) | protease (H. sapiens) | | | | |

The homology between these and other sequences is shown graphically in the ClustalW analysis shown in Table 1K. In the ClustalW alignment of the NOV1 proteins, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/ interpro). DOMAIN results for NOV1 as disclosed in Tables 1L-1M, were collected from the Conserved Domain Database (CDD) with Reverse Position Specific BLAST analyses. This BLAST analysis software samples domains found in the Smart and Pfam collections. For Table 1K and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Tables 1L-M list the domain descriptions from DOMAIN analysis results against NOV1a. This indicates that the NOV1a sequence has properties similar to those of other proteins known to contain this domain.

TABLE 1L
Domain Analysis of NOV1a

gnl|Smart|smart00020, Tryp_SPc, Trypsin-like serine protease; Many of
these are synthesised as inactive precursor zymogens that are cleaved
during limited proteolysis to generate their active forms .A few,
however, are active as single chain molecules, and others are inactive
due to substitutions of the catalytic triad residues. (SEQ ID NO:66)
CD-Length = 230 residues, 100.0% aligned
Score = 262 bits (669), Expect = 3e − 71

| | | | |
|---|---|---|---|
| Query: | 187 | RIVQGRETAMEGEWPWQASLQLIGSGHQCGASLISNTWLLTAAHCFWKNKDPTQWIATFG | 246 |
| | | \|\|\| \| \| + \| +\|\|\| \|\|\| \| \| \|\| \|\|\|\| \|+\|\|\|\|\|\| + \|+ \| | |
| Sbjct: | 1 | RIVGGSEANI-GSFPWQVSLQYRGGRHFCGGSLISPRWVLTAAHCVY-GSAPSSIRVRLG | 58 |
| Query: | 247 | AT---ITPPAVVRKIILHYRETNENDIALVQLSTGVEPSNIVQRVCLPDSSIKJL | 303 |
| | | + \| \|+\|+\| \|\|+ \| +\|\|\|\|\|++\|\| \| ‘\|+ \|+ +\|\|\| \| + | |
| Sbjct: | 59 | SHDLSSGEETQTVKVSKVIVHPNYNPSTYDNDIALLKLSEPVTLSDTVRPICLPSSGYNV | 118 |
| Query: | 304 | PPKTSVFVTGFGSI-VDDGPIQNTLRQARVETISTDVCNRKDVYDGLITPGMLCAGFMEG | 362 |
| | | \| \|+ \|+\|+\| \| + +\|\|++ \| +\| \| \| \|\| \|\|\|\|\| +\|\| | |
| Sbjct: | 119 | PAGTTCTVSGWGRTSESSGSLPDTLQEVNVPIVSNATCRRAYSGGPAITDNMLCAGGLET | 178 |
| Query: | 363 | KIDACKGDSGGPLVYDNHDIWYIVGIVSWG-QSCALPKKPGVYTRVTKYRDWI | 414 |
| | | \|\|\|+\|\|\|\|\|\|\|\| ++ \| +\|\|\|\|\|\|\| \|\| \| \|\|\|\|\|\|\|\|+ \| \|\|\| | |
| Sbjct: | 179 | GKDACQGDSGGPLVCNDP-RWVLVGIVSWGSYGCARPNKPGVYTRVSSYLDWI | 230 |

TABLE 1M
Domain Analysis of NOV1a

gn1|Pfam|pfam00089, trypsin, Trypsin. Proteins recognized include all
proteins in families S1, S2A, S2B, S2C, and S5 in the classification
of peptidases. Also included are proteins that are clearly members,
but that lack peptidase activity, such as haptoglobin and protein z
(PRTZ*). (SEQ ID NO:67)
CD-Length = 217 residues, 100.0% aligned
Score = 204 bits (518), Expect = 1e − 53

| | | | |
|---|---|---|---|
| Query: | 188 | IVQGRETAMEGEWPWQASLQLIGSGHQCGASLISNTWLLTAAHCFWKNKDPTQWIATFGA | 247 |
| | | \|\| \|\|\| \| +\|\|\| \|\|\| + \|\|\| \|\| \|\|\|\| \|+\|\|\|\|\|\| + | |
| Sbjct: | 1 | IVGGREAQA-GSFPWQVSLQ-VSSGHFCGGSLISENWVLTAAHCVSGASSVRVVLGEHNL | 58 |
| Query: | 248 | TITPPAV-KRNVRKIILHENYHRETNENDIALVQLSTGVEFSNIVQRVCLPDSSIKLPPK 306 | |
| | | \| \| +\|+\|\|\|+\| \|\|+ +\|\| \|\|\|\|++\| + \| + \|+ +\|\|\| +\| \|\| | |
| Sbjct: | 59 | GTTEGTEQKFDVKKIIVHPNYNPDTN--DIALLKLKSPVTLGDTVRPICLPSASSDLPVG | 116 |
| Query: | 307 | TSVFVTGFGSIVDDGPIQNTLRQARVETISTDVCNRKDVYDGLITPGMLCAGFMEGKIDA | 366 |
| | | \|+ \|+\|+\| + \| \|\|++ \| +\| + \| \| \| +\| \|+\|\|\| + \|\| \|\| | |
| Sbjct: | 117 | TTCSVSGWGRTKNLGTSD-TLQEVVVPIVSRETCRS--AYGGTVTDTMICAGALGGK-DA | 172 |
| Query: | 367 | CKGDSGGPLVYDNHDIWYIVGIVSWGQSCALPKKPGVYTRVTKYRDWI | 414 |
| | | \|+\|\|\|\|\|\|\|\|\| + +\|\|\|\|\|\|\| \|\|+ \|\|\|\|\|\|\|++\| \|\|\| | |
| Sbjct: | 173 | CQGDSGGPLVCSDG---ELVGIVSWGYGCAVGNYPOVYTRVSRYLDWI | 217 |

Human airway trypsin-like protease (HAT) from human sputum is related to the prevention of fibrin deposition in the airway lumen by cleaving fibrinogen. In mucoid sputum samples from patients with chronic airway diseases, the concentration of fibrinogen, as measured by ELISA, was in the range of 2-20 micrograms/ml, and trypsin-like activity, as measured by spectrofluorometry was in the range of 10-50 milliunits (mU)/ml. The trypsin-like activity of mucoid sputum was mainly due to HAT. As shown by SDS-polyacrylamide gel electrophoresis, HAT cleaved fibrinogen, especially its alpha-chain, regardless of the concentration of fibrinogen. Pretreatment of fibrinogen with HAT resulted in a decrease or complete loss of its thrombin-induced clotting capacity, depending on the duration of pretreatment with HAT and the concentration of HAT. HAT may participate in the anticoagulation process within the airway, especially at the level of the mucous membrane, by cleaving fibrinogen transported from the blood stream. PMID: 9864967, UI: 99082486

A novel trypsin-like protease has been purified to homogeneity from the sputum of patients with chronic airway diseases, by sequential chromatographic procedures. The enzyme migrated on SDS-polyacrylamide gel electrophoresis to a position corresponding to a molecular weight of 28 kDa under both reducing and non-reducing conditions, and showed an apparent molecular weight of 27 kDa by gel filtration, indicating that it exists as a monomer. It had an NH2-terminal sequence of Ile-Leu-Gly-Gly-Thr-Glu-Ala-Glu-Glu-Gly-Ser-Trp-Pro-Trp-Gln-Val-Ser-Leu-Arg-Leu, which differed from that of any known protease. Studies with model peptide substrates showed that the enzyme preferentially cleaves the COOH-terminal side of arginine residues at the P1 position of certain peptides, cleaving Boc-Phe-Ser-Arg4-methylcoumaryl-7-amide most efficiently and having an optimum pH of 8.6 with this substrate. The enzyme was strongly inhibited by diisopropyl fluorophosphate, leupeptin, antipain, aprotinin, and soybean trypsin inhibitor, but hardly inhibited by secretory leukocyte protease inhibitor at 10 microM. An immunohistochemical study indicated that the enzyme is located in the cells of the submucosal serous glands of the bronchi and trachea. These results suggest that the enzyme is secreted from submucosal serous glands onto the mucous membrane in patients with chronic airway diseases. PMID: 9070615, UI: 97224034

A novel trypsin-like protease associated with rat bronchiolar epithelial Clara cells, named Tryptase Clara, has been purified to homogeneity from rat lung by a series of standard chromatographic procedures. The enzyme has apparent molecular masses of 180+/−16 kDa on gel filtration and 30+/−1.5 kDa on sodium dodecyl sulfate-polyacrylamide gel electrophoresis under reducing conditions. Its isoelectric point is pH 4.75. Studies with model peptide substrates showed that the enzyme preferentially recognizes a single arginine cleavage site, cleaving Boc-Gln-Ala-Arg4-methylcoumaryl-7-amide most efficiently and having a pH optimum of 7.5 with this substrate. The enzyme is strongly inhibited by aprotinin, diisopropylfluorophosphate, antipain, leupeptin, and Kunitz-type soybean trypsin inhibitor, but inhibited only slightly by Bowman-Birk soybean trypsin inhibitor, benzamidine, and alpha 1-antitrypsin. Immunohistochemical studies indicated that the enzyme is located exclusively in the bronchiolar epithelial Clara cells and colocalized with surfactant. An immunoreactive protein with a molecular mass of 28.5 kDa was also detected in airway secretions by Western blotting analyses, suggesting that the 30-kDa protease in Clara cells is processed before or after its secretion. Proteolytic cleavage of the hemagglutinin of influenza virus is a prerequisite for the virus to become infectious. Tryptase Clara was shown to cleave the hemagglutinin and activate infectivity of influenza A virus in a dose-dependent way. These results suggest that the enzyme is a possible activator of inactive viral fusion glycoprotein in the respiratory tract and thus responsible for pneumopathogenicity of the virus. PMID: 1618859, UI: 92317085

The disclosed NOV1 nucleic acid of the invention encoding a Airway Trypsin-Like Protease-like protein includes the nucleic acid whose sequence is provided in Table 1A, 1C, 1E, 1G or a fragment thereof. The invention also includes a mutant or variant nucleic acid any of whose bases may be changed from the corresponding base shown in Table 1A, 1C, 1E, or 1G while still encoding a protein that maintains its Airway Trypsin-Like Protease-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 31% percent of the bases may be so changed.

The disclosed NOV1 protein of the invention includes the Airway Trypsin-Like Protease-like protein whose sequence is provided in Table 1B, 1D, 1F, or 1H. The invention also includes a mutant or variant protein any of whose residues may be changed from the corresponding residue shown in Table 1B, 1D, 1F, or 1H while still encoding a protein that maintains its Airway Trypsin-Like Protease-like activities and physiological functions, or a functional fragment thereof. In the mutant or variant protein, up to about 54% percent of the residues may be so changed.

The invention further encompasses antibodies and antibody fragments, such as F_{ab} or (F_{ab})₂, that bind immunospecifically to any of the proteins of the invention.

The above defined information for this invention suggests that this Airway Trypsin-Like Protease-like protein (NOV1) may function as a member of a “Airway Trypsin-Like Protease family”. Therefore, the NOV1 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV1 nucleic acids and proteins of the invention are useful in potential therapeutic applications implicated in cancer including but not limited to various pathologies and disorders as indicated below. For example, a cDNA encoding the Airway Trypsin-Like Protease-like protein (NOV1) may be useful in gene therapy, and the Airway Trypsin-Like Protease-like protein (NOV1) may be useful when administered to a subject in need thereof.

By way of nonlimiting example, the compositions of the present invention will have efficacy for treatment of patients suffering from chronic airway diseases such as asthma and cystic fibrosis, allergies, emphysema, bronchitis, lung cancer, or other pathologies or conditions. The NOV1 nucleic acid encoding the Airway Trypsin-Like Protease-like protein of the invention, or fragments thereof, may further be useful in diagnostic applications, wherein the presence or amount of the nucleic acid or the protein are to be assessed.

NOV1 nucleic acids and polypeptides are further useful in the generation of antibodies that bind immuno-specifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1 proteins have multiple hydrophilic regions, each of which can be used as an immunogen. In one embodiment, a contemplated NOV1 epitope is from about amino acids 40 to 225. In another embodiment, a NOV1 epitope is from about amino acids 240 to 270. In other embodiments, a NOV1 epitope is from about amino acids 320 to 340, from about amino acids 360 to 370, and from about amino acids 390 to 410. These novel proteins can be used in assay systems for functional analysis of various human disorders, which will help in understanding of pathology of the disease and development of new drug targets for various disorders.

NOV2

A disclosed NOV2 nucleic acid of 1476 nucleotides (also referred to as CG55782-01) encoding a novel P450-like protein is shown in Table 2A. An open reading frame was identified beginning with an ATG initiation codon at nucleotides 1-3 and ending with a TAA codon at nucleotides 1474-1476. A The start and stop codons are in bold letters in Table 2A.

TABLE 2A
NOV2 nucleotide sequence (SEQ ID NO:9).

ATGGACAGCATTAAGCACAGCCATCTTACTCCTGCTCCTGGCTCTCGTCTGTCTGTCCTGACCCTAAGCTCA
AGAGATAAGGGAAAGCTGCCTCCGGGACCCAGACCCCTCTCAATCCTGGGAAACCTGCTGCTGCTTTGCTCC
CAAGACATGCTGACTTCTCTCACTAAGCTGAGCAAGGAGTATGGCTCCATGTACACAGTGCACCTGGGACCC
AGGCGGGTGGTGGTCCTCAGCGGGTACCAAGCTGTGAAGGAGGCCCTGGTGGACCAGGGAGAGGAGTTTAGT
GGCCGCGGTGACTACCCTGCCTTTTTCAACTTTACCAAGGGCAATGGCATCGCCTTCTCCAGTGGGGATCGA
TGGAAGGTCCTGAGACAGTTCTCTATCCAGATTCTACGGAATTTCGGGATGGGGAAGAGAAGCATTGAGGAG
CGAATCCTAGAGGAGGGCAGCTTCCTGCTGGCGGAGCTGCGGAAAACTGAAGGCGAGCCCTTTGACCCCACG
TTTGTGCTGAGTCGCTCAGTGTCCAACATTATCTGTTCCGTGCTCTCGGCAGCCGCTTTCGACTATGATGAT
GAGCGTCTGCTCACCATTATCCGCCTTATCAATGACAACTTCCAAATCATGAGCAGCCCCTGGGGCGAGTTG
TACGACATCTTCCCGAGCCTCCTGGACTGGGTGCCTGGGCCGCACCAACGCATCTTCCAGAACTTCAAGTGC
CTGAGAGACCTCATCGCCCACAGCGTCCACGACCACCAGGCCTCGCTAGACCCCAGATCTCCCCGGGACTTC
ATCCAGTGCTTCCTCACCAAGATGGCAGAGGAGAAGGAGGACCCACTGAGCCACTTCCACATGGATACCCTG
CTGATGACCACACATAACCTGCTCTTTGGCGGCACCAAGACGGTGAGCACCACGCTGCACCACGCCTTCCTG
GCACTCATGAAGTACCCAAAAGTTCAAGCCCGCGTGCAGGAGGAGATCGACCTCGTGGTGGGACGCGCGCGG
CTGCCGGCGCTGAAGGAACCGCGCGGCCATGCCTTACACAGACGCGGTGATCCACGAGGTGCACGCTTTGCA
GACATCATCCCCATGAACTTGCCGCACCGCGTCACTAGGGACACGGCCTTTCGCGGCTTCCTGATACCCAGG
GGCACCGATGTCATCACCCTCCTTAACACCGTCCACTACGACCCCAGCCAGTTCCTGACGCCCCAGGAGTTC
AACCCCGAGCATTTTTTGGATGCCAATCAGTCCTTCAAGAAGAGTCCAGCCTTCATGCCCTTCTCAGCTGGG
CGCCGTCTGTGCCTGGGAGAGTCGCTGGCGCGCATGGAGCTCTTTCTGTACCTCACCGCCATCCTGCAGAGC
TTTTCGCTGCAGCCGCTGGGTGCGCCCGAGGACATCGACCTGACCCCACTCAGCTCAGGTCTTGGCAATTTG
CCGCGGCCTTTCCAGCTGTGCCTGCGCCCGCGCTAA

The disclosed NOV2 nucleic acid sequence, localized to chromsome 19, has 1419 of 1476 bases (96%) identical to a gb:GENBANK-ID:HUMCYPIIF|acc:J02906.1 mRNA from Homo sapiens (Human cytochrome P450IIF1 protein (CYP2F) mRNA, complete cds) (E=7.5e⁻³⁰¹).

A NOV2 polypeptide (SEQ ID NO: 10) encoded by SEQ ID NO: 9 has 492 amino acid residues and is presented using the one-letter code in Table 2B. Signal P, Psort and/or Hydropathy results predict that NOV2 contains a signal peptide and is likely to be localized to the endoplasmic reticulum (membrane) with a certainty of 0.8200. In other embodiments, NOV2 may also be localized to the microbody (peroxisome) with a certainty of 0.2824, the plasma membrane with a certainty of 0.1900, or the endoplasmic reticulum (lumen) with a certainty of 0.1000. The most likely cleavage site for NOV2 is between positions 24 and 25: LSS-RD.

TABLE 2B
Encoded NOV2 protein sequence (SEQ ID NO:10).

MDSISTAILLLLLALVCLLLTLSSRDKGKLPPGPRPLSILGNLLLLCSQDMLTSLTKLSKEYGSMYTVHLGP
RRVVVLSGYQAVKEALVDQGEEFSGTGDYPAFFNFTKGNGIAFSSGDRWKVLRQFSIQILRNFGMGKRSIEE
RILEEGSFLLAELRKTEGEPFDPTFVLSRSVSNIICSVLFGSRFDYDDERLLTIIRLINDNFQIMSSPWGEL
YDIFPSLLDWVPGPHQRIFQNFKCLRDLIARSVHDHQASLDPRSPRDFIQCFLTKMAEEKEDPLSHFHMDTL
LMTTHNLLFGGTKTVSTTLRHAFLAMKYPKVQARVQEEIDLVVGRARLPALKDRAAMPYTDAVIHEVQRFAI
DIIPMNLPHRVTRDTAFRGFLIPKGTDVITLLNTVHYDPSQFLTPQEFNPEHFLDANQSFKKSPAFMPFSAG
RRLCLGESLARMELFLYLTAILQSFSLQPLGAPEDIDLTPLSSGLGNLPRPFQLCLRPRX

The disclosed NOV2 amino acid sequence has 484 of 491 amino acid residues (98%) identical to, and 486 of 491 amino acid residues (98%) similar to, the 491 amino acid residue ptnr:SWISSPROT-ACC:P24903 protein from Homo sapiens (Human) (Cytochrome P450 2F1 (EC 1.14.14.1) (CYPIIF1)) (E=1.1e⁻²⁵⁷).

NOV2 is expressed in at least lung. This information was derived by determining the tissue sources of the sequences that were included in the invention including but not limited to SeqCalling sources, Public EST sources, Literature sources, and/or RACE sources.

NOV2 also has homology to the amino acid sequences shown in the BLASTP data listed in Table 2C.

TABLE 2C
BLAST results for NOV2

| Gene Index/ | Protein/ | Length | Identity | Positives | |
|---|---|---|---|---|---|
| Identifier | Organism | (aa) | (%) | (%) | Expect |
| gi\|14786875\|ref\|XP_{—} | cytochrome | 495 | 460/495 | 460/495 | 0.0 |
| 012782.4\| | P450, | | (92%) | (92%) | |
| (XM_012782) | subfamily | | | | |
| | IIF, | | | | |
| | polypeptide 1 | | | | |
| | [Homo sapiens | | | | |
| gi\|4503225\|ref\|NP_{—} | cytochrome | 491 | 460/495 | 462/495 | 0.0 |
| 000765.1\| | P450, | | (92%) | (92%) | |
| (NM_000774) | subfamily | | | | |
| | IIF, | | | | |
| | polypeptide 1; | | | | |
| | microsomal | | | | |
| | monooxygenase; | | | | |
| | xenobiotic | | | | |
| | monooxygenase; | | | | |
| | flavoprotein- | | | | |
| | linked | | | | |
| | monooxygenase | | | | |
| | [Homo sapiens] | | | | |
| gi\|5915805\|sp\|O18809 | CYTOCHROME | 491 | 397/491 | 438/491 | 0.0 |
| \|C2F3_CAPHI | P450 2F3 | | (80%) | (88%) | |
| | (CYPIIF3 | | | | |
| gi\|9506531\|ref\|NP_{—} | Cytochrome | 491 | 391/491 | 431/491 | 0.0 |
| 062176.1\| | P450, | | (79%) | (87%) | |
| (NM_019303) | subfamily | | | | |
| | IIF, | | | | |
| | polypeptide 1 | | | | |
| | [Rattus norvegicus] | | | | |
| gi\|461829\|sp\|P33267\| | CYTOCHROME | 491 | 385/491 | 427/491 | 0.0 |
| C2F2_MOUSE | P450 2F2 | | (78%) | (86%) | |
| | (CYPIIF2) | | | | |
| | (NAPHTHALENE | | | | |
| | DEHYDROGENASE) | | | | |
| | (NAPHTHALENE | | | | |
| | HYDROXYLASE) | | | | |
| | (P450-NAH-2) | | | | |

The homology of these sequences is shown graphically in the ClustalW analysis shown in Table 2D.

[image]

[image]

[image]

Table 2E lists the domain description from DOMAIN-analysis results against NOV2. This indicates that the NOV2 sequence has properties similar to those of other proteins known to contain this domain.

TABLE 2E
Domain Analysis of NOV2

gn1|Pfam|pfam00067, p450, Cytochrome P450. Cytochrome P450s are
involved in the oxidative degradation of various compounds.
Particularly well known for their role in the degradation of
environmental toxins and mutagens. Structure is mostly alpha, and
hinds a heme cofactor. (SEQ ID NO:73)
CD-Length = 445 residues, 100.0% aligned
Score = 453 bits (1165) , Expect =1e − 128

| | | | |
|---|---|---|---|
| Query: | 31 | PPGPRPLSILGNLLLLCSQDMLTSLTKLSKEYGSMYTVHLGPRRVVVLSGYQAVKEALVD | 90 |
| | | \|\|\|\| \|\| ++\|\|\|\| \| + \|\|\|+\| \|+\|\| ++\|++\|\|\|\| \|\|\|++\| +\|\|\|\| \|+\| | |
| Sbjct: | 1 | PPGPPPLPLIGNLLQLGRCPIH-SLTELRKKYGPVFTLYLGPRPVVVVTGPEAVKEVLID | 59 |
| Query: | 91 | QGEEFSGRGDYPAFFNFThGNGIAFSSGDRWKVLRQFSIQILRNFGMGKRS-IEERILEE | 149 |
| | | +\|\|\|\|+\|\|\|\|+\| \| \| \|\| \|\|+\| \|\|+ \|\|+ + \|\| \|\|\|\|\|\|\| +\|\|\|\| \|\| | |
| Sbjct: | 60 | KGEEFAGRGDFPVFPWL--GYGILFSNGPRWRQLRR--LLTLRFFGMGKRSKLEERIQEE | 115 |
| Query: | 150 | GSFLLAELRKTEGEPFDPTFVLSRSVSNIICSVLFGSRFDYDDERLLTIIRLINDNFQIM | 209 |
| | | \|+ \|\|\| +\| \| \| \| +\|+ + \|+\|\|\|+\|\|\| \|\|\|\|+\| \| +\| +\|+ \| ++ | |
| Sbjct: | 116 | ARDLVERLRKEQGSPIDITELLAPAPLNVICSLLFGVRFDYEDPEFLKLIDKLNELFFLV | 175 |
| Query: | 210 | SSPWGELYDIFPSLLDWVPGPHQRIFQNFKCLRDLIAHSVHDHQASLDPRSPRDFIQCFL | 269 |
| | | \| \|\|\|+\| \| \| ++\|\| \|++ \|+ \| \|+\| + + + + +\|+\| \|\|\|\|+ \| | |
| Sbjct: | 176 | S-PWGQLLDFFR----YLPGSHRKAFKAAKDLKDYLDKLIEERRETLEPGDPRDFLDSLL | 230 |
| Query: | 270 | TKMAEEKEDPLSHFHMDTLLMTTENLLFCGTKTVSTTLHHAFLALMKYPKVQARVQEEID | 329 |
| | | + \| \| + \| \| +\|\|\| \|\| \| \|+\|\| \| \| \|+\|+\|\|\|+++\|\|\|\| | |
| Sbjct: | 231 | IEAKREGG---SELTDEELKATVLDLLFAGTDTTSSTLSWALYLLAKHPEVQAKLREEID | 287 |
| Query: | 330 | LVVGRARLPALKDRAAMPYTDAVIHEVQRFADIIPMNLPHRVTRDTAFRGFLIPKGTDVI | 389 |
| | | \|+51 \| \| \| \|\|\| \|\|\| \|\|\|\| \| \| ++\|+ \|\| \| \|\| \|+\|\|\|\|\|\| \|\| | |
| Sbjct: | 288 | EVIGRDRSPTYDDRANNPYLDAVIKETLRLHPVVPLLLPRVATEDTEIDGYLIPKGTLVI | 347 |
| Query: | 390 | TLLNTVHYDPSQFLTPQEFNPEHFLDANQSFKKSPAFMPFSAGRRLCLGESLARMELFLY | 449 |
| | | \| ++\| \|\| \| \|+\|\|+\|\| \|\|\| \| \|\|\|\| \|\|+\|\| \|\| \| \|\|\|\| \|\|\|\|\|\|\|\|+ | |
| Sbjct: | 348 | VNLYSLHRDPKVFPNPEEFDPERFLDENGKFKKSYAFLPFGAGPRNCLGERLARMELFLF | 407 |
| Query: | 450 | LTAILQSFSLQPLGAPEDIDLTPLSSGLGNLPRPFQLCL | 488 |
| | | \| +\|\| \| \|+ + \| \|\| \|\|\| \|\| + \| +\|\| | |
| Sbjct: | 408 | LATLLQRFELELVP-PGDIPLTPKPLGLPSKPPLYQLRA | 445 |

The P450 gene superfamily is a biologically diverse class of oxidase enzymes; members of the class are found in all organisms. P450 proteins are clinically and toxicologically important in humans; they are the principal enzymes in the metabolism of drugs and xenobiotic compounds, as well as in the synthesis of cholesterol, steroids and other lipids. Induction of some P450 genes can also be a risk factor for several types of cancer. This diversity of function is mirrored in the diversity of nucleotide and protein sequences; there are currently over 100 human P450 forms described. Allelic forms of many cytochrome P450 genes have been identified as causing quantitatively different rates of drug metabolism, and hence are important to consider in the development of safe and effective human pharmaceutical therapies. [reviewed in E. Tanaka, J Clinical Pharmacy & Therapeutics 24:323-329, 1999].

The disclosed NOV2 nucleic acid of the invention encoding a P450-like protein includes the nucleic acid whose sequence is provided in Table 2A or a fragment thereof. The invention also includes a mutant or variant nucleic acid any of whose bases may be changed from the corresponding base shown in Table 2A while still encoding a protein that maintains its P450-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 4% percent of the bases may be so changed.

The disclosed NOV2 protein of the invention includes the P450-like protein whose sequence is provided in Table 2B. The invention also includes a mutant or variant protein any of whose residues may be changed from the corresponding residue shown in Table 2B while still encoding a protein that maintains its P450-like activities and physiological functions, or a functional fragment thereof. In the mutant or variant protein, up to about 22% percent of the residues may be so changed.

The NOV2 nucleic acids and proteins of the invention are useful in potential therapeutic applications implicated in various pathologies and disorders.

NOV2 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 protein has multiple hydrophilic regions, each of which can be used as an immunogen. In one embodiment, a contemplated NOV2 epitope is from about amino acids 75 to 160. In another embodiment, a NOV2 epitope is from about amino acids 170 to 270. In additional embodiments, and from about amino acids 400 to 430. These novel proteins can be used in assay systems for functional analysis of various human disorders, which are useful in understanding of pathology of the disease and development of new drug targets for various disorders.

NOV3

NOV3 includes three novel Apolipoprotein A-I precursor-like proteins disclosed below. The disclosed sequences have been named NOV3a and NOV3b.

NOV3a

A disclosed NOV3a nucleic acid of 818 nucleotides (also referred to as CG557,71-01) encoding a novel Apolipoprotein A-I precursor-like protein is shown in Table 3A. An open reading frame was identified beginning with a ATG initiation codon at nucleotides 36-38 and ending with a TAA codon at nucleotides 756-758. The start and stop codons are in bold letters, and the 5′ and 3′ untranslated regions are underlined.

TABLE 3A
NOV3a Nucleotide Sequence (SEQ ID NO:11)

TGGCTGAAGGCGGAGGTCCCCACGGCCCTTCAGGATGAAAGCTGCGGTGCTGACCTTGGCCGTGCTCATTC
CTGACGGGGAGCCAGGCTCGGCATTTCTGGCAGCAAGATGAACCCCCCAGAGCCCCTGGGATCGAGTAGAA
GGACCTGGCCACTGTGTACGTGGATGTGCTCAAAGACAGCGTGACCTCCACCTTCAGCAAGCTGCGCGAAC
AGCTCGGCCCTGTGACCCAGGAGTTCTGGGATAACCTGGAAAAGGAGACAGAGGGCCTGAGGCAGGAGATG
AGCAAGGATCTGGAGGAGGTGAAGGCCAAGGTGCAGCCCTACCTGGACGACTTCCAGAAGAAGTGGCAGGA
GGAGATGGAGCTCTACCGCCAGAAGGTGGAGCCGCTGCGCGCAGAGCTCCAAGAGGGCGCGCGCCAGAAGC
TGCACGAGCTGCAAGAGAAGCTGAGCCCACTGGGCGAGGAGATGCGCGACCGCGCGCGCGCCCATGTGGAC
GCGCTGCGCACGCATCTGGCCCCTGACAGCGACGAGCTGCGCCAGCGCTTGGCCGCGCGCCTTGAGGCTCT
CAAGGAGAACGGCGGCGCCAGACTGGCCGAGTATCACGCCAAGGCCACCGAGCATCTGAGCACGCTCAGCG
AGAAGGCCAAGCCCGCGCTCGAGGACCTCCGCCAAGGCCTGCTGCCCGTGCTGGAGAGCTTCAAGGTCAGC
TTCCTCAGCGCTCTCGAGGAGTACACTAAGAAGCTCAACACCCAGTGAGGCGCCCGCGCCGCCCCCCTTCC
CGGTGCTCAGAATAAACGTTTCCAAAGTGGGAAAAAA

The disclosed NOV3a nucleic acid sequence maps to chromosome 11 and has 640 of 643 bases (99%) identical to a gb:GENBANK-ID:HSAPOAIB|acc:X02162.1 mRNA from Homo sapiens (Human mRNA for apolipoprotein AI (apo AI)) (E=9.5e⁻¹³⁸).

A disclosed NOV3a protein (SEQ ID NO: 12) encoded by SEQ ID NO: 11 has 240 amino acid residues, and is presented using the one-letter code in Table 3B. Signal P, Psort and/or Hydropathy results predict that NOV3a does have a signal peptide, and is likely to be localized to extracellularly with a certainty of 0.3700. In other embodiments NOV3a is also likely to be localized endoplasmic reticulum (membrane) with a certainty of 0.1000, to the endoplasmic reticulum (lumen) with a certainty of 0.1000, or to the microbody (peroxisome) with a certainty of 0.1000. The most likely cleavage site for NOV3a is between positions 18 and 19, (SQA-RH).

TABLE 3B
Encoded NOV3a protein sequence (SEQ ID NO:12).

MKAAVLTAVLFLTGSQARHFWQQDEPPQSPWDRVKDLATVYVDVLKDSVTSTFSKLREQLGPVTQEFWADN
LEKETEGLRQEMSKDLEEVKAKVQPYLDDFQKKWQEEMELYRQKVEPLRAELQEGARQKLHELQEKLASPL
EEMRDRARAHVDALRTHLAPYSDELRQRLAARLEALKENGGARLAEYHAKATEHLSTLSEKAKPALEDLRQ
GLLPVLESPKVSFLSALEEYTKKLNTQ

The disclosed NOV3a amino acid has 193 of 193 amino acid residues (100%) identical to, and 193 of 193 amino acid residues (100%) similar to, the 267 amino acid residue ptnr:SWISSPROT-ACC:P02647 protein from Homo sapiens (Human) (Apolipoprotein A-I Precursor (APO-Al)) (E=7.1e⁻⁹⁸).

NOV3 is expressed in at least Colon, Gall Bladder, Heart, Liver, Lung, Lymph node, Lymphoid tissue, Ovary, Placenta, Spleen, Testis, Thymus, and Whole Organism. This information was derived by determining the tissue sources of the sequences that were included in the invention including but not limited to SeqCalling sources, Public EST sources, Literature sources, and/or RACE sources.

NOV3b

In NOV3b, the target sequence identified previously, NOV3a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV3b. This differs from the previously identified sequence NOV3a in having 2 internal splice regions.

A disclosed NOV3b nucleic acid of 677 nucleotides (also referred to as Curagen Accession No. CG55771-02) encoding a novel Apolipoprotein A-1 Precursor-like protein is shown in Table 3C. An open reading frame was identified beginning with an ATG initiation codon at nucleotides 1-3 and ending with a TGA codon at nucleotides 634-636. A putative untranslated region downstream from the termination codon are underlined in Table 3C. The start and stop codons are in bold letters.

TABLE 3C
NOV3b nucleotide sequence (SEQ ID NO:13).

ATGAAAGCTGCGGTGCTGACCTTGGCCGTGCTCTTCCTGACGGGTGGGAGCCAGGCTCGGCATTTCTGGCAG
CAAGATGAACCCCCCCAGAGCCCCTGGGATCGAGTGAAGGACCTGGCCACTGTGTACGTCGATGTGCTCAAA
GACAGCGGCGACAGCGTGACCTCCACCTTCAGCAAGCTGCGCGAACAGCTCGGCCCTGTGACCCAGGAGTTC
TGGGATAACCTGGAAAAGGAGACAGAGGGCCTGAGGCAGGAGATGAGCAAGGATCTCGAGGACGTGAATGCC
AAGGTGCAGCCCTACCTGGACGACTTCCAGAAGAAGTGGCAGGAGGAGATGGAGCTCTACCGCCAGAAGGTG
GAGCCGCTGCGCGCAGAGCTCCAAQAGGGCGCGCGCCAGAAGCTGCACGAGCTGCGCCAGCGCTTGGCCGAG
CGCCTTGAGGCTCTCAAGGAGAACGGCGGCGCCAGACTGGCCGAGTACCACGCCAAGGCCACCGAGCATCTG
AGCACGCTCAGCGAGAAGGCCAAGCCCGCGCTCGAGGACCTCCGCCAAGGCCTGCTGCCCGTGCTGGAGAGC
TTCAAGGTCAGCTTCCTGAGCGCTCTCGAGGAGTACACTAAAAGCTCAACACCCACTGAGGCGCCCCGCCGC
CGCCCCCCTTCCCGGTGCTCAGAATAAAC

In a search of public sequence databases, the NOV3b nucleic acid sequence, located on chromosome 11, has 491 of 676 bases (72%) identical to a gb:GENBANK-ID:HSAPOAIT|acc:X07496.1 mRNA from Homo sapiens (Human Tangier apoA-I gene) (E=3.1e⁻⁶⁷). Public nucleotide databases include all GenBank databases and the GeneSeq patent database.

The disclosed NOV3b polypeptide (SEQ ID NO: 14) encoded by SEQ ID NO: 13 has 211 amino acid residues and is presented in Table 3B using the one-letter amino acid code. Signal P, Psort and/or Hydropathy results predict that NOV3b has a signal peptide and is likely to be localized extracellularly with a certainty of 0.3798. In other embodiments, NOV3b may also be localized to the microbody (peroxisome) with a certainty of 0.1141, in the endoplasmic reticulum (membrane) with a certainty of 0.1000, or in the endoplasmic reticulum (lumen) with a certainty of 0.1000. The most likely cleavage site for NOV3b is between positions 19 and 20, SQA-RH.

TABLE 3D
Encoded NOV3b protein sequence (SEQ ID NO:14).

MKAAVLTLAVLFLTGGSQARHWQQDEPPQSPWDRVKDLATVYVDVLKDSGDSVTSTFSKLRAEQLGPVTQEF
WDNLEKETEGLRQEMSKDLEEVKAKVQPYLDDFQKKWQEEMELYRQKVEPLRAELQEGARQKLHELRQRLAE
RLEALKENCGARLAEYHAKATEHLSTLSEKAKPALEDLRQGLLPVLESFKVSFLSALEEYTKKLNTQ

A search of sequence databases reveals that the NOV3b amino acid sequence has 106 of 161 amino acid residues (65%) identical to, and 121 of 161 amino acid residues (75%) similar to, the 267 amino acid residue ptnr:SWISSPROT-ACC:P02647 protein from Homo sapiens (Human) (Apolipoprotein A-I Precursor (APO-AI)) (E=5.6e⁻⁴⁷). Public amino acid databases include the GenBank databases, SwissProt, PDB and PIR.

NOV3b is expressed in at least Liver, Spleen, Ovary. Expression information was derived from the tissue sources of the sequences that were included in the derivation of the sequence of CuraGen Acc. No. CG55771-02.

NOV3a also has homology to the amino acid sequences shown in the BLASTP data listed in Table 3E.

TABLE 3E
BLAST results for NOV3a

| Gene Index/ | Protein/ | Length | Identity | Positives | |
|---|---|---|---|---|---|
| Identifier | Organism | (aa) | (%) | (%) | Expect |
| gi\|2119390\|pir\|\|I55 | proapo-A-I | 267 | 212/267 | 213/267 | 4e−95 |
| 236 | protein - human | | (79%) | (79%) | |
| gi\|4557321\|ref\|NP_0 | apolipoprote | 267 | 213/267 | 213/267 | 4e−95 |
| 00030.1\| | in A-I | | (79%) | (79%) | |
| (NM_000039) | precursor | | | | |
| | [Homo sapiens | | | | |
| gi\|178775\|gb\|AAA517 | proapolipoprotein | 249 | 207/249 | 207/249 | 2e−91 |
| 47.1\|(M29068) | [Homo sapiens | | (83%) | (83%) | |
| gi\|399042\|sp\|P15568\| | APOLIPOPROTE | 267 | 202/267 | 207/267 | 2e−90 |
| APA1_MACFA | IN A-I | | (75%) | (76% | |
| | PRECURSOR (APO-AI) | | | | |
| gi\|86614\|pir\|\|A26529 | apolipoprote | 267 | 202/267 | 207/267 | 2e−90 |
| | in A-I | | (75%) | (76%) | |
| | precursor - | | | | |
| | crab-eating | | | | |
| | macaque | | | | |

The homology of these sequences is shown graphically in the ClustalW analysis shown in Table 3F.

[image]

[image]

Table 3G lists the domain description from DOMAIN analysis results against NOV3a. This indicates that the NOV3a sequence has properties similar to those of other proteins known to contain this domain.

TABLE 3G
Domain Analysis of NOV3a

gnl|Pfam|pfam01442, Apolipoprotein, Apolipoprotein A1/A4/E family.
These proteins contain several 22 residue repeats which form a pair of
alpha helices. This family includes: Apolipoprotein A-I,
Apolipoprotein A-IV, and Apolipoprotein E. (SEQ ID NO:79)
CD-Length=262 residues, 95.0% aligned
Score=182 bits (461), Expect=2e−47

| | | | |
|---|---|---|---|
| Query: | 15 | GSQARHFWQQDEPPQSPWDRVKDLATVYVDVLKDS-------------------------- | 49 |
| | | \| \|\|\| \|\|\| \|\|\| \|\| \|\|+\|\|\| \|\|+ +\|\|\| | |
| Sbjct: | 14 | GCQAR-FWQADEP-QSQWDQVDKDRFWVYLRQVKDSADQAVEQLESSQVTQELNLLLQDNL | 71 |
| Query: | 50 | --VTSTFSKLREQLGPVTQEFWDNLEKETEGLRQEMSKDLEEVKAKVQPYLDDFQKKWQE | 107 |
| | | + \| +\|+\|\|\|\|\|\| \|\|\|\| \| \|\|\|+ \|\| \|+ \|\|\|\|+ + ++ \|\| \|+ \|+ + | |
| Sbjct: | 72 | DELKSYAEELQEQLGPVAQEFWARLSKETQALRAELGKDLEDVRNRLAPYRDELQQMLGQ | 131 |
| Query: | 108 | EMELYRQKVEPLRAELQEGARQKLHELQEKLSPLGEEMRDRARAHVDALRTHLAPYSDEL | 167 |
| | | +\| \|\|\|\|+\|\|\| \|\|++ \|+ \|\|\|++\|+\| \|\|+\|+\|\| +\|\|\|\|\|\| \| \|\| ++\| | |
| Sbjct: | 132 | NIEEYRQKLEPLARELRKRLRRDAEELQKRLAPYAEELRERAERNVDALRTRLGPYVEQL | 191 |
| Query: | 168 | RQRLAARLEALKENGGARLAEYHAKATEHLSTLSEKAKPALEDLRQGLLPVLESFKVSFL | 227 |
| | | \|\|+\| \|\|\| \|+\| \|\| + \| \|\| \| \|\| \| \|\|\|++ \| \|\|\|\| \| | |
| Sbjct: | 192 | RQKLTQRLEELRERAQPYAEEYKEQLEEQLSELREKLAPLREDLQEVLNPVLEQLKTQAE | 251 |
| Query: | 228 | SALEEYTKKLN | 238 |
| | | + \|\| \| | |
| Sbjct: | 252 | AFQEELKSWLE | 262 |

Apolipoprotein A-I is the major apoprotein of HDL and is a relatively abundant plasma protein with a concentration of 1.0-1.5 mg/ml. It is a single polypeptide chain with 243 amino acid residues of known primary amino acid sequence (Brewer et al., 1978). ApoA-I is a cofactor for LCAT (245900), which is responsible for the formation of most cholesteryl esters, in plasma. ApoA-I also promotes efflux of cholesterol from cells. The liver and small intestine are the sites of synthesis of apoA-I. The primary translation product of the APOAI gene contains both a pre and a pro segment, and posttranslational processing of apoA-I may be involved in the formation of the functional plasma apoA-I isoproteins. Dayhoff (1976) pointed to sequence homologies of A-I, A-II, C-I, and C-III.

Yui et al. (1988) found that apoA-I is identical to serum PGI(2) stabilizing factor (PSF). PGI(2), or prostacyclin, is synthesized by the vascular endothelium and smooth muscle, and functions as a potent vasodilator and inhibitor of platelet aggregation. The stabilization of PGI(2) by HDL and apoA-I may be an important protective action, against the accumulation of platelet thrombi at sites of vascular damage. The beneficial effects of HDL in the prevention of coronary artery disease may be partly explained by this effect. A-I(Milano) and A-I(Marburg) give rise to HDL deficiency. Other HDL deficiency states are Tangier disease (HDLDT1; 205400), LCAT deficiency (245900), and ‘fish-eye’ disease (136120).

Breslow et al. (1982) isolated and characterized cDNA clones for human apoA-I. Rees et al. (1983) studied the cloned APOAI gene and a DNA polymorphism 3-prime to it. In a healthy control population, the frequency of heterozygotes was about 5%. Among hypertriglyceridemic subjects, 34% were heterozygotes and about 6% were homozygotes for the variant. The primary gene transcript encodes a preproapoA-I containing 24 amino acids on the amino terminus of the mature plasma apoA-I (Law et al., 1983).

Law et al. (1984) assigned the APOA1 gene to 11p 11-q13 by filter hybridization analysis of human-mouse cell hybrid DNAs. The genes for apoA-I and apoC-III are on chromosome 9 in the mouse. Mouse homologs of other genes on human 11p (insulin, beta-globin, LDHA, HRAS) are situated on mouse chromosome 7. Using a cDNA probe to detect apoA-I structural gene sequences in human-Chinese hamster cell hybrids, Cheung et al. (1984) assigned the gene to the region 11q13-qter. Since other information had suggested 11p11-q13 as the location, the SRO becomes 11q13. It is noteworthy that in the mouse and in man, APOA1 and PGBD (called Ups in the mouse) are syntenic. Both are on chromosome 11 in man and chromosome 9 in the mouse. Bruns et al. (1984) localized the genes for apoA-I and apoC-III (previously shown to be in a 3-kb segment of the genome; Breslow et al., 1982; Shoulders et al., 1983) to chromosome 11 by Southern blot analysis of DNA from human-rodent cell hybrids. Because in the mouse apoA-I is on chromosome 9 and apoA-II is on chromosome 1 (Lusis et al., 1983), the gene for human apoA-II is probably not on chromosome 11. Indeed, APOA2 (107670) is on human chromosome 1. On the basis of data provided by Pearson (1987), the APOA1 locus was assigned to 11q23-qter by HGM9. This would place APOC3 and APOA4 in the same region. Because the XmnI genotype at the APOA1 locus was heterozygous in a boy with partial deletion of the long arm of chromosome 11, del(11)(q23.3-qter), Arinami et al. (1990) localized the gene to 11q23 by excluding the region 11q24-qter.

Haddad et al. (1986) found that in the rat, as in man, the APOA1, APOC3 and APOA4 genes are closely linked. Indeed, their direction of transcription, size, relative location and intron-exon organization were found to be remarkably similar to those of the corresponding human genes.

There are 8 well-characterized apolipoproteins: apoA-I, apoA-II, apoA-IV, apoB, apoC-I, apoC-II, apoC-III, and apoe. The APOA1 and APOC3 genes are oriented ‘foot-to-foot,’ i.e., the 3-prime end of APOA1 is followed after an interval of about 2.5 kb by the 3-prime end of APOC3 (Karathanasis et al., 1983).

In 4 generations of a Norwegian kindred, Schamaun et al. (1983) found, by 2-D electrophoresis, a variant of apolipoprotein A-I. Codominant inheritance was displayed. One homozygote was identified. There was no obvious cardiovascular disease, even in the homozygote. Karathanasis et al. (1983) found that a group of severely hypertriglyceridemic patients with types IV and V hyperlipoproteinemia had an increased frequency of an RFLP associated with the apoA-I gene. Rees et al. (1985) found a strong correlation between hypertriglyceridemia and a DNA sequence polymorphism located in or near the 3-prime noncoding region of APOC3 and revealed by digestion of human DNA with the restriction enzyme Sst-1 and hybridization with an APOA1 cDNA probe. In 74 hypertriglyceridemic Caucasians, 3 were homozygous and 23 were heterozygous for the polymorphism, giving a gene frequency of 0.19; none of 52 normotriglyceridemics had the polymorphism, although it was frequent in Africans, Chinese, Japanese, and Asian Indians. No differences in high density lipoprotein or in apolipoproteins A-I and C-III phenotypes were found in persons with or without the polymorphism. Ferns et al. (1985) found an uncommon allelic variant (called S2) of the apoA-I/C-III gene cluster in 10 of 48 postmyocardial infarction patients (21%). In 47 control subjects it was present in only 2 and in none of those who were normotriglyceridemic. (The S2 allele, a DNA polymorphism, is characterized by SstI restriction fragments of 5.7 and 3.2 kb length, whereas the common S1 allele produces fragments of 5.7 and 4.2 kb length.) Ferns et al. (1985) found no difference in the distribution of alleles in the highly polymorphic region of 11p near the insulin gene. Kessling et al. (1985) failed to find an association between any allele of several RFLPs studied and hypertriglyceridemia. Buraczynska et al. (1985) found association between an EcoRI polymorphism of the APOA1 gene and noninsulin-dependent diabetes mellitus.

Familial hypoalphalipoproteinemia, by far the most common of the forms of primary depression of HDL cholesterol, has been thought to be an autosomal dominant. It is associated with premature coronary artery disease and stroke (Vergani and Bettale, 1981; Third et al., 1984; Daniels et al., 1982). Using a PstI polymorphism at the 3-prime end of the APOA1 gene, Ordovas et al. (1986) found the rarer allele (‘3.3-kb band’) in 4.1% of 123 randomly selected control subjects and 3.3% of 30 subjects with no angiographic evidence of coronary artery disease. In contrast, among 88 patients who had severe coronary artery disease before age 60, as documented by angiography, the frequency was 32%. It was also found in 8 of 12 index cases of kindreds with familial hypoalphalipoproteinemia. Among all patients with coronary artery disease, 58% had HDL cholesterol levels below the 10th percentile; however, this frequency increased to 73% when patients with the 3.3-kb band were considered. Borecki et al. (1986) studied 16 kindreds ascertained through probands clinically determined to have primary hypoalphalipoproteinemia characterized by low HDL cholesterol but otherwise normal blood lipids. They concluded that ‘these families provided clear evidence for a major gene.’ Moll et al. (1986) measured apoA-I levels in families ascertained through cases of hypertension or early coronary artery disease. They concluded that the findings supported ‘a major effect of a single genetic locus on the quantitative variation of plasma apoA-I in a sample of pedigrees enriched for individuals at risk for coronary artery disease.’ Using a radioimmunoassay, Moll et al. (1989) measured plasma apoA-I levels in 1,880 individuals from 283 pedigrees. Complex segregation analysis suggested heterogeneous etiologies for the individual differences in adjusted apoA-I levels observed. The authors concluded that environmental factors and polygenic loci account for 32 and 65%, respectively, of the adjusted variation in a subset of 126 families. In the other 157 pedigrees, segregation analysis strongly supported the presence of a single locus accounting for 27% of the adjusted variation. In Japanese, Rees et al. (1986) found association of triglyceridemia with a different haplotype of the A-I/C-III region than that found in Caucasians.

Ferns et al. (1986) found a common allele of the APOA2 locus which showed a weak association with hypertriglyceridemia; in contrast, an uncommon allele of the APOA1-APOC3-APOA4 gene cluster demonstrated a stronger relationship with hypertriglyceridemia. Ferns et al. (1986) found higher levels of serum triglycerides with possession of both disease-related alleles than with either singly. Fager et al. (1981) found an inverse relationship between serum apoA-II and a risk of myocardial infarction. Hayden et al. (1987) found an association between certain RFLPs and familial combined hyperlipidemia (FCH; 144250). APOA1 is linked to THY1 (188230) at a distance of about 1 cM (Gatti, 1987); thus, the more distal location of this apolipoprotein cluster as suggested by other evidence may be true. In certain patients with premature atherosclerosis, Karathanasis et al. (1987) demonstrated a DNA inversion containing portions of the 3-prime ends of the APOA1 and APOC3 genes, including the DNA region between these genes. The breakpoints of this DNA inversion were found to be located between the fourth exon of the APOA1 gene and the first intron of the APOC3 gene; thus, the inversion results in reciprocal fusion of the 2 gene transcriptional units. The absence of transcripts with correct mRNA sequences causes deficiency of both apolipoproteins in the plasma of these patients, leading to atherosclerosis. Bojanovski et al. (1987) found that both proapolipoprotein A-I and the mature protein are metabolized abnormally rapidly in Tangier disease. Thompson et al. (1988) investigated the seeming paradox that 2 RFLPs at the A-I/C-III cluster were in strong linkage disequilibrium while a third variant, located between the 2 other markers, appeared to be in linkage equilibrium with these 2 ‘outside’ markers. Thompson et al. (1988) showed that, for the gene frequencies encountered, very large sample sizes would be required to demonstrate negative (i.e., repulsion-phase) linkage disequilibrium. Such numbers are usually difficult to attain in human studies. Therefore, failure to demonstrate linkage disequilibrium by conventional methods does not necessarily imply its absence.

Kessling et al. (1988) studied the high density lipoprotein-cholesterol concentrations along with restriction fragment length polymorphisms in the APOA2 and APOA1-APOC3-APOA4 gene cluster in 109 men selected from a random sample of 1,910 men aged 45 to 59 years. They found no significant difference in allelic frequencies at either locus between the groups of individuals with high and low HDL-cholesterol levels. They did find an association between a PstI RFLP associated with apoA-I and genetic variation determining the plasma concentration of apoA-I. No significant association was found between alleles for the apoA-II MspI RFLP and apoA-II or HDL concentrations. ApoA-I has 243 amino acids of known sequence. It is secreted into the bloodstream by the liver and intestine as a protein that is rapidly converted to mature apoA-I. Two major isoforms of mature, normal A-I, which arise by deamidation, can be separated in human serum. Antonarakis et al. (1988) studied DNA polymorphism of a 61-kb segment of 11q that contains the APOA1, APOC3, and APOA4 genes within a 15-kb stretch. Eleven RFLPs located within the 61-kb segment were used by haplotype analysis. Considerable linkage disequilibrium was found. Several haplotypes had arisen by recombination and the rate of recombination within the gene cluster was estimated to be at least 4 times greater than that expected based on uniform recombination. Taken individually, the polymorphism information content (PIC) of each of the 11 polymorphisms ranged from 0.053 to 0.375, while that of their haplotypes ranged between 0.858 and 0.862. (The PIC value, which was introduced by Botstein et al. (1980) in their classic paper on the use of RFLPs: as linkage markers, represents the sum of the frequency of each possible mating multiplied by the probability that an offspring will be informative.) By genetic linkage analysis using RFLPs in the APOA1/C3/C4 gene cluster,

Kastelein et al. (1990) showed that the mutation causing familial hypoalphalipoproteinemia (familial HDL deficiency) in a family of Spanish descent was not located in this cluster.

Smith et al. (1992) investigated the common G/A polymorphism in the APOA1 gene promoter at a position 76 bp upstream of the transcriptional start site (−76). Of 54 subjects whose apoA-I production rates had been determined by turnover studies, 35 were homozygous for a guanosine at this locus and 19 were heterozygous for a guanosine and adenosine (G/A). The apoA-I production rates were significantly lower (by 11%) in the G/A heterozygotes than in the G homozygotes (P=0.025). However, no effect on HDL cholesterol or apoA-I levels were noted. Differential gene expression of the 2 alleles was tested by linking each of the alleles to the reporter gene chloramphenicol acetyltransferase and determining relative promoter efficiencies after transfection into the human HepG2 hepatoma cell line. The A allele, as well as the G allele, expressed only 68%.

In addition to its ability to remove cholesterol from cells, HDL also delivers cholesterol to cells through a poorly defined process in which cholesteryl esters are selectively transferred from HDL particles into the cell without the uptake and degradation of the lipoprotein particle. In steroidogenic cells of rodents, the selective uptake pathway accounts for 90% or more of the cholesterol destined for steroid production or cholesteryl ester accumulation. To test the importance of the 3 major HDL proteins in determining cholesteryl ester accumulation in steroidogenic cells of the adrenal gland, ovary, and testis, Plump et al. (1996) used mice which had been rendered deficient in apoA-I, apoA-II, or apoE by gene targeting in embryonic stem cells. ApoE and apoA-II deficiencies were found to have only modest effects on cholesteryl ester accumulation. In contrast, apoA-I deficiency caused an almost complete failure to accumulate cholesteryl ester in steroidogenic cells. Plump et al. (1996) interpreted these results as indicating that apoA-I is essential for the selective uptake of HDL-cholesteryl esters. They stated that the lack of apoA-I has a major impact on adrenal gland physiology, causing diminished basal corticosteroid production, a blunted steroidogenic response to stress, and increased expression of compensatory pathways to provide cholesterol substrate for steroid production.

In studies of 3 restriction enzyme polymorphisms in the AI-CII-AIV gene cluster, Dallinga-Thie et al. (1997) analyzed haplotypes and showed an association with severe hyperlipidemia in subjects with FCH. Furthermore, nonparametric sib pair linkage analysis revealed significant linkage between these markers in the gene cluster and the FCH phenotype. The findings confirmed that the AI-CIII-AIV gene cluster contributes to the FCH phenotype, but this contribution is genetically complex. An epistatic interaction between different haplotypes of the gene cluster was demonstrated. They concluded that 2 different susceptibility loci exist in the gene cluster.

Naganawa et al. (1997) reported 2 haplotypes due to 5 polymorphisms in the intestinal enhancer region of the APOA1 gene in endoscopic biopsy samples from healthy volunteers. The mutant haplotype had a population frequency of 0.44; frequency of wildtype was 0.53. APOA1 mRNA levels were 49% lower in mutant haplotype homozygotes than in wildtype homozygotes, while APOA1 synthesis was 37% lower than wildtype in individuals homozygous for the mutant allele. Heterozygotes had 28% and 41% reductions of mRNA levels and APOA1 synthesis, respectively, as compared to wildtype homozygotes. Expression studies in Caco-2 cells showed a 46% decrease in transcriptional activity in cells containing the mutant constructs, and binding of Caco-2 nuclear proteins in mutant, but not wildtype, sequences. Naganawa et al. (1997) concluded that intestinal APOA1 transcription and protein synthesis were reduced in the presence of common mutations which induced nuclear protein binding.

Genschel et al. (1998) counted 4 naturally occurring mutant forms of apoA-I that were known at that time to result in amyloidosis. The most important feature of all variants was the very similar formation of N-terminal fragments found in the amyloid deposits. They summarized the specific features of all known amyloidogenic variants of APOA1 and speculated about the metabolic pathway involved.

To determine the frequency of de novo hypoalphalipoproteinemia in the general population due to mutation of the APOA1 gene, Yamakawa-Kobayashi et al. (1999) analyzed sequence variations in the APOA1 gene in 67 children with a low high-density lipoprotein (HDL) cholesterol level. These children were selected from 1,254 school children through a school survey. Four different mutations with deleterious potentia, 3 frameshifts and I splice site mutation, were identified in 4 subjects. The plasma apoA-I levels of the 4 children with these mutations were reduced to approximately half of the normal levels and were below the first percentile of the general population distribution (80 mg/dl). The frequency of hypoalphalipoproteinemia due to a mutant APOA1 gene was estimated at 6% in subjects with low HLD cholesterol levels and 0.3% in the Japanese population generally.

High density lipoprotein deficiency is also caused by mutations in the ABC1 gene (600046), which lead to reductions in cellular cholesterol efflux. The disorder is clinically and biochemically severe in the case of the recessively inherited Tangier disease, whereas it is milder in the dominantly inherited type 2 familial high density lipoprotein deficiency (604091).

The disclosed NOV3 nucleic acid of the invention encoding a Apolipoprotein A-I precursor-like protein includes the nucleic acid whose sequence is provided in Table 3A, 3C, or a fragment thereof. The invention also includes a mutant or variant nucleic acid any of whose bases may be changed from the corresponding base shown in Table 3A, or 3C while still encoding a protein that maintains its Apolipoprotein A-I precursor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1% percent of the bases may be so changed.

The disclosed NOV3 protein of the invention includes the Apolipoprotein A-I precursor-like protein whose sequence is provided in Table 3B, or 3D. The invention also includes a mutant or variant protein any of whose residues may be changed from the corresponding residue shown in Table 3B, or 3D while still encoding a protein that maintains its Apolipoprotein A-I precursor-like activities and physiological functions, or a functional fragment thereof. In the mutant or variant protein, up to about 25 percent of the residues may be so changed.

The protein similarity information, expression pattern, and map location for the Apolipoprotein A-I precursor-like protein and nucleic acid (NOV3) disclosed herein suggest that NOV3 may have important structural and/or physiological functions characteristic of the citron kinase-like family. Therefore, the NOV3 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV3 nucleic acids and proteins of the invention are useful in potential diagnostic and therapeutic applications implicated in various diseases and disorders described below. For example, the compositions of the present invention will have efficacy for treatment of patients suffering from coronary artery disease, stroke, hypertriglyceridemia, hypoalphalipoproteinemia, hyperlipidemia, Tangier disease, LCAT deficiency, ‘fish-eye’ disease, noninsulin-dependent diabetes mellitus, hypertension, myocardial infarction, atherosclerosis, and/or other pathologies.

NOV3 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV3 protein have multiple hydrophilic regions, each of which can be used as an immunogen. In one embodiment, contemplated NOV3 epitope is from about amino acids 20 to 40. In another embodiment, a NOV3 epitope is from about amino acids 50 to 220. In additional embodiments, NOV3 epitopes are from about amino acids 240 to 260. This novel protein also has value in development of powerful assay system for functional analysis of various human disorders, which will help in understanding of pathology of the disease and development of new drug targets for various disorders.

NOV4

NOV4 includes three novel HSP90 co-chaperone-like proteins disclosed below. The disclosed sequences have been named NOV4a, NOV4b, and NOV4c.

NOV4a

A disclosed NOV4a nucleic acid of 513 nucleotides (designated CuraGen Acc. No. CG55700-01) encoding a novel HSP90 co-chaperone-like protein is shown in Table 4A. An open reading frame was identified beginning with an ATG initiation codon at nucleotides 54-56 and ending with a TAA codon at nucleotides 444-446. A putative untranslated region downstream from the termination codon is underlined in Table 4A, and the start and stop codons are in bold letters.

TABLE 4A
NOV4a Nucleotide Sequence (SEQ ID NO:15)

CATTTGCTGTCTCCTCTGCTCACCAGTTCGCCCGTCCCCCTGCCCCGTTC
ACAATGCAGCCTGCTTCTGCAAAGTGGTACGATCGAAGGGACTATGTCTT
CATTGAATTTTGTGTTGAAGACAGTAAGGATGTTAATGTAAATTTTGAAA
AATCCAAACTTACATTCAGTTGTCTCGGAGGAAGTGATAATTTTAAGCAT
TTAAATGAAATTGATCTTTTTCACTGTATTGATCCAAATGATTCCAAGCA
TAAAAGAACGGACAGATCAATTTTATGTTGTTTACGAAAAGGAGAATCTG
GCCAGTCATGGCCAAGGTTAACAAAAGAAAGGGCAAAGATGATGAACAAC
ATGGGTGGTGATGAGGATGTAGATTTACCAGAAGTAGATGGAGCAGATGA
TGATTCACAAGACAGTGATGATGAAAAAATGCCAGATCTGGAGTAAGGAA
TATTGTCATCACCTGGATTTTGAGAAAGAAAAATAACTTCTCTGCAAGAT
TTCATAATTGAGA

The nucleic acid sequence of 354 of 388 bases (91%) identical to a gb:GENBANK-ID:HUMPRA|acc:L24804.1 mRNA from Homo sapiens (Human (p23) mRNA, complete cds) (E=3.3e⁻⁶⁶).

A NOV4a polypeptide (SEQ ID NO: 16) encoded by SEQ ID NO: 15 is 130 amino acid residues and is presented using the one letter code in Table 4B. Signal P, Psort and/or Hydropathy results predict that NOV4a has no signal peptide and is likely to be localized at the nucleus with a certainty of 0.4600. In other embodiments, NOV4a may also be localized to the microbody (peroxisome) with a certainty of 0.3000, the mitochondrial membrane space with a certainty of 0.1000, or the lysosome (lumen) with a certainty of 0.1000.

TABLE 4B
NOV4a protein sequence (SEQ ID NO:16)

MQPASAKWYDRRDYVFIEFCVEDSKDVNVNFEKSKLTFSCLGGSDNFKHL
NEIDLFHCIDPNDSKHKRTDRSILCCLRKGESGQSWPRLTKERAKMMNNM
GGDEDVDLPEVDGADDDSQDSDDEKMPDLE

The full amino acid sequence of the protein of the invention was found to have 101 of 122 amino acid residues (82%) identical to, and 107 of 122 amino acid residues (87%) similar to, the 160 amino acid residue ptnr:SWISSNEW-ACC:Q15185 protein from Homo sapiens (Human) (HSP90 Co-Chaperone (Progesterone Receptor Complex P23)) (E=7.9e⁻⁵¹).

NOV4 is expressed in at least Adrenal Gland/Suprarenal gland, Amnion, Amygdala, Aorta, Appendix, Ascending Colon, Bone, Bone Marrow, Brain, Bronchus, Brown adipose, Cartilage, Cervix, Chorionic Villus, Cochlea, Colon, Cornea, Coronary Artery, Dermis, Duodenum, Epidermis, Foreskin, Gall Bladder, Gastro-intestinal/Digestive System, Hair Follicles, Heart, Hippocampus, Islets of Langerhans, Kidney, Kidney Cortex, Larynx, Left cerebellum, Liver, Lung, Lung Pleura, Lymph node, Lymphoid tissue, Mammary gland/Breast, Muscle, Ovary, Oviduct/Uterine Tube/Fallopian tube, Pancreas, Parathyroid Gland, Parietal Lobe, Parotid Salivary glands, Peripheral Blood, Pharynx, Pituitary Gland, Placenta, Prostate, Retina, Right Cerebellum, Salivary Glands, Skin, Small Intestine, Spinal Chord, Spleen, Stomach, Substantia Nigra, Temporal Lobe, Testis, Thalamus, Thymus, Thyroid, Tonsils, Trachea, Umbilical Vein, Urinary Bladder, Uterus, Vein, Vulva, Whole Organism. This information was derived by determining the tissue sources of the sequences that were included in the invention including but not limited to SeqCalling sources, Public EST sources, Literature sources, and/or RACE sources.

NOV4b

In the present invention, the target sequence identified previously, NOV4a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequences reported below, which are designated NOV4b .

A disclosed NOV4b nucleic acid of 520 nucleotides (designated CuraGen Acc. No. CG55700-02) encoding a novel HSP90 Co-Chaperone (Progesterone Receptor Complex P23)-like protein is shown in Table 4C. An open reading frame was identified beginning with an ATG initiation codon at nucleotides 1-3 and ending with a TAA codon at nucleotides 481-483. A putative untranslated region downstream from the termination codon is underlined in Table 4C, and the start and stop codons are in bold letters.

TABLE 4C
NOV4b Nucleotide Sequence (SEQ ID NO:17)

ATGCAGCCTGCTTCTGCAAAGTGGTACGATCGAAGGGACTATGTCTTCAT
TGAATTTTGTGTTGAAGACAGTAAGGATGTTAATGTAAATTTTGAAAAAT
CCAAACTTACATTCAGTTGTCTCGGAGGAAGTGATAATTTTAAGCATTTA
AATGAAATTGATCTTTTTCACTGTATTGATCCAAATGATTCCAAGCATAA
AAGAACGGACAGATCAATTTTATGTTGTTTACGAAAAGGAGAATCTGGCC
AGTCATGGCCAAGGTTAACAAAAGAAAGGGCAAAGCTTAATTGGCTTAGT
GTCGACTTCAATAATTGGAAAGACTGGGAAGATGATTCAGATGAAGACAT
GTCTAATTTTGATCGTTTCTCTGAGATGATGAACAACATGGGTGGTGATG
AGGATGTAGATTTACCAGAAGTAGATGGAGCAGATGATGATTCACAAGAC
AGTGATGATGAAAAAATGCCAGATCTGGAGTAAGGAATATTGTCATCAC
CTGGATTTTGAGAAAGAAAAA

A NOV4b polypeptide (SEQ ID NO: 18) encoded by SEQ ID NO: 17 is 160 amino acid residues and is presented using the one letter code in Table 4D.

TABLE 4D
NOV4b protein sequence (SEQ ID NO:18)

MQPASAKWYDRRDYVFIEFCVEDSKDVNVNFEKSKLTFSCLGGSDNFKHL
NEIDLFHCIDPNDSKHKRTDRSILCCLRKGESGQSWPRLTKERAKLNWLS
VDFNNWKDWEDDSDEDMSNFDRFSEMMNNMGGDEDVDLPEVDGADDDSQD
SDDEKMPDLE

The human cDNA encodes a protein of 160 amino acids that does not show homology to previously identified proteins. The chicken and human cDNAs are 88% identical at the DNA level and 96.3% identical at the protein level. p23 is a highly acidic phosphoprotein with an aspartic acid-rich carboxy-terminal domain. Bacterially overexpressed human p23 was used to raise several monoclonal antibodies to p23. These antibodies specifically immunoprecipitate p23 in complex with hsp90 in all tissues tested and can be used to immunoaffinity isolate progesterone receptor complexes from chicken oviduct cytosol.

NOV4c

In the present invention, the target sequence identified previously NOV4a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp, In addition, sequence traces were evaluated manually and edited for corrections if appropriate. These procedures provide the sequences reported below, which are designated Accession Number NOV4c

A disclosed NOV4c nucleic acid of 426 nucleotides (designated CuraGen Acc. No. CG55700-03) encoding a novel HSP90 co-chaperone -like protein is shown in Table 4E. An open reading frame was identified beginning with a CCT initiation codon at nucleotides 1-3 and ending at nucleotides 424-426. The start codon is in bold letters in Table 4E. Because the initiation codon is not a traditional initiation codon, and the lack of a termination codon, NOV4c could be a partial reading frame that could be extended in the 5′ or 3′ directions.

TABLE 4E
NOV4c Nucleotide Sequence (SEQ ID NO:19)

CCTGCTTCTGCAAAGTGGTACGATCGAAGGGACTATGTCTTCATTGAATT
TTGTGTTGAAGACAGTAAGGATGTTAATGTAAATTTTGAAAAATCCAAAC
TTACATTCAGTTGTCTCGGAGGAAGTGATAATTTTAAGCATTTAAATGAA
ATTGATCTTTTTCACTGTATTGATCCAAATGATTCCAAGCATAAAAGAAC
GGACAGATCAATTTTATGTTGTTTACGAAAAGGAGAATCTGGCCAGTCAT
GGCCAAGGTTAACAAAAGAAAGGGCAAAGCTTAATTGGCTTAGTGTCGAC
TTCAATAATTGGAAAGACTGGGAAGATGATTCAGATGAAGACATGTCTAA
TTTTGATCGTTTCTCTGAGAAATGCCAGATCTGGAGTAAGGAATATTGTC
ATCACCTGGATTTGAAGAAAGAAAAA

The nucleic acid sequence of NOV4, localized to chromosome 12, has 399 of 423 bases (94%) identical to a gb:GENBANK-ID:HUMPRA|acc:L24804.1 mRNA from Homo sapiens (Human (p23) mRNA, complete cds) (E=7.0e⁻⁷⁸).

A NOV4c polypeptide (SEQ ID NO: 20) encoded by SEQ ID NO: 19 is 142 amino acid residues and is presented using the one letter code in Table 4F. Signal P, Psort and/or Hydropathy results predict that NOV4c has no signal peptide and is likely to be localized at the microbody (peroxisome) with a certainty of 0.7015. In other embodiments, NOV4c may also be localized to the nucleus with a certainty of 0.4600, the mitochondrial membrane space with a certainty of 0.1000, or the lysosome (lumen) with a certainty of 0.1000.

TABLE 4F
NOV4c protein sequence (SEQ ID NO:20)

PASAKWYDRRDYVFIEFCVEDSKDVNVNFEKSKLTFSCLGGSDNFKHLNE
IDLFHCIDPNDSKHKRTDRSILCCLRKGESGQSWPRLTKERAKLNWLSVD
FNNWKDWEDDSDEDMSNFDRFSEKCQIWSKEYCHHLDLKKEK

The full amino acid sequence of the protein of the invention was found to have 123 of 123 amino acid residues (100%) identical to, and 123 of 123 amino acid residues (100%) similar to, the 160 amino acid residue ptnr:SWISSNEW-ACC:Q1 5185 protein from Homo sapiens (Human) (HSP90 Co-Chaperone (Progesterone Receptor Complex P23)) (E=1.5e⁻⁶⁷).

NOV4c is expressed in at least liver, pancreas, lymph node, hepatocellular carcinoma. Expression information was derived from the tissue sources of the sequences that were included in the derivation of the sequence of CuraGen Acc. No. CG55700-03.

NOV4a also has homology to the amino acid sequences shown in the BLASTP data listed in Table 4G.

TABLE 4G
BLAST results for NOV4a

| Gene Index/ | | Length | Identity | Positives | |
|---|---|---|---|---|---|
| Identifier | Protein/Organism | (aa) | (%) | (%) | Expect |
| gi\|1362904\|pir\| | progesterone | 160 | 121/160 | 121/160 | 2e−55 |
| \|A56211 | receptor-related | | (75%) | (75% | |
| | protein p23 - | | | | |
| | human | | | | |
| gi\|8928249\|sp\| | TELOMERASE- | 160 | 119/160 | 121/160 | 2e−54 |
| Q9R0Q7\|P23_MOUSE | BINDING | | (74%) | (75%) | |
| | PROTEIN P23 | | | | |
| | (HSP90 | | | | |
| | CO-CHAPERONE) | | | | |
| | (PROGESTERONE | | | | |
| | RECEPTOR | | | | |
| | COMPLEX P23) | | | | |
| gi\|5081800\|gb\| | telomerase binding | 160 | 117/160 | 119/160 | 9e−53 |
| AAD39543.1\| | protein p23 [ | | (73%) | (74%) | |
| AF153479_1 | Mus musculus] | | | | |
| (AF153479) | | | | | |
| gi\|1362727\|pir\|\| | progesterone | 160 | 116/160 | 120/160 | 2e−52 |
| B56211 | receptor-related | | (72%) | (74%) | |
| | protein p23 - | | | | |
| | chicken | | | | |
| gi\|9257073\|pdb\|1EJF\|A | Chain A, Crystal | 125 | 95/96 | 96/96 | 4e−47 |
| | Structure Of The | | (98%) | (99%) | |
| | Human Co-Chaperone | | | | |
| | P23 | | | | |

The homology of these sequences is shown graphically in the ClustalW analysis shown in Table 4H.

[image]

Using immunoprecipitation of unactivated avian progesterone receptor, Johnson et al. (Mol Cell Biol 1994; 14:1956-63) purified hsp90, hsp70, and three additional proteins, p54, p50, and p23. p23 is also present in immunoaffinity-purified hsp9o complexes along with hsp70 and another protein, p60. Antibody and cDNA probes for p23 were prepared in an effort to elucidate the significance and function of this protein. Antibodies to p23 detect similar levels of p23 in all tissues tested and cross-react with a protein of the same size in mice, rabbits, guinea pigs, humans, and Saccharomyces cerevisiae, indicating that p23 is a conserved protein of broad tissue distribution. These antibodies were used to screen a chicken brain cDNA library, resulting in the isolation of a 468-bp partial cDNA clone encoding a sequence containing four sequences corresponding to peptide fragments isolated from chicken p23. This partial clone was subsequently used to isolate a full-length human cDNA clone. The human cDNA encodes a protein of 160 amino acids that does not show homology to previously identified proteins. The chicken and human cDNAs are 88% identical at the DNA level and 96.3% identical at the protein level. p23 is a highly acidic phosphoprotein with an aspartic acid-rich carboxy-terminal domain. Bacterially overexpressed human p23 was used to raise several monoclonal antibodies to p23. These antibodies specifically immunoprecipitate p23 in complex with hsp90 in all tissues tested and can be used to immunoaffinity isolate progesterone receptor complexes from chicken oviduct cytosol.

The disclosed NOV4 nucleic acid of the invention encoding a HSP90 co-chaperone-like protein includes the nucleic acid whose sequence is provided in Table 4A, 4C, 4E or a fragment thereof. The invention also includes a mutant or variant nucleic acid any of whose bases may be changed from the corresponding base shown in Table 4A, 4C, or 4E while still encoding a protein that maintains its HSP90 co-chaperone -like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 9% percent of the bases may be so changed.

The disclosed NOV4 protein of the invention includes the HSP90 co-chaperone-like protein whose sequence is provided in Table 4B, 4D, or 4F. The invention also includes a mutant or variant protein any of whose residues may be changed from the corresponding residue shown in Table 4B, 4D, or 4F while still encoding a protein that maintains its HSP90 co-chaperone -like activities and physiological functions, or a functional fragment thereof. In the mutant or variant protein, up to about 28% percent of the residues may be so changed.

The protein similarity information, expression pattern, and map location for the HSP90 co-chaperone-like protein and nucleic acid (NOV4) disclosed herein suggest that this NOV4 protein may have important structural and/or physiological functions characteristic of the HSP90 co-chaperone family. Therefore, the NOV4 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV4 nucleic acids and proteins of the invention are useful in potential diagnostic and therapeutic applications implicated in various diseases and disorders described below. For example, the compositions of the present invention will have efficacy for treatment of patients suffering from adrenoleukodystrophy, congenital adrenal hyperplasia, hemophilia, hypercoagulation, idiopathic thrombocytopenic purpura, autoimmune disease, allergies, asthma, immunodeficiencies, transplantation, graft versus host disease, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, arthritis, tendonitis, fertility, atherosclerosis, aneurysm, hypertension, fibromuscular dysplasia, stroke, scleroderma, obesity, myocardial infarction, embolism, cardiovascular disorders, bypass surgery, cirrhosis, inflammatory bowel disease, diverticular disease, Hirschsprung's disease, Crohn's Disease, appendicitis, ulcers, diabetes, renal artery stenosis, interstitial nephritis, glomerulonephritis, polycystic kidney disease, systemic lupus erythematosus, renal tubular acidosis, IgA nephropathy, laryngitis, emphysema, ARDS, lymphedema, muscular dystrophy, myasthenia gravis, endometriosis, pancreatitis, hyperparathyroidism, hypoparathyroidism, growth and reproductive disorders, xerostomia, psoriasis, actinic keratosis, acne, hair growth/loss, allopecia, pigmentation disorders, endocrine disorders, tonsillitis, cystitis, incontinence, and/or other pathologies. The NOV4 nucleic acids, or fragments thereof, may further be useful in diagnostic applications, wherein the presence or amount of the nucleic acid or the protein are to be assessed.

NOV4 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example, the disclosed NOV4 protein has multiple hydrophilic regions, each of which can be used as an immunogen. In one embodiment, a contemplated NOV4 epitope is from about amino acids 5 to 125. These novel proteins can be used in assay systems for functional analysis of various human disorders, which will help in understanding of pathology of the disease and development of new drug targets for various disorders.

NOV5

A disclosed NOV5 nucleic acid of 2993 nucleotides (also referred to as CG55706-01) encoding a novel Type III adenylyl cyclase-like protein is shown in Table SA. An open reading frame was identified beginning with an ATG initiation codon at nucleotides 148-150 and ending with a TAG codon at nucleotides 2431-2433. Putative untranslated regions upstream from the initiation codon and downstream from the termination codon are underlined in Table 5A, and the start and stop codons are in bold letters.

TABLE 5A
NOV5 Nucleotide Sequence (SEQ ID NO:21)

GCTGGAGGTGGCCTCCCCTCCGCCCCAGACAAGAAGAGGCCCTCAGCCCT
CCCCCGGTCTCAGAGAGCCCTGAGAGGAGGCCCAGTCCAGAGCTCTTCCT
CCGTTCCCAGTCCACTTCTCTAGGGCCAGTAGCAGACACCAGCCAGTATG
CCGAGGAACCAGGGCTTCTCCGAGCCCGAATACTCGGCCGAGTACTCAGC
CGAGTACTCCGTCAGCCTGCCCTCGGACCCTGACCGCGGGGTGGGCCGGA
CCCATGAAATCTCGGTCCGGAACTCGGGCTCCTGCCTGTGCCTGCCTCGC
TTCATGCGGCGCGGCTCTGCGGGGAGCAGCCCTCGGGCGCGCCGAGCTCT
CCCGCCCCAGCCCGCGCGGGGACCGTCCCGGAGCACGCGGTGGCCGAGTT
CCCGCACAGTTCTAGCTGATCAGTGCTACCTGTGCTCTGGAAACCCGCTC
TGCGTTCCTGCTGGAGGTGGCCTCCCCTTCGCCCCAGACAAGAAGAGGCC
CTCAGCCCTCCCCCGGTCTCAGAGAGCCCTGAGAGGAGGCCCAGTCCAGA
GCTCTTCCTCAAAGTCCAGCTCCCCTGCCCTCATTGAGACCAAGGAGCCC
AACGGGAGTGCCCACAGCAGTGGGTCCACGTCGGAGAAGCCCGAGGAGCA
GGATGCCCAGGCCGACAACCCCTCATTCCCCAACCCACGCCGGAGGCTGC
GCCTGCAGGACCTGGCTGACCGAGTGGTGGATGCCTCTGAAGATGAGCAC
GAGCTCAACCAGCTGCTCAACGAGGCCCTGCTTGAGCGAGAGTCCGCCCA
AGTAGTAAAGAAGAGAAACACCTTCCTCTTGTCCATGCGGTTCATGGACC
CCGAGATGGAAACCCGCTACTCGGTGGAGAAGGAGAAGCAGAGTGGGGCT
GCCTTCAGCTGCTCCTGCGTCGTCCTGCTCTGCACGGCCCTGGTCGAGAT
ACTCATCGACCCCTGGCTAATGACAAACTATGTGACCTTCATGGTGGGGG
AGATTCTGCTCCTCATCCTGACCATCTGCTCCCTGGCTGCCATCTTTCCC
CGGGCCTTTCCTAAGAAGCTTGTGGCCTTCTCAACTTGGATTGACCGGAC
CCGCTGGGCCAGGAACACCTGGGCCATGCTCGCCATCTTCATCCTGGTGA
TGGCAAATGTCGTGGACATGCTCAGCTGTCTCCAGTACTACACGGGACCC
AGCAATGCAACGGCAGGGATGGAGACGGAGGGCAGCTGCCTGGAGAACCC
CAAGTATTACAACTATGTGGCCGTGCTGTCCCTCATCGCCACCATCATGC
TGGTGCAGGTCAGCCACATGGTGAAGCTCACGCTCATGCTGCTCGTCGCA
GGCGCCGTGGCCACCATCAACCTCTATGCCTGGCGTCCCGTCTTTGATGA
ATACGACCACAAGCGTTTTCGGGAGCACGACTTACCTATGGTGGCCTTAG
AGCAGATGCAAGGATTCAACCCTGGGCTCAATGGCACTGACAGGCTGCCC
CTGGTGCCTTCCAAGTACTCTATGACGGTGATGGTGTTCCTCATGATGCT
CAGCTTCTACTACTTCTCCCGCCACGTAGAAAAACTGGCACGGACACTTT
TCTTGTGGAAGATTGAGGTCCACGACCAGAAGGAACGTGTCTATGAGATG
CGACGCTGGAACGAGGCCTTGGTCACCAACATGTTGCCTGAGCACGTGGC
ACGCCATTTCCTGGGGTCCAAGAAGAGAGATGAGGAGCTGTATAGCCAGA
CGTATGATGAGATTGGAGTCATGTTTGCCTCCCTGCCCAACTTTGCTGAC
TTCTACACAGAGGAGAGCATCAACAATGGTGGTATTGAGTGTCTGCGTTT
CCTCAATGAAATCATCTCAGATTTTGACTCTCTCCTGGACAATCCCAAGT
TCCGGGTGATCACCAAGATCAAAACCATTGGCAGCACGTATATGGCGGCT
TCAGGAGTCACCCCCGATGTCAACACCAATGGCTTTGCCAGCTCCAACAA
GGAAGACAAGTCCGAGAGAGAGCGCTGGCAGCACCTGGCTGACCTGGCCG
ACTTCGCGCTGGCCATGAAGGATACGCTCACCAACATCAACAACCAGTCC
TTCAATAACTTCATGCTGCGCATAGGCATGAACAAAGGCGGGGTTCTGGC
TGGGGTCATCGGAGCCCGGAAACCACACTACGACATCTGGGGCAATACAG
TCAATGTAGCCAGCAGGATGGAGTCCACGGGGGTCATGGGCAACATTCAG
GTGGTAGAAGAAACCCAAGTCATCCTCCGAGAGTACGGCTTCCGCTTTGT
GAGGCGAGGCCCCATCTTTGTGAAGGGGAAGGGGGAGCTGCTGACCTTCT
TCTTGAAGGGGCGGGATAAGCTAGCCACCTTCCCCAATGGCCCCTCTGTC
ACACTGCCCCACCAGGTGGTGGACAACTCCTGAATGGCCTCGAGCCTGAA
ACAGTCCAAACCGGAAGGGAGAATTTATTTTTTGAAACTGAAGGAAGTC
CCGACCTTCCTGGATTGAAGTGCACACTCATGGACTTTAGGTTTAGAAAC
CTCCTCAGCCTTCATTTGTTCGTGGATGTGTGAGCTCTGAGGGTGGCCCT
GCTATTCCTCTGCGTGCCTGTAGTGTCCCCAGCATAGGGGTCTTAGGCAT
AGGGCTGAACAGTCCTTCCAGAGCCCTCGTTCCAATCCCTGCCGTCCTTG
CCCCTGAGGGGCCCTGACCACTGTGAGCAGGAGGGTGGCAGAGCTGGGAC
AAAGCTGCCTTTGCCGCTGGGCTTTCCGGGACTGTGGAGGGAGCACAGGC
GGGGAAGCTCCACTTCAGACAGGGCTTGGTGGGGCAGGACATGGCTCCCA
TTTTGAAGGGAGGTCTCCATGTGGTCCGAGTGAGGTGAGACGGCCCTCGT
CCTGGTGTTCCTGATCATCTTGAAAGGTTCTTCTGGAACTCCTGTCCCCT
TAGTCATGAGAACAGAAAGTGCAATATTTCCTTTCACCTGGCCC

The NOV5 nucleic acid was identified on the p22-p24 region of chromosome 2 and has 2489 of 2526 bases (98%) identical to a gb:GENBANK-ID:AF033861|acc:AF033861.1 mRNA from Homo sapiens (Homo sapiens type III adenylyl cyclase (AC-III) mRNA, complete cds) (E=0.0).

A disclosed NOV5 polypeptide (SEQ ID NO: 22) encoded by SEQ ID NO: 21 is 761 amino acid residues and is presented using the one-letter code in Table 5B. Signal P, Psort and/or Hydropathy results predict that NOV5 has no signal peptide and is likely to be localized in the plasma membrane with a certainty of 0.6000. In other embodiments, NOV5 may also be localized to the Golgi body with acertainty of 0.4000, the endoplasmic reticulum with a certainty of 0.3000, or the mitochondrial inner membrane with a certainty of 0.0300.

TABLE 5B
Encoded NOV5 protein sequence (SEQ ID NO:22)

MPRNQGFSEPEYSAEYSAEYSVSLPSDPDRGVGRTHEISVRNSGSCLCLP
RFMRRGSAGSSPRARRALPPQPARGPSRSTRWPSSRTVLADQCYLCSGNP
LCVPAGGGLPFAPDKKRPSALPRSQRALRGGPVQSSSSKSSSPALIETKE
PNGSAHSSGSTSEKPEEQDAQADNPSFPNPRRRLRLQDLADRVVDASEDE
HELNQLLNEALLERESAQVVKKRNTFLLSMRFMDPEMETRYSVEKEKQSG
AAFSCSCVVLLCTALVEILIDPWLMTNYVTFMVGEILLLILTICSLAAIF
PRAFPKKLVAFSTWIDRTRWARNTWAMLAIFILVMANVVDMLSCLQYYTG
PSNATAGMETEGSCLENPKYYNYVAVLSLIATIMLVQVSHMVKLTLMLLV
AGAVATINLYAWRPVFDEYDHKRFREHDLPMVALEQMQGFNPGLNGTDRL
PLVPSKYSMTVMVFLMMLSFYYFSRHVEKLARTLFLWKIEVHDQKERVYE
MRRWNEALVTNMLPEHVARHFLGSKKRDEELYSQTYDEIGVMFASLPNFA
DFYTEESINNGGIECLRFLNEIISDFDSLLDNPKFRVITKIKTIGSTYMA
ASGVTPDVNTNGFASSNKEDKSERERWQHLADLADFALAMKDTLTNINNQ
SFNNFMLRIGMNKGGVLAGVIGARKPHYDIWGNTVNVASRMESTGVMGNI
QVVEETQVILREYGFRFVRRGPIFVKGKGELLTFFLKGRDKLATFPNGPS
VTLPHQVVDNS

The disclosed NOV5 amino acid sequence has 628 of 641 amino acid residues (97%) identical to, and 632 of 641 amino acid residues (98%) similar to, the 1144 amino acid residue ptnr:SPTREMBL-ACC:060266 protein from Homo sapiens (Human) (Type III ADENYLYL Cyclase (KIAA0511 Protein)) (E=0.0).

NOV5 is expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. This information was derived by determining the tissue sources of the sequences that were included in the invention including but not limited to SeqCalling sources, Public EST sources, and/or RACE sources.

In addition, the sequence is predicted to be expressed in human islet, brain, liver, and lung because of the expression pattern of (GENBANK-ID: gb:GENBANK-ID:AF033861|acc:AF033861.1) a closely related Homo sapiens type III adenylyl cyclase (AC-III) mRNA, complete cds homolog.

NOV5 also has homology to the amino acid sequences shown in the BLASTP data listed in Table 5C.

TABLE 5C
BLAST results for NOV5

| Gene Index/ | | Length | Identity | Positives | |
|---|---|---|---|---|---|
| Identifier | Protein/Organism | (aa) | (%) | (%) | Expect |
| gi\|117787\|sp\|P21932\| | ADENYLATE CYCLASE | 1144 | 549/648 | 574/648 | 0.0 |
| CYA3_RAT | TYPE III | | (84%) | (87%) | |
| | (ADENYLATE | | | | |
| | CYCLASE, | | | | |
| | OLFACTIVE TYPE) | | | | |
| | (ATP | | | | |
| | PYROPHOSPHATELYASE) | | | | |
| | (ADENYLYL | | | | |
| | CYCLASE) (AC-III) | | | | |
| | (AC3) | | | | |
| gi\|4757724\|ref\|NP_{—} | adenylate cyclase | 1144 | 588/619 | 588/619 | 0.0 |
| 004027.1\| | 3; adenylyl | | (94%) | (94%) | |
| (NM_004036) | cyclase, type III; | | | | |
| | ATP | | | | |
| | pyrophosphatelyase | | | | |
| | [Homo sapiens] | | | | |
| gi\|7437177\|pir\| | adenylate cyclase | 1167 | 216/574 | 324/574 | 4e−99 |
| \|T13927 | (EC 4.6.1.1) | | (37%) | (55%) | |
| | isoform 39E - | | | | |
| | fruit fly | | | | |
| | (Drosophila melanogaster) | | | | |
| gi\|7302124\|gb\| | Ac3 gene product | 1167 | 216/574 | 324/574 | 5e−99 |
| AAF57223.1\| | [Drosophila melanogaster] | | (37%) | (55%) | |
| (AE003781) | | | | | |
| gi\|6752978\|ref\|NP_{—} | adenylate cyclase | 1249 | 199/536 | 307/536 | 3e−91 |
| 033753.1\| | 8 [Mus musculus] | | (37%) | (57%) | |
| (NM_009623) | | | | | |

The homology of these sequences is shown graphically in the ClustalW analysis shown in Table 5D.

[image]

[image]

[image]

Tables 5E-F list the domain description from DOMAIN analysis results against NOV5. This indicates that the NOV5 sequence has properties similar to those of other proteins known to contain this domain.

TABLE 5E
Domain Analysis of NOV5

gnl|Pfam|pfam00211, guanylate_cyc, Adenylate and Guanylate cyclase
catalytic domain. (SEQ ID NO:90)
CD-Length=185 residues, 100.0% aligned
Score=204 bits (518), Expect=2e−53

| | | | |
|---|---|---|---|
| Query: | 531 | LYSQTYDEIGVMFASLPNFADFYTEESINNGGIECLRFLNEIISDFDSLLDNPKFRVITK | 590 |
| | | +\|++ \|\|\|+ ++\|\| + \| \| \| +\| \|\|\|+ + \|\| \|+\| \| | |
| Sbjct: | 1 | VYAERYDEVTILFADIVGFTALSERHSP----EEVVRLLNELFTRFDELVDAHG---GYK | 53 |
| Query: | 591 | IKTIGSTYMAASGVTPDVNTNGFASSNKEDKSERERWQHLADLADFALAMKDTLTNINNQ | 650 |
| | | +\|\|\|\| \|\|\|\|\|\|+ \| \| \| \|\|\|\|\|\|\|\| + \| +\| | |
| Sbjct: | 54 | VKTIGDAYMAASGLPPA------------------SAAHAAKLADFALAMVEALEEVNVG | 95 |
| Query: | 651 | SFNNFMLRIGMNKGGVLAGVIGARKPHYDIWGNTVNVASRMESTGVMGNIQVVEETQVIL | 710 |
| | | \|\|\|\|++ \| \|+\|\|\|\|\|\|++\| \|\|+\|\|+\|\|\|\|\|\|\|\|\|\| \|\| \| \| \| \| \| +\| | |
| Sbjct: | 96 | HTEPLRLRIGIHTGPVVAGVIGAKRPRYDVWGDTVNVASRMESLGPGKIHVSESTYRLL | 155 |
| Query: | 711 | -REYGFRF-VRRGPIFVKGKGE-LLTFFLK | 737 |
| | | \|+\| \|\| + \|\|\|\|\|+ + \|+\|\| | |
| Sbjct: | 156 | NGLESFQFRFPRGEVSVKGKGKPMKTYFLH | 185 |

TABLE 5F
Domain Analysis of NOV5

gnl|Smart|smart00044, CYCc, Adenylyl-/guanylyl cyclase, catalytic
domain; Present in two copies in mammalian adenylyl cyclases.
Eubacterial homologues are known. Two residues (Asn, Arg) are thought
to be involved in catalysis. These cyclases have important roles in a
diverse range of cellular processes. (SEQ ID NO:91)
CD-Length=194 residues, 99.5% aligned
Score=174 bits (442), Expect=1e−44

| | | | |
|---|---|---|---|
| Query: | 500 | EMRRWNEALVTNMLPEHVARHFLGSKKRDEELYSQTYDEIGVMFASLPNFADFYTEESIN | 559 |
| | | \| +\| \|+ \|+ + \|\| \|\| \| + + +\|\|\|+ ++\| + \| + | |
| Sbjct: | 1 | EEKRKNDRLLDQLLPASVAESLKRGG---EPVPAPSYDEVTILFTDIVGFTALSSA---- | 53 |
| Query: | 560 | NGGIECLRFLNEIISDFDSLLDNPKFRVITKIKTIGSTYMAASGVTPDVNTNGFASSNKE | 619 |
| | | + + \|\|++ \| \|\| ++\| \|+\|\|\|\| \|\| \|\|+ | |
| Sbjct: | 54 | ATPEQVVTLLNDLYSRFDRIIDRHG---GYKVKTIGDAYMVVSGLPTAAL---------- | 100 |
| Query: | 620 | DKSERERWQHLADLADFALAMKDTLTNINNQ-SFNNFMLRIGMNKGGVLAGVIGARKPHY | 678 |
| | | \|\| \| \|\| \| ++\| + \| \| +\|\|\|++ \| \|+\|\|\|+\| \| \| | |
| Sbjct: | 101 | -------VQHAELAALEALDMVESLKTVLVQHRGNGLRVRIGIHTGPVVAGVVGITMPRY | 153 |
| Query: | 679 | DIWGNTVNVASRMESTGVMGNIQVVEETQVILREYGFRFV | 718 |
| | | ++\|+\|\|\|+\|\|\|\|\|\| \| \| \|\|\| \|\|\| +\|\| +\| | |
| Sbjct: | 154 | CLFGDTVNLASRMESVGDPGQIQVSEETYSLLRRRSGQFE | 193 |

Adenylyl cyclase (AC) is an enzyme that synthesizes cyclic adenosine monophosphate or cyclic AMP from adenosine triphosphate (ATP), an important player of some intracellular signaling pathways. Adenylyl cyclases are integral membrane proteins that consist of two bundles of six transmembrane segments and two catalytic domains extending as loops into the cytoplasm. There are at least nine isoforms of adenylyl cyclase, based on cloning of full-length cDNAs. These enzymes differ considerably in regulatory properties and are differentially expressed among tissues. Recently, type 3 adenylyl cyclase (AC-III) overexpression has been implicated in reversing the defect of spontaneous diabetics in Goto-Kakizaki (GK) rat. More recently, cDNA of the human AC-III homologue has been cloned with an open reading frame encoding 1144 amino acids containing 12 transmembrane-spanning domains. Human AC-III gene shows 95% homology with the rat sequence and is widely expressed in different tissues (Busfield et al., 2000, Genomics vol. 66: 213-216; Yang et al., 1999, Biochem Biophy Res commun, vol. 254: 548-551).

The disclosed NOV5 nucleic acid of the invention encoding a Type m adenylyl cyclase-like protein includes the nucleic acid whose sequence is provided in Table 5A or a fragment thereof. The invention also includes a mutant or variant nucleic acid any of whose bases may be changed from the corresponding base shown in Table 5A while still encoding a protein that maintains its Type III adenylyl cyclase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2% percent of the bases may be so changed.

The disclosed NOV5 protein of the invention includes the Type III adenylyl cyclase-like protein whose sequence is provided in Table 5B. The invention also includes a mutant or variant protein any of whose residues may be changed from the corresponding residue shown in Table 5B while still encoding a protein that maintains its Type III adenylyl cyclase-like activities and physiological functions, or a functional fragment thereof. In the mutant or variant protein, up to about 63% percent of the residues may be so changed.

The NOV5 nucleic acids and proteins of the invention are useful in potential therapeutic applications implicated in diabetes, heart failure, neurological diseases such as epilepsy, sleep disorder, parkinsonism, Huntington's disease, Alzheimer's disease, depression, schizophrenia, and/or other diseases, disorders and conditions of the like. The NOV5 nucleic acid, or fragments thereof, may further be useful in diagnostic applications, wherein the presence or amount of the nucleic acid or the protein are to be assessed.

NOV5 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV5 protein have multiple hydrophilic regions, each of which can be used as an immunogen. In one embodiment, contemplated NOV5 epitope is from about amino acids 5 to 270. In other embodiments, NOV5 epitope is from about amino acids 400 to 450, and from about amino acids 470 to 770. This novel protein also has value in development of powerful assay system for functional analysis of various human disorders, which will help in understanding of pathology of the disease and development of new drug targets for various disorders.

NOV6

NOV6 includes three novel Airway Trypsin-Like Protease-like proteins disclosed below. The disclosed sequences have been named NOV6a, NOV6b, and NOV6c.

NOV6a

A disclosed NOV6a nucleic acid of 1769 nucleotides (also referred to as CG50389-02) encoding a novel Interleukin 1 receptor related protein-like protein is shown in Table 6A. An open reading frame was identified beginning with an ATG initiation codon at nucleotides 386-388 and ending with a TAG codon at nucleotides 1619-1621. A putative untranslated region upstream from the initiation codon and downstream from the termination codon is underlined in Table 6A, and the start and stop codons are in bold letters.

TABLE 6A
NOV6a Nucleotide Sequence (SEQ ID NO:23)

CGCCCGCCCACGGCGGCGGGGAAATACCTAGGCATGGAAGTGGCATGACA
GGGCTCGTGTCCCTGTCATATTTTCCACTCTCCACGAGGTCCTGCGCGCT
TCAATCCTGCAGGCAGCCCGGTTTGGGGATGTGGTCCTTGCTGCTCTGCG
GGTTGTCCATCGCCCTTCCACTGTCTGTCACAGCAGATGGATGCAAGGAC
ATTTTTATGAAAAATGAGATACTTTCAGCAAGCCAGCCTTTTGCTTTTAA
TTGTACATTCCCTCCCATAACATCTGGGGAAGTCAGTGTAACATGGTATA
AAAATTCTAGCAAAATCCCAGTGTCCAAAATCATACAGTCTAGAATTCAC
CAGGACGAGACTTGGATTTTGTTTCTCCCCATGGAATGGGGGGACTCAGG
AGTCTACCAATGTGTTATAAAGACTGTAACGAGATTAAAGGGGAGCGGTT
CACTGTTTTGGAAACCAGGCTTTTGGTGAGCAATGTCTCGGCAGAGGACA
GAGGGAACTACGCGTGTCAAGCCATACTGACACACTCAGGGAAGCAGTAC
GAGGTTTTAAATGGCATCACTGTGAGCATTACAGAAAGAGCTGGATATGG
AGGAAGTGTCCCTAAAATCATTTATCCAAAAAATCATTCAATTGAAGTAC
AGCTTGGTACCACTCTGATTGTGGACTGCAATGTAACAGACACCAAGGAT
AATACAAATCTACGATGCTGGAGAGTCAATAACACTTTGGTGGATGATTA
CTATGATGAATCCAAACGAATCAGAGAAGGGGTGGAAACCCATGTCTCTT
TTCGGGAACATAATTTGTACACAGTAAACATCACCTTCTTGGAAGTGAAA
ATGGAAGATTATGGCCTTCCTTTCATGTGCCACGCTGGAGTGTCCACAGC
ATACATTATATTACAGCTCCCAGCTCCGGATTTTCGAGCTTACTTGATAG
GAGGGCTTATCGCCTTGGTGGCTGTGGCTGTGTCTGTTGTGTACATATAC
AACATTTTTAAGATCGACATTGTTCTTTGGTATCGAAGTGCCTTCCATTC
TACAGAGACCATAGTAGATGGGAAGCTGTATGACGCCTATGTCTTATACC
CCAAGCCCCACAAGGAAAGCCAGAGGCATGCCGTGGATGCCCTGGTGTTG
AATATCCTGCCCGAGGTGTTGGAGAGACAATGTGGATATAAGTTGTTTAT
ATTCGGCAGAGATGAATTCCCTGGACAAGCCGTGGCCAATGTCATCGATG
AAAACGTTAAGCTGTGCAGGAGGCTGATTGTCATTGTGGTCCCCGAATCG
CTGGGCTTTGGCCTGTTGAAGAACCTGTCAGAAGAACAAATCGCGGTCTA
CAGTGCCCTGATCCAGGACGGGATGAAGGTTATTCTCATTGAGCTGGAGA
AAATCGAGGACTACACAGTCATGCCAGAGTCAATTCAGTACATCAAACAG
AAGCATGGTGCCATCCGGTGGCATGGGGACTTCACGGAGCAGTCACAGTG
TATGAAGACCAAGTTTTGGAAGACAGTGAGATACCACATGCCGCCCAGAA
GGTGTCGGCCGTTTCTCCGGTCCACGTGCCGCAGCACACACCTCTGTACC
GCACCGCAGGCCCAGAACTAGGCTCAAGAAGAAAGAAGTGTACTCTCACG
ACTGGCTAAGACTTGCTGGACTGACACCTATGGCTGGAAGATGACTTGTT
TTGCTCCATGTCTCCTCATTCCTACACCTATTTTCTGCTGCAGGATGAGG
CTAGGGTTAGCATTCTAGA

The disclosed NOV6a nucleic acid sequence, located on the q12 region of chromosome 2, has 1363 of 1370 bases (99%) identical to a gb:GENBANK-ID:HSU49065|acc:U49065.1 mRNA from Homo sapiens (Human interleukin-1 receptor-related protein mRNA, complete cds) (E=7.0e⁻³⁰¹).

A disclosed NOV6a polypeptide (SEQ ID NO: 24) encoded by SEQ ID NO: 23 is 411 amino acid residues and is presented using the one-letter amino acid code in Table 6B. Signal P, Psort and/or Hydropathy results predict that NOV6a contains no signal peptide and is likely to be localized in the plasma membrane with a certainty of 0.7300. In other embodiments, NOV6A is also likely to be localized to the endoplasmic reticulum (membrane) with a certainty of 0.2000, or to the mitochondrial inner membrane with a certainty of 0.1000

TABLE 6B
Encoded NOV6a protein sequence (SEQ ID NO:24).

MGGLRSLPMCYKDCNEIKGERFTVLETRLLVSNVSAEDRGNYACQAILTH
SGKQYEVLNGITVSITERAGYGGSVPKIIYPKNHSIEVQLGTTLIVDCNV
TDTKDNTNLRCWRVNNTLVDDYYDESKRIREGVETHVSFREHNLYTVNIT
FLEVKMEDYGLPFMCHAGVSTAYIILQLPAPDFRAYLIGGLIALVAVAVS
VVYIYNIFKIDIVLWYRSAFHSTETIVDGKLYDAYVLYPKPHKESQRHAV
DALVLNILPEVLERQCGYKLFIFGRDEFPGQAVANVIDENVKLCRRLIVI
VVPESLGFGLLKNLSEEQIAVYSALIQDGMKVILIELEKIEDYTVMPESI
QYIKQKHGAIRWHGDFTEQSQCMKTKFWKTVRYHMPPRRCRPFLRSTCRS
THLCTAPQAQN

The disclosed NOV6a amino acid sequence has 401 of 401 amino acid residues (100%) identical to, and 401 of 401 amino acid residues (100%) similar to, the 562 amino acid residue ptnr:SPTREMBL-ACC:Q13525 protein from Homo sapiens (Human) (Interleukin-1 Receptor-Related Protein) (E=3.8e⁻²¹⁸).

NOV6a is expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. This information was derived by determining the tissue sources of the sequences that were included in the invention including but not limited to SeqCalling sources, Public EST sources, and/or RACE sources.

NOV6b

A disclosed NOV6b nucleic acid of 1827 nucleotides (also referred to as CG50389-03) encoding a novel Interleukin 1 receptor related protein-like protein is shown in Table 6C. An open reading frame was identified beginning with an ATG initiation codon at nucleotides 65-67 and ending with a TAA codon at nucleotides 1715-1717. A putative untranslated region upstream from the initiation codon and downstream from the termination codon is underlined in Table 6C, and the start and stop codons are in bold letters.

TABLE 6C
NOV6b Nucleotide Sequence (SEQ ID NO:25)

GTCATATTTTCCACTCTCCACGAGGTCCTGCGCGCTTCAATCCTGCAGGC
AGCCCGGTTTGGGGATGTGGTCCTTGCTGCTCTGCGGGTTGTCCATCGCC
CTTCCACTGTCTGTCACAGCAGATGGATGCAAGGACATTTTTATGAAAAA
TGAGATACTTTCAGCAAGCCAGCCTTTTGCTTTTAATTGTACATTCCCTC
CCATAACATCTGGGGAAGTCAGTGTAACATGGTATAAAAATTCTAGCAAA
ATCCCAGTGTCCAAAATCATACAGTCTAGAATTCACCAGGACGAGACTTG
GATTTTGTTTCTCCCCATGGAATGGGGGGACTCAGGAGTCTACCAATGTG
TTATAAAGGGTAGAGACAGCTGTCATAGAATACATGTAAACCTAACTGTT
TTTGAAAAACATTGGTGTGACACTTCCATAGGTGGTTTACCAAATTTATC
AGATGAGTACAAGCAAATATTACATCTTGGAAAAGATGATAGTCTCACAT
GTCATCTGCACTTCCCGAAGAGTTGTGTTTTGGGTCCAATAAAGTGGTAT
AAAGACTGTAACGAGATTAAAGGGGAGCGGTTCACTGTTTTGGAAACCAG
GCTTTTGGTGAGCAATGTCTCGGCAGAGGACAGAGGGAACTACGCGTGTC
AAGCCATACTGACACACTCAGGGAAGCAGTACGAGGTTTTAAATGGCATC
ACTGTGAGCATTAGTACCACTCTGATTGTGGACTGCAATGTAACAGACAC
CAAGGATAATACAAATCTACGATGCTGGAGAGTCAATAACACTTTGGTGG
ATGATTACTATGATGAATCCAAACGAATCAGAGAAGGGGTGGAAACCCAT
GTCTCTTTTCGGGAACATAATTTGTACACAGTAAACATCACCTTCTTGGA
AGTGAAAATGGAAGATTATGGCCTTCCTTTCATGTGCCACGCTGGAGTGT
CAACAGCATACATTATATTACAGCTCCCAGCTCCGGATTTTCGAGCTTAC
TTGATAGGAGGGCTTATCGCCTTGGTGGCTGTGGCTGTGTCTGTTGTGTA
CATATACAACATTTTTAAGATCGACATTGTTCTTTGGTATCGAAGTGCCT
TCCATTCTACAGAGACCATAGTAGATGGGAAGCTGTATGACGCCTATGTC
TTATACCCCAAGCCCCACAAGGAAAGCCAGAGGCATGCCGTGGATGCCCT
GGTGTTGAATATCCTGCCCGAGGTGTTGGAGAGACAATGTGGATATAAGT
TGTTTATATTCGGCAGAGATGAATTCCCTGGACAAGCCGTGGCCAATGTC
ATCGATGAAAACGTTAAGCTGTGCAGGAGGCTGATTGTCATTGTGGTCCC
CGAATCGCTGGGCTTTGGCCTGTTGAAGAACCTGTCAGAAGAACAAATCG
CGGTCTACAGTGCCCTGATCCAGGACGGGATGAAGGTTATTCTCGTTGAG
CTGGAGAAAATCGAGGACTACACAGTCATGCCAGAGTCAATTCAGTACAT
CAAACAGAAGCATGGTGCCATCCGGTGGCATGGGGACTTCACGGAGCAGT
CACAGTGTATGAAGACCAAGTTTTGGAAGACAGTGAGATACCACATGCCA
CCCAGAAGGTGTCGGCCGTTTCCTCCGGTCCAGCTGCTGCAGCACACACC
TTGCTGCCGCACCGCAGGCCCAGAACTAGGCTCAAGAAGAAAGAAGTGTA
CTCTCACGACTGGCTAAGACTTGCTGGACTGACACCTATGGCTGGAAGAT
GACTTGTTTTGCTCCATGTCTCCTCATTCCTACACCTATTTTCTGCTGCA
GGATGAGGCTAGGGTTAGCATTCTAGA

The disclosed NOV6b nucleic acid sequence, located on the p12 region of chromosome 2, has 1118 of 1121 bases (99%) identical to a gb:GENBANK-ID:AF284434|acc:AF284434.1 mRNA from Homo sapiens (Homo sapiens IL-1Rrp2 mRNA, complete cds) (E=0.0).

A disclosed NOV6b polypeptide (SEQ ID NO: 26) encoded by SEQ ID NO: 25 is 550 amino acid residues and is presented using the one-letter amino acid code in Table 6D. Signal P, Psort and/or Hydropathy results predict that NOV6b contains a signal peptide and is likely to be localized in the plasma membrane with a certainty of 0.4600. In other embodiments, NOV6B is also likely to be localized to the endoplasmic reticulum (membrane) with a certainty of 0.1000, the endoplasmic reticulum (lumen) with a certainty of 0.1000, or extracellularly with a certainty of 0.1000. The most likely cleavage site for NOV6b is between positions 19 and 20: VTA-DG.

TABLE 6D
Encoded NOV6b protein sequence (SEQ ID NO:26).

MWSLLLCGLSTALPLSVTADGCKDIFMKNEILSASQPFAFNCTFPPITSG
EVSVTWYKNSSKIPVSKIIQSRIHQDETWILFLPMEWGDSGVYQCVIKGR
DSCHRIHVNLTVFEKHWCDTSIGGLPNLSDEYKQILHLGKDDSLTCHLHF
PKSCVLGPIKWYKDCNEIKGERFTVLETRLLVSNVSAEDRGNYACQAILT
HSGKQYEVLNGITVSISTTLIVDCNVTDTKDNTNLRCWRVNNTLVDDYYD
ESKRIREGVETHVSFREHNLYTVNITFLEVKMEDYGLPFMCHAGVSTAYI
ILQLPAPDFRAYLIGGLIALVAVAVSVVYIYNIFKIDIVLWYRSAFHSTE
TIVDGKLYDAYVLYPKPHKESQRHAVDALVLNILPEVLERQCGYKLFIFG
RDEFPGQAVANVIDENVKLCRRLIVIVVPESLGFGLLKNLSEEQIAVYSA
LIQDGMKVILVELEKIEDYTVMPESIQYIKQKHGAIRWHGDFTEQSQCMK
TKFWKTVRYHMPPRRCRPFPPVQLLQHTPCCRTAGPELGSRRKKCTLTTG

The disclosed NOV6b amino acid sequence has 336 of 345 amino acid residues (97%) identical to, and 338 of 345 amino acid residues (97%) similar to, the 575 amino acid residue ptnr:TREMBLNEW-ACC:AAG21368 protein from Homo sapiens (Human) (IL-1RRP2) (E=1.7e⁻³⁰⁴).

NOV6b is expressed in at least the following tissues: amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the sequence of NOV6b.

NOV6c

A disclosed NOV6c nucleic acid of 1897 nucleotides (also referred to as CG50389-04) encoding a novel Interleukin 1 receptor related protein-like protein is shown in Table 6E. An open reading frame was identified beginning with an ATG initiation codon at nucleotides 51-53 and ending with a TAA codon at nucleotides 1785-1787. A putative untranslated region upstream from the initiation codon and downstream from the termination codon is underlined in Table 6E, and the start and stop codons are in bold letters.

TABLE 6E
NOV6c Nucleotide Sequence (SEQ ID NO:27)

GAATTCCGCCCGCCCACGGCGGCGGGGAAATACCTAGGCATGGAAGTGGC
ATGACAGGGCTCGTGTCCCTGTCATATTTTCCACTCTCCACGAGGTCCTG
CGCGCTTCAATCCTGCAGGCAGCCCGGTTTGGGGATGTGGTCCTTGCTGC
TCTGCGGGTTGTCCATCGCCCTTCCACTGTCTGTCACAGCAGATGGATGC
AAGGACATTTTTATGAAAAATGAGATACTTTCAGCAAGCCAGCCTTTTGC
TTTTAATTGTACATTCCCTCCCATAACATCTGGGGAAGTCAGTGTAACAT
GGTATAAAAATTCTAGCAAAATCCCAGTGTCCAAAATCATACAGTCTAGA
ATTCACCAGGACGAGACTTGGATTTTGTTTCTCCCCATGGAATGGGGGGA
CTCAGGAGTCTACCAATGTGTTATAAAGGGTAGAGACAGCTGTCATAGAA
TACATGTAAACCTAACTGTTTTTGAAAAACATTGGTGTGACACTTCCATA
GGTGGTTTACCAAATTTATCAGATGAGTACAAGCAAATATTACATCTTGG
AAAAGATGATAGTCTCACATGTCATCTGCACTTCCCGAAGAGTTGTGTTT
TGGGTCCAATAAAGTGGTATAAAGACTGTAACGAGATTAAAGGGGAGCGG
TTCACTGTTTTGGAAACCAGGCTTTTGGTGAGCAATGTCTCGGCAGAGGA
CAGAGGGAACTACGCGTGTCAAGCCATACTGACACACTCAGGGAAGCAGT
ACGAGGTTTTAAATGGCATCACTGTGAGCATTAGTACCACTCTGATTGTG
GACTGCAATGTAACAGACACCAAGGATAATACAAATCTACGATGCTGGAG
AGTCAATAACACTTTGGTGGATGATTACTATGATGAATCCAAACGAATCA
GAGAAGGGGTGGAAACCCATGTCTCTTTTCGGGAACATAATTTGTACACA
GTAAACATCACCTTCTTGGAAGTGAAAATGGAAGATTATGGCCTTCCTTT
CATGTGCCACGCTGGAGTGTCAACAGCATACATTATATTACAGCTCCCAG
CTCCGGATTTTCGAGCTTACTTGATAGGAGGGCTTATCGCCTTGGTGGCT
GTGGCTGTGTCTGTTGTGTACATATACAACATTTTTAAGATCGACATTGT
TCTTTGGTATCGAAGTGCCTTCCATTCTACAGAGACCATAGTAGATGGGA
AGCTGTATGACGCCTATGTCTTATACCCCAAGCCCCACAAGGAAAGCCAG
AGGCATGCCGTGGATGCCCTGGTGTTGAATATCCTGCCCGAGGTGTTGGA
GAGACAATGTGGATATAAGTTGTTTATATTCGGCAGAGATGAATTCCCTG
GACAAGCCGTGGCCAATGTCATCGATGAAAACGTTAAGCTGTGCAGGAGG
CTGATTGTCATTGTGGTCCCCGAATCGCTGGGCTTTGGCCTGTTGAAGAA
CCTGTCAGAAGAACAAATCGCGGTCTACAGTGCCCTGATCCAGGACGGGA
TGAAGGTTATTCTCGTTGAGCTGGAGAAAATCGAGGACTACACAGTCATG
CCAGAGTCAATTCAGTACATCAAACAGAAGCATGGTGCCATCCGGTGGCA
TGGGGACTTCACGGAGCAGTCACAGTGTATGAAGACCAAGTTTTGGAAGA
CAGTGAGATACCACATGCCACCCAGAAGGTGTCGGCCGTTTCCTCCGGTC
CAGCTGCTGCAGCACACACCTTGCTGCCGCACCGCAGGCCCAGAACTAGG
CTCAAGAAGAAAGAAGTGTACTCTCACGACTGGCTAAGACTTGCTGGACT
GACACCTATGGCTGGAAGATGACTTGTTTTGCTCCATGTCTCCTCATTCC
TACACCTATTTTCTGCTGCAGGATGAGGCTAGGGTTAGCATTCTAGA

The disclosed NOV6c nucleic acid sequence, located on the p12 region of chromosome 2, has 1118 of 1121 bases (99%) identical to a gb:GENBANK-ID:AF284434|acc:AF284434.1 mRNA from Homo sapiens (Homo sapiens IL-1Rrp2 mRNA, complete cds) (E=0.0).

A disclosed NOV6c polypeptide (SEQ ID NO: 28) encoded by SEQ ID NO: 27 is 578 amino acid residues and is presented using the one-letter amino acid code in Table 6F. Signal P, Psort and/or Hydropathy results predict that NOV6c contains a signal peptide and is likely to be localized in the mitochondrial inner membrane with a certainty of 0.8546. In other embodiments, NOV6c is also likely to be localized to the plasma membrane with a certainty of 0.6000, the Golgi body with a certainty of 0.4000, or in the mitochondrial inner membrane space with a certainty of 0.3386. The most likely cleavage site for NOV6c is between positions 47 and 48: VTA-DG.

TABLE 6F
Encoded NOV6c protein sequence (SEQ ID NO:28).

MTGLVSLSYFPLSTRSCALQSCRQPGLGMWSLLLCGLSTALPLSVTADGC
KDIFMKNEILSASQPFAFNCTFPPITSGEVSVTWYKNSSKIPVSKIIQSR
IHQDETWILFLPMEWGDSGVYQCVIKGRDSCHRIHVNLTVFEKHWCDTSI
GGLPNLSDEYKQILHLGKDDSLTCHLHFPKSCVLGPIKWYKDCNEIKGER
FTVLETRLLVSNVSAEDRGNYACQAILTHSGKQYEVLNGITVSISTTLIV
DCNVTDTKDNTNLRCWRVNNTLVDDYYDESKRIREGVETHVSFREHNLYT
VNITFLEVKMEDYGLPFMCHAGVSTAYIILQLPAPDFRAYLIGGLIALVA
VAVSVVYIYNIFKIDIVLWYRSAFHSTETIVDGKLYDAYVLYPKPHKESQ
RHAVDALVLNILPEVLERQCGYKLFIFGRDEFPGQAVANVIDENVKLCRR
LIVIVVPESLGFGLLKNLSEEQIAVYSALIQDGMKVILVELEKIEDYTVM
PESIQYIKQKHGAIRWHGDFTEQSQCMKTKFWKTVRYHMPPRRCRPFPPV
QLLQHTPCCRTAGPELGSRRKKCTLTTG

The disclosed NOV6c amino acid sequence has 336 of 345 amino acid residues (97%) identical to, and 338 of 345 amino acid residues (97%) similar to, the 575 amino acid residue ptnr:TREMBLNEW-ACC:AAG21368 protein from Homo sapiens (Human) (IL-IRRP2) (E=1.7e⁻³⁰⁴).

NOV6c is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the sequence of NOV6c.

NOV6a also has homology to the amino acid sequences shown in the BLASTP data listed in Table 6G.

TABLE 6G
BLAST results for NOV6a

| Gene Index/ | | Length | Identity | Positives | |
|---|---|---|---|---|---|
| Identifier | Protein/Organism | (aa) | (%) | (%) | Expect |
| gi\|4504663\|ref\|NP_{—} | interleukin 1 | 562 | 382/401 | 382/401 | 0.0 |
| 003845.1\| | receptor-like 2 | | (95%) | (95%) | |
| (NM_003854) | [Homo sapiens] | | | | |
| gi\|13637728\|ref\|XP_{—} | similar to IL-1Rrp2 | 603 | 356/375 | 356/375 | 0.0 |
| 002685.3\| | (H. sapiens) | | (94%) | (94%) | |
| (XM_002685) | [Homo sapiens] | | | | |
| gi\|10644686\|gb\| | IL-1Rrp2 | 575 | 355/375 | 356/375 | 0.0 |
| AAG21368.1\| | [Homo sapiens] | | (94%) | (94%) | |
| AF284434_1 | | | | | |
| (AF284434) | | | | | |
| gi\|1236081\|gb\| | interleukin-1 | 561 | 262/380 | 304/380 | e−155 |
| AAB53238.1\| | receptor-related | | (68%) | (79%) | |
| (U49066) | protein | | | | |
| | [Rattus norvegicus] | | | | |
| gi\|10644684\|gb\| | IL-1Rrp2 [Mus musculus] | 574 | 262/380 | 301/380 | e−153 |
| AAG21367.1\| | | | (68%) | (78%) | |
| AF284433_1 | | | | | |
| (AF284433) | | | | | |

The homology of these sequences is shown graphically in the ClustalW analysis shown in Table 6H.

[image]

[image]

[image]

Tables 61-J list the domain description from DOMAIN analysis results against NOV6. This indicates that the NOV6 sequence has properties similar to those of other proteins known to contain this domain.

TABLE 61
Domain Analysis of NOV6
gnl|Pfam|pfam01582, TIR, TIR domain. The TIR domain is an
intracellular signaling domain found in MyD88, interleuicin 1 receptor
and the Toll receptor. Called TIR (by SMART?) for Toll - Interleukin -
Resistance. (SEQ ID NO:97)
CD-Length = 141 residues, 100.0% aligned
Score = 128 bits (322), Expect = 6e−31

| | | | |
|---|---|---|---|
| Query: | 234 | AYVLYPKPHKESQRHAVDALVLNILPEVLERQCGYKLFIFGRDEFPGQAVANVIDENVKL | 293 |
| | | \|++ + \| \| ++\| \| \|\| + \| \|\|\|\| \|\|\| \|\|+++ + \| ++ | |
| Sbjct: | 1 | AFISFSGKDDR------DTFVSHLLKE-LEEKPGIKLFIDDRDELPGESILENLFEAIEK | 53 |
| Query: | 294 | CRRLIVIVVPESLGFGLLKNLSEEQIAVYSALIQDGMKVILIELEKIEDYTVMPESIQYI | 353 |
| | | \|\| \|\|\|+ \| \| \|\| \|\| \| \|\|\|\| \|++ \| +\| ++ | |
| Sbjct: | 54 | SRRAIVILSSNYASSSW--CLDELVEAVKLALEQGNKKVILPEFYKVDPSDVRKQSGKFG | 111 |
| Query: | 354 | KQKHGAIRWHGDFTEQSQCMRTKFWKTVRYHMPP 387 | |
| | | \| ++\| \|\| \| \| + +\|\|\| \| \|\| | |
| Sbjct: | 112 | KAFLKTLKWFGDKTSQ----RIRFWKKALYAMPV 141 | |

TABLE 6J
Domain Analysis of NOV6
gnl|Smart|smart00255, TIR, Toll - interleukin 1 - resistance (SEQ ID NO:98)
CD-Length = 140 residues, 99.3% aligned
Score = 102 bits (254), Expect = 4e−23

| | | | |
|---|---|---|---|
| Query: | 232 | YDAYVLYPKPHKESQRHAVDALVLNILPEVLERQCGYKLFIFGRDEFPGQAVANVIDENV | 291 |
| | | \|\| ++ \| + + \| +\|\|+ \|\|\|\| +\| \| \|\| \|\|\| + | |
| Sbjct: | 2 | YDVFISYSG---------DEDVRNEFLSHLLEQLRGYKLCVFIDDFEPGGGDLENIDEAI | 52 |
| Query: | 292 | KLCRRLIVIVVPESLGFGLLKNLSEEQIAVYSALIQDGMKVILIELEKI-EDYTVMPESI | 350 |
| | | + \| \|\|++ \| + \| \|+ +\|\| \| \|++\|\| \| \| \| \| \| \| | |
| Sbjct: | 53 | EKSRIAIVVLSPNYAESEWCLD--ELVAALENALEQGGLRVIPIFYEVIPSDVRKQPGSF | 110 |
| Query: | 351 | QYIKQKHGAIRWHGDFTEQSQCMKTKFWKTVRYHMPPR 388 | |
| | | + + +\|+ ++\| \| ++ \|\|\| \| +\| + | |
| Sbjct: | 111 | RKVFKKN-YLKWTEDEKDR-------FWKKALYAVPSK 140 | |

Interleukin-1 (IL-1) is a central regulator of the immune and inflammatory responses. Recently, a family of proteins have been described that share significant homology in their signaling domains with the Type I IL-1receptor (IL-1RI), which includes the IL-1receptor-related protein. The members of IL-1RI are clustered within 450 kb on human chromosome 2q and all of them are important in host responses to injury and infection. The remarkable conservation between diverse species indicates that the IL-1system represents an ancient signaling machine critical for responses to environmental stresses and attack by pathogens (O'Neill L. A., Greene, C., 1998, J. Leukoc Biol., vol. 63: 650-657, Busfield et al., 2000, Genomics vol. 66:213-216).

The disclosed NOV6 nucleic acid of the invention encoding a Interleukin 1 receptor related protein-like protein includes the nucleic acid whose sequence is provided in Table 6A, 6C, 6E or a fragment thereof. The invention also includes a mutant or variant nucleic acid any of whose bases may be changed from the corresponding base shown in Table 6A, 6C, or 6E while still encoding a protein that maintains its Interleukin 1 receptor related protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1% percent of the bases may be so changed.

The disclosed NOV6 protein of the invention includes the Interleukin 1 receptor related protein-like protein whose sequence is provided in Table 6B, 6D, or 6F. The invention also includes a mutant or variant protein any of whose residues may be changed from the corresponding residue shown in Table 6B, 6D, or 6F while still encoding a protein that maintains its Interleukin 1 receptor related protein-like activities and physiological functions, or a functional fragment thereof. In the mutant or variant protein, up to about 32% percent of the residues may be so changed.

The above defined information for this invention suggests that these Interleukin 1 receptor related protein-like proteins (NOV6) may function as a member of a “Interleukin 1 receptor related protein family”. Therefore, the NOV6 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those. defined here.

The nucleic acids and proteins of NOV6 are useful in any inflammatory diseases such as uveitis and corneal fibroblast proliferation, allergic encephalomyelitis, amyotrophic lateral sclerosis, acute pancreatitis, cerebral cryptococcosis, autoimmune disease including Type 1 diabetes mellitus (DM), experimental allergic encephalomyelitis (EAE), systemic lupus erythematosus (SLE), colitis, thyroiditis and various forms of arthritis, cancer such as AML, bacterial infections, and/or other pathologies and disorders.

NOV6 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV6 protein have multiple hydrophilic regions, each of which can be used as an immunogen. In one embodiment, contemplated NOV6 epitope is from about amino acids 80 to 150. In other embodiments, NOV6 epitope is from about amino acids 200 to 250, or from about amino acids 330 to 420. This novel protein also has value in development of powerful assay system for functional analysis of various human, disorders, which will help in understanding of pathology of the disease and development of new drug targets for various disorders.

NOV7

A disclosed NOV7 nucleic acid of 1769 nucleotides (also referred to CG50389-01) encoding a novel Interleukin 1 receptor related protein-like protein is shown in Table 7A. An open reading frame was identified beginning with an ATG initiation codon at nucleotides 45-47 and ending with a TGA codon at nucleotides 477-479. In Table 7A, the 5′ and 3′ untranslated regions are underlined and the start and stop codons are in bold letters.

TABLE 7A
NOV7 Nucleotide Sequence

(SEQ ID NO:29)

CGCCCGCCCACGGCGGCGGGGAAATACCTAGGCATGGAAGTGGCATGACAGGGCTCGTGTCCCTGTCATAT
TTTCCACTCTCCACGAGGTCCTGCGCGCTTCAATCCTGCAGGCAGCCCGGTTTGGGGATGTGGTCCTTGCT
GCTCTGCGGGTTGTCCACGCCCTTCCACTGTCTGTCACAGCAGATGGATGCAAGGACATTTTTATGAAAAA
ATGAGATACTTTCAGCAAGCCAGCCTTTTGCTTTTAATTGTACATTCCCTCCCATAACATCTGGGGAAGTC
AGTGTAACATGGTATAAAAATTCTAGCAAAATCCCAGTGTCCAAAATCATACAGTCTAGAATTCACCAGGA
CGAGACTTGGATTTTGTTTCTCCCCATGGAATGGGGGGACTCAGGAGTCTACCAATGTGTTATAAAGACTG
TAACGAGATTAAAGGGGAGCGGTTCACTGTTTTGGAAACCAGGCTTTTGGTGAGCAATGTCTCGGCAGAGG
ACAGAGGGAACTACGCGTGTCAAGCCATACTGACACACTCAGGGAAGCAGTACGAGGTTTTAAATGGCATC
ACTGTGAGCATTACAGAAAGAGCTGGATATGGAGGAAGTGTCCCTAAAATCATTTATCCAAAAAATCATTC
AATTGAAGTACAGCTTGGTACCACTCTGATTGTGGACTGCAATGTAACAGACACCAAGGATAATACAAATC
TACGATGCTGGAGAGTCAATAACACTTTGGTGGATGATTACTATGATGAATCCAAACGAATCAGAGAAGGG
GTGGAAACCCATGTCTCTTTTCGGGAACATAATTTGTACACAGTAAACATCACCTTCTTGGAAGTGAAAAT
GGAAGATTATGGCCTTCCTTTCATGTGCCACGCTGGAGTGTCCACAGCATACATTATATTACAGCTCCCAG
CTCCGGATTTTCGAGCTTACTTGATAGGAGGGCTTATCGCCTTGGTGGCTGTGGCTGTGTCTGTTGTGTAC
ATATACAACATTTTTAAGATCGACATTGTTCTTTGGTATCGAAGTGCCTTCCATTCTACAGAGACCATAGT
AGATGGGAAGCTGTATGACGCCTATGTCTTATACCCCAAGCCCCACAAGGAAAGCCAGAGGCATGCCGTGG
ATGCCCTGGTGTTGAATATCCTGCCCGAGGTGTTGGAGAGACAATGTGGATATAAGTTGTTTATATTCGGC
AGAGATGAATTCCCTGGACAAGCCGTGGCCAATGTCATCGATGAAAACGTTAAGCTGTGCAGGAGGCTGAT
TGTCATTGTGGTCCCCGAATCGCTGGGCTTTGGCCTGTTGAAGAACCTGTCAGAAGAACAAATCGCGGTCT
ACAGTGCCCTGATCCAGGACGGGATGAAGGTTATTCTCATTGAGCTGGAGAAAATCGAGGACTACACAGTC
ATGCCAGAGTCAATTCAGTACATCAAACAGAAGCATGGTGCCATCCGGTGGCATGGGGACTTCACGGAGCA
GTCACAGTGTATGAAGACCAAGTTTTGGAAGACAGTGAGATACCACATGCCGCCCAGAAGGTGTCGGCCGT
TTCTCCGGTCCACGTGCCGCAGCACACACCTCTGTACCGCACCGCAGGCCCAGAACTAGGCTCAAGAAGAA
AGAAGTGTACTCTCACGACTGGCTAAGACTTGCTGGACTGACACCTATGGCTGGAAGATGACCTGTTTTGC
TCCATGTCTCCTCATTCCTACACCTATTTTCTGCTGCAGGATGAGGCTAGGGTTAGCATTCTAGA

The disclosed NOV7 nucleic acid sequence, localized to the q12 region of chromosome 2, has 1363 of 1370 bases (99%) identical to a gb:GENBANK-ID:HSU49065|acc:U49065.1 mRNA from Homo sapiens (Human interleukin-1 receptor-related protein mRNA, complete cds) (E=7.0e⁻³⁰¹).

A disclosed NOV7 polypeptide (SEQ ID NO: 30) encoded by SEQ ID NO: 29 is 144 amino acid residues and is presented using the one-letter amino acid code in Table 7B. Signal P, Psort and/or Hydropathy results predict that NOV7 has a signal peptide and is likely to be localized in the plasma membrane with a certainty of 0.6500. In other embodiments, NOV7 is also likely to be localized to the microbody (peroxisome) with a certainty of 0.6400, to the mitochondrial inner membrane with a certainty of 0.5762, or the mitochondrial intermembrane space with a certainty of 0.3386. The most likely cleavage site for a NOV7 peptide is between amino acids 47 and 48, at: VTA-DG.

TABLE 7B
Encoded NOV7 protein sequence.

(SEQ ID NO:30)

MTGLVSLSYFPLSTRSCALQSCRQPGLGMWSLLLCGLSIALPLSVTADGCKDIFMKNEILSASQPFAFNCT
FPPITSGEVSVTWYKNSSKIPVSKIIQSRIHQDETWILFLPMEWGDSGVYQCVIKTVTRLKGSGSLFWKPG
FW

The disclosed NOV7 amino acid sequence has 129 of 144 amino acid residues (99%) identical to 129 of 563 amino acid residues gb:GENBANK-ID:HSU49065|acc:U49065.1 protein from Homo sapiens (Human interleukin-1 receptor-related protein mRNA, complete cds).

NOV7 also has homology to the amino acid sequence shown in the BLASTP data listed in Table 7C.

TABLE 7C
BLAST results for NOV7

| Gene Index/ | | Length | Identity | Positives | |
|---|---|---|---|---|---|
| Identifier | Protein/Organism | (aa) | (%) | (%) | Expect |
| gi\|13637728\|ref\|XP_{—} | similar to IL- | 603 | 126/126 | 126/126 | 3e−72 |
| 002685.3\| | 1Rrp2 (H. sapiens) | | (100%) | (100%) | |
| (XM_002685) | [Homo sapiens] | | | | |
| gi\|4504663\|ref\|NP_{—} | interleukin 1 | 562 | 98/98 | 98/98 | 5e−55 |
| 003845.1\|(NM_{—} | receptor-like 2 | | (100%) | (100%) | |
| 003854) | [Homo sapiens | | | | |
| gi\|10644684\|gb\| | IL-1Rrp2 | 574 | 59/100 | 73/100 | 3e−30 |
| AAG21367.1\| | [Mus musculus] | | (59%) | (73%) | |
| AF284433_1 | | | | | |
| (AF284433) | | | | | |
| gi\|1236081\|gb\| | interleukin-1 | 561 | 54/100 | 73/100 | 4e−29 |
| AAB53238.1\| | receptor-related | | (54%) | (73%) | |
| (U49066) | protein [Rattus norvegicus] | | | | |
| gi\|400047\|sp\|Q02955\| | INTERLEUKIN-1 | 576 | 35/102 | 55/102 | 7e−09 |
| IL1R_RAT | RECEPTOR, TYPE I | | (34%) | (53%) | |
| | PRECURSOR | | | | |
| | (IL-1R-1) (P80) | | | | |

The homology of these sequences is shown graphically in the ClustalW analysis shown in Table 7D.

[image]

[image]

Tables 7E-F list the domain description from DOMAIN analysis results against NOV7. This indicates that the NOV7 sequence has properties similar to those of other proteins known to contain this domain.

TABLE 7E
Domain Analysis of NOV7
gnl|Smart|smart00408, IGc2, Immunoglobulin C-2 Type (SEQ ID NO:100)
C-Length = 63 residues, 85.7% aligned
Score = 40.0 bits (92), Expect = 9e−05

| | | | |
|---|---|---|---|
| Query: | 64 | QPFAFNCTFPPITSGEVSVTWYKNSSKIPVSKIIQSRIHQDETWILFLPMEWGDSGVYQC | 123 |
| | | + \| \| ++\|\| \|+ +\| +\|\|+ + + + \|\|\|+\| \| | |
| Sbjct: | 4 | ESVTLTC--PASGDPVPNITWLKDGKPLP-----ESRVVASGSTLTIKNVSLEDSGLYTC | 56 |
| Query: | 124 | V 124 | |
| | | \| | |
| Sbjct: | 57 | V 57 | |

TABLE 7F
Domain Analysis of NOV7
gnl|Pfam|pfam00047, ig, Immunoglobulin domain. Members of the
immunoglobulin superfamily are found in hundreds of proteins of
different functions. Examples include antibodies, the giant muscle
kinase titin and receptor tyrosine kinases. Immunoglobulin-like
domains may be involved in protein-protein and protein-ligand
interactions. The Pfam alignments do not include the first and last
strand of the imnunoglobulin-like domain. (SEQ ID NO:101)
CD-Length = 68 residues, 97.1% aligned
Score = 36.6 bits (83), Expect = 0.001

| | | | |
|---|---|---|---|
| Query: | 64 | QPFAFNCTFPPITSGEVSVTWYKNSSKIPVSKIIQSRIHQDETW------ILFLPMEWGD | 117 |
| | | + \|+ + +\|\|\| ++ +\| + +\|\|+ + + + \| | |
| Sbjct: | 2 | ESVTLTCSVSG-YPPDPTVTWLRDGKEIELLGSSESRVSSGGRFSISSLSLTISSVTPED | 60 |
| Query: | 118 | SGVYQCV 124 | |
| | | \|\| \| \|\| | |
| Sbjct: | 61 | SGTYTCV 67 | |

Interleukin-1 (IL-1) is a central regulator of the immune and inflammatory responses. Recently, a family of proteins have been described that share significant homology in their signaling domains with the Type I IL-1receptor (IL-1RI), which includes the IL-1receptor-related protein. The members of IL-1RI are clustered within 450 kb on human chromosome 2q and all of them are important in host responses to injury and infection. The remarkable conservation between diverse species indicates that the IL-1 system represents an ancient signaling machine critical for responses to environmental stresses and attack by pathogens (O'Neill L. A., Greene, C., 1998, J. Leukoc Biol., vol. 63: 650-657, Busfield et al., 2000, Genomics vol. 66:213-216).

The disclosed NOV7 nucleic acid of the invention encoding a Interleukin 1 receptor related protein-like protein includes the nucleic acid whose sequence is provided in Table 7A or a fragment thereof. The invention also includes a mutant or variant nucleic acid any of whose bases may be changed from the corresponding base shown in Table 7A while still encoding a protein that maintains its Interleukin 1 receptor related protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1% percent of the bases may be so changed.

The disclosed NOV7 protein of the invention includes the Interleukin 1 receptor related protein-like protein whose sequence is provided in Table 7B. The invention also includes a mutant or variant protein any of whose residues may be changed from the corresponding residue shown in Table 7B while still encoding a protein that maintains its Interleukin 1 receptor related protein-like activities and physiological functions, or a functional fragment thereof. In the mutant or variant protein, up to about 66% percent of the residues may be so changed.

The protein similarity information, expression pattern, and map location for the Interleukin 1 receptor related protein-like protein and nucleic acid (NOV7) disclosed herein suggest that NOV7 may have important structural and/or physiological functions characteristic of the Interleukin 1 receptor related protein-like family. Therefore, the NOV7 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV7 nucleic acids and proteins of the invention are useful in potential diagnostic and therapeutic applications implicated in various diseases and disorders described below and/or other pathologies. For example, the compositions of the present invention will have efficacy for treatment of patients suffering from uveitis and corneal fibroblast proliferation, allergic encephalomyelitis, amyotrophic lateral sclerosis, acute pancreatitis, cerebral cryptococcosis, autoimmune disease including Type 1 diabetes mellitus (DM), experimental allergic encephalomyelitis (EAE), systemic lupus erythematosus (SLE), colitis, thyroiditis and various forms of arthritis, cancer such as AML, bacterial infectionss, and/or other pathologies/disorders. The NOV7 nucleic acid, or fragments thereof, may further be usefull in diagnostic applications, wherein the presence or amount of the nucleic acid or the protein are to be assessed.

NOV7 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV7 protein have multiple hydrophilic regions, each of which can be used as an immunogen. In one embodiment, contemplated NOV7 epitope is from about amino acids 15 to 30. In another embodiment, a contemplated NOV7 epitope is from about amino acids 70 to 135. This novel protein also has value in development of powerful assay system for functional analysis of various human disorders, which will help in understanding of pathology of the disease and development of new drug targets for various disorders.

NOV8

A disclosed NOV8 nucleic acid of 954 nucleotides (also referred to as CG50387-02) encoding a novel Connexin GJA3-like protein is shown in Table 8A. An open reading frame was identified beginning with an ATG initiation codon at nucleotides 1-3 and ending with a TGA codon at nucleotides 952-954. A putative untranslated region upstream from the initiation codon is underlined in Table 8A. The start and stop codons are in bold letters.

TABLE 8A
NOV8 nucleotide sequence.

(SEQ ID NO:31)

ATGGGCGACTGGAGCTTTCTGGGAAGACTCTTAGAAAATGCACAGGAGCACTCCACGGTCATCGGCAAGGTT
TGGCTGACCGTGCTGTTCATCTTCCGCATTTTGGTGCTGGGGGCCGCGGCCGAGGACGTGTGCGGCGATGAG
CAGTCAGACTTCACCTGCAACACCCAGCAGCCGGOCTGCGAGAACGTCTGCTACGACAGGGCCTTCCCCATC
TCCCACATCCGCTTCTGGGCGCTGCAGATCATCTTCGTGTCCACGCCCACCCTCATCTACCTGGGCCACGTG
CTGCACATCGTGCGCATGGAGGAGAAGAAGAAAGAGAGGGACGAGGAGGAGCAGCTGAAGAGAGAGAGCCCC
AGCCCCAAGGAGCCACCGCAGGACAATCCCTCGTCGCGGGACGACCGCCGCAGGGTGCGCATCGCCGGCGCG
CTCCTCCCCACCTACCTCTTCAACATCATCTTCAGAGGGTCTTCCACCTCCCCTTCATCCCCCCCCCACTAC
TTTCTGTACGGCTTCGAGCTGAAGCCGCTCTACCGCTGCGACCGCTGGCCCTGCCCCAACACGGTGGACTGC
TTCATCTCCAGGCCCACGGAGAAGACCATCTTCATCATCTTCATGCTGGCGGTGGCCTGCGCGTCACTGCTG
CTCAACATGCTGGAGATATACCACCTGGGCTGGAAGAAGCTCAAGCAGGGCGTGACCAGCCGCCTCGGCCCG
GACGCCTCCGAGGCCCCGCTGGGGACAGCCGATCCCCCGCCCCTGCTGCTGGATGGGAGCGGCAGCAGTCTG
GAGGGGAGCGCCCTGGCAGGGACCCCCGAGGAGGAGGAGCAGGCCGTCACCACCGCCGCCCAGATGCACCAG
CCGCCCTTGCCCCTCGGAGACCCAGGTCGGGCCAGCAAGGCCAGCAGGGCCAGCAGCGGGCGGGCCAGACCG
GAGGACTTGGCCATCTAG

The NOV8 nucleic acid sequence is located on chromsome 13, has 766 of 766 bases (100%) identical to a gb:GENBANK-ID:AF075290|acc:AF075290.1 mRNA from Homo sapiens (Homo sapiens gap-junction protein alpha 3 (GJA3) gene, complete cds) (E=1.7e⁻²¹⁰)

The disclosed NOV8 polypeptide (SEQ ID NO: 32) encoded by SEQ ID NO: 31 has 317 amino acid residues and is presented in Table 8B using the one-letter amino acid code. Signal P, Psort and/or Hydropathy results predict that NOV8 has a signal peptide and is likely to be localized to the plasma membrane with a certainty of 0.6000. In other embodiments, NOV8 may also be localized to the Golgi body with a certainty of 0.4000, the endoplasmic reticulum (membrane) with a certainty of 0.3000, or the microbody (peroxisome) with a certainty of 0.3000. The most likely cleavage site for NOV8 is between positions 41 and 42, AAA-ED.

TABLE 8B
Encoded NOV8 protein sequence.

(SEQ lID NO:32)

MGDWSFLGRLLENAQEHSTVIGKVWLTVLFIFRILVLGAAAEDVWGDEQSDFTcNTQQPGCENVCYDRAFPI
SHIRFWALQIIFVSTPTLIYLGHVLMIVRMEEKKKEREEEEQLKRESPSPKEPPQDNPSSRDDRGRVRMAGA
LLRTYVFNIIFKTLFEVGFIAGQYFLYGFELKPLYRCDRWPCPNTVDCFISRPTEKTIFIIFMIAVACASLL
LNMLEIYHLGWKKLKOGVTSRLGPDASEAPLGTADPPPLLLDGSGSSLEGSALAGTPEEEEQAVTTAAQMHQ
PPLPLGDPGRASKASRASSGRARPEDLAI

A search of sequence databases reveals that the NOV8 amino acid sequence has 255 of 255 amino acid residues (100%) identical to, and 255 of 255 amino acid residues (100%) similar to, the 435 amino acid residue ptnr:TREMBLNEW-ACC:CAC16957 protein from Homo sapiens (Human) (BA264J4.3 (Novel Connexin (Gap Junction Protein)) (E=5.8e⁻¹⁷²).

NOV8 is expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus, lung. This information was derived by determining the tissue sources of the sequences that were included in the invention including but not limited to SeqCalling sources, Public EST sources, Literature sources, and/or RACE sources.

In addition, the sequence is predicted to be expressed in lens fiber cells because of the expression pattern of (GENBANK-ID: gb:GENBANK-ID:AF075290|acc:AF075290.1) a closely related Homo sapiens gap-junction protein alpha 3 (GJA3) gene, complete cds homolog in species Homo sapiens.

NOV8 also has homology to the amino acid sequence shown in the BLASTP data listed in Table 8C.

TABLE 8C
BLAST results for NOV8

| Gene Index/ | | Length | Identity | Positives | |
|---|---|---|---|---|---|
| Identifier | Protein/Organism | (aa) | (%) | (%) | Expect |
| gi\|13489110\|ref\|NP_{—} | gap junction | 435 | 233/249 | 233/249 | e−134 |
| 068773.1\| (NM_021954) | protein, alpha 3, | | (93%) | (93%) | |
| | 46kD (connexin46) | | | | |
| | [Homo sapiens] | | | | |
| gi\|14753411\|ref\|XP_{—} | gap junction | 435 | 233/249 | 233/249 | e−134 |
| 051651.1\| (XM_051651) | protein, alpha 3, | | (93%) | (93%) | |
| | 46kD (connexin46) | | | | |
| | [Homo sapiens] | | | | |
| gi\|8393440\|ref\|NP_{—} | gap junction | 417 | 208/256 | 219/256 | e−116 |
| 058671.1\| (NM_016975) | membrane channel | | (81% | (85%) | |
| | protein alpha 3; | | | | |
| | connexin 46; | | | | |
| | alpha 3 connexin | | | | |
| | [Mus musculus | | | | |
| gi\|13242279\|ref\|NP_{—} | connexin 46 | 416 | 207/255 | 218/255 | e−116 |
| 077352.1\| (NM_024376) | [Rattus norvegicus] | | (81%) | (85% | |
| gi\|5919130\|gb\| | connexin 44 | 413 | 202/249 | 214/249 | e−113 |
| AAD56220.1\| | protein [Ovis aries] | | (81%) | (85%) | |
| (AF177912) | | | | | |

The homology of these sequences is shown graphically in the ClustalW analysis shown in Table 8D.

[image]

[image]

Tables 8E-F list the domain description from DOMAIN analysis results against NOV8. This indicates that the NOV8 sequence has properties similar to those of other proteins known to contain this domain.

TABLE 8E
Domain Analysis of NOV8

gnl|Pfam|pfam00029, connexin, Connexin. (SEQ ID NO:107)
CD-Length=218 residues, 99.5% aligned
Score=355 bits (912), Expect=2e−99

| | | | |
|---|---|---|---|
| Query: | 3 | DWSFLGRLLENAQEHSTVIGKVWLTVLFIFRILVLGAAAEDVWGDEQSDFTCNTQQPGCE | 62 |
| | | \|\|\|\|\|\|\|\|\| +\|\|\| \|\|\|+\|\|+\|\|\|\|\|\|\|\|\|\|\| \|\|\| \|\|\|\|\|\|\|\|\| \|\|\|\|\|\|\|\|\| | 62 |
| Sbjct: | 2 | DWSFLGRLLEGVNKHSTAIGKIWLSVLFIFRILVLGVAAESVWGDEQSDFVCNTQQPGCE | 61 |
| Query: | 63 | NVCYDRAFPISHIRFWALQIIFVSTPTLIYLGHVLHIVRMEEKKKEREEEEQLKRESPSP | 122 |
| | | \|\|\|\|\|+ \|\|\|\|\|+\| \| \|\|+\|\|\|\|\|\|+\|+\|\|\|\|\| + \|\| \|\|\| +\|+\|\|\| \| | |
| Sbjct: | 62 | NVCYDQFFPISHVRLWVLQLIFVSTPSLLYLGHVAYRVRREEKLREKEEEHSKGLYSEEA | 121 |
| Query: | 123 | KEPPQDNPSSRDDRGRVRMAGALLRTYVFNIIFKTLFEVGFIAGQYFLYGFELKPLYRCD | 182 |
| | | \|+ + \|+\|\|+ \| \| \|\|\|\|+\|\|\|\|++\|\|\|\|\|+ \|\|\| \|\|\|\| + \|\| \| | |
| Sbjct: | 122 | KK------RCGSEDGKVRIRGGLWWTYVFSIIFKSIFEVGFLYGQYLLYGFTMSPLVVCS | 175 |
| Query: | 183 | RWPCPNTVDCFISRPTEKTIFIIFMLAVACASLLLNMLEIYHL | 225 |
| | | \| \|\|\|+\|\|\|\|\|+\|\|\|\|\|\|\|\|\|\|+\|\|\| \|+ \|\|\|\|+ \|+++\| | |
| Sbjct: | 176 | RAPCPHTVDCFVSRPTEKTIFIVFMLVVSAICLLLNLAELFYL | 218 |

TABLE 8F
Domain Analysis of NOV8

gnl|Smart|smart00037, CNX, Connexin homologues;
Connexin channels participate in the regulation of
signaling between developing and differentiated
cell types. (SEQ ID NO:108)
CD-Length=34 residues, 97.1% aligned
Score=83.2 bits (204), Expect=2e−17

| | | | |
|---|---|---|---|
| Query: | 44 | VWGDEQSDFTCNTQQPGCENVCYDRAFPISHIR | 76 |
| | | \|\|\|\|\|\|\|\|\|\|\|\|\|\|\|\|\|\|\|\|\|\|\|\|+ \|\|\|\|\|+\| | |
| Sbjct: | 2 | VWGDEQSDFTCNTQQPGCENVCYDQFFPISHVR | 34 |

The connexins are a family of integral membrane proteins that oligomerise to form intercellular channels that are clustered at gap junctions. These channels are specialised sites of cell-cell contact that allow the passage of ions, intracellular metabolites and messenger molecules from the cytoplasm of one cell to its apposing neighbours. They are found in almost all vertebrate cell types, and somewhat similar proteins have been cloned from plant species. Invertebrates utilise a different family of molecules, innexins, that share a similar predicted secondary structure to the vertebrate connexins, but have no sequence identity to them.

Vertebrate gap junction channels are thought to participate in diverse biological functions. For instance, in the heart they permit the rapid cell-cell transfer of action potentials, ensuring coordinated contraction of the cardiomyocytes. They are also responsible for neurotransmission at specialised ‘electrical’ synapses. In non-excitable tissues, such as the liver, they may allow metabolic cooperation between cells. In the brain, glial cells are extensively-coupled by gap junctions; this allows waves of intracellular Ca²⁺ to propagate through nervous tissue, and may contribute to their ability to spatially-buffer local changes in extracellular K⁺ concentration.

The connexin protein family is encoded by at least 13 genes in rodents, with many homologues cloned from other species. They show overlapping tissue expression patterns, most tissues expressing more than one connexin type. Their conductances, permeability to different molecules, phosphorylation and voltage-dependence of their gating, have been found to vary. Possible communication diversity is increased further by the fact that gap junctions may be formed by the association of different connexin isoforms from apposing cells. However, in vitro studies have shown that not all possible combinations of connexins produce active channels.

Hydropathy analysis predicts that all cloned connexins share a common transmembrane (TM) topology. Each connexin is thought to contain 4 TM domains, with two extracellular and three cytoplasmic regions. This model has been validated for several of the family members by in vitro biochemical analysis. Both N- and C-termini are thought to face the cytoplasm, and the third TM domain has an amphipathic character, suggesting that it contributes to the lining of the formed-channel. Amino acid sequence identity between the isoforms is ˜50-80%, with the TM domains being well conserved. Both extracellular loops contain characteristically conserved cysteine residues, which likely form intramolecular disulphide bonds. By contrast, the single putative intracellular loop (between TM domains 2 and 3) and the cytoplasmic C-terminus are highly variable among the family members. Six connexins are thought to associate to form a hemi-channel, or connexon. Two connexons then interact (likely via the extracellular loops of their connexins) to form the complete gap junction channel.

Two sets of nomenclature have been used to identify the connexins. The first, and most commonly used, classifies the connexin molecules according to molecular weight, such as connexin43 (abbreviated to Cx43), indicating a connexin of molecular weight close to 43 kD. However, studies have revealed cases where clear functional homologues exist across species that have quite different molecular masses; therefore, an alternative nomenclature was proposed based on evolutionary considerations, which divides the family into two major subclasses, alpha and beta, each with a number of members. Due to their ubiquity and overlapping tissue distributions, it has proved difficult to elucidate the functions of individual connexin isoforms. To circumvent this problem, particular connexin-encoding genes have been subjected to targeted-disruption in mice, and the phenotype of the resulting animals investigated. Around half the connexin isoforms have been investigated in this manner. Further insight into the functional roles of connexins has come from the discovery that a number of human diseases are caused by mutations in connexin genes. For instance, mutations in Cx32 give rise to a form of inherited peripheral neuropathy called X-linked dominant Charcot-Marie-Tooth disease. Similarly, mutations in Cx26 are responsible for both autosomal recessive and dominant forms of nonsyndromic deafness, a disorder characterised by hearing loss, with no apparent effects on other organ systems.

Gap junction alpha-3 (GJA3) protein (also called connexin46, or Cx46) is a connexin of ˜435 amino acid residues. The bovine form is slightly shorter (401 residues) and is hence known as Cx44, having a molecular mass of ˜44 kD. Cx46 (together with Cx50) is a connexin isoform expressed in the lens fibres of the eye. Here, gap junctions join the cells into a functional syncytium, and also couple the fibres to the epithelial cells on the anterior surface of the lens. The lens fibres depend on this epithelium for their metabolic support, since they lose their intra-cellular organelles, and accumulate high concentrations of crystallins, in order to produce their optical transparency. Genetically-engineered mice deficient in Cx46 demonstrate the importance of Cx46 in forming lens fibre gap junctions; these mice develop normal lenses, but subsequently develop early onset senile-type cataracts that resemble human nuclear cataracts. Aberrant proteolysis of crystallin proteins has been observed in the lenses of Cx46-null mice.

The disclosed NOV8 nucleic acid of the invention encoding a Connexin GJA3-like protein includes the nucleic acid whose sequence is provided in Table 8A, or a fragment thereof The invention also includes a mutant or variant nucleic acid any of whose bases may be changed from the corresponding base shown in Table 8A while still encoding a protein that maintains its Connexin GJA3-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV8 protein of the invention includes the Connexin GJA3-like protein whose sequence is provided in Table 8B. The invention also includes a mutant or variant protein any of whose residues may be changed from the corresponding residue shown in Table 2 while still encoding a protein that maintains its Connexin GJA3-like activities and physiological functions, or a functional fragment thereof. In the mutant or variant protein, up to about 66% percent of the residues may be so changed.

The invention further encompasses antibodies and antibody fragments, such as F_{ab} or (F_{ab})₂, that bind immunospecifically to any of the proteins of the invention.

The above defined information for this invention suggests that this Connexin GJA3-like protein (NOV8) may function as a member of a “Connexin GJA3 family”. Therefore, the NOV8 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV8 nucleic acids and proteins of the invention are useful in potential therapeutic applications implicated in nonsyndromic deafness, keratinization disorders, gap-junction-related neuropathies and other pathological conditions of the nervous system, where dysfunctions of junctional communication are considered to play a casual role, demyelinating neuropathies (including Charcot-Marie-Tooth disease), erythrokeratodermia variabilis (EKV), atrioventricular (AV) conduction defects such as arrhythmia, lens cataracts and/or other diseases or pathologies.

NOV8 nucleic acids and polypeptides are further useful in the generation of antibodies that bind immuno-specifically to the novel NOV8 substances for use in therapeutic or diagnostic methods. These antibodies may be generated according to methods known in the art, using prediction from hydrophobicity charts, as described in the “Anti-NOVX Antibodies” section below. The disclosed NOV8 protein has multiple hydrophilic regions, each of which can be used as an immunogen. In one embodiment, a contemplated NOV8 epitope is from about amino acids 40 to 80. In another embodiment, a NOV8 epitope is from about amino acids 90 to 150, from about amino acids 170 to 200, or from about amino acids 220 to 320. These novel proteins can be used in assay systems for functional analysis of various human disorders, which will help in understanding of pathology of the disease and development of new drug targets for various disorders.

NOV9

A disclosed NOV9 nucleic acid of 967 nucleotides (also referred to as CG50271-01) encoding a novel Olfactory Receptor-like protein is shown in Table 9A. An open reading frame was identified beginning with an ATG initiation codon at nucleotides 12-14 and ending with a TGA codon at nucleotides 948-950. A putative untranslated region upstream from the initiation codon and downstream from the termination codon is underlined in Table 9A. The start and stop codons are in bold letters.

TABLE 9A
NOV9 nucleotide sequence.

| | |
|---|---|
| ACTAACAAAGAATGGATCAGAAAAATGGAAGTTCTT | (SEQ ID NO:33) |
| TCACTGGATTTATCCTACTGGGTTTCTCTGACAGGC | |
| CTCAGCTGGAGCTAGTCCTCTTTGTGGTTCTTTTGA | |
| TCTTCTATATCTTCACTTTGCTGGGGAACAAAACCA | |
| TCATTGTATTATCTCACTTGGACCCACATCTTCACA | |
| ATCCTATGTATTTTTTCTTCTCCAACCTAAGCTTTT | |
| TGGATCTGTGTTACACAACCGGCATTGTTCCACAGC | |
| TCCTGGTTAATCTCAGGGGAGCAGACAAATCAATCT | |
| CCTATGGTGGTTGTGTAGTTCAGCTGTACATCTCTC | |
| TAGGCTTGGGATCTACAGAATGCGTTCTCTTAGGAG | |
| TGATGGCATTTGACCGCTATGCAGCTGTTTGCAGGC | |
| CCCTCCACTACACAGTAGTCATGCACCCTTGTCTGT | |
| ATGTGCTGATGGCTTCTACTTCATGGGTCATTGGTT | |
| TTGCCAACTCCCTATTGCAGACGGTGCTCATCTTGC | |
| TTTTAACACTTTGTGGAAGAAATAAATTAGAACACT | |
| TTCTTTGTGAGGTTCCTCCATTGCTCAAGCTTGCCT | |
| GTGTTGACACTACTATGAATGAATCTGAACTCTTCT | |
| TTGTCAGTGTCATTATTCTTCTTGTACCTGTTGCAT | |
| TAATCATATTCTCCTATAGTCAGATTGTCAGGGCAG | |
| TCATGAGGATAAAGTCAGCAACAGGGCAGAGAAAAG | |
| TGTTTGGGACATGTGGCTCCCACCTCACAGTGGTTT | |
| CCCTGTTCTACGGCACAGCTATCTATGCTTACCTCC | |
| AGCCCGGCAACAACTACTCTCAGGATCAGGGCAAGT | |
| TCATCTCTCTCTTCTACACCATCATTACACCCATGA | |
| TCAACCCCCTCATATATACACTGAGGAACAAGGATG | |
| TGAAAGGAGCACTTAAGAAGGTGCTCTGGAAGAACT | |
| ACGACTCCAGATGACTTGGAGAGAAAGACAT | |

The disclosed NOV9 polypeptide (SEQ ID NO: 30) encoded by SEQ ID NO: 29 has 312 amino acid residues, a molecular weight of 34977.1 and is presented in Table 9B using the one-letter amino acid code. Signal P, Psort and/or Hydropathy results predict that NOV9 has a signal peptide and is likely to be localized in the plasma membrane with a certainty of 0.6400. I The most likely ceavage site for NOV9 is between positions 41 and 42, LLG-NK.

TABLE 9B
Encoded NOV9 protein sequence.

| | |
|---|---|
| MDQKNGSSFTGFILLGFSDRPQLELVLFVVLLIFYI | (SEQ ID NO:34) |
| FTLLGNKTIIVLSHLDPHLHNPMYFFFSNLSFLDLC | |
| YTTGIVPQLLVNLRGADKSISYGGCVVQLYISLGLG | |
| STECVLLGVMAFDRYAAVCRPLHYTVVMHPCLYVLM | |
| ASTSWVIGFANSLLQTVLILLLTLCGRNKLEHFLCE | |
| VPPLLKLACVDTTMNESELFFVSVIILLVPVALIIF | |
| SYSQIVRAVMRIKSATGQRKVFGTCGSHLTVVSLFY | |
| GTAIYAYLQPGNNYSQDQGKFISLFYTIITPMINPL | |
| IYTLRNKDVKGALKKVLWKNYDSR | |

A BLASTX of NOV9 shows a 55% (identities) and 72% (positives) similarity to a Mouse Odorant Receptor MOR18 protein (E=1.2e⁻¹⁰¹).

The disclosed NOV9 polypeptide has homology to the amino acid sequences shown in the BLASTP data listed in Table 9C.

TABLE 9C
BLAST results for NOV9

| Gene Index/ | | Length | Identity | Positives | |
|---|---|---|---|---|---|
| Identifier | Protein/Organism | (aa) | (%) | (%) | Expect |
| gi\|17464665\|ref\|XP_{—} | similar to | 312 | 265/312 | 265/312 | e−143 |
| 069524.1\| | olfactory | | (84%) | (84%) | |
| (XM_069524) | receptor, family | | | | |
| | 2, subfamily W | | | | |
| gi\|17455398\|ref\|XP_{—} | similar to | 252 | 221/249 | 222/249 | e−119 |
| 069445.1\| | olfactory | | (88%) | (88%) | |
| (XM_069445) | receptor | | | | |
| | (H. sapiens) | | | | |
| | [Homo sapiens] | | | | |
| gi\|17445400\|ref\|XP_{—} | similar to | 309 | 169/301 | 205/301 | 1e−87 |
| 060573.1\| | olfactory | | (56%) | (67%) | |
| (XM_060573) | receptor 15 | | | | |
| | (H. sapiens) | | | | |
| | [Homo sapiens] | | | | |
| gi\|14423800\|sp\| | OLFACTORY | 357 | 170/308 | 207/308 | 2e−87 |
| Q9GZK3\|O2B2_{—} | RECEPTOR 2B2 | | (55%) | (67%) | |
| HUMAN | (OLFACTORY | | | | |
| | RECEPTOR 6-1) | | | | |
| | (OR6-1) | | | | |
| | (HS6M1-10) | | | | |
| gi\|13624329\|ref\|NP_{—} | olfactory | 320 | 167/305 | 202/305 | 3e−87 |
| 112165.1\| | receptor, family | | (54%) | (65%) | |
| (NM_030903) | 2, subfamily W, | | | | |
| | member 1 [Homo sapiens] | | | | |

The homology between these and other sequences is shown graphically in the ClustalW analysis shown in Table 9D. In the ClustalW alignment of the NOV9 protein, as well as all other ClustalW analyses herein, the black outlined amino acid residues indicate regions of conserved sequence (i e., regions that may be required to preserve structural or functional properties), whereas non-highlighted amino acid residues are less conserved and can potentially be altered to a much broader extent without altering protein structure or function.

[image]

[image]

Table 9E lists the domain description from DOMAIN analysis results against NOV9. This indicates that the NOV9 sequence has properties similar to those of other proteins known to contain this domain.

TABLE 9E
Domain Analysis of NOV9

gnl|Pfam|pfam00001, 7tm_1, 7 transmembrane receptor (rhodopsin
family). (SEQ ID NO:114)
CD-Length 254 residues, 100.0% aligned
Score=88.2 bits (217), Expect=6e−19

| | | | |
|---|---|---|---|
| Query: | 41 | GNKTIIVLSHLDPHLHNPMYFFFSNLSFLDLCYTTGIVPQLLVNLRGADKSISYGGCVVQ | 100 |
| | | \|\| +\|++ \| \| \| \|\|+ \|\| + + \| \| \| \| \| \| + | |
| Sbjct: | 1 | GNLLVILVILRTKKLRTPTNIFLLNLAVADLLFLLTLPPWALYYLVGGDWVFGDALCKLV | 60 |
| Query: | 101 | LYISLGLGSTECVLLGVMAFDRYAAVCRPLHYTVVMHPCLYVLMASTSWVIGFANSLLQT | 160 |
| | | + + \| +\|\| ++ \|\|\| \|+ \|\| \| + \| ++ \|\|+ \|\| | |
| Sbjct: | 61 | GALFVVNGYASILLLTAISIDRYLAIVHPLRYRRIRTPRRAKVLILLVWVLALLLSLPPL | 120 |
| Query: | 161 | VLILLLTLCGRNKLEHFLCEVPPLLKLACVDTTMNESELFFVSVIILLVPVALIIFSYSQ | 220 |
| | | + \| \|+ \| + \| + ++ \| +\|+ +\|+ \|++ | |
| Sbjct: | 121 | LFSWLRTVEEGNTTVCLID--FPEESVKRSYVLLSTLVGFV-------LPLLVILVCYTR | 171 |
| Query: | 221 | IVRAVMR---------IKSATGQRKVFGTCGSHLTVVSLFYGTAIYAYLQPGNNYS---- | 267 |
| | | \|+\| + + +\|++ ++ + \| + \| \| \| | |
| Sbjct: | 172 | ILRTLRKRARSQRSLKRRSSSERKAAKMLLVVVVVFVLCWLPYHIVLLLDSLCLLSIWRV | 231 |
| Query: | 268 | QDQGKFISLFYTIITPMINPLIY | 290 |
| | | \|+\|+ + +\|\|+\|\| | |
| Sbjct: | 232 | LPTALLITLWLAYVNSCLNPIIY | 254 |

G-Protein Coupled Receptor (GPCRs) have been identified as an extremely large family of protein receptors in a number of species. At the phylogenetic level they can be classified into four major subfamilies. These receptors share a seven transmembrane domain structure with many neurotransmitter and hormone receptors. They are likely to be involved in the recognition and transduction of various signals mediated by G-Proteins, hence their name G-Protein Coupled Receptors. The human GPCR genes are generally intron-less and belong to four gene subfamilies, displaying great sequence variability. These genes are dominantly expressed in olfactory epithelium.

Olfactory receptors (ORs) have been identified as an extremely large family of GPCRs in a number of species. As members of the GPCR family, these receptors share a seven transmembrane domain structure with many neurotransmitter and hormone receptors, and are likely to underlie the recognition and G-protein-mediated transduction of odorant signals. Like GPCRs, the ORs can be expressed in a variety of tissues where they are thought to be involved in recognition and transmission of a variety of signals. The human OR genes are typically intron-less and belong to four different gene subfamilies, displaying great sequence variability. These genes are dominantly expressed in olfactory epithelium.

A BLASTX of the Olfactory Receptor-like protein CG50271-01 described in this invention shows a 55% (identities) and 72% (positives) similarity to a Mouse Odorant Receptor MOR18 protein.

Tsuboi et al. (J Neurosci 1999; 19:8409-18) characterized two separate odorant receptor (OR) gene clusters to examine how olfactory neurons expressing closely linked and homologous OR genes project their axons to the olfactory bulb. Murine OR genes, MOR28, MOR10, and MOR83, share 75-95% similarities in the amino acid sequences and are tightly linked on chromosome 14. In situ hybridization has demonstrated that the three genes are expressed in the same zone, at the most dorsolateral and ventromedial portions of the olfactory epithelium, and are rarely expressed simultaneously in individual neurons. Furthermore, they have found that olfactory neurons expressing MOR28, MOR10, or MOR83 project their axons to very close but distinct subsets of glomeruli on the medial and lateral sides of the olfactory bulb. Similar results have been obtained with another murine OR gene cluster for A16 and MOR18 on chromosome 2, sharing 91% similarity in the amino acid sequences. These results may indicate an intriguing possibility that olfactory neurons expressing homologous OR genes within a cluster tend to converge their axons to proximal but distinct subsets of glomeruli. These lines of study will shed light on the molecular basis of topographical projection of olfactory neurons to the olfactory bulb.

The disclosed NOV9 nucleic acid of the invention encoding a Olfactory Receptor-like protein includes the nucleic acid whose sequence is provided in Table 9A, or a fragment thereof. The invention also includes a mutant or variant nucleic acid any of whose bases may be changed from the corresponding base shown in Table 9A while still encoding a protein that maintains its Olfactory Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

The disclosed NOV9 protein of the invention includes the Olfactory Receptor-like protein whose sequence is provided in Table 9B. The invention also includes a mutant or variant protein any of whose residues may be changed from the corresponding residue shown in Table 2 while still encoding a protein that maintains its Olfactory Receptor-like activities and physiological functions, or a functional fragment thereof. In the mutant or variant protein, up to about 46% percent of the residues may be so changed.

The invention further encompasses antibodies and antibody fragments, such as F_{ab} or (F_{ab})₂, that bind immunospecifically to any of the proteins of the invention.

The above defined information for this invention suggests that this Olfactory Receptor-like protein (NOV9) may function as a member of a “Olfactory Receptor family”. Therefore, the NOV9 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV9 nucleic acids and proteins of the invention are useful in potential therapeutic applications implicated in various diseases and pathologies.

NOV9 nucleic acids and polypeptides are further useful in the generation of antibodies that bind immuno-specifically to the novel NOV9 substances for use in therapeutic or diagnostic methods. These antibodies may be generated according to methods known in the art, using prediction from hydrophobicity charts, as described in the “Anti-NOVX Antibodies” section below. The disclosed NOV9 protein has multiple hydrophilic regions, each of which can be used as an immunogen. These novel proteins can be used in assay systems for functional analysis of various human disorders, which will help in understanding of pathology of the disease and development of new drug targets for various disorders.

NOV10

A disclosed NOV10 nucleic acid of 1596 nucleotides (also referred to as CG55844-01) encoding a novel P450-like protein is shown in Table 10A. An open reading frame was identified beginning with an ATG initiation codon at nucleotides 549-551 and ending with a TGA codon at nucleotides 1594-1596. A putative untranslated region upstream from the initiation codon and downstream from the termination codon is underlined in Table 10A. The start and stop codons are in bold letters.

TABLE 10A
NOV10 nucleotide sequence.

| | |
|---|---|
| ATGCTGCCCATCACAGACCGCCTGCTGCACCTCCTG | (SEQ ID NO:35) |
| GGGCTGGAGAAGACGGCGTTCCGCATATACGCGGTG | |
| TCCACCCTTCTCCTCTTCCTGCTCTTCTTCCTGTTC | |
| CGCCTGCTGCTGCGGTTCCTGAGGCTCTGCAGGAGC | |
| TTCTACATCACCTGCCGCCGGCTGCGCTGCTTCCCC | |
| CAGCCTCCCCGGCGCAACTGGCTGCTGGGCCACCTG | |
| GGCATGTACCTTCCAAATGAGGCGGGCCTTCAAGAT | |
| GAGAAGAAGGTACTGGACAACATGCACCATGTACTC | |
| TTGGTATGGATGGGACCTGTCCTGCCGCTGTTGGTT | |
| CTGGTGCACCCTGATTACATCAAACCCCTTTTGGGA | |
| GCCTCAGCTGCCATCGCCCCCAAGGATGACCTCTTC | |
| TATGGCTTCCTAAAACCTTGGCTAGGGGATGGGCTG | |
| CTGCTCAGCAAAGGTGACAAGTGGAGCCGGCACCGT | |
| CGCCTGCTGACACCCGCCTTCCACTTTGACATCCTG | |
| AAGCCTTACATGAAGATCTTCAACCAGAGCGCTGAC | |
| ATTATGCATGCTAAATGGCGGCATCTGGCAGAGGGC | |
| TCAGCGGTCTCCCTTGATATGTTTGAGCATATCAGC | |
| CTCATGACCCTGGACAGTCTTCAGAAATGTGTCTTC | |
| AGCTACAACAGCAACTGCCAAGAGAAGATGAGTGAT | |
| TATATCTCCGCTATCATTGAACTGAGCGCTCTGTCT | |
| GTCCGGCGCCAGTATCGCTTGCACCACTACCTCGAC | |
| TTCATTTACTACCGCTCGGCGGATGGGCGGAGGTTC | |
| CGGCAGGCCTGTGACATGGTGCACCACTTCACCACT | |
| GAAGTCATCCAGGAACGGCGGCGGGCACTGCGTCAG | |
| CAGGGGGCCGAGGCCTGGCTTAAGGCCAAGCAGGGG | |
| AAGACCTTGGACTTTATTGATGTGCTGCTCCTGGCC | |
| AGGGATGAAGATGGAAAGGAACTGTCAGACGAGGAT | |
| ATCCGAGCCGAAGCAGACACCTTCATGTTTGAGGGT | |
| CACGACACAACATCCAGTGGGATCTCTTGGATGCTG | |
| TTCAATTTGGCAAAGGATCCGGAATACCAGGAGAAA | |
| TGCCGAGAAGAGATTCAGGAAGTCATGAAAGGCCGG | |
| GAGCTGGAGGAGCTCGAGTGGGACGATCTGACTCAG | |
| CTGCCCTTTACAACTATGTGCATTAAGGAGAGCCTG | |
| CGCCAGTACCCACCTGTCACTCTTGTCTCTCGCCAA | |
| TGCACGGAGGACATCAAGCTCCCAGATGGGCGCATC | |
| ATCCCCAAAGGAATCATCTGCTTGGTCAGCATCTAT | |
| GGAACCCACCACAACCCCACAGTGTGGCCTGACTCC | |
| AAGGTGTACAACCCCTACCGCTTTGACCCGGACAAC | |
| CCACAGCAGCGCTCTCCACTGGCCTATGTGCCCTTC | |
| TCTGCAGGACCCAGGAATTGCATCGGACAGAGCTTC | |
| GCCATGGCCGAGTTGCGCGTGGTTGTGGCACTAACA | |
| CTGCTACGTTTCCGCCTGAGCGTGGACCGAACGCGC | |
| AAGGTGCGGCGGAAGCCGGAGCTCATACTGCGCACG | |
| GAGAACGGGCTCTGGCTCAAGGTGGAGCCGCTGCCT | |
| CCGCGGGCCTGA | |

In a search of public sequence databases, the NOV10 nucleic acid sequence, localized to chromosome 19, has 1111 of 1578 bases (70%) identical to a gb:GENBANK-ID:HSU02388|acc:U02388.2 mRNA from Homo sapiens (Homo sapiens cytochrome P450 4F2 (CYP4F2) mRNA, complete cds) (E=7.4e⁻¹⁴⁷). Public nucleotide databases include all GenBank databases and the GeneSeq patent database.

The disclosed NOV10 polypeptide (SEQ ID NO: 36) encoded by SEQ ID NO: 35 has 532 amino acid residues and is presented in Table 10B using the one-letter amino acid code. Signal P, Psort and/or Hydropathy results predict that NOV10 has no signal peptide and is likely to be localized in the mitochondrial inner membrane with a certainty of 0.7491. In other embodiments, NOV10 may also be localized to the plasma membrane with a certainty of 0.6000, the Golgi body with a certainty of 0.4000, or in the endoplasmic reticulum (membrane) with a certainty of 0.3000. The most likely cleavage site for NOV10 is between positions 48 and 49: CRS-FY.

TABLE 10B
Encoded NOV10 protein sequence.

| | |
|---|---|
| MLPITDRLLHLLGLEKTAFRIYAVSTLLLFLLFFLF | (SEQ ID NO:36) |
| RLLLRFLRLCRSFYITCRRLRCFPQPPRRNWLLGHL | |
| GMYLPNEAGLQDEKKVLDNMHHVLLVWMGPVLPLLV | |
| LVHPDYIKPLLGASAAIAPKDDLFYGFLKPWLGDGL | |
| LLSKGDKWSRHRRLLTPAFHFDILKPYMKIFNQSAD | |
| IMHAKWRHLAEGSAVSLDMFEHISLMTLDSLQKCVF | |
| SYNSNCQEKMSDYISAIIELSALSVRRQYRLHHYLD | |
| FIYYRSADGRRFRQACDMVHHFTTEVIQERRRALRQ | |
| QGAEAWLKAKQGKTLDFIDVLLLARDEDGKELSDED | |
| IRAEADTFMFEGHDTTSSGISWMLFNLAKYPEYQEK | |
| CREEIQEVMKGRELEELEWDDLTQLPFTTMCIKESL | |
| RQYPPVTLVSRQCTEDIKLPDGRIIPKGIICLVSIY | |
| GTHHNPTVWPDSKVYNPYRFDPDNPQQRSPLAYVPF | |
| SAGPRNCIGQSFAMAELRVVVALTLLRFRLSVDRTR | |
| KVRRKPELILRTENFLWLKVEPLPPRAX | |

A search of sequence databases reveals that the NOV10 amino acid sequence has 339 of 505 amino acid residues (67%) identical to, and 415 of 505 amino acid residues (82%) similar to, the 520 amino acid residue ptnr:SWISSPROT-ACC:P78329 protein from Homo sapiens (Human) (Cytochrome P450 4F2 (EC 1.14.13.30) (CYPIVF2) (Leukotriene-B4 Omega-Hydroxylase) (Leukotriene-B4 20-Monooxygenase) (Cytochrome P450-LTB-Omega))(E=9.8e⁻¹⁸⁸). Public amino acid databases include the GenBank databases, SwissProt, PDB and PIR.

The Novel P450 disclosed in this invention is expressed in at least lung. This information was derived by determining the tissue sources of the sequences that were included in the invention including but not limited to SeqCalling sources, Public EST sources, Literature sources, and/or RACE sources.

In addition, the sequence is predicted to be expressed in colon and liver because of the expression pattern of (GENBANK-ID: gb:GENBANK-ID:HSU02388|acc:U02388.2) a closely related Homo sapiens cytochrome P450 4F2 (CYP4F2) mRNA, complete cds homolog.

The disclosed NOV10 polypeptide has homology to the amino acid sequences shown in the BLASTP data listed in Table 10C.

TABLE 10C
BLAST results for NOV10

| Gene Index/ | | Length | Identity | Positives | |
|---|---|---|---|---|---|
| Identifier | Protein/Organism | (aa) | (%) | (%) | Expect |
| gi\|14767705\|ref\|XP_{—} | cytochrome P450, | 520 | 309/481 | 378/481 | 0.0 |
| 029072.1\| | subfamily IVF, | | (64%) | (78%) | |
| (XM_029072) | polypeptide 3 | | | | |
| | [Homo sapiens] | | | | |
| gi\|2997737\|gb\| | cytochrome P-450 | 520 | 305/481 | 379/481 | 0.0 |
| AAC08589.1\| | [Homo sapiens] | | (63%) | (78%) | |
| (AF054821) | | | | | |
| gi\|4503241\|ref\| | cytochrome P450, | 520 | 308/481 | 378/481 | 0.0 |
| NP_000887.1\| | subfamily IVF, | | (64%) | (78%) | |
| (NM_000896) | polypeptide 3; | | | | |
| | leukotriene B4 | | | | |
| | omega | | | | |
| | hydroxylase; | | | | |
| | leukotriene-B4 | | | | |
| | 20-monooxygenase; | | | | |
| | cytochrome P450- | | | | |
| | LTB-omega | | | | |
| | [Homo sapiens] | | | | |
| gi\|13435391\|ref\|NP_{—} | cytochrome P450, | 520 | 304/481 | 380/481 | 0.0 |
| 001073.3\| | subfamily IVF, | | (63%) | (78%) | |
| (NM_001082) | polypeptide 2; | | | | |
| | leukotriene B4 omega- | | | | |
| | hydroxylase; | | | | |
| | leukotriene-B4 | | | | |
| | 20-monooxygenase | | | | |
| | [Homo sapiens] | | | | |
| gi\|4519535\|dbj\| | Leukotriene B4 | 520 | 303/481 | 380/481 | 0.0 |
| BAA75823.1\| | omega-hydroxylase | | (62%) | (78%) | |
| (AB015306) | [Homo sapiens] | | | | |

The homology between these and other sequences is shown graphically in the ClustalW analysis shown in Table 10D. In the ClustalW alignment of the NOV10 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Tables 01E-10F lists the domain description from DOMAIN analysis results against NOV10. This indicates that the NOV10 sequence has properties similar to those of other proteins known to contain this domain.

TABLE 10E
Domain Analysis of NOV10

gnl|Pfam|pfam00067, p450, Cytochrome P450. Cytochrome P450s are
involved in the oxidative degradation of various compounds.
Particularly well known for their role in the degradation of
environmental toxins and mutagens. Structure is mostly alpha, and
binds a heme cofactor. (SEQ ID NO:73)
CD-Length = 445 residues, 80.0% aligned
Score = 282 bits (722). Expect = 3e − 77

| | | | |
|---|---|---|---|
| Query: | 152 | WSRHRRLLTPAFHFDILKPYMKIFNQSADIMHAKWRHLAEGSAVSLDMFEHISLMTLDSL | 211 |
| | | \| + \|\|\|\|\| \| \| + \| \|+ + + \| + +\|+ \| ++ \|+ + | |
| Sbjct: | 88 | WRQLRRLLTLRF-FGMGKRS-KEERIQEEARDLVERLRKEQGSPIDITELLAPAPLNVI | 145 |
| Query: | 212 | QKCVFSYNSNCQEKMSDYISAIIELSALSVRRQYRLHHYLDFIYYRSADGRRFRQACDMV | 271 |
| | | +\| + ++ +++ \| +\|+ \| \|\|\| \| \|+ +\| + | |
| Sbjct: | 146 | CSLLFGVRFDYED--PEFLKLIDKLNELFFLVSPW-GQLLDFFRYLPGSHRKAFKAAKDL | 202 |
| Query: | 272 | HHFTTEVIQERRRALRQQGAEAWLKAKQGKTLDFIDVLLL-ARDEDGKELSDEDIRAEAD | 330 |
| | | + ++\|+\|\|\| \| \| \|\|+\| \|\|+ \|+ \| \| \|\|+\|\|+++\| | |
| Sbjct: | 203 | KDYLDKLIEERRETLEP-----------GDPRDFLDSLLIEAKREGGSELTDEELKATVL | 251 |
| Query: | 331 | TFMFEGHDTTSSGISWMLFNLAKYPEYQEKCREEIQEVMKGRELEELEWDDLTQLPFTTM | 390 |
| | | +\| \| \|\|\|\|\| +\|\| \|+ \|\|\|+\|\| \| \| \|\|\|\| \|\|+ +\|\| +\|+ | |
| Sbjct: | 252 | DLLFAGTDTTSSTLSWALYLLAKHPEVQAKLREEIDEVI--GRDRSPTYDDRANMPYLDA | 309 |
| Query: | 391 | CIKESLRQYPPV-TLVSRQCTEDIKLPDGRIIPKGIICLVSIYGTHHNPTVWPDSKVYNP | 449 |
| | | \|\|\|+\|\| +\| \| \|+ \| \|\|\| ++ \|\| +\|\|\|\| + +\|++\| \| +\| \|+\|+ + ++\| | |
| Sbjct: | 310 | VIKETLRLHPVVPLLLPRVATEDTEI-DGYLIPKGTLVIVNLYSLHRDPKVFPNPEEFDP | 368 |
| Query: | 450 | YRFDPDNPQQRSPLAYVPFSAGPRNCIGQSFAMAELRVVVALTLLRFRLSVDRTRKVRRK | 509 |
| | | \|\| +\| + + \|++\|\| \|\|\|\|\|\|+\|+ \| \|\| + +\| \| \|\| \| + + | |
| Sbjct: | 369 | ERFLDENGKFKKSYAFLPFGAGPRNCLGERLARMELFLFLATLLQRFELELVPPGDIPLT | 428 |
| Query: | 510 | PELILRTENGLWLKV | 524 |
| | | \|+ + ++ | |
| Sbjct: | 429 | PKPLGLPSKPPLYQL | 443 |

The P450 gene superfamily is a biologically diverse class of oxidase enzymes; members of the class are found in all organisms. P450 proteins are clinically and toxicologically important in humans; they are the principal enzymes in the metabolism of drugs and xenobiotic compounds, as well as in the synthesis of cholesterol, steroids and other lipids. Induction of some P450 genes can also be a risk factor for several types of cancer. This diversity of function is mirrored in the diversity of nucleotide and protein sequences; there are currently over 100 human P450 forms described. Allelic forms of many cytochrome P450 genes have been identified as causing quantitatively different rates of drug metabolism, and hence are important to consider in the development of safe and effective human pharmaceutical therapies. [reviewed in E. Tanaka, J Clinical Pharmacy & Therapeutics 24:323-329, 1999].

The disclosed NOV10 nucleic acid of the invention encoding a P450-like protein includes the nucleic acid whose sequence is provided in Table 10A or a fragment thereof. The invention also includes a mutant or variant nucleic acid any of whose bases may be changed from the corresponding base shown in Table 10A while still encoding a protein that maintains its P450-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 30% percent of the bases may be so changed.

The disclosed NOV10 protein of the invention includes the P450-like protein whose sequence is provided in Table 10B. The invention also includes a mutant or variant protein any of whose residues may be changed from the corresponding residue shown in Table 10B while still encoding a protein that maintains its P450-like activities and physiological functions, or a functional fragment thereof. In the mutant or variant protein, up to about 33% percent of the residues may be so changed.

The invention further encompasses antibodies and antibody fragments, such as F_{ab} or (F_{ab})₂, that bind immunospecifically to any of the proteins of the invention.

The above defined information for this invention suggests that this P450-like protein (NOV10) may function as a member of a “P450 family”. Therefore, the NOV10 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV10 nucleic acids and proteins of the invention are useful in potential therapeutic applications implicated in cancer including but not limited to various pathologies and disorders.

NOV10 nucleic acids and polypeptides are further useful in the generation of antibodies that bind immuno-specifically to the novel NOV10 substances for use in therapeutic or diagnostic methods. These antibodies may be generated according to methods known in the art, using prediction from hydrophobicity charts, as described in the “Anti-NOVX Antibodies” section below. The disclosed NOV10 protein has multiple hydrophilic regions, each of which can be used as an immunogen. In one embodiment, a contemplated NOV10 epitope is from about amino acids 50 to 100. In another embodiment, a NOV10 epitope is from about amino acids 120 to 180. In further embodiments, a NOV10 epitope is from about amino acids 200 to 420, from about amino acids 450 to 480, or from about amino acids 490 to 510. These novel proteins can be used in assay systems for functional analysis of various human disorders, which will help in understanding of pathology of the disease and development of new drug targets for various disorders.

NOV11

NOV11 includes three novel Integrin-like FG-GAP domain containing novel protein-like proteins disclosed below. The disclosed sequences have been named NOV11 a and NOV11b.

NOV11a

A disclosed NOV11nucleic acid of 3025 nucleotides (also referred to as CG55752-01) encoding a novel Alpha Glucosidase 2, Alpha Neutral Subunit-like protein is shown in Table 11A. An open reading frame was identified beginning with an ATG initiation codon at nucleotides 28-30 and ending with a TGA codon at nucleotides 2929-2931. A putative untranslated region upstream from the initiation codon is underlined in Table 11A. The start and stop codons are in bold letters.

TABLE 11A
NOV11a nucleotide sequence.

| | |
|---|---|
| ACAGGTGCCTGGGGGTCAGGCTTCCGCATGCGGGCT | (SEQ ID NO:37) |
| GCAGTTGCTGGCATTGCCTTCCGCAGGAGGCGTCAG | |
| AAACAGTGGCTTTCCAAGAAGTCCACCTATCAGGCA | |
| TTATTGGATTCAGTCACAACAGATGAAGACAGCACC | |
| AGGTTCCAAATCATCAATGAAGCAAGTAAGGTGAGG | |
| CGTCAGAAACAGTGGCTTTCCAAGAAGTCCACCTAT | |
| CAGGCATTATTGGATTCAGTCACAACAGATGAAGAC | |
| AGCACCAGGTTCCAAATCATCAATGAAGCAAGTAAG | |
| GTGCCTCTCCTGGCTGAAATTTATGGTATAGAAGGA | |
| AACATTTTCAGGCTTAAAATTAATGAAGAGACTCCT | |
| CTAAAACCCAGATTTGAAGTTCCGGATGTCCTCACA | |
| AGCAAGCCAAGCACTGTAAGGATTTCATGCTCTGGG | |
| GACACAGGCAGTCTGATATTGGCAGATGGAAAAGGA | |
| GACCTGAAGTGCCATATCACAGCAAACCCATTCAAG | |
| GTAGACTTGGTGTCTGAAGAAGAGGTTGTGATTAGC | |
| ATAAATTCCCTGGGCCAATTATACTTTGAGCATGGC | |
| AGGGCCCCTAGGGTCTCTTTCTCGGATAAGGTTAAT | |
| CTCACGCTTGGTAGCATATGGGATAAGATCAAGAAC | |
| CTTTTCTCTAGGCAAGGATCAAAAGACCCAGCTGAG | |
| GGCGATGGGGCCCAGCCTGAGGAAACACCCAGGGAT | |
| GGCGACAAGCCAGAGGAGACTCAGGGGAAGGCAGAG | |
| AAAGATGAGCCAGGAGCCTGGGAGGAGACATTCAAA | |
| ACTCACTCTGACAGCAAGCCGTATGGCCCTTCTTCT | |
| ATTGGTTTGGATTTCTCCTTGCATGGATTTGAGCAT | |
| CTTTATGGGATCCCACAACATGCAGAATCACACCAA | |
| CTTAAAAATACTGGGGATGGAGATGCTTACCGTCTT | |
| TATAACCTGGATGTCTATGGATACCAAATATATGAT | |
| AAAATGGGCATTTATGGTTCAGTACCTTATCTCCTG | |
| GCCCACAAACTGGGCAGAACTATAGGTATTTTCTGG | |
| CTGAATGCCTCGGAAACACTGGTGGAGATCAATACA | |
| GAGCCTGCAGGGATAGTCATCTTTGGTCCTGTCTCT | |
| TTGATTTATCAAAGCCAGGGAGATACACCTCTAACA | |
| ACTCATGTGCACTGGATGTCAGAGAGTGGCATCATT | |
| GATGTTTTTCTGCTGACAGGACCTACACCTTCTGAT | |
| GTCTTCAAACAGTACTCACACCTTACAGGTACACAA | |
| GCCATGCCCCCTCTTTTCTCTTTGGGATACCACCAG | |
| TGCCGCTGGAACTATGAAGATGAGCAGGATGTAAAA | |
| GCAGTGGATGCAGGGTTTGATGAGCATGACATTCCT | |
| TATGATGCCATGTGGCTGGACATAGAGCACACTGAG | |
| GGCAAGAGGTACTTCACCTGGGACAAAAACAGATTC | |
| CCAAACCCCAAGAGGATGCAAGAGCTGCTCAGGAGC | |
| AAAAAGCGTAAGCTTGTGGTCATCAGTGATCCCCAC | |
| ATCAAGATTGAACCTGACTACTCAGTATATGTGAAG | |
| GCCAAAGATCAGGGCTTCTTTGTGAAGAATCAGGAA | |
| GGGGAAGACTTTGAAGGGGTGTGTTGGCCAGGTATG | |
| AAATCATACCTGGATTTCACCAATCCCAAGGTCAGA | |
| GAGTGGTATTCAAGTATGTTCAGTTCCAATTGTGAT | |
| GGATCTACGGACATCCTCTTCCTTTGGAATGACATG | |
| AATGAGCCTTCTGTCTTTAGAGGGCCAGAGCAAACC | |
| ATGCAGAAGAATGCCATTCATCATGGCAATTGGGAG | |
| CACAGAGAGCTCCACAACATCTACGGTTTTTATATG | |
| GCTACTGCAGAAGGACTGATAAAACGATCTAAAGGG | |
| AAGGAGAGACCCTTTGTTCTTACACGTTCTTTCTTT | |
| GCTGGATCACAAAAGTATGGTGCCGTGTGGACAGGC | |
| GACAACACAGCAGAATGGAGCAACTTGAAAATTTCT | |
| ATCCCAATGTTACTCACTCTCAGCATTACTGGGATC | |
| TCTTTTTGCGGAGCTGACATAGGCGGGTTCATTGGG | |
| AATCCAGAGACAGAGCTGCTAGTGCGTTGGTACCAG | |
| GCTGGAGCCTACCAGCCCTTCTTCCGTGGCCATGCC | |
| ACCATGAACACCAAGCGACGAGAGCCCTGGCTCTTT | |
| GGGGAGGAACACACCCGACTCATCCGAGAAGCCATC | |
| AGAGAGCGCTATGGCCTCCTGCCATATTGGTATTCT | |
| CTGTTCTACCATGCACACGTGGCTTCCCAACCTGTC | |
| ATGAGGCCTCTGTGGGTAGAGTTCCCTGATGAACTA | |
| AAGACTTTTGATATGGAAGATGAATACATGTTAGGG | |
| AGTGCATTATTGGTTCATCCAGTCACAGAACCAAAA | |
| GCCACCACAGTTGATGTGTTTCTTCCAGGATCAAAT | |
| GAGGTAGTCTGGTATGACTATAAGACATTTGCTCAT | |
| TGGGAAGGAGGGTGTACTGTAAAGATCCCAGTACTG | |
| TTACAGATTCCAGTGTTTCAGCGAGGTGGAAGTGTG | |
| ATACCAATAAAGACAACTGTAGGAAAATCCACAGGC | |
| TGGATGACTGAATCCTCCTATGGACTCCGGGTTGCT | |
| CTAAGCACTCTCCAGGGTTCTTCAGTGGGTGAGTTA | |
| TATCTTGATGATGGCCATTCATTCCAATACCTCCAC | |
| CAGAAGCAATTTTTGCACAGGAAGTTTTCATTCTGT | |
| TCCAGTGTTCTGGTGGCCTCCTCTCCAGTATCTCAA | |
| GGACACTTACATACCCCACTCAGCATGACAAAAGCC | |
| CTGCTTTTCACTGTATCGTCTCCAGCCAGCGTGAAA | |
| ATGCGGCTTCACTACAGCCCAGAGAAAAGGGCCAGG | |
| TTTAGTCATTGTGCCAAAACATCCATCCTGAGCCTG | |
| GAGAAGCTCTCACTCAACATTGCCACTGACTGGGAG | |
| GTCCGCATCATATGACAAAGAACTGCCCCTGGTGAT | |
| GTGAGCAGGGACCTGCCTGCCCCTTTCAACCTTTCC | |
| CCTCACCTTTTTTGAGATTTTTGCTGCAATCTGTTT | |
| G | |

In a search of public sequence databases, the NOV11a nucleic acid sequence, located on chromosome 15 has 1839 of 2742 bases (67%) identical to a gb:GENBANK-ID:AF144074|acc:AF144074.1 mRNA from Homo sapiens (Homo sapiens glucosidase II alpha subunit mRNA, complete cds) (E=2.7e⁻²⁰⁵). Public nucleotide databases include all GenBank databases and the GeneSeq patent database.

The disclosed NOV11a polypeptide (SEQ ID NO: 38) encoded by SEQ ID NO: 37 has 967 amino acid residues and is presented in Table 11B using the one-letter amino acid code. Signal P, Psort and/or Hydropathy results predict that NOV11a has no signal peptide and is likely to be localized in the microbody (peroxisome) with a certainty of 0.7480. hn other embodiments, NOV11a may also be localized to the mitochondrial inner membrane with acertainty of 0.7070, the mitochondrial intermembrane space with a certainty of 0.6143, or in the mitochondrial matrix space with a certainty of 0.5762.

TABLE 11B
Encoded NOV11a protein sequence.

| | |
|---|---|
| MRAAVAGIAFRRRRQKQWLSKKSTYQALLDSVTTDE | (SEQ ID NO:38) |
| DSTRFQIINEASKVRRQKQWLSKKSTYQALLDSVTT | |
| DEDSTRFQIINEASKVPLLAEIYGIEGNIFRLKINE | |
| ETPLKPRFEVPDVLTSKPSTVRISCSGDTGSLILAD | |
| GKGDLKCHITANPFKVDLVSEEEVVISINSLGQLYF | |
| EHGRAPRVSFSDKVNLTLGSIWDKIKNLFSRQGSKD | |
| PAEGDGAQPEETPRDGDKPEETQGKAEKDEPGAWEE | |
| TFKTHSDSKPYGPSSIGLDFSLHGFEHLYGIPQHAE | |
| SHQLKNTGDGDAYRLYNLDVYGYQIYDKMGIYGSVP | |
| YLLAHKLGRTIGIFWLNASETLVEINTEPAGIVIFG | |
| PVSLIYQSQGDTPLTTHVHWMSESGIIDVFLLTGPT | |
| PSDVFKQYSHLTGTQAMPPLFSLGYHQCRWNYEDEQ | |
| DVKAVDAGFDEHDIPYDAMWLDIEHTEGKRYFTWDK | |
| NRFPNPKRMQELLRSKKRKLVVISDPHIKIEPDYSV | |
| YVKAKDQGFFVKNQEGEDFEGVCWPGMKSYLDFTNP | |
| KVREWYSSMFSSNCDGSTDILFLWNDMNEPSVFRGP | |
| EQTMQKNAIHHGNWEHRELHNIYGFYMATAEGLIKR | |
| SKGKERPFVLTRSFFAGSQKYGAVWTGDNTAEWSNL | |
| KISIPMLLTLSITGISFCGADIGGFIGNPETELLVR | |
| WYQAGAYQPFFRGHATMNTKRREPWLFGEEHTRLIR | |
| EAIRERYGLLPYWYSLFYHAHVASQPVMRPLWVEFP | |
| DELKTFDMEDEYMLGSALLVHPVTEPKATTVDVFLP | |
| GSNEVVWYDYKTFAHWEGGCTVKIPVLLQIPVFQRG | |
| GSVIPIKTTVGKSTGWMTESSYGLRVALSTLQGSSV | |
| GELYLDDGHSFQYLHQKQFLHRKFSFCSSVLVASSP | |
| VSQGHLHTPLSMTKALLFTVSSPASVKMRLHYSPEK | |
| RARFSHCAKTSILSLEKLSLNIATDWEVRII | |

A search of sequence databases reveals that the NOV11a amino acid sequence has 551 of 964 amino acid residues (57%) identical to, and 709 of 964 amino acid residues (73%) similar to, the 966 amino acid residue ptnr:SPTREMBL-ACC:Q9P0X0 protein from Homo sapiens (Human) (Glucosidase II Alpha Subunit) (E=9.7e⁻³⁰⁷). Public amino acid databases include the GenBank databases, SwissProt, PDB and PIR.

NOV11a is expressed in at least Adrenal Gland/Suprarenal gland, Aorta, Brain, Hippocampus, Kidney, Lung, Lymph node, Ovary, Parathyroid Gland, Prostate, Salivary Glands, Thyroid, Tonsils, Trachea, Uterus, Whole Organism. This information was derived by determining the tissue sources of the sequences that were included in the invention including but not limited to SeqCalling sources, Public EST sources, Literature sources, and/or RACE sources.

In addition, the sequence is predicted to be expressed in Brain, Hippocampus, Kidney, Lung because of the expression pattern of (GENBANK-ID: gb:GENBANK-ID:AF144074|acc: AF144074.1) a closely related Homo sapiens glucosidase II alpha subunit mRNA, complete cds homolog.

NOV11b

A disclosed NOV11b nucleic acid of 4483 nucleotides (also referred to as CG55752-02) encoding a novel Alpha Glucosidase 2-like protein is shown in Table 11C. An open reading frame was identified beginning with an ATG initiation codon at nucleotides 204-206 and ending with a TGA codon at nucleotides 2946-2948. A putative untranslated region upstream from the initiation codon is underlined in Table 11C. The start and stop codons are in bold letters.

TABLE 11C
NOV11b nucleotide sequence.

| | |
|---|---|
| AACGCTAGTTTGGGCCTGAAAAATTCCAGGAGCAAG | (SEQ ID NO:39) |
| AGTCAAGATTTGTCACTCCATGAGAATCTGGAGGGG | |
| ACTCCCTTCCCAGAAACTTGACGATGAAGTACTGGT | |
| TGTAATTTTAGAAAGACACCCAATCGGCTTTTTTAA | |
| AAGATCGCCCAGGGCCCTTGTCCTGAGAGCTGGGAG | |
| CTGGTCGGAGTGACAGAGAAGCCATGGAAGCAGCAG | |
| TGAAAGAGGAAATAAGTGTTGAAGATGAAGCTGTAG | |
| ATAAAAACATTTTCAGAGACTGTAACAAGATCGCAT | |
| TTTACAGGCGTCAGAAACAGTGGCTTTCCAAGAAGT | |
| CCACCTATCAGGCATTATTGGATTCAGTCACAACAG | |
| ATGAAGACAGCACCAGGTTCCAAATCATCAATGAAG | |
| CAAGTAAGGTTCCTCTCCTGGCTGAAATTTATGGTA | |
| TAGAAGGAAACATTTTCAGGCTTAAAATTAACGAAG | |
| AGACTCCTCTAAAACCCAGATTTGAAGTTCCGGATG | |
| TCCTCACAAGCAAGCCAAGCACTGTAAGGCTGATTT | |
| CATGCTCTGGGGACACAGGCAGTCTGATATTGGCAG | |
| ATGGAAAAGGAGACCTGAAGTGCCATATCACAGCAA | |
| ACCCATTCAAGGTAGACTTGGTGTCTGAAGAAGAGG | |
| TTGTGATTAGCATAAATTCCCTGGGCCAATTATACT | |
| TTGAGCATCTACAGATTCTTCACAAACAAAGAGCTG | |
| CTAAAGAAAATGAGGAGGAGACATCAGTGGACACCT | |
| CTCAGGAAAATCAAGAAGATCTGGGCCTGTGGGAAG | |
| AGAAATTTGGAAAATTTGTGGATATCAAAGCTAATG | |
| GCCCTTCTTCTATTGGTTTGGATTTCTCCTTGCATG | |
| GATTTGAGCATCTTTATGGGATCCCACAACATGCAG | |
| AATCACACCAACTTAAAAATACTGGTGATGGAGATG | |
| CTTACCGTCTTTATAACCTGGATGTCTATGGATACC | |
| AAATATATGATAAAATGGGCATTTATGGTTCAGTAC | |
| CTTATCTCCTGGCCCACAAACTGGGCAGAACTATAG | |
| GTATTTTCTGGCTGAATGCCTCGGAAACACTGGTGG | |
| AGATCAATACAGAGCCTGCAGTAGAGTACACACTGA | |
| CCCAGATGGGCCCAGTTGCTGCTAAACAAAAGGTCA | |
| GATCTCGCACTCATGTGCACTGGATGTCAGAGAGTG | |
| GCATCATTGATGTTTTTCTGCTGACAGGACCTACAC | |
| CTTCTGATGTCTTCAAACAGTACTCACACCTTACAG | |
| GCACACAAGCCATGCCCCCTCTTTTCTCTTTGGGAT | |
| ACCACCAGTGCCGCTGGAACTATGAAGATGAGCAGG | |
| ATGTAAAAGCAGTGGATGCAGGGTTTGATGAGCATG | |
| ACATTCCTTATGATGCCATGTGGCTGGACATAGAGC | |
| ACACTGAGGGCAAGAGGTACTTCACCTGGGACAAAA | |
| ACAGATTCCCAAACCCCAAGAGGATGCAAGAGCTGC | |
| TCAGGAGCAAAAAGCGTAAGCTTGTGGTCATCAGTG | |
| ATCCCCACATCAAGATTGATCCTGACTACTCAGTAT | |
| ATGTGAAGGCCAAAGATCAGGGCTTCTTTGTGAAGA | |
| ATCAGGAAGGGGAAGACTTTGAAGGGGTGTGTTGGC | |
| CAGGTCTCTCCTCTTACCTGGATTTCACCAATCCCA | |
| AGGTCAGAGAGTGGTATTCAAGTCTTTTTGCTTTCC | |
| CTGTTTATCAGGGATCTACGGACATCCTCTTCCTTT | |
| GGAATGACATGAATGAGCCTTCTGTCTTTAGAGGGC | |
| CAGAGCAAACCATGCAGAAGAATGCCATTCATCATG | |
| GCAATTGGGAGCACAGAGAGCTCCACAACATCTACG | |
| GTTTTTATCATCAAATGGCTACTGCAGAAGGACTGA | |
| TAAAACGATCTAAAGGGAAGGAGAGACCCTTTGTTC | |
| TTACACGTTCTTTCTTTGCTGGATCACAAAAGTATG | |
| GTGCCGTGTGGACAGGCGACAACACAGCAGAATGGA | |
| GCAACTTGAAAATTTCTATCCCAATGTTACTCACTC | |
| TCAGCATTACTGGGATCTCTTTTTGCGGAGCTGACA | |
| TAGGCGGGTTCATTGGGAATCCAGAGACAGAGCTGC | |
| TAGTGCGTTGGTACCAGGCTGGAGCCTACCAGCCCT | |
| TCTTCCGTGGCCATGCCACCATGAACACCAAGCGAC | |
| GAGAGCCCTGGCTCTTTGGGGAGGAACACACCCGAC | |
| TCATCCGAGAAGCCATCAGAGAGCGCTATGGCCTCC | |
| TGCCATATTGGTATTCTCTGTTCTACCATGCACACG | |
| TGGCTTCCCAACCTGTCATGAGGCCTCTGTGGGTAG | |
| AGTTCCCTGATGAACTAAAGACTTTTGATATGGAAG | |
| ATGAATACATGCTGGGGAGTGCATTATTGGTTCATC | |
| CAGTCACAGAACCAAAAGCCACCACAGTTGATGTGT | |
| TTCTTCCAGGATCAAATGAGGTCTGGTATGACTATA | |
| AGACATTTGCTCATTGGGAAGGAGGGTGTACTGTAA | |
| AGATCCCAGTAGCCTTGGACACTATTCCAGTGTTTC | |
| AGCGAGGTGGAAGTGTGATACCAATAAAGACAACTG | |
| TAGGAAAATCCACAGGCTGGATGACTGAATCCTCCT | |
| ATGGACTCCGGGTTGCTCTAAGCACTAAGGGTTCTT | |
| CAGTGGGTGAGTTATATCTTGATGATGGCCATTCAT | |
| TCCAATACCTCCACCAGAAGCAATTTTTGCACAGGA | |
| AGTTTTCATTCTGTTCCAGTGTTCTGATCAATAGTT | |
| TTGCTGACCAGAGGGGTCATTATCCCAGCAAGTGTG | |
| TGGTGGAGAAGATCTTGGTCTTAGGCTTCAGGAAGG | |
| AGCCATCTTCTGTGACTACCCACTCATCTGATGGTA | |
| AAGATCAGCCTGTGGCTTTTACGTATTGTGCCAAAA | |
| CATCCATCCTGAGCCTGGAGAAGCTCTCACTCAACA | |
| TTGCCACTGACTGGGAGGTCCGCATCATATGACAAA | |
| GAACTGCCCCTGGTGATGTGAGCAGGGACCTGCCTG | |
| CCCCTTTCAACCTTTCCCCTCACCTTTTTTGAGATT | |
| TTTGCTGCAATCTGTTTGCCTTCCCTGAATCAAAAT | |
| AATCTTTCATTCGTCACCATTATACTAATGAACAAT | |
| AGATTTCATGTTTCAAAATTTCAGATTTTACATGTT | |
| AAGATGTACTAACAATATTCCTTGTATCAAACATCT | |
| CCTTTTCTCCCTGATACATAGCCCTGAGACATTTAT | |
| AGCGTTCAGGAGTCTTCTATTGCTTCCATTCCTTCA | |
| GCAGGGCTGCGTGGGTCTGTTTTAACGTGGGCCAAG | |
| CCTACCTGGGCAGCCCATTTGCCAGGGCTTGCCTCA | |
| GGCCATGCAGCATTGGCGCTCTGGCTGCAGCAGCTG | |
| AGTTGCTCAAGGCCAGTGTCCAAGTGGACAGCAGCC | |
| TCTGGTACTCCCCCCAGTTATCTTCCACCCACATGG | |
| ACTGGGCAGAGCAGCCCTCTTCTGTGTGCACTGCAT | |
| ACGCTGCAGCCGTGGGAGTTATTCTCCCCTAGAGAT | |
| CGACTTGGCAGCACGAAGGATTCTTTTCTCTTTCAT | |
| GCTTCTCAGGCTCAATAGTTTCTAATTAATCTTAAA | |
| ATCCATGTCTTTTACATTGTTTTTTTAATTAAGTGC | |
| TGTTTACTAACCAAATAATATTTATAACATGAGTAA | |
| GCTATAATTAATAACAATGAAATAAATACCCATGTA | |
| CCCACCACTGGACTTCAGAAGTAGAACTCATGACTG | |
| GGACTAGGATGAGGCAAGGGAGACCCTGGCCTTGGG | |
| CACAAAATGTAAGGGATGCCAAAAAAATACAGTAAT | |
| CAAAGTAAGTAATATTTCAATCCAATATTTTTAAAA | |
| ATCAGAATTAATGCAAAAAAAACCATGATGAACAAA | |
| ATATTAAAATTTAAAATAAAGACAGGATTAGTATTA | |
| CTGAGTTTTCCTTTTGTCCCAGGCTTTAATATGGCT | |
| TGGCATGGGGCAGAACATTACAACATACCAGTCGTG | |
| TCATGGTGCCCAAGGCTCCACAGACCTCAGTGGCTC | |
| CCTGCTGCCTGCCACAGCATCTGTTTTAGCAGCCTC | |
| GACTCCTCAGCACTCCTCAGCACACACCTCTTCTTA | |
| TCAGGCTTCCTCCACTTAGCAACTTGCTAACGGCCA | |
| CCTCTGTGCCTTCTGATCCCTGGGCGCCAATATCCT | |
| CCTGCCCTTACCATCCTTCCAGGCCCAACTTAAATC | |
| CCACTTTCCCATGAAGCCTAACTGCGTGAACACCCC | |
| TACCCCCATACCCATTAGCAGTGATTTTGCCCTTCC | |
| CCGTAATGCTGTCCCACTTATAACTGTGCTCTACTT | |
| AGCATTCTCAGGGATCATACCTTAATGTTTTCAGTA | |
| TGTCTGCGTTCTCCTACTAGATTGTATGTCCCTCAA | |
| GAGCATGTTCTGTTTCTCTTCTGTCTGACAGAGCAC | |
| TATTATACCTGACTTTCAGTAACTGTTAGCTGTGAT | |
| TAGTTAGCTGGTGGATTTAATTGATTAAAAAATTAC | |
| GATTGAATGTAAAAAAAAA | |

In a search of public sequence databases, the NOV11b nucleic acid sequence, located on chromosome 15 has 1459 of 2258 bases (64%) identical to a gb:GENBANK-ID:MMU92793|acc:U92793.1 mRNA from Mus musculus (Mus musculus alpha glucosidase II alpha subunit mRNA, complete cds) (E=7.2e⁻¹⁴⁷). Public nucleotide databases include all GenBank databases and the GeneSeq patent database.

The disclosed NOV11b polypeptide (SEQ ID NO: 40) encoded by SEQ ID NO: 39 has 914 amino acid residues and is presented in Table 11D using the one-letter amino acid code. Signal P, Psort and/or Hydropathy results predict that NOV11b has no signal peptide and is likely to be localized in the endoplasmic reticulum (membrane) with a certainty of 0.8500. In other embodiments, NOV11b may also be localized to the microbody (peroxisome) with a certainty of 0.7480, the plasma membrane with a certainty of 0.4400, or in the mitochondrial inner membrane with a certainty of 0.1000.

TABLE 11D
Encoded NOV11b protein sequence.

| | |
|---|---|
| MEAAVKEEISVEDEAVDKNIFRDCNKIAFYRRQKQW | (SEQ ID NO:40) |
| LSKKSTYQALLDSVTTDEDSTRFQIINEASKVPLLA | |
| EIYGIEGNIFRLKINEETPLKPRFEVPDVLTSKPST | |
| VRLISCSGDTGSLILADGKGDLKCHITANPFKVDLV | |
| SEEEVVISINSLGQLYFEHLQILHKQRAAKENEEET | |
| SVDTSQENQEDLGLWEEKFGKFVDIKANGPSSIGLD | |
| FSLHGFEHLYGIPQHAESHQLKNTGDGDAYRLYNLD | |
| VYGYQIYDKMGIYGSVPYLLAHKLGRTIGIFWLNAS | |
| ETLVEINTEPAVEYTLTQMGPVAAKQKVRSRTHVHW | |
| MSESGIIDVFLLTGPTPSDVFKQYSHLTGTQAMPPL | |
| FSLGYHQCRWNYEDEQDVKAVDAGFDEHDIPYDAMW | |
| LDIEHTEGKRYFTWDKNRFPNPKRMQELLRSKKRKL | |
| VVISDPHIKIDPDYSVYVKAKDQGFFVKNQEGEDFE | |
| GVCWPGLSSYLDFTNPKVREWYSSLFAFPVYQGSTD | |
| ILFLWNDMNEPSVFRGPEQTMQKNAIHHGNWEHREL | |
| HNIYGFYHQMATAEGLIKRSKGKERPFVLTRSFFAG | |
| SQKYGAVWTGDNTAEWSNLKISIPMLLTLSITGISF | |
| CGADIGGFIGNPETELLVRWYQAGAYQPFFRGHATM | |
| NTKRREPWLFGEEHTRLIREAIRERYGLLPYWYSLF | |
| YHAHVASQPVMRPLWVEFPDELKTFDMEDEYMLGSA | |
| LLVHPVTEPKATTVDVFLPGSNEVWYDYKTFAHWEG | |
| GCTVKIPVALDTIPVFQRGGSVIPIKTTVGKSTGWM | |
| TESSYGLRVALSTKGSSVGELYLDDGHSFQYLHQKQ | |
| FLHRKFSFCSSVLINSFADQRGHYPSKCVVEKILVL | |
| GFRKEPSSVTTHSSDGKDQPVAFTYCAKTSILSLEK | |
| LSLNIATDWEVRII | |

A search of sequence databases reveals that the NOV11b amino acid sequence has 466 of 912 amino acid residues (51%) identical to, and 640 of 912 amino acid residues (70%) similar to, the 944 amino acid residue ptnr:SPTREMBL-ACC:P79403 protein from Sus scrofa (Pig) (Glucosidase II) (E=7.1e⁻²⁶⁰). Public amino acid databases include the GenBank databases, SwissProt, PDB and PIR.

NOV11b is expressed in at least Adrenal Gland/Suprarenal gland, Aorta, Brain, Hippocampus, Kidney, Lung, Lymph node, Ovary, Parathyroid Gland, Prostate, Salivary Glands, Thyroid, Tonsils, Trachea, Uterus. Expression information was derived from the tissue sources of the sequences that were included in the derivation of the sequence of NOV11b. The sequence is predicted to be expressed in T cells because of the expression pattern of (GENBANK-ID: gb:GENBANK-ID:MMU92793|acc:U92793.1) a closely related Mus musculus alpha glucosidase II alpha subunit mRNA, complete cds.

NOV11c

A disclosed NOV11c nucleic acid of 3015 nucleotides (also referred to as CG55752-03) encoding a novel Glucosidase II-like protein is shown in Table 11E. An open reading frame was identified beginning with an ATG initiation codon at nucleotides 204-206 and ending with a TGA codon at nucleotides 2946-2948. A putative untranslated region upstream from the initiation codon is underlined in Table 11E. The start and stop codons are in bold letters.

TABLE 11A
NOV11c nucleotide sequence.

| | |
|---|---|
| AACGCTAGTTTGGGCCTGAAAAATTCCAGGAGCAAG | (SEQ ID NO:41) |
| AGTCAAGATTTGTCACTCCATGAGAATCTGGAGGGG | |
| ACTCCCTTCCCAGAAACTTGACGATGAAGTACTGGT | |
| TGTAATTTTAGAAAGACACCCAATCGGCTTTTTTAA | |
| AAGATCGCCCAGGGCCCTTGTCCTGAGAGCTGGGAG | |
| CTGGTCGGAGTGACAGAGAAGCCATGGAAGCAGCAG | |
| TGAAAGAGGAAATAAGTGTTGAAGATGAAGCTGTAG | |
| ATAAAAACATTTTCAGAGACTGTAACAAGATCGCAT | |
| TTTACAGGCGTCAGAAACAGTGGCTTTCCAAGAAGT | |
| CCACCTATCAGGCATTATTGGATTCAGTCACAACAG | |
| ATGAAGACAGCACCAGGTTCCAAATCATCAATGAAG | |
| CAAGTAAGGTTCCTCTCCTGGCTGAAATTTATGGTA | |
| TAGAAGGAAACATTTTCAGGCTTAAAATTAACGAAG | |
| AGACTCCTCTAAAACCCAGATTTGAAGTTCCGGATG | |
| TCCTCACAAGCAAGCCAAGCACTGTAAGGCTGATTT | |
| CATGCTCTGGGGACACAGGCAGTCTGATATTGGCAG | |
| ATGGAAAAGGAGACCTGAAGTGCCATATCACAGCAA | |
| ACCCATTCAAGGTAGACTTGGTGTCTGAAGAAGAGG | |
| TTGTGATTAGCATAAATTCCCTGGGCCAATTATACT | |
| TTGAGCATCTACAGATTCTTCACAAACAAAGAGCTG | |
| CTAAAGAAAATGAGGAGGAGACATCAGTGGACACCT | |
| CTCAGGAAAATCAAGAAGATCTGGGCCTGTGGGAAG | |
| AGAAATTTGGAAAATTTGTGGATATCAAAGCTAATG | |
| GCCCTTCTTCTATTGGTTTGGATTTCTCCTTGCATG | |
| GATTTGAGCATCTTTATGGGATCCCACAACATGCAG | |
| AATCACACCAACTTAAAAATACTGGTGATGGAGATG | |
| CTTACCGTCTTTATAACCTGGATGTCTATGGATACC | |
| AAATATATGATAAAATGGGCATTTATGGTTCAGTAC | |
| CTTATCTCCTGGCCCACAAACTGGGCAGAACTATAG | |
| GTATTTTCTGGCTGAATGCCTCGGAAACACTGGTGG | |
| AGATCAATACAGAGCCTGCAGTAGAGTACACACTGA | |
| CCCAGATGGGCCCAGTTGCTGCTAAACAAAAGGTCG | |
| GATCTCGCACTCATGTGCACTGGATGTCAGAGAGTG | |
| GCATCATTGATGTTTTTCTGCTGACAGGACCTACAC | |
| CTTCTGATGTCTTCAAACAGTACTCACACCTTACAG | |
| GCACACAAGCCATGCCCCCTCTTTTCTCTTTGGGAT | |
| ACCACCAGTGCCGCTGGAACTATGAAGATGAGCAGG | |
| ATGTAAAAGCAGTGGATGCAGGGTTTGATGAGCATG | |
| ACATTCCTTATGATGCCATGTGGCTGGACATAGAGC | |
| ACACTGAGGGCAAGAGGTACTTCACCTGGGACAAAA | |
| ACAGATTCCCAAACCCCAAGAGGATGCAAGAGCTGC | |
| TCAGGAGCAAAAAGCGTAAGCTTGTGGTCATCAGTG | |
| ATCCCCACATCAAGATTGATCCTGACTACTCAGTAT | |
| ATGTGAAGGCCAAAGATCAGGGCTTCTTTGTGAAGA | |
| ATCAGGAAGGGGAAGACTTTGAAGGGGTGTGTTGGC | |
| CAGGTCTCTCCTCTTACCTGGATTTCACCAATCCCA | |
| AGGTCAGAGAGTGGTATTCAAGTCTTTTTGCTTTCC | |
| CTGTTTATCAGGGATCTACGGACATCCTCTTCCTTT | |
| GGAATGACATGAATGAGCCTTCTGTCTTTAGAGGGC | |
| CAGAGCAAACCATGCAGAAGAATGCCATTCATCATG | |
| GCAATTGGGAGCACAGAGAGCTCCACAACATCTACG | |
| GTTTTTATCATCAAATGGCTACTGCAGAAGGACTGA | |
| TAAAACGATCTAAAGGGAAGGAGAGACCCTTTGTTC | |
| TTACACGTTCTTTCTTTGCTGGATCACAAAAGTATG | |
| GTGCCGTGTGGACAGGCGACAACACAGCAGAATGGA | |
| GCAACTTGAAAATTTCTATCCCAATGTTACTCACTC | |
| TCAGCATTACTGGGGTCTCTTTTTGCGGAGCTGACA | |
| TAGGCGGGTTCATTGGGAATCCAGAGACAGAGCTGC | |
| TAGTGCGTTGGTACCAGGCTGGAGCCTACCAGCCCT | |
| TCTTCCGTGGCCATGCCACCATGAACACCAAGCGAC | |
| GAGAGCCCTGGCTCTTTGGGGAGGAACACACCCGAC | |
| TCATCCGAGAAGCCATCAGAGAGCGCTATGGCCTCC | |
| TGCCATATTGGTATTCTCTGTTCTACCATGCACACG | |
| TGGCTTCCCAACCTGTCATGAGGCCTCTGTGGGTAG | |
| AGTTCCCTGATGAACTAAAGACTTTTGATATGGAAG | |
| ATGAATACATGCTGGGGAGTGCATTATTGGTTCATC | |
| CAGTCACAGAACCAAAAGCCACCACAGTTGATGTGT | |
| TTCTTCCAGGATCAAATGAGGTCTGGTATGACTATA | |
| AGACATTTGCTCATTGGGAAGGAGGGTGTACTGTAA | |
| AGATCCCAGTAGCCTTGGACACTATTCCAGTGTTTC | |
| AGCGAGGTGGAAGTGTGATACCAATAAAGACAACTG | |
| TAGGAAAATCCACAGGCTGGATGACTGAATCCTCCT | |
| ATGGACTCCGGGTTGCTCTAAGCACTAAGGGTTCTT | |
| CAGTGGGTGAGTTATATCTTGATGATGGCCATTCAT | |
| TCCAATACCTCCACCAGAAGCAATTTTTGCACAGGA | |
| AGTTTTCATTCTGTTCCAGTGTTCTGATCAATAGTT | |
| TTGCTGACCAGAGGGGTCACTATCCCAGCAAGTGTG | |
| TGGTGGAGAAGATCTTGGTCTTAGGCTTCAGGAAGG | |
| AGCCATCTTCTGTGACTACCCACTCATCTGATGGTA | |
| AAGATCAGCCTGTGGCTTTTACGTATTGTGCCAAAA | |
| CATCCATCCTGAGCCTGGAGAAGCTCTCACTCAACA | |
| TTGCCACTGACTGGGAGGTCCGCATCATATGACAAA | |
| GAACTGCCCCTGGTGATGTGAGCAGGGACCTGCCTG | |
| CCCCTTTCAACCTTTCCCCTCACCTTT | |

In a search of public sequence databases, the NOV11c nucleic acid sequence, located on chromosome 15 has 1459 of 2258 bases (64%) identical to a gb:GENBANK-ID:MMU92793|acc:U92793.1 mRNA from Mus musculus (Mus musculus alpha glucosidase II alpha subunit mRNA, complete cds) (E=7.2e⁻¹⁴⁷). Public nucleotide databases include all GenBank databases and the GeneSeq patent database.

The disclosed NOV11c polypeptide (SEQ ID NO: 42) encoded by SEQ ID NO: 41 has 914 amino acid residues and is presented in Table 11F using the one-letter amino acid code. Signal P, Psort and/or Hydropathy results predict that NOV11c has no signal peptide and is likely to be localized in the microbody (peroxisome) with a certainty of 0.7480. In other embodiments, NOV11c may also be localized to the nucleus with a certainty of 0.3000, the mitochondrial membrane space with a certainty of 0.1000, or in the lysosome (lumen) with a certainty of 0.1000.

TABLE 11F
Encoded NOV11c protein sequence.

| | |
|---|---|
| MEAAVKEEISVEDEAVDKNIFRDCNKIAFYRRQKQW | (SEQ ID NO:42) |
| LSKKSTYQALLDSVTTDEDSTRFQIINEASKVPLLA | |
| EIYGIEGNIFRLKINEETPLKPRFEVPDVLTSKPST | |
| VRLISCSGDTGSLILADGKGDLKCHITANPFKVDLV | |
| SEEEVVISINSLGQLYFEHLQILHKQRAAKENEEET | |
| SVDTSQENQEDLGLWEEKFGKFVDIKANGPSSIGLD | |
| FSLHGFEHLYGIPQHAESHQLKNTGDGDAYRLYNLD | |
| VYGYQIYDKMGIYGSVPYLLAHKLGRTIGIFWLNAS | |
| ETLVEINTEPAVEYTLTQMGPVAAKQKVGSRTHVHW | |
| MSESGIIDVFLLTGPTPSDVFKQYSHLTGTQAMPPL | |
| FSLGYHQCRWNYEDEQDVKAVDAGFDEHDIPYDAMW | |
| LDIEHTEGKRYFTWDKNRFPNPKRMQELLRSKKRKL | |
| VVISDPHIKIDPDYSVYVKAKDQGFFVKNQEGEDFE | |
| GVCWPGLSSYLDFTNPKVREWYSSLFAFPVYQGSTD | |
| ILFLWNDMNEPSVFRGPEQTMQKNAIHHGNWEHREL | |
| HNIYGFYHQMATAEGLIKRSKGKERPFVLTRSFFAG | |
| SQKYGAVWTGDNTAEWSNLKISIPMLLTLSITGVSF | |
| CGADIGGFIGNPETELLVRWYQAGAYQPFFRGHATM | |
| NTKRREPWLFGEEHTRLIREAIRERYGLLPYWYSLF | |
| YHAHVASQPVMRPLWVEFPDELKTFDMEDEYMLGSA | |
| LLVHPVTEPKATTVDVFLPGSNEVWYDYKTFAHWEG | |
| GCTVKIPVALDTIPVFQRGGSVIPIKTTVGKSTGWM | |
| TESSYGLRVALSTKGSSVGELYLDDGHSFQYLHQKQ | |
| FLHRKFSFCSSVLINSFADQRGHYPSKCVVEKILVL | |
| GFRKEPSSVTTHSSDGKDQPVAFTYCAKTSILSLEK | |
| LSLNIATDWEVRII | |

A search of sequence databases reveals that the NOV11c amino acid sequence has 467 of 912 amino acid residues (51%) identical to, and 640 of 912 amino acid residues (70%) similar to, the 944 amino acid residue ptnr:SPTREMBL-ACC:P79403 protein from Sus scrofa (Pig) (Glucosidase II) (E=7.3e⁻²⁶⁰). Public amino acid databases include the GenBank databases, SwissProt, PDB and PIR.

NOV11c is expressed in at least Adrenal Gland/Suprarenal gland, Aorta, Brain, Hippocampus, Kidney, Lung, Lymph node, Ovary, Parathyroid Gland, Prostate, Salivary Glands, Thyroid, Tonsils, Trachea, Uterus. Expression information was derived from the tissue sources of the sequences that were included in the derivation of the sequence of NOV11c.

NOV11d

A disclosed NOV11d nucleic acid of 3102 nucleotides (also referred to as CG55752-04) encoding a novel Glucosidase II-like protein is shown in Table 11G. An open reading frame was identified beginning with an ATG initiation codon at nucleotides 103-105 and ending with a TGA codon at nucleotides 2839-2841. A putative untranslated region upstream from the initiation codon is underlined in Table 11G. The start and stop codons are in bold letters.

TABLE 11G
NOV11d nucleotide sequence.

| | |
|---|---|
| TACTGGTTGTAATTTTAGAAAGACACCCAATCGGCT | (SEQ ID NO:43) |
| TTTTTAAAAGATCGCCCAGGGCCCTTGTCCTGAGAG | |
| CTGGGAGCTGGTCGGAGTGACAGAGAAGCCATGGAA | |
| GCAGCAGTGAAAGAGGAAATAAGTGTTGAAGATGAA | |
| GCTGTAGATAAAAACATTTTCAGAGACTGTAACAAG | |
| ATCGCATTTTACAGGCGTCAGAAACAGTGGCTTTCC | |
| AAGAAGTCCACCTATCGGGCATTATTGGATTCAGTC | |
| ACAACAGATGAAGACAGCACCAGGTTCCAAATCATC | |
| AATGAAGCAAGTAAGGTTCCTCTCCTGGCTGAAATT | |
| TATGGTATAGAAGGAAACATTTTCAGGCTTAAAATT | |
| AACGAAGAGACTCCTCTAAAACCCAGATTTGAAGTT | |
| CCGGATGTCCTCACAAGCAAGCCAAGCACTGTAAGG | |
| CTGATTTCATGCTCTGGGGACACAGGCAGTCTGATA | |
| TTGGCACATGGAAAAGGAGACCTGAAGTGCCATATC | |
| ACAGCAAACCCATTCAAGGTAGACTTGGTGTCTGAA | |
| GAAGAGGTTGTGATTAGCATAAATTCCCTGGGCCAA | |
| TTATACTTTGAGCATCTACAGATTCTTCACAAACAA | |
| AGAGCTGCTAAAGAAAATGAGGAGGAGACATCAGTG | |
| GACACCTCTCAGGAAAATCAAGAAGATCTGGGCCTG | |
| TGGGAAGAGAAATTTGGAAAATTTGTGGATATCAAA | |
| GCTAATGGCCCTTCTTCTATTGGTTTGGATTTCTCC | |
| TTGCATGGATTTGAGCATCTTTATGGGATCCCACAA | |
| CATGCAGAATCACACCAACTTAAAAATACTGGAGAT | |
| GCTTACCGTCTTTATAACCTGGATGTCTATGGATAC | |
| CAAATATATGATAAAATGGGCATTTATGGTTCAGTA | |
| CCTTATCTCCTGGCCCACAAACTGGGCAGAACTATA | |
| GCTATTTTCTGGCTGAATGCCTCGGAAACACTGGTG | |
| GAGATCAATACAGAGCCTGCAGTAGAGTACACACTG | |
| ACCCAGATGGGCCCAGTTGCTGCTAAACAAAAGGTC | |
| AGATCTCGCACTCATGTGCACTGGATGTCAGAGAGT | |
| GGCATCATTGATGTTTTTCTGCTGACAGGACCTACA | |
| CCTTCTGATGTCTTCAAACAGTACTCACACCTTACA | |
| GGTACGCAAGCCATGCCCCCTCTTTTCTCTTTGGGA | |
| TACCACCAGTGCCGCTGGAACTATGAAGATGAGCAG | |
| GATGTAAAAGCAGTGGATGCAGGGTTTGATGAGCAT | |
| GACATTCCTTATGATGCCATGTGGCTGGACATAGAG | |
| CACACTGAGGGCAAGAGGTACTTCACCTGGGACAAA | |
| AACAGATTCCCAAACCCCAAGAGGATGCAAGAGCTG | |
| CTCAGGAGCAAAAAGCGTAAGCTTGTGGTCATCAGT | |
| GATCCCCACATCAAGATTGAACCTGACTACTCAGTA | |
| TATGTGAAGGCCAAAGATCAGGGCTTCTTTGTGAAG | |
| AATCAGGAAGGGGAAGACTTTGAAGGGGTGTGTTGG | |
| CCAGGTCTCTCCTCTTACCTGGATTTCACCAATCCC | |
| AAGGTCAGAGAGTGGTATTCAAGTCTTTTTGCTTTC | |
| CCTGTTTATCAGGGATCTACGGACATCCTCTTCCTT | |
| TGGAATGACATGAATGAGCCTTCTGTCTTTAGAGGG | |
| CCAGAGCAAACCATGCAGAAGAATGCCATTCATCAT | |
| GGCAATTGGGAGCACAGAGAGCTCCACAACATCTAC | |
| GGTTTTTATCATCAAATGGCTACTGCAGAAGGACTG | |
| ATAAAACGATCTAAAGGGAAGGAGAGACCCTTTGTT | |
| CTTACACGTTCTTTCTTTGCTGGATCACAAAAGTAT | |
| GGTGCCGTGTGGACAGGCGACAACACAGCAGAATGG | |
| AGCAACTTGAAAATTTCTATCCCAATGTTACTCACT | |
| CTCAGCATTACTGGGATCTCTTTTTGCGGAGCTGAC | |
| ATAGGCGGGTTCATTGGGAATCCAGAGACAGAGCTG | |
| CTAGTGCGTTGGTACCAGGCTGGAGCCTACCAGCCC | |
| TTCTTCCGTGGCCATGCCACCATGAACACCAAGCGA | |
| CGAGAGCCCTGGCTCTTTGGGGAGGAACACACCCGA | |
| CTCATCCGAGAAGCCATCAGAGAGCGCTATGGCCTC | |
| CTGCCATATTGGTATTCTCTGTTCTACCATGCACAC | |
| GTGGCTTCCCAACCTGTCATGAGGCCTCTGTGGGTA | |
| GAGTTCCCTGATGAACTAAAGACTTTTGATATGGAA | |
| GATGAATACATGTTAGGGAGTGCATTATTGGTTCAT | |
| CCAGTCACAGAACCAAAAGCCACCACAGTTGATGTG | |
| TTTCTTCCAGGATCAAATGAGGTATGGTATGACTAT | |
| AAGACATTTGCTCATTGGGAAGGAGGGTGTACTGTA | |
| AAGATCCCAGTAGCCTTGGACACTATTCCAGTGTTT | |
| CAGCGAGGTGGAAGTGTGATACCAATAAAGACAACT | |
| GTAGGAAAATCCACAGGCTGGATGACTGAATCCTCC | |
| TATGGACTCCGGGTTGCTCTAAGCACTCAGGGTTCT | |
| TCAGTGGGTGAGTTATATCTTGATGATGGCCATTCA | |
| TTCCAATACCTCCACCAGAAGCAATTTTTGCACAGG | |
| AAGTTTTCATTCTGTTCCAGTGTTCTGATCAATAGT | |
| TTTGCTGACCAGAGGGGTCATTATCCCAGCAAGTGT | |
| GTGGTGGAGAAGATCTTGGTCTTAGGCTTCAGGAAG | |
| GAGCCATCTTCTGTGACTACCCACTCATCTGATGGT | |
| AAAGATCAGCCTGTGGCTTTTACGTATTGTGCCAAA | |
| ACATCCATCCTGAGCCTGGAGAAGCTCTCACTCAAC | |
| ATTGCCACTGACTGGGAGGTCCGCATCATATGACAA | |
| AGAACTGCCCCTGGTGATGTGAGCAGGGACCTGCCT | |
| GCCCCTTTCAACCTTTCCCCTCACCTTTTTTGAGAT | |
| TTTTGCTGCAATCTGTTTGTCTTCCCTGAATCAAAA | |
| TAATCTTTCATTCGTCACCATTATACTAATGAACAA | |
| TAGATTTCATGTTTCAAAATTTCAGATTTTACATGT | |
| TAAGATGTACTAACAATATTCCTTGTATCAAACATC | |
| TCCTTTTCTCCCTGATACATAGCCCTGAGACATTAT | |
| AGCGTC | |

In a search of public sequence databases, the NOV11d nucleic acid sequence, located on chromosome 15 has 1427 of 2214 bases (64%) identical to a gb:GENBANK-ID:MMU92793|acc:U92793.1 mRNA from Mus musculus (Mus musculus alpha glucosidase II alpha subunit mRNA, complete cds) (E=5.9e⁻¹⁴⁴). Public nucleotide databases include all GenBank databases and the GeneSeq patent database.

The disclosed NOV11d polypeptide (SEQ ID NO: 44) encoded by SEQ ID NO: 43 has 912 amino acid residues and is presented in Table 11H using the one-letter amino acid code. Signal P, Psort and/or Hydropathy results predict that NOV11d has no signal peptide and is likely to be localized in the endoplasmic reticulum (membrane) with a certainty of 0.8500. In other embodiments, NOV11d may also be localized to the microbody (peroxisome) with a certainty of 0.7480, the plasma membrane with a certainty of 0.4400, or in the mitochondrial inner membrane with a certainty of 0.1000.

TABLE 11H
Encoded NOV11d protein sequence.

| | |
|---|---|
| MEAAVKEEISVEDEAVDKNIFRDCNKIAFYRRQKQW | (SEQ ID NO:44) |
| LSKKSTYRALLDSVTTDEDSTRFQIINEASKVPLLA | |
| EIYGIEGNIFRLKINEETPLKPRFEVPDVLTSKPST | |
| VRLISCSGDTGSLILADGKGDLKCHITANPFKVDLV | |
| SEEEVVISINSLGQLYFEHLQILHKQRAAKENEEET | |
| SVDTSQENQEDLGLWEEKFGKFVDIKANGPSSIGLD | |
| FSLHGFEHLYGIPQHAESHQLKNTGDAYRLYNLDVY | |
| GYQIYDKMGIYGSVPYLLAHKLGRTIGIFWLNASET | |
| LVEINTEPAVEYTLTQMGPVAAKQKVRSRTHVHWMS | |
| ESGIIDVFLLTGPTPSDVFKQYSHLTGTQAMPPLFS | |
| LGYHQCRWNYEDEQDVKAVDAGFDEHDIPYDAMWLD | |
| IEHTEGKRYFTWDKNRFPNPKRMQELLRSKKRKLVV | |
| ISDPHIKIEPDYSVYVKAKDQGFFVKNQEGEDFEGV | |
| CWPGLSSYLDFTNPKVREWYSSLFAFPVYQGSTDIL | |
| FLWNDMNEPSVFRGPEQTMQKNAIHHGNWEHRELHN | |
| IYGFYHQMATAEGLIKRSKGKERPFVLTRSFFAGSQ | |
| KYGAVWTGDNTAEWSNLKISIPMLLTLSITGISFCG | |
| ADIGGFIGNPETELLVRWYQAGAYQPFFRGHATMNT | |
| KRREPWLFGEEHTRLIREAIRERYGLLPYWYSLFYH | |
| AHVASQPVMRPLWVEFPDELKTFDMEDEYMLGSALL | |
| VHPVTEPKATTVDVFLPGSNEVWYDYKTFAHWEGGC | |
| TVKIPVALDTIPVFQRGGSVIPIKTTVGKSTGWMTE | |
| SSYGLRVALSTQGSSVGELYLDDGHSFQYLHQKQFL | |
| HRKFSFCSSVLINSFADQRGHYPSKCVVEKILVLGF | |
| RKEPSSVTTHSSDGKDQPVAFTYCAKTSILSLEKLS | |
| LNIATDWEVRII | |

A search of sequence databases reveals that the NOV11d amino acid sequence has 636 of 653 amino acid residues (97%) identical to, and 644 of 653 amino acid residues (98%) similar to, the 653 amino acid residue ptnr:TREMBLNEW-ACC:BAB39324 protein from Macaca fascicularis (Crab eating macaque) (Cynomolgus monkey) (Hypothetical 74.7 KDA Protein) (E=0.0). Public amino acid databases include the GenBank databases, SwissProt, PDB and PIR.

NOV11d is expressed in at least the adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the sequence of NOV11d.

The disclosed NOV11 polypeptide has homology to the amino acid sequences shown in the BLASTP data listed in Table 11I.

TABLE 11I
BLAST results for NOV11

| Gene Index/ | | Length | Identity | Positives | |
|---|---|---|---|---|---|
| Identifier | Protein/Organism | (aa) | (%) | (%) | Expect |
| gi\|7672977\|gb\| | glucosidase II | 966 | 547/969 | 706/969 | 0.0 |
| AAF66685.1\| | alpha subunit | | (56%) | (72%) | |
| (AF144074) | [Homo sapiens] | | | | |
| gi\|6679891\|ref\|NP_{—} | alpha glucosidase | 966 | 538/969 | 707/969 | 0.0 |
| 032086.1\| | 2, alpha neutral | | (55%) | (72%) | |
| (NM_008060) | subunit [Mus musculus] | | | | |
| gi\|7661898\|ref\|NP_{—} | KIAA0088 protein; | 944 | 524/969 | 684/969 | 0.0 |
| 055425.1\| | likely ortholog | | (54%) | (70%) | |
| (NM_014610) | of mouse G2an | | | | |
| | alpha glucosidase | | | | |
| | 2, alpha neutral | | | | |
| | subunit [Homo sapiens] | | | | |
| gi\|577295\|dbj\| | The ha1225 gene product | 943 | 524/969 | 684/969 | 0.0 |
| BAA07642.1\| | related to is | | (54%) | (70%) | |
| (D42041) | human | | | | |
| | alpha- | | | | |
| | glucosidase. | | | | |
| | [Homo sapiens] | | | | |
| gi\|1890664\|gb\| | glucosidase II | 944 | 525/969 | 684/969 | 0.0 |
| AAB49757.1\| | [Sus scrofa] | | (54%) | (70%) | |
| (U71273) | | | | | |

The homology between these and other sequences is shown graphically in the ClustalW analysis shown in Table 11J. In the ClustalW alignment of the NOV11 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

Table 1K lists the domain description from DOMAIN analysis results against NOV11. This indicates that the NOV11 sequence has properties similar to those of other proteins known to contain this domain.

TABLE 11K
Domain Analysis of NOV11

gnl|Pfam|pfam01055, Glyco_hydro_31, Glycosyl hydrolases family 31.
Glycosyl hydrolases are key enzymes of carbohydrate metabolism. Family
31 comprises of enzymes that are, or similar to, alpha-
galactosidases. (SEQ ID NO:125)
CD-Length = 707 residues, 91.9% aligned
Score = 642 bits (1657), Expect = 0.0

| | | | |
|---|---|---|---|
| Query: | 244 | KDEPGAWEETFKTHSDSKPYGPSSIGLDFSLHGFEHLYGIPQHAESHQLKNTGDGDAYRL | 303 |
| | | ++ \|\| + + \| \|\| \|\|+ +\|\| ++\| + \| \| | |
| Sbjct: | 33 | STGDVLFDTTFGP----LVFSDQFLQLSTSLPSEYI-YGLGEHAHKLFRRDTNE--TYTL | 85 |
| Query: | 304 | YNLDVYGYQIYDKMGIYGSVPYLLAHK-LGRTIGIFWLNASETLVEINTEPAGIVIFGPV | 362 |
| | | +\| \|\| \| + + \|\|\| \|+ ++ + \| \|+\| \|\|++ \|+\| \|\| | |
| Sbjct: | 86 | WNRDVGPYSGDNNL--YGSHPFYMSLEDSGNAHGVFLLNSNAMEVDIGPGPA-------- | 135 |
| Query: | 363 | SLIYQSQGDTPLTTHVHWMSESGIIDVFLLTGPTPSDVFKQYSHLTGTQAMPPLFSLGYH | 422 |
| | | + + \|\|+\| + \|\|\|\| \|\| +\|\|+ \| \| \|+\|\| +\|\|\|+\| | |
| Sbjct: | 136 | ---------------LTYRVIGGILDFYFFLGPTPEDVLQQYTELIGRPALPPYWSLGFH | 180 |
| Query: | 423 | QCRWNYEDEQDVKAVDAGFDEHDIPYDAMWLDIEHTEGKRYFTWDKNRFPNPKRMQELLR | 482 |
| | | \|\|\| \| + +\|\| \| \| + +\|\| \| \|\|\|\|++ +\| + \|\|\|\| \|\|\| \|+ + \| | |
| Sbjct: | 181 | LCRWGYTNVSEVKTVVDGMRKANIPLDVQWLDIDYMDGYKDFTWDPVRFPGPEDFVKKLH | 240 |
| Query: | 483 | SKKRKLVVISDPHIKIEPD-YSVYVKAKDQGFFVKNQEGEDFEGVCWPGMKSYLDFTNPK | 541 |
| | | +\| +\| \|\|\| \|\| \| ++ \| \| + \|++\| \|\|\|\| \| \|+ \| \|\|\| ++ \|\|\|\|\|+ | |
| Sbjct: | 241 | AKGQKYVVILDPAISVDSASYYPYERGKEKGVFVKNPNGSDYIGEVWPGYTAFPDFTNPE | 300 |
| Query: | 542 | VREWYSSMFSSNCDGSTDILFLWNDMNEPSVFRGP----------------------EQT | 579 |
| | | \|+\|++ \| \| +\| \|\|\|\|\|\| \| \| +\| | |
| Sbjct: | 301 | ARKWWADEIKDFHD-SLPFDGIWIDMNEPSSFSEPGPNDSNLNYPPYAPNDGDGPLSSKT | 359 |
| Query: | 580 | MQKNAIHHGNWEHRELHNIYGFYM--ATAEGLIKRSKGKERPFVLTRSFFAGSQKYGAVW | 637 |
| | | \| +\|+\|+\| \|\| ++\|\|+\|\| \|\| \| \| \| + \|\| \|\|\|\|\|+\|\| \|\|\|\| +\| \| | |
| Sbjct: | 360 | MCMDAVHYGGVEHYDVHNLYGLSEAKATYEALKKVTGGK-RPFVLSRSTFAGSGRYAGHW | 418 |
| Query: | 638 | TGDNTAEWSNLKISIPMLLTLSITGISFCGADIGGFIGNPETELLVRWYQAGAYQPFFRG | 697 |
| | | \|\|\|\|\|\| \| +\|\| \|\|\| +\|+ ++ \|\| \| \|\|\|\| \|\| \|\| \|\| \|\|\| \| \|\|+ \|\| \| | |
| Sbjct: | 419 | TGDNTASWDDLKYSIPGVLSFNLFGIPFVGADICGFNGNTTEELCVRWMQLGAFYPFSRN | 478 |
| Query: | 698 | HATMNTKRREPWLFGEEHTRLIREAIRERYGLLPYWYSLFYHAHVASQPVMRPLWVEFPD | 757 |
| | | \| + \| +\|\|\|\|\| \|+\|+ \|\| \|\|\|\| \|+\|\|+ \|\|\|+ \|\|\|\|\|\|+ \|\|\|\| | |
| Sbjct: | 479 | HNHLGTIPQEPWLFDSVAAEASRKALNLRYTLLPYLYTLFHEAHVSGLPVMRPLFFEFPD | 538 |
| Query: | 758 | ELKTFDMEDEYMLGSALLVHPVTEPKATTVDVFLPGSNEVVWYDYKTFA--HWEGGCTVK | 815 |
| | | + +\|+\|++ +++ \|\|\|\|\|\| \|\| \|\| \|\|+\| +\|\|\| \|\|\| \| \| \|\| | |
| Sbjct: | 539 | DAETYDIDRQFLWGSALLVAPVLEPGATSVKAYLPGGR---WYDLYTGAGEASRGGNVTL | 595 |
| Query: | 816 | IPVLLQIPVFQRGGSVIPIKTTVGKSTGWMTESSYGLRVALSTLQGSSVGELYLDDGHSF | 875 |
| | | \| +\|\|\| \|\|\|\|+\|\| + +\| ++ + \| \|\|\| \|++ \|\|\|\|\|\|\|\| \| | |
| Sbjct: | 596 | SAPLDKIPVHVRGGSIIPTQEP-ALTTTESRDNPFHLLVALDD-NGTASGELYLDDGESI | 653 |
| Query: | 876 | QYLHQKQFLHRKFSFCSSVLVASSPVSQGH 905 | |
| | | + +\| +\|\| ++ \| + \|+ + | |
| Sbjct: | 654 | DTQ-RGDYLLVQFSANNNTLTGTEVVTGYY 682 | |

The gene sequence of invention described herein encodes for a novel member of the glucosidase family of enzymes. Specifically, the sequence encodes a novel alpha-glucosidase2 neutral subunit-like protein. Processing glycosidases also play a role in the folding of newly formed glycoproteins and in endoplasmic reticulum quality control. Glucosidases are also useful for the treatment of diabetes. By inhibiting the glucosidase enzymes of the golgi, the requirement for insulin decreases. Therefore the novel Alpha-Glucosidase2, Alpha Neutral Subunit-like protein could be useful for the treatment of metabolic and endocrine disorders such as diabetes type I and II.

Alpha-glucosidase which active at neutral pH appears as a doublet of enzyme activity on native gel electrophoresis and was termed neutral alpha-glucosidase AB. Neutral alpha-glucosidase AB is synonymous with the glycoprotein processing enzyme glucosidase II. A mutant mouse lymphoma line which is deficient in glucosidase II is also deficient in neutral alpha-glucosidase AB, as defined electrophoretically and quantitatively (less than 0.5% of parental). In contrast, both mutant and parental cell lines exhibited several lysosomal hydrolases which are processed by glucosidase II. Both glucosidase II and neutral alpha-glucosidase AB are high-molecular mass (greater than 200,000 dalton) anionic glycoproteins which bind to concanavalin A, have a broad pH optima (5.5-8.5), and have a similar Km for maltose (4.8 versus 2.1 mM) and the artificial substrate 4-methylumbelliferyl-alpha-D-glucopyranoside (35 versus 19 microM). Similar to human neutral alpha-glucosidase AB, purified rat glucosidase II migrates as a doublet of enzyme activity on native gel electrophoresis. Although rat glucosidase II has been reported to have a subunit size of 67 kDa, pig glucosidase II has been found to have a subunit size of 100 kDa, like the 98-kDa major protein in purified human neutral alpha-glucosidase A. glucosidase II is localized to the long arm of human chromosome II.PMID: 3881423, UI: 85104919

Processing glycosidases play an important role in N-glycan biosynthesis in mammalian cells by trimming Glc(3)Man(9)GlcNAc(2) and thus providing the substrates for the formation of complex and hybrid structures by Golgi glycosyltransferases. Membrane-bound alpha-glucosidase I and soluble alpha-glucosidase II of the endoplasmic reticulum remove the alpha1,2-glucose and alpha1,3-glucose residues, respectively, beginning immediately following transfer of Glc(3)Man(9)GlcNAc(2) to nascent polypeptides. The alpha-glucosidases participate in glycoprotein folding mediated by calnexin and calreticulin by forming the monoglucosylated high mannose oligosaccharides required for the interaction with the chaperones. In some mammalian cells, Golgi endo alpha-mannosidase provides an alternative pathway for removal of glucose residues. Removal of alpha1,2-linked mannose residues begins in the endoplasmic reticulum where trimming of mannose residues in the endoplasmic reticulum has been implicated in the targeting of malfolded glycoproteins for degradation. Removal of mannose residues continues in the Golgi with the action of alpha1,2-mannosidases IA and IB that can form Man(5)GlcNAc(2) and of alpha-mannosidase II that removes the alpha1,3- and alpha1,6-linked mannose from GlcNAcMan(5)GlcNAc(2) to form GlcNAcMan(3)GlcNAc(2). These membrane-bound Golgi enzymes have been cloned and shown to have very distinct patterns of tissue-specific expression. There are also broad specificity alpha-mannosidases that can trim Man(4-9)GlcNAc(2) to Man(3)GlcNAc(2), and provide an alternative pathway toward complex oligosaccharide formation. Cloning of the remaining alpha-mannosidases will be required to evaluate their specific functions in glycoprotein maturation. PMID: 10580131, UI: 20047733

Several new pharmacological agents have recently been developed to optimize the management of type 2 (non-insulin-dependent) diabetes mellitus. There are three general therapeutic modalities relevant to diabetes care. The first modality is lifestyle adjustments aimed at improving endogenous insulin sensitivity or insulin effect. This can be achieved by increased physical activity and bodyweight reduction with diet and behavioral modification, and the use of pharmacological agents or surgery. This first modality is not discussed in depth in this article. The second modality involves increasing insulin availability by the administration of exogenous insulin, insulin analogues, sulphonylureas and the new insulin secretagogue, repaglinide. The most frequently encountered adverse effect of these agents is hypoglycaemia. Bodyweight gain can also be a concern, especially in patients who are obese. The association between hyperinsulinaemia and premature atherosclerosis is still a debatable question. The third modality consists of agents such as biguanides and thiazolidinediones which enhance insulin sensitivity, or agents that decrease insulin requirements like the alpha-glucosidase inhibitors. Type 2 diabetes mellitus is a heterogeneous disease with multiple underlying pathophysiological processes. Therapy should be individualised based on the degree of hyperglycaemia, hyperinsulinaemia or insulin deficiency. In addition, several factors have to be considered when prescribing a specific therapeutic agent. These factors include efficacy, safety, affordability and ease of administration. PMID: 10929931, UI: 20383756

The prevalence of Type 2 diabetes rises steeply with age and involves beta-cell dysfunction and diminished sensitivity to insulin. beta-cell dysfunction is important in the development of hyperglycaemia while insulin resistance seems to play a major role in the atherogenic process resulting in cardiovascular disease. Current therapeutic options include lifestyle adjustments (exercise and diet), oral hypoglycaemic agents (sulphonylureas, newer beta-cell mediated insulin releasing drugs, alpha-glucosidase inhibitors, biguanides and thiazolidinediones) and insulin treatment. Oral hypoglycaemic agents are effective only temporarily in maintaining good glycaemic control, their efficacy should be determined from changes in fasting and postprandial glucose levels. Recent studies have shown that the early initiation of insulin therapy can establish good glycaemic control. PMID: 10383606, UI: 99315525

Genetic deficiency of lysosomal acid alpha-glucosidase (acid maltase) results in the autosomal recessive disorder glycogen storage disease type II (GSDII) in which intralysosomal accumulation of glycogen primarily affects function of skeletal and cardiac muscle. This report identifies 2 of 35 GSDII patients with co-occurence of cleft lip, considerably greater than the estimated frequency of nonsyndromic cleft lip with or without cleft palate of 1 in 700 to 1,000. Because several lines of evidence support a minor cleft lip/palate (Cl/P) locus on chromosome 17q close to the locus for GSDII. Patient I (of Dutch descent) was homozygous and the parents heterozygous for an intragenic deletion of exon 18 (deltaex 18), common in Dutch patients. Patient II was heterozygous for delta525T, a mutation also common in Dutch patients and a novel nonsense mutation (172 degrees C.-->T; Gln58Stop) in exon 2, the first coding exon. The mother was heterozygous for the delta525T and the father for the 172 degrees C.-->T; Gln58Stop. The finding that both patients carried intragenic mutations eliminates a contiguous gene syndrome. Whereas the presence of cleft lip/cleft palate in a patient with GSDII could be coincidental, these co-occurences could represent a modifying action of acid alpha-glucosidase deficiency on unlinked or linked genes that result in increased susceptibility for cleft lip. PMID: 10377006, UI: 99303499

Diabetes mellitus is the most common endocrine disease, accounting for over 200 million people affected worldwide. It is characterized by a lack of insulin secretion and/or increased cellular resistance to insulin, resulting in hyperglycemia and other metabolic disturbances. People with diabetes suffer from increased morbidity and premature mortality related to cardiovascular, microvascular and neuropathic complications. The Diabetes Control and Complication Trial (DCCT) has convincingly demonstrated the relationship of hyperglycemia to the development and progression of complications and showed that improved glycemic control reduced these complications. Although the DCCT exclusively studied patients with Type 1 diabetes, there is ample evidence to support the belief that the same relationship between metabolic control and clinical outcome exists in patients with Type 2 diabetes. Therefore, a major effort should be made to develop and implement more effective treatment regimes. This article reviews those novel drugs that have been recently introduced for the management of Type 2 diabetes, or that have reached an advanced level of study and will soon be proposed for preliminary clinical trials. They include: (i) compounds that promote the synthesis/secretion of insulin by the beta-cell; (ii) inhibitors of the alpha-glucosidase activity of the small intestine; (iii) substances that enhance the action of insulin at the level of the target tissues; and (iv) inhibitors of free fatty acid oxidation. PMID: 9816470, UI: 99033258

The disclosed NOV11 nucleic acid of the invention encoding a Alpha Glucosidase 2, Alpha Neutral Subunit-like protein includes the nucleic acid whose sequence is provided in Table 11A, 11C, 11E or a fragment thereof. The invention also includes a mutant or variant nucleic acid any of whose bases may be changed from the corresponding base shown in Table 11A, 11C, or 11E while still encoding a protein that maintains its Alpha Glucosidase 2, Alpha Neutral Subunit-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 33% percent of the bases may be so changed.

The disclosed NOV11 protein of the invention includes the Alpha Glucosidase 2, Alpha Neutral Subunit-like protein whose sequence is provided in Table 11B. 11D, or 11F. The invention also includes a mutant or variant protein any of whose residues may be changed from the corresponding residue shown in Table 11B, 11D, or 11F while still encoding a protein that maintains its Alpha Glucosidase 2, Alpha Neutral Subunit-like activities and physiological functions, or a functional fragment thereof. In the mutant or variant protein, up to about 43% percent of the residues may be so changed.

The invention further encompasses antibodies and antibody fragments, such as Fab or (F_{ab})₂, that bind immunospecifically to any of the proteins of the invention.

The above defined information for this invention suggests that this Alpha Glucosidase 2, Alpha Neutral Subunit-like protein (NOV11) may function as a member of a “Alpha Glucosidase 2, Alpha Neutral Subunit family”. Therefore, the NOV11 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV11 nucleic acids and proteins of the invention are useful in potential therapeutic applications implicated in various diseases and pathologies.

NOV11 nucleic acids and polypeptides are further useful in the generation of antibodies that bind immuno-specifically to the novel NOV11 substances for use in therapeutic or diagnostic methods. These antibodies may be generated according to methods known in the art, using prediction from hydrophobicity charts, as described in the “Anti-NOVX Antibodies” section below. The disclosed NOV11 protein has multiple hydrophilic regions, each of which can be used as an immunogen. In one embodiment, a contemplated NOV11 epitope is from about amino acids 5 to 90. In another embodiment, a NOV11 epitope is from about amino acids 180 to 350. In additional embodiments, a NOV11 epitope is from about amino acids 400 to 670, from about amino acids 680 to 780, from about amino acids 860 to 900, and from about amino acids 920 to 950. These novel proteins can be used in assay systems for functional analysis of various human disorders, which will help in understanding of pathology of the disease and development of new drug targets for various disorders.

NOV12

NOV12 includes three novel Mechanical stress induced protein-like proteins disclosed below. The disclosed sequences have been named NOV12a, NOV12b, and NOV12c.

NOV12a

A disclosed NOV12 nucleic acid of 7876 nucleotides (also referred to as Curagen Accession No. CG55776-01) encoding a novel Mechanical stress induced protein-like protein is shown in Table 12A. An open reading frame was identified beginning with an ATG initiation codon at nucleotides 6-8 and ending with a TGA codon at nucleotides 7857-7859. Putative untranslated regions upstream from the initiation codon and downstream of the termination codon are underlined in Table 12A. The start and stop codons are in bold letters.

TABLE 12A
NOV12 nucleotide sequence (SEQ ID NO:45).

TCAGGATGAAGGTAAAAGGCAGAGGAATCACCTGCTTGCTGGTCTCCTTT
GCTGTGATCTGCCTGGTCGCCACCCCTGGGGGCAAGGCCTGTCCTCGCCG
CTGTGCCTGTTATATGCCTACGGAGGTACACTGCACATTTCGGTACCTGA
CTTCCATCCCAGACAGCATCCCGCCCAATGTGGAACGCATCAATTTAGGG
TACAACAGCTTGGTTAGATTGATGGAAACAGATTTTTCTGGCCTGACCAA
ACTGGAGTTACTCATGCTTCACAGCAATGGCATTCACACAATCCCTGACA
AGACCTTCTCAGATTTGCAGGCCTTGCAGGTGAGACTGATGGTCTTAAAA
ATGAGCTATAATAAAGTCCGAAAACTTCAGAAAGATACTTTTTATGGCCT
CAGGAGCTTGACACGATTGCACATGGACCACAACAATATTGAGTTTATAA
ACCCAGAGGTTTTTTATGGGCTCAACTTTCTCCGCCTGGTGCACTTGGAA
GGAAATCAGCTCACTAAGCTCCACCCAGATACATTTGTCTCTTTGAGCTA
CCTCCAGATATTTAAAATCTCTTTCATTAAGTTCCTATACTTGTCTGATA
ACTTCCTGACCTCCCTCCCTCAAGAGATGGTCTCCTATATGCCTGACCTA
GACAGCCTTTACCTGCATGGAAACCCATGGACCTGTGATTGCCATTTAAA
GTGGTTGTCTGACTGGATACAGGAGAAGCCAGGTATCTATATTGTNTTAC
CAGATGTAATAAAATGCAAAAAAGATAGAAGTCCCTCTAGTGCTCAGCAG
TGTCCACTTTGCATGAACCCTAGGACTTCTAAAGGCAAGCCGTTAGCTAT
GGTCTCAGCTGCAGCTTTCCAGTGTGCCAAGCCAACCATTGACTCATCCC
TGAAATCAAAGAGCCTGACTATTCTGGAAGACAGTAGTTCTGCTTTCATC
TCTCCCCAAGGTTTCATGGCACCCTTTGGCTCCCTCACTTTGAATATGAC
AGATCAGTCTGGAAATGAAGCTAACATGGTCTGCAGTATTCAAAAGCCCT
CAAGGACATCACCCATTGCATTCACTGAAGAAAATGACTACATCGTGCTA
AATACTTCATTTTCAACATTTTTGGTGTGCAACATAGATTACGGTCACAT
TCAGCCAGTGTGGCAAATTTTGGCTTTGTACAGTGATTCTCCTCTGATAC
TAGAAAGGAGCCACTTGCTTAGTGAAACACCGCAGCTCTATTACAAATAT
AAACAGGTGGCTCCTAAGCCTGAAGACATTTTTACCAACATAGAGGCAGA
TCTCAGAGCAGATCCCTCTTGGTTAATGCAAGACCAAATTTCCTTGCAGC
TGAACAGAACTGCCACCACATTCAGTACATTACAGATCCAGTACTCCAGT
GATGCTCAAATCACTTTACCAAGAGCAGAGATGAGGCCAGTGAAACACAA
ATGGACTATGATTTCAAGGGATAACAATACTAAGCTGGAACATACTGTCT
TGGTAGGTGGAACCGTTGGCCTGAACTGCCCAGGCCAAGGAGACCCCACC
CCACACGTGGATTGGCTTCTAGCTGATGGAAGTAAAGTGAGAGCCCCTTA
TGTCAGTGAGGATGGACGGATCCTAATAGACAAAAGTGGAAAATTGGAAC
TCCAGATGGCTGATAGTTTTGACACAGGCGTATATCACTGTATAAGCAGC
AATTATGATGATGCAGATATTCTCACCTATAGGATAACTGTGGTAGAACC
TTTGGTCGAAGCCTATCAGGAAAATGGGATTCATCACACAGTTTTCATTG
GTGAAACACTTGATCTTCCATGCCATTCTACTGGTATCCCAGATGCCTCT
ATTAGCTGGGTTATTCCAGGAAACAATGTGCTCTATCAGTCATCAAGAGA
CAAGAAAGTTCTAAACAATGGCACATTAAGAATATTACAGGTCACCCCGA
AAGACCAAGGTTATTATCGCTGTGTGGCAGCCAACCCATCAGGGGTTGAT
TTTTTGATTTTCCAAGTTTCAGTCAAGATGAAAGGACAAAGGCCCTTGGA
GCATGATGGAGAAACAGAGGGATCTGGACTTGATGAGTCCAATCCTATTG
CTCATCTTAAGGAGCCACCAGGTGCACAACTCCGTACATCTGCTCTGATG
GAGGCTGAGGTTGGAAAACACACCTCAAGCACAAGTAAGAGGCACAACTA
TCGGGAATTAACACTCCAGCGACGTGGAGATTCAACACATCGACGTTTTA
GGGAGAATAGGAGGCATTTCCCTCCCTCTGCTAGGAGAATTGACCCACAA
CATTGGGCGGCACTGTTGGAGAAAGCTAAAAAGAATGCTATGCCAGACAA
GCGAGAAAATACCACAGTGAGCCCACCCCCAGTGGTCACCCAACTCCCAA
ACATACCTGGTGAAGAAGACGATTCCTCAGGCATGCTCGCTCTACATGAG
GAATTTATGGTCCCGGCCACTAAAGCTTTGAACCTTCCAGCAAGGACAGT
GACTGCTGACTCCAGAACAATATCTGATAGTCCTATGACAAACATAAATT
ATGGCACAGAATTCTCTCCTGTTGTGAATTCACAAATACTACCACCTGAA
GAACCCACAGATTTCAAACTGTCTACTGCTATTAAAACTACAGCCATGTC
AAAGAATATAAACCCAACCATGTCAAGCCAAATACAAGGCACAACCAATC
AACATTCATCCACTGTCTTTCCACTGCTACTTGGAGCAACTGAATTTCAG
GACTCTGACCAGATGGGAAGAGGAAGAGAGCATTTCCAAAGTAGACCCCC
AATAACAGTAAGGACTATGATCAAAGATGTCAATGTCAAAATGCTTAGTA
GCACCACCAACAAACTATTATTAGAGTCAGTAAATACCACAAATAGTCAT
CAGACATCTGTAAGAGAAGTGAGTGAACCCAGGCACAATCACTTCTATTC
TCACACTACTCAAATACTTAGCACCTCCACGTTCCCTTCAGATCCACACA
CAGCTGCTCATTCTCAGTTTCCGATCCCTAGAAATAGTACAGTTAACATC
CCGCTGTTCAGACGCTTTGGGAGGCAGAGGAAAATTGGCGGAAGGGGGCG
GATTATCAGCCCATATAGAACTCCAGTTCTGCGACGGCATAGATACAGCA
TTTTCAGGTCAACAACCAGAGGTTCTTCTGAAAAAAGCACTACTGCATTC
TCAGCCACAGTGCTCAATGTGACATGTCTGTCCTGTCTTCCCAGGGAGAG
GCTCACCACTGCCACAGCAGCATTGTCTTTTCCAAGTGCTGCTCCCATCA
CCTTCCCCAAAGCTGACATTGCTAGAGTCCCATCAGAAGAGTCTACAACT
CTAGTCCAGAATCCACTATTACTACTTGAGAACAAACCCAGTGTAGAGAA
AACAACACCCACAATAAAATATTTCAGGACTGAAATTTCCCAAGTGACTC
CAACTGGTGCAGTCATGACATATGCTCCAACATCCATACCCATGGAAAAA
ACTCACAAAGTAAACGCCAGTTACCCACGTGTGTCTAGCACCAATGAAGC
TAAAAGAGATTCAGTGATTACATCGTCACTTTCAGGTGCTATCACCAAGC
CACCAATGACTATTATAGCCATTACAAGGTTTTCAAGAAGGAAAATTCCC
TGGCAACAGAACTTTGTAAATAACCATAACCCAAAAGGCAGATTAAGGAA
TCAACATAAAGTTAGTTTACAAAAAAGCACAGCTGTGATGCTTCCTAAAA
CATCTCCTGCTTTACCACAGAGACAAAGTCTCCCCTCGCACCACACTACG
ACCAAAACACACAATCCTGGAAGTCTTCCAACAAAGAAGGAGCTTCCCTT
CCCACCCCTTAACCCTATGCTTCCTAGTATTATAAGCAAAGACTCAAGTA
CAAAAAGCATCATATCAACGCAAACAGCAATACCAGCAACAACTCCTACC
TTCCCTGCATCTGTCATCACTTATGAAACCCAAACAGAGAGATCTAGAGC
ACAAACAATACAAAGAGAACAGGAGCCTCAAAAGAAGAACAGGACTGACC
CAAACATCTCTCCAGACCAGAGTTCTGGCTTCACTACACCCACTGCTATG
ACACCTCCTGTTCTAACCACAGCCGAAACTTCAGTCAAGCCCAGTGTCTC
TGCATTCACTCATTCCCCACCAGAAAACACAACTGGGATTTCAAGCACAA
TCAGTTTTCATTCAAGAACTCTTAATCTGACAGATGTGATTGAAGAACTA
GCCCAAGCAAGTACTCAGACTTTGAAGAGCACAATTGCTTCTGAAACAAC
TTTGTCCAGCAAATCACACCAGAGTACCACAACTAGGAAAGCAATCATTA
GACACTCAACCATACCACCATTCTTGAGCAGCAGTGCTACTCTAATGCCA
GTTCCCATCTCCCCTCCCTTTACTCAGAGAGCAGTTACTGACAACGTGGC
GACTCCCATTTCCGGGCTTATGACAAATACAGTGGTCAAGCTGCACGAAT
CCTCAAGGCACAATGCTAAACCACAGCAATTAGTAGCAGAGGTTGCAACA
TCCCCCAAGGTTCACCCAAATGCCAAGTTCACAATTGGAACCACTCACTT
CATCTACTCTAATCTGTTACATTCTACTCCCATGCCAGCACTAACAACAG
TTAAATCACAGAATTCTAAATTAACTCCATCTCCCTGGGCAGAAAACCAA
TTTTGGCACAAACCATACTCAGAAATTGCTGAAAAAGGCAAAAAGCCAGA
AGTAAGCATGTTGGCTACTACAGGCCTGTCCGAGGCCACCACTCTTGTTT
CAGATTGGGATGGACAGAAGAACACAAAGAAGAGTGACTTTGATAAGAAA
CCAGTTCAAGAAGCAACAACTTCCAAACTCCTTCCCTTTGACTCTTTGTC
TAGGTATATATTTGAAAAGCCCAGGATAGTTGGAGGAAAAGCTGCAAGTT
TTACTATTCCAGCTAACTCAGATGCCTTTCTTCCCTGTGAAGCTGTTGGA
AATCCCCTGCCCACCATTCATTGGACCAGAGTCCCATCAGGTATGTCAGG
ACTTGATTTATCTAAGAGGAAACAGAATAGCAGGGTCCAGGTTCTCCCCA
ATGGTACCCTGTCCATCCAGAGGGTGGAAATTCAGGACCGCGGACAGTAC
TTGTGTTCCGCATCCAATCTGTTTGGCACAGACCACCTTCATGTCACCTT
GTCTGTGGTTTCCTATCCTCCCAGGATCCTGGAGAGACGTACCAAAGAGA
TCACAGTTCATTCCGGAAGCACTGTGGAACTGAAGTGCAGAGCAGAAGGT
AGGCCAAGCCCTACAGTTACCTGGATTCTTGCAAACCAAACAGTTGTCTC
AGAATCATCCCAGGGAAGTAGGCAGGCTGTGGTGACGGTTGACGGAACAT
TGGTCCTCCACAATCTCAGTATTTATGACCGTGGCTTTTACAAATGTGTG
GCCAGCAACCCAGGTGGCCAGGATTCACTGCTGGTTAAAATACAAGTCAT
TGCAGCACCACCTGTTATTCTAGAGCAAAGGAGGCAAGTCATTGTAGGCA
CTTGGGGTGAAAGTTTAAAACTGCCCTGTACTGCAAAAGGAACTCCTCAG
CCCAGCGTTTACTGGGTCCTCTCTGATGGCACTGAAGTGAAACCATTACA
GTTTACCAATTCCAAGTTGTTCTTATTTTCAAATGGGACTTTGTATATAA
GAAACCTAGCCTCTTCAGACAGGGGCACTTATGAATGCATTGCTACCAGT
TCCACTGGTTCGGAGCGAAGAGTAGTAATGCTTACAATGGAAGAGCGAGT
GACCAGCCCCAGGATAGAAGCTGCATCCCAGAAAAGGACTGAAGTGAATT
TTGGGGACAAATTACTACTGAACTGCTCAGCCACTGGGGAGCCCAAACCC
CAAATAATGTGGAGGTTACCATCCAAGGCTGTGGTCGACCAGCAGCATAG
GGTGGGCAGCTGGATCCACGTCTACCCTAATGGATCCCTGTTTATTGGAT
CAGTAACAGAAAAAGACAGTGGTGTCTACTTGTGTGTGGCAAGAAACAAA
ATGGGGGATGATCTGATACTGATGCATGTTAGCCTAAGACTGAAACCTGC
CAAAATTGACCACAAGCAGTATTTTAGAAAGCAAGTGCTCCATGGGAAAG
ATTTCCAAGTAGATTGCAAAGCTTCCGGCTCCCCAGTGCCAGAGATATCT
TGGAGTTTGCCTGATGGAACCATGATCAACAATGCAATGCAAGCCGATGA
CAGTGGCCACAGGACTAGGAGATATACCCTTTTCAACAATGGAACTTTAT
ACTTCAACAAAGTTGGGGTAGCGGAGGAAGGAGATTATACTTGCTATGCC
CAGAACACCCTAGGGAAAGATGAAATGAAGGTCCACTTAACAGTTATAAC
AGCTGCTCCCCGGATAAGGCAGAGTAACAAAACCAACAAGAGAATCAAAG
CTGGAGACACAGCTGTCCTTGACTGTGAGGTCACTGGGGATCCCAAACCA
AAAATATTTTGGTTGCTGCCTTCCAATGACATGATTTCCTTCTCCATTGA
TAGGTACACATTTCATGCCAATGGGTCTTTGACCATCAACAAAGTGAAAC
TGCTCGATTCTGGAGAGTACGTATGTGTAGCCCGAAATCCCAGTGGGGAT
GACACCAAAATGTACAAACTGGATGTGGTCTCTAAACCTCCATTAATCAA
TGGTCTGTATACAAACAGAACTGTTATTAAAGCCACAGCTGTGAGACATT
CCAAAAAACACTTTGACTGCAGAGCTGAAGGGACACCATCTCCTGAAGTC
ATGTGGATCATGCCAGACAATATTTTCCTCACAGCCCCATACTATGGAAG
CAGAATCACAGTCCATAAAAATGGAACCTTGGAAATTAGGAATGTGAGGC
TTTCAGATTCAGCCGACTTTATCTGTGTGGCCCGAAATGAAGGTGGAGAG
AGCGTGTTGGTAGTACAGTTAGAAGTACTGGAAATGCTGAGAAGACCGAC
ATTTAGAAATCCATTTAATGAAAAAATAGTTGCCCAGCTGGGAAAGTCCA
CAGCATTGAATTGCTCTGTTGATGGTAACCCACCACCTGAAATAATCTGG
ATTTTACCAAATGGCACACGATTTTCCAATGGACCACAAAGTTATCAGTA
TCTGATAGCAAGCAATGGTTCTTTTATCATTTCTAAAACAACTCGGGAGG
ATGCAGGAAAATATCGCTGTGCAGCTAGGAATAAAGTTGGCTATATTGAG
AAATTAGTCATATTAGAAATTGGCCAGAAGCCAGTTATTCTTACCTATGC
ACCAGGGACAGTAAAAGGCATCAGTGGAGAATCTCTATCACTGCATTGTG
TGTCTGATGGAATCCCTAAGCCAAATATCAAATGGACTATGCCAAGTGGT
TATGTAGTAGACAGGCCTCAAATTAATGGGAAATACATATTGCATGACAA
TGGCACCTTAGTCATTAAAGAAGCAACAGCTTATGACAGAGGAAACTATA
TCTGTAAGGCTCAAAATAGTGTTGGTCATACACTGATTACTGTTCCAGTA
ATGATTGTAGCCTACCCTCCCCGAATTACAAATCGTCCACCCAGGAGTAT
TGTCACCAGGACAGGGGCAGCCTTTCAGCTCCACTGTGTGGCCTTGGGAG
TTCCCAAGCCAGAAATCACATGGGAGATGCCTGACCACTCCCTTCTCTCA
ACGGCAAGTAAAGAGAGGACACATGGAAGTGAGCAGCTTCACTTACAAGG
TACCCTAGTCATTCAGAATCCCCAAACCTCCGATTCTGGGATATACAAAT
GCACAGCAAAGAACCCACTTGGTAGTGATTATGCAGCAACGTATATTCAA
GTAATCTGACATGAAATAATAAAGTC

In a search of public sequence databases, the NOV12 nucleic acid sequence has 2304 of 2856 bases (80%) identical to a gb:GENBANK-ID: GENSEQ|acc:Z36321 mRNA from Rattus species (Rat mechanical stress induced cDNA encoding protein 608) (E=0.0). Public nucleotide databases include all GenBank databases and the GeneSeq patent database.

The disclosed NOV12 polypeptide (SEQ ID NO: 46) encoded by SEQ ID NO: 45 has 2617 amino acid residues and is presented in Table 12B using the one-letter amino acid code. Signal P, Psort and/or Hydropathy results predict that NOV12 has a signal peptide and is likely to be localized extracellularly with a certainty of 0.8200. In other embodiments, NOV12 may also be localized to the lysosome (lumen) with acertainty of 0.1900, the nucleus with a certainty of 0.1080, or to the endoplasmic reticulum (membrane) with a certainty of 0.1000. The most likely cleavage site for NOV12 is between positions 28 and 29: GKA-CP.

TABLE 12B
Encoded NOV12a protein sequence (SEQ ID NO:46).

MKVKGRGITCLLVSFAVICLVATPGGKACPRRCACYMPTEVHCTFRYLTS
IPDSIPPNVERINLGYNSLVRLMETDFSGLTKLELLMLHSNGIHTIPDKT
FSDLQALQVRLMVLKMSYNKVRKLQKDTFYGLRSLTRLHMDHNNIEFINP
EVFYGLNFLRLVHLEGNQLTKLHPDTFVSLSYLQIFKISFIKFLYLSDNF
LTSLPQEMVSYMPDLDSLYLHGNPWTCDCHLKWLSDWIQEKPGIYIVLPD
VIKCKKDRSPSSAQQCPLCMNPRTSKGKPLAMVSAAAFQCAKPTIDSSLK
SKSLTILEDSSSAFISPQGFMAPFGSLTLNMTDQSGNEANMVCSIQKPSR
TSPIAFTEENDYIVLNTSFSTFLVCNIDYGHIQPVWQILALYSDSPLILE
RSHLLSETPQLYYKYKQVAPKPEDIFTNIEADLRADPSWLMQDQISLQLN
RTATTFSTLQIQYSSDAQITLPRAEMRPVKHKWTMISRDNNTKLEHTVLV
GGTVGLNCPGQGDPTPHVDWLLADGSKVRAPYVSEDGRILIDKSGKLELQ
MADSFDTGVYHCISSNYDDADILTYRITVVEPLVEAYQENGIHHTVFIGE
TLDLPCHSTGIPDASISWVIPGNNVLYQSSRDKKVLNNGTLRILQVTPKD
QGYYRCVAANPSGVDFLIFQVSVKMKGQRPLEHDGETEGSGLDESNPIAH
LKEPPGAQLRTSALMEAEVGKHTSSTSKRHNYRELTLQRRGDSTHRRFRE
NRRHFPPSARRIDPQHWAALLEKAKKNAMPDKRENTTVSPPPVVTQLPNI
PGEEDDSSGMLALHEEFMVPATKALNLPARTVTADSRTISDSPMTNINYG
TEFSPVVNSQILPPEEPTDFKLSTAIKTTAMSKNINPTMSSQIQGTTNQH
SSTVFPLLLGATEFQDSDQMGRGREHFQSRPPITVRTMIKDVNVKMLSST
TNKLLLESVNTTNSHQTSVREVSEPRHNHFYSHTTQILSTSTFPSDPHTA
AHSQFPIPRNSTVNIPLFRRFGRQRKIGGRGRIISPYRTPVLRRHRYSIF
RSTTRGSSEKSTTAFSATVLNVTCLSCLPRERLTTATAALSFPSAAPITF
PKADIARVPSEESTTLVQNPLLLLENKPSVEKTTPTIKYFRTEISQVTPT
GAVMTYAPTSIPMEKTHKVNASYPRVSSTNEAKRDSVITSSLSGAITKPP
MTIIAITRFSRRKIPWQQNFVNNHNPKGRLRNQHKVSLQKSTAVMLPKTS
PALPQRQSLPSHHTTTKTHNPGSLPTKKELPFPPLNPMLPSIISKDSSTK
SIISTQTAIPATTPTFPASVITYETQTERSRAQTIQREQEPQKKNRTDPN
ISPDQSSGFTTPTAMTPPVLTTAETSVKPSVSAFTHSPPENTTGISSTIS
FHSRTLNLTDVIEELAQASTQTLKSTIASETTLSSKSHQSTTTRKAIIRH
STIPPFLSSSATLMPVPISPPFTQRAVTDNVATPISGLMTNTVVKLHESS
RHNAKPQQLVAEVATSPKVHPNAKFTIGTTHFIYSNLLHSTPMPALTTVK
SQNSKLTPSPWAENQFWHKPYSEIAEKGKKPEVSMLATTGLSEATTLVSD
WDGQKNTKKSDFDKKPVQEATTSKLLPFDSLSRYIFEKPRIVGGKAASFT
IPANSDAFLPCEAVGNPLPTIHWTRVPSGMSGLDLSKRKQNSRVQVLPNG
TLSIQRVEIQDRGQYLCSASNLFGTDHLHVTLSVVSYPPRILERRTKEIT
VHSGSTVELKCRAEGRPSPTVTWILANQTVVSESSQGSRQAVVTVDGTLV
LHNLSIYDRGFYKCVASNPGGQDSLLVKIQVIAAPPVILEQRRQVIVGTW
GESLKLPCTAKGTPQPSVYWVLSDGTEVKPLQFTNSKLFLFSNGTLYIRN
LASSDRGTYECIATSSTGSERRVVMLTMEERVTSPRIEAASQKRTEVNFG
DKLLLNCSATGEPKPQIMWRLPSKAVVDQQHRVGSWIHVYPNGSLFIGSV
TEKDSGVYLCVARNKMGDDLILMHVSLRLKPAKIDHKQYFRKQVLHGKDF
QVDCKASGSPVPEISWSLPDGTMINNAMQADDSGHRTRRYTLFNNGTLYF
NKVGVAEEGDYTCYAQNTLGKDEMKVHLTVITAAPRIRQSNKTNKRIKAG
DTAVLDCEVTGDPKPKIFWLLPSNDMISFSIDRYTFHANGSLTINKVKLL
DSGEYVCVARNPSGDDTKMYKLDVVSKPPLINGLYTNRTVIKATAVRHSK
KHFDCRAEGTPSPEVMWIMPDNIFLTAPYYGSRITVHKNGTLEIRNVRLS
DSADFICVARNEGGESVLVVQLEVLEMLRRPTFRNPFNEKIVAQLGKSTA
LNCSVDGNPPPEIIWILPNGTRFSNGPQSYQYLIASNGSFIISKTTREDA
GKYRCAARNKVGYIEKLVILEIGQKPVILTYAPGTVKGISGESLSLHCVS
DGIPKPNIKWTMPSGYVVDRPQINGKYILHDNGTLVIKEATAYDRGNYIC
KAQNSVGHTLITVPVMIVAYPPRITNRPPRSIVTRTGAAFQLHCVALGVP
KPEITWEMPDHSLLSTASKERTHGSEQLHLQGTLVIQNPQTSDSGIYKCT
AKNPLGSDYAATYIQVI

A search of sequence databases reveals that the NOV12 amino acid sequence has 1584 of 2617 amino acid residues (63%) identical to, and 1891 of 2617 amino acid residues (75%) similar to, the 2507 of 2597 amino acid residue ptnr: patp-ACC:Y53664 protein from Rattus species (Rat mechanical stress induced protein 608) (E=0.0). Public amino acid databases include the GenBank databases, SwissProt, PDB and PIR.

NOV12 is expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. This information was derived by determining the tissue sources of the sequences that were included in the invention including but not limited to SeqCalling sources, Public EST sources, and/or RACE sources.

In addition, the sequence is predicted to be expressed in osteoblasts because of the expression pattern of (GENBANK-ID: Z36321) a closely related homolog in Rattus species (Rat mechanical stress induced cDNA encoding protein 608).

NOV12b

A disclosed NOV12b nucleic acid of 771 nucleotides (also referred to as Curagen Accession No. 174124289) encoding a novel Mechanical stress induced protein-like protein is shown in Table 12C. An open reading frame was identified beginning with an AAG initiation codon at nucleotides 1-3 and ending at nucleotides 769-771. The start codon is in bold letters in Table 12E. Because NOV12b has no traditional initiation or termination codons, NOV12b could be a partial reading frame extending into the 5′ and 3′ directions.

TABLE 12C
NOV12b nucleotide sequence (SEQ ID NO:47).

AAGCTTGCCTGTCCTCGCCGCTGTGCCTGTTATATGCCTACGGAGGTACA
CTGCACATTTCGGTACCTGACTTCCATCCCAGACAGCATCCCGCCCAATG
TGGAACGCATCAATTTAGGATACAACAGCTTGGTTAGATTGATGGAAACA
GATTTTTCTGGCCTGACCAAACTGGAGTTACTCATGCTTCACAGCAATGG
CATTCACACAATCCCTGACAAGACCTTCTCAGATTTGCAGGCCTTGCAGG
TCTTAAAAATGAGCTATAACAAAGTCCGAAAACTTCAGAAAGATACTTTT
TATGGCCTCAGGAGCTTGGCACGATTGCACATGGACCACAACAATATTGA
GTTTATAAACCCAGAGGTTTTTGATGGGCTCAACTTTCTCCGCCTGGTGC
ACTTGGAAGGAAATCAGCTCACTAAGCTCCACCCAGATACATTTGTCTCT
TTGAGCTACCTCCAGATATTTAAAATCTCTTTCATTAAGTTCCTATACTT
GTCTGATAACTTCCTGACCTCCCTCCCTCAAGAGATGGTCTCCTATATGC
CTGACCTAGACAGCCTTTACCTGCATGGAAACCCATGGACCTGTGATTGC
CATTTAAAGTGGTTGTCTGACTGGATACAGGAGAAGCCAGATGTAATAAA
ATGCAAAAAAGATAGAAGTCCCTCTAGTGCTCAGCAGTGTCCACTTTGCA
TGAACCCTAGGACTTCTAAAGGCAAGCCGTTAGCTATGGTCTCAGCTGCA
GCTTTCCAGTGTGCCCTCGAG

The disclosed NOV12b polypeptide (SEQ ID NO: 48) encoded by SEQ ID NO: 47 has 257 amino acid residues and is presented in Table 12D using the one-letter amino acid code.

TABLE 12D
Encoded NOV12b protein sequence (SEQ ID NO:48).

KLACPRRCACYMPTEVHCTFRYLTSIPDSIPPNVERINLGYNSLVRLMET
DFSGLTKLELLMLHSNGIHTIPDKTFSDLQALQVLKMSYNKVRKLQKDTF
YGLRSLARLHMDHNNIEFINPEVFDGLNFLRLVHLEGNQLTKLHPDTFVS
LSYLQIFKISFIKFLYLSDNFLTSLPQEMVSYMPDLDSLYLHGNPWTCDC
HLKWLSDWIQEKPDVIKCKKDRSPSSAQQCPLCMNPRTSKGKPLAMVSAA
AFQCALE

NOV12c

A disclosed NOV12c nucleic acid of 771 nucleotides (also referred to as Curagen Accession No. 174124313) encoding a novel Mechanical stress induced protein-like protein is shown in Table 12E. An open reading frame was identified beginning with an AAG initiation codon at nucleotides 1-3 and ending with nucleotides 769-771. The start codon is in bold letters in Table 12E. Because NOV12b has no traditional initiation or termination codons, NOV12c could be a partial, reading frame extending into the 5′ and 3′ directions.

TABLE 12E
NOV12c nucleotide sequence (SEQ ID NO:49).

AAGCTTGCCTGTCCTCGCCGCTGTGCCTGTTATATGCCTACGGAGGTACA
CTGCACATTTCGGTACCTGACTTCCATCCCAGACAGCATCCCGCCCAATG
TGGAACGCATCAATTTAGGATACAACAGCTTGGTTAGATTAATGGAAACA
GATTTTTCTGGCCTGACCAAACTGGAGTTACTCATGCTTCACAGCAATGG
CATTCACACAATCCCTGACAAGACCTTCTCAGATTTGCAGGCCTTGCAGG
TCTTAAAAATGAGCTATAATAAAGTCCGAAAACTTCAGAAAGATACTTTT
TATGGCCTCAGGAGCTTGACACGATTGCACATGGACCACAACAATATTGA
GTTTATAAACCCAGAGGTTTTTTATGGGCTCAACTTTCTCCGCCTGGTGC
ACTTGGAAGGAAATCAGCTCACTAAGCTCCACCCAGATACATTTGTCTCT
TTGAGCTACCTCCAGATATTTAAAATCTCTTTCATTAAGTTCCTATACTT
GTCTGATAACTTCCTGACCTCCCTCCCTCAAGAGATGGTCTCCTATATGC
CTGACCTAGACAGCCTTTACCTGCATGGAAACCCATGGACCTGTGATTGC
CATTTAAAGTGGTTGTCTGACTGGATACAGGAGAAGCCAGATGTAATAAA
ATGCAAAAAAGATAGAAGTCCCTCTAGTGCTCAGCAGTGTCCACTTTGCA
TGAACCCTAGGACTTCTAAAGGCAAGCCGTTAGCTATGGTCTCAGCTGCA
GCTTTCCAGTGTGCCCTCGAG

The disclosed NOV12c polypeptide (SEQ ID NO: 50) encoded by SEQ ID NO: 49 has 257 amino acid residues and is presented in Table 12F using the one-letter amino acid code.

TABLE 12F
Encoded NOV12c protein sequence (SEQ ID NO:50).

KLACPRRCACYMPTEVHCTFRYLTSIPDSIPPNVERINLGYNSLVRLMET
DFSGLTKLELLMLHSNGIHTIPDKTFSDLQALQVLKMSYNKVRKLQKDTF
YGLRSLTRLHMDHNNIEFINPEVFYGLNFLRLVHLEGNQLTKLHPDTFVS
LSYLQIFKISFIKFLYLSDNFLTSLPQEMVSYMPDLDSLYLHGNPWTCDC
HLKWLSDWIQEKPDVIKCKKDRSPSSAQQCPLCMNPRTSKGKPLAMVSAA
AFQCALE

NOV12d

A disclosed NOV12d nucleic acid of 771 nucleotides (also referred to as Curagen Accession No. 174124322) encoding a novel Mechanical stress induced protein-like protein is shown in Table 12G. An open reading frame was identified beginning with an AAG initiation codon at nucleotides 1-3 and ending with nucleotides 769-771. The start codon is in bold letters in Table 12G. Because NOV12d has no traditional initiation or termination codons, NOV12d could be a partial reading frame extending into the 5′ and 3′ directions.

TABLE 12G
NOV12d nucleotide sequence (SEQ ID NO:51).

AAGCTTGCCTGTCCTCGCCGCTGTGCCTGTTATATGCCTACGGAGGTACA
CTGCACATTTCGGTACCTGACTTCCATCCCAGACAGCATCCCGCCCAATG
TGGAACGCATCAATTTAGGATACAACAGCTTGGTTAGATTGATGGAAACA
GATTTTTCTGGCCTGACCAAACTGGAGTTACTCATGCTTCACAGCAATGG
CATTCACACAATCCCTGACAAGACCTTCTCAGATTTGCAGGCCTTGCAGG
TCTTAAAAATGAGCTATAACAAAGTCCGAAAACTTCAGAAAGATACTTTT
TATGGCCTCAGGAGCTTGACACGATTGCACATGGACCACAACAATATTGA
GTTTATAAACCCAGAGGTTTTTGATGGGCTCAACTTTCTCCGCCTGGTGC
ACTTGGAAGGAAATCAGCTCACTAAGCTCCACCCAGATACATTTGTCTCT
TTGAGCTACCTCCAGATATTTAAAATCTCTTTCATTAAGTTCCTATACTT
GTCTGATAACTTCCTGACCTCCCTCCCTCAAGAGATGGTCTCCTATATGC
CTGACCTAGACAGCCTTTACCTGCATGGAAACCCATGGACCTGTGATTGC
CATTTAAAGTGGTTGTCTGACTGGATACAGGAGAAGCCAGATGTAATAAA
ATGCAAAAAAGATAGAAGTCCCTCTAGTGCTCAGCAGTGTCCACTTTGCA
TGAACCCTAGGACTTCTAAAGGCAAGCCGTTAGCTATGGTCTCAGCTGCA
GCTTTCCAGTGTGCCCTCGAG

The reverse complement og NOV12d is shown in Table 12H.

TABLE 12H
NOV12d reverse complement nucleotide sequence
(SEQ ID NO:60).

CTCGAGGGCACACTGGAAAGCTGCAGCTGAGACCATAGCTAACGGCTTGC
CTTTAGAAGTCCTAGGGTTCATGCAAAGTGGACACTGCTGAGCACTAGAG
GGACTTCTATCTTTTTTGCATTTTATTACATCTGGCTTCTCCTGTATCCA
GTCAGACAACCACTTTAAATGGCAATCACAGGTCCATGGGTTTCCATGCA
GGTAAAGGCTGTCTAGGTCAGGCATATAGGAGACCATCTCTTGAGGGAGG
GAGGTCAGGAAGTTATCAGACAAGTATAGGAACTTAATGAAAGAGATTTT
AAATATCTGGAGGTAGCTCAAAGAGACAAATGTATCTGGGTGGAGCTTAG
TGAGCTGATTTCCTTCCAAGTGCACCAGGCGGAGAAAGTTGAGCCCATCA
AAAACCTCTGGGTTTATAAACTCAATATTGTTGTGGTCCATGTGCAATCG
TGTCAAGCTCCTGAGGCCATAAAAAGTATCTTTCTGAAGTTTTCGGACTT
TGTTATAGCTCATTTTTAAGACCTGCAAGGCCTGCAAATCTGAGAAGGTC
TTGTCAGGGATTGTGTGAATGCCATTGCTGTGAAGCATGAGTAACTCCAG
TTTGGTCAGGCCAGAAAAATCTGTTTCCATCAATCTAACCAAGCTGTTGT
ATCCTAAATTGATGCGTTCCACATTGGGCGGGATGCTGTCTGGGATGGAA
GTCAGGTACCGAAATGTGCAGTGTACCTCCGTAGGCATATAACAGGCACA
GCGGCGAGGACAGGCAAGCTT

The disclosed NOV12d polypeptide (SEQ ID NO: 52) encoded by SEQ ID NO: 51 has 257 amino acid residues and is presented in Table 121 using the one-letter amino acid code.

TABLE 12I
Encoded NOV12d protein sequence (SEQ ID NO:52).

KLACPRRCACYMPTEVHCTFRYLTSIPDSIPPNVERINLGYNSLVRLMET
DFSGLTKLELLMLHSNGIHTIPDKTFSDLQALQVLKMSYNKVRKLQKDTF
YGLRSLTRLHMDHNNIEFINPEVFDGLNFLRLVHLEGNQLTKLHPDTFVS
LSYLQIFKISFIKFLYLSDNFLTSLPQEMVSYMPDLDSLYLHGNPWTCDC
HLKWLSDWIQEKPDVIKCKKDRSPSSAQQCPLCMNPRTSKGKPLAMVSAA
AFQCALE

NOV12e

A disclosed NOV12e nucleic acid of 771 nucleotides (also referred to as Curagen Accession No. 174124322) encoding a novel Mechanical stress induced protein-like protein is shown in Table 12J. An open reading frame was identified beginning with an AAG initiation codon at nucleotides 1-3 and ending with nucleotides 769-771. The start codon is in bold letters in Table 12J. Because NOV12e has no traditional initiation or termination codons, NOV12e could be a partial reading frame extending into the 5′ and 3′ directions.

TABLE 12J

| | |
|---|---|
| NOV12e nucleotide sequence. | (SEQ ID NO:53) |

AAGCTTGCCTGTCCTCGCCGCTGTGCCTGTTATATGCCTACGGAGGTACACTGCACATTTCCGTACCTGACT
TCCATCCCAGACAGCATCCCGCCCAATGTGGAACGCATCAATTTAGGATACAACAGCTTGGTTAGATTGATG
GAAACAGATTTTTCTGGCCTGACCAAACTGGAGTTACTCATGCTTCACAGCAATGGCATTCACACAATCCCT
GGCAAGACCTTCTCAGATTTGCAGGCCTTGCAGGTCTTAAAAATGAGCTATAACAAAGTCCGAAAACTTCAG
AAAGATACTTTTTATGGCCTCAGGAGCTTGACACGATTGCACATGGACCACAACAATATTGAGTTTATAAAC
CCAGAGGTTTTTGATGGGCTCAACTTTCTCCGCCTGGTGCACTTGGAAGGAAATCAGCTCACTAAGCTCCAC
CCAGATACATTTGTCTCTTTGAGCTACCTCCAGATATTTAAAATCTCTTTCATTAAGTTCCTATACTTGTCT
GATAACTTCCTGACCTCCCTCCCTCAAGAGATGGTCTCCTATATGCCTGACCTAGACAGCCTTTACCTGCAT
GGAAACCCATGGACCTGTGATTGCCATTTAAAGTGGTTGTCTGACTGGATACAGGAGAAGCCAGATGTAATA
AAATGCAAAAAAGATAGAAGTCCCTCTAGTGCTCAGCAGTGTCCACTTTGCATGAACCCTAGGACTTCTAAA
GGCAAGCCGTTAGCTATGGTCTCAGCTGCAGCTTTCCAGTGTGCCCTCGAG

The disclosed NOV12e polypeptide (SEQ ID NO: 54) encoded by SEQ ID NO: 53 has 257 amino acid residues and is presented in Table 12K using the one-letter amino acid code.

TABLE 12K

| | |
|---|---|
| Encoded NOV12e protein sequence. | (SEQ m NO:54) |

KLACPRRCACYMPTEVHCTFRYLTSIPDSIPPNVERINLGYNSLVRLMETDFSGLTKLELLMLHSNGIHTIP
GKTFSDLQALQVLKMSYNKVRKLQKDTFYGLRSLTRLHMDHNNIEFINPEVFDGLNFLRLVHLEGNQLTKLH
PDTFVSLSYLQIFKISFIKFLYLSDNFLTSLPQEMVSYMPDLDSLYLHGNPWTCDCHLKWLSDWIQEKPDVI
KCKKDRSPSSAQQCPLCMNPRTSKGKPLANVSAAAFQCALE

NOV12f

A disclosed NOV12f nucleic acid of 8270 nucleotides (also referred to as Curagen Accession No. CG55776-03) encoding a novel Mechanical stress induced protein-like protein is shown in Table 12L. An open reading frame was identified beginning with an ATG initiation codon at nucleotides 6-8 and ending with a TGA codon at nucleotides 7779-7781. Putative untranslated regions upstream from the initiation codon and downstream of the termination codon are underlined in Table 12L. The start and stop codons are in bold letters.

TABLE 12L

| | |
|---|---|
| NOV12 nucleotide sequence. | (SEQ ID NO:55) |

TCAGGATGAAGGTAAAAGGCAGAGGAATCACCTGCTTGCTGGTCTCCTTTGCTGTGATCTGCCTGGTCGCCA
CCCCTGCGGGCAAGGCCTGTCCTCGCCGCTGTGCCTGTTATATGCCTACGGAGGTACACTGCACATTTCGGT
ACCTGACTTCCATCCCAGACAGCATCCCGCCCAATGTGGAACGCATCAATTTAGGGTACAACAGCTTGGTTA
GATTGATGGAAACAGATTTTTCTGGCCTGACCAAACTGGAGTTACTCATGCTTCACAGCAATGGCATTCACA
CAATCCCTGACAAGACCTTCTCAGATTTGCAGGCCTTGCAGGTGAGACTGATGGTCTTAAAAATGAGCTATA
ATAAAGTCCGAAAACTTCAGAAAGATACTTTTTATGGCCTCAGGAGCTTTACACGATTGCACATGGACCACA
ACAATATTGAGTTTATAAACCCAGAGGTTTTTTATGGGCTCAACTTTCTCCGCCTGGTGCACTTGGAAGGAA
ATCACCTCACTAAGCTCCACCCAGATACATTTGTCTCTTTGAGCTACCTCCAGATATTTAAAATCTCTTTCA
TTAAGTTCCTATACTTGTCTGATAACTTCCTGACCTCCCTCCCTCAAGAGATGGTCTCCTATATGCCTGACC
TAGACAGCCTTTACCTGCATGGAAACCCATGGACCTGTGATTGCCATTTAAAGTGGTTGTCTGACTGGATAC
AGGAGAAGCCAGGTATCTATATTGTNTTACCAGATGTAATAAAATGCAAAAAAGATAGAAGTCCCTCTAGTG
CTCAGCAGTGTCCACTTTGCATGAACCCTAGGACTTCTAAAGGCAAGCCGTTACCTATGGTCTCAGCTGCAG
CTTTCCAGTGTGCCAAGCCAACCATTGACTCATCCCTGAAATCAAAGAGCCTGACTATTCTGGAAGACAGTA
GTTCTGCTTTCATCTCTCCCCAACGTTTCATGGCACCCTTTGGCTCCCTCACTTTOAATATGACAGATCAGT
CTGGAAATGAAGCTAACATGGTCTGCAGTATTCAAAAGCCCTCAAGGACATCACCCATTGCATTCACTGAAG
AAAATGACTACATCGTGCTAAATACTTCATTTTCAACATTTTTCGTGTGCAACATAGATTACGGTCACATTC
AGCCAGTGTGGCAAATTTTGGCTTTGTACAGTGATTCTCCTCTGATACTAGAAAGGACCCACTTGCTTAGTG
AAACACCGCAGCTCTATTACAAATATAAACAGGTGGCTCCTAAGCCTGAAGACATTTTTACCAACATAGAGG
CAGATCTCAGAGCAGATCCCTCTTGGTTAATGCAAGACCAAATTTCCTTGCAGCTGAACAGAACTGCCACCA
CATTCACTACATTACAGATCCAGTACTCCAGTGATGCTCAAATCACTTTACCAAGAGCAGAGATGAGGCCAG
TGAAACACAAATGGACTATGATTTCAAGGGATAACAATACTAAGCTGGAACATACTGTCTTGGTAGGTGGAA
CCGTTGGCCTGAACTGCCCAGGCCAAGGAGACCCCACCCCACACGTGGATTGGCTTCTAGCTGATGGAAGTA
AAGTGAGAGCCCCTTATGTCAGTGAGGATGGACCGATCCTAATAGACAAAAGTGGAAAATTGGAACTCCAGA
TGGCTGATAGTTTTGACACAGGCGTATATCACTGTATAAGCAGCAATTATGATGATGCAGATATTCTCACCT
ATAGGATAACTCTGGTAGAACCTTTGGTCGAAGCCTATCAGGAAAATGGGATTCATCACACAGTTTTCATTG
GTGAAACACTTGATCTTCCATGCCATTCTACTGGTATCCCAGATGCCTCTATTAGCTGGGTTATTCCAGGAA
ACAATGTGCTCTATCAGTCATCAAGAGACAAGAAAGTTCTAAACAATGGCACATTAAGAATATTACAGGTCA
CCCCGAAAGACCAAGGTTATTATCGCTGTGTGGCAGCCAACCCATCAGGGGTTGATTTTTTGATTTTCCAAG
TTTCAGTCAAGATGAAAGGACAAAGGCCCTTGGAGCATGATGOAGAAACAGAGGGATCTGGACTTGATGAGT
CCAATCCTATTGCTCATCTTAAGGAGCCACCAGGTCCACAACTCCGTACATCTGCTCTGATGGAGGCTGAGG
TTGGAAAACACACCTCAAGCACAAGTAAGAGGCACAACTATCGGGAATTAACACTCCAGCGACGTGGAGATT
CAACACATCGACGTTTTAGCGAGAATAOGAGGCATTTCCCTCCCTCTGCTAGGAGAATTGACCCACAACATT
GGGCOGCACTGTTGGAGAAAGCTAAAAAGAATGCTATGCCAGACAAGCGAGAAAATACCACAGTGAGCCCAC
CCCCAGTGGTCACCCAACTCCCAAACATACCTGGTGAAGAAGACGATTCCTCAGGCATGCTCGCTCTACATG
AGGAATTTATGGTCCCGGCCACTAAAGCTTTGAACCTTCCAGCAAGGACAGTGACTGCTGACTCCAGAACAA
TATCTGATAGTCCTATGACAAACATAAATTATGGCACAGAATTCTCTCCTGTTGTGAATTCACAAATACTAC
CACCTGAAGAACCCACAGATTTCAAACTGTCTACTGCTATTAAAACTACAGCCATGTCAAAGAATATAAACC
CAACCATGTCAAGCCAAATACAAGOCACAACCAATCAACATTCATCCACTGTCTTTCCACTGCTACTTGGAG
CAACTGAATTTCAGGACTCTGACCAGATGGGAAGAGGAAGAGAGCATTTCCAAAGTAGACCCCCAATAACAG
TAAGGACTATGATCAAAGATGTCAATGTCAAAATGCTTAGTAGCACCACCAACAAACTATTATTAGAGTCAG
TAAATACCACAAATAGTCATCAGACATCTGTAAGAGAAGTGAGTGAACCCACGCACAATCACTTCTATTCTC
ACACThCTCAAATACTTAGCACCTCCACGTTCCCTTCAGATCCACACACAGCTGCTCATTCTCAGTTTCCGA
TCCCTAGAAATAGTACAGTTAACATCCCGCTGTTCAGACGCTTTGGGAGGCAGACGAAAATTGGCGGAACGG
GGCGGATTATCAGCCCATATAGAACTCCAGTTCTGCGACGGCATAGATACAGCATTTTCAGGTCAACAACCA
GACGTTCTTCTGAAAAAAGCACTACTGCATTCTCAGCCACAGTGCTCAATGTGACATGTCTGTCCTGTCTTC
CCACGGAGACGCTCACCACTGCCACAGCAGCATTGTCTTTTCCAAGTGCTGCTCCCATCACCTTCCCCAAAG
CTGACATTGCTAGAGTCCCATCAGAAGAGTCTACAACTCTAGTCCAGAATCCACTATTACTACTTGAGAACA
AACCCAGTGTAGAGAAAACAACACCCACAATAAAATATTTCAGGACTGAAATTTCCCAAGTGACTCCAACTG
GTGCAGTCATGACATATGCTCCAACATCCATACCCATGGAAAAAACTCACAAAGTAAACCCCAGTTACCCAC
GTGTGTCTAGCACCAATGAAGCTAAAAGAGATTCAGTGATTACATCGTCACTTTCAGGTGCTATCACCAAGC
CACCAATGACTATTATAGCCATTACAAGGTTTTCAAGAAGGAAAATTCCCTGGCAACAGAACTTTGTAAATA
ACCATAACCCAAAAGGCAGATTAAGGAATCAACATAAAGTTAGTTTACAAAAAAGCACAGCTGTGATGCTTC
CTAAAACATCTCCTGCTTTACCACAGAGACAAAGTCTCCCCTCGCACCACACTACGACCAAAACACACAATC
CTGGAAGTCTTCCAACAAAGAAGGAGCTTCCCTTCCCACCCCTTAACCCTATGCTTCCTAGTATTATAAGCA
AAGACTCAAGTACAAAAAGCATCATATCAACGCAAACAGCAATACCAGCAACAACTCCTACCTTCCCTGCAT
CTGTCATCACTTATGAAACCCAAACAGAGAGATCTAGAGCACAAACAATACAAAGAGAACACGAGCCTCAAA
AGAAGAACAGGACTGACCCAAACATCTCTCCAGACCAGAGTTCTGGCTTCACTACACCCACTGCTATGACAC
CTCCTGTTCTAACCACAGCCGAAACTTCAGTCAAGCCCAGTGTCTCTGCATTCACTCATTCCCCACCAGAAA
ACACAACTGGGATTTCAAGCACAATCAGTTTTCATTCAAGAACTCTTAATCTGACAGATGTGATTGAAGAAC
TAGCCCAAGCAAGTACTCAGACTTTGAAGAGCACAATTGCTTCTGAAACAACTTTGTCCAGCAAATCACACC
AGAGTACCACAACTAGGAAAGCAATCATTAGACACTCAACCATACCACCATTCTTGAGCAGCAGTCCTACTC
TAATGCCAGTTCCCATCTCCCCTCCCTTTACTCAGAGAGCAGTTACTGACAACGTOGCGACTCCCATTTCCG
CGCTTATGACAAATACAGTGGTCAAGCTCCACGAATCCTCAACGCACAATGCTAAACCACAGCAATTAGTAG
CAGAGGTTGCAACATCCCCCAAGGTTCACCCAAATGCCAAGTTCACAATTGGAACCACTCACTTCATCTACT
CTAATCTGTTACATTCTACTCCCATCCCAGCACTAACAACAGTTAAATCACAGAATTCTAAATTAACTCCAT
CTCCCTGGGCAGAAAACCAATTTTGGCACAAACCATACTCAGAAATTGCTGAAAAAGGCAAAAAGCCAGAAG
TAAGCATGTTGGCTACTACAGGCCTGTCCGAGGCCACCACTCTTGTTTCAGATTGGGATGGACAGAAGAACA
CAAAGAAGAGTGACTTTGATAAGAAACCAGTTCAAGAAGCAACAACTTCCAAACTCCTTCCCTTTGACTCTT
TGTCTAGGTATATATTTGAAAAGCCCAGGATAGTTGGAGGAAAAGCTGCAAGTTTTACTATTCCAGCTAACT
CAGATGCCTTTCTTCCCTGTGAAGCTGTTGGAAATCCCCTGCCCACCATTCATTGGACCAGAGTCCCATCAG
GTATGTCAGGACTTGATTTATCTAAGAGGAAACAGAATAGCAGGGTCCAGGTTCTCCCCAATGGTACCCTGT
CCATCCAGAGGGTGGAAATTCAGGACCGCGGACAGTACTTGTGTTCCGCATCCAATCTGTTTGGCACAGACC
ACCTTCATGTCACCTTGTCTGTGGTTTCCTATCCTCCCAGGATCCTGGAGAGACGTACCAAAGAGATCACAG
TTCATTCCGGAAGCACTGTGGAACTGAAGTGCAGAGCAGAAGGTAGGCCAAGCCCTACAGTTACCTGGATTC
TTGCAAACCAAACAGTTGTCTCAGAATCATCCCAGGGAAGTAGGCAGGCTGTGGTGACGGTTGACGGAACAT
TGGTCCTCCACAATCTCAGTATTTATGACCGTGGCTTTTACAAATGTGTGGCCAGCAACCCAGGTGGCCAGG
ATTCACTGCTGGTTAAAATACAACTCATTGCAGCACCACCTGTTATTCTAGAGCAAAGGAGGCAAGTCATTG
TAGGCACTTGGGGTGAAAGTTTAAAACTGCCCTGTACTGCAAAAGGAACTCCTCAGCCCAGCGTTTACTGGG
TCCTCTCTGATGGCACTGAAGTGAAACCATTACAGTTTACCAATTCCAAGTTGTTCTTATTTTCAAATGGGA
CTTTGTATATAAGAAACCTAGCCTCTTCAGACAGGGGCACTTATGAATGCATTGCTACCAGTTCCACTGGTT
CGGAGCGAAGAGTAGTAATGCTTACAATGGAAGAGCGAGTGACCAGCCCCAGGATAGAAGCTGCATCCCAGA
AAAGGACTGAAGTGAATTTTGGGGACAAATTACTACTGAACTGCTCAGCCACTGGGGAGCCCAAACCCCAAA
TAATGTGGAGGTTACCATCCAAGGCTGTGGTCGACCAGCAGCATAGAGTGGGCACGTGGATCCACGTCTACC
CTAATGGATCCCTGTTTATTGGATCAGTAACAGAAAAAGACAGTGGTGTCTACTTGTGTGTGGCAAGAAACA
AAATGGGGGATGATCTGATACTGATGCATGTTAGCCTAGAACTGAAACCTGCCAAAATTGACCACAAGCAGT
ATTTTAGAAAGCAAGTGCTCCATGGGAAAGATTTCCAAGTAGATTGCAAAGCTTCCGGCTCCCCAGTGCCAG
AGATATCTTGGAGTTTGCCTGATGGAACCATGATCAACAATGCAATGCAAGCCGATGACAGTGGCCACAGGA
CTAGGAGATATACCCTTTTCAACAATGGAACTTTATACTTCAACAAAGTTGGGGTAGCGGAGGAAGGAGATT
ATACTTGCTATGCCCAGAACACCCTAGGGAAAGATGAAATGAAGGTCCACTTAACAGTTATAACAGCTGCTC
CCCGGATAAGGCAGAGTAACAAAACCAACAAGAGAATCAAAGCTGGAGACACAGCTGTCCTTGACTGTGAGG
TCATTCATGCCAATGGGTCTTTGACCATCAACAAAGTGAAACTGCTCGATTCTGGAGAGTACGTATGTGTAG
CCCGAATCCCAGTGGGGATGACACCAAAATGTACAAACTGGATGTGGTCTCTAAACCTCCATTAATCAAATG
GTCTGTATACAAATAGAACTGTTATTAAAGCCACAGCTGTGAGACATTCCAAAAAACACTTTGACTGCAGAG
CTGAAGGGACACCATCTCCTGAAGTCATGTGGATCATGCCAGACAATATTTTCCTCACAGCCCCATACTATG
GAAGCAGAATCACAGTCCATAAAAATGGAACCTTGGAAATTAGGAATGTGAGGCTTTCAGATTCAGCCGACT
TTATCTGTGTGGCCCGAAATGAAGGTGGAGAGAGCGTGTTGGTAGTACAGTTAGAAGTACTGGAAATGCTGA
GAAGACCGACATTTAGAAATCCATTTAATGAAAAAATAGTTGCCCAGCTGGGAAAGTCCACAGCATTGAATT
GCTCTGTTGATGGTAACCCACCACCTGAAATAATCTGGATTTTACCAAATGGCACACGATTTTCCAATGGAC
CACAAAGTTATCAGTATCTGATAGCAAGCAATCGTTCTTTTATCATTTCTAAAACAACTCGGGAGGATGCAG
GAAAATATCGCTGTGCAGCTAGGAATAAAGTTGGCTATATTGAGAAATTAGTCATATTAGAAATTGGCCAGA
AGCCAGTTATTCTTACCTATGCACCAGGGACAGTAAAAGGCATCAGTGGAGAATCTCTATCACTGCATTGTG
TGTCTGATGGAATCCCTAAGCCAAATATCAAATGGACTATGCCAAGTGGTTATGTAGTAGACAGGCCTCAAA
TTAATGGGAAATACATATTGCATGACAATGGCACCTTAGTCATTAAAGAAGCAACAGCTTATGACAGAGGAA
ACTATATCTGTAAGGCTCAAAATAGTGTTGGTCATACACTGATTACTGTTCCAGTAATGATTGTAGCCTACC
CTCCCCGAATAACAAATCGTCCACCCAGGAGTATTGTCACCAGGACAGGGGCAGCCTTTCAGCTCCACTGTG
TGGCCTTGGGAGTTCCCAAGCCAGAAATCACGTGGGAGATGCCTGACCACTCCCTTCTCTCAACGGCAAGTA
AAGAGAGGACACATGGAAGTGAGCAGCTTCACTTACAAGGTACCCTAGTCATTCAGAATCCCCAAACCTCCG
ATTCTGGGATATACAAATGCACAGCAAAGAACCCACTTGGTAGTGATTATGCAGCAACGTATATTCAAGTAA
TCTGACATGAAATAATAAAGTCAACAACATCTGGGCAGAATTTATTTTTTGGAAGAAGTTTAATCAAAGGCA
GCCATAGGCATGTAAATGAATTTGAATACATTTACAGTATTAAATTTACAATGAACATGCAAAATAAAAGGA
CTTGTAAATAAATGCATTATGAACTGATGATACTGATTTATTTAATGGATCTCAAAACAAACTTTTAACTTA
AGGCACTTTTATTTTGCCAACAAATAACAATAAACAAACATTGAAACGGTTCACTATAAAATAACAAATGGC
TAATGTACCTGAATTTTTCAGTAAAAAAATGAACTTCTAATACCAGTTGCCTAGTGTCCACCTCCTATCAAT
GTTACAAGCATGGCACTCAGAACAGAGACAATGGAAAATATTAAATCTGCAATCTTTATGATGTAAATTTAC
CATCCTGATGTATAAATATTTTGTGGTTTATAAATTTTTTTGCTAAAACCTAAAAAAA

In a search of public sequence databases, the NOV12f nucleic acid sequence has 879 of 1446 bases (60%) identical to a gb:GENBANK-ID:AF245505|acc:AF245505.1 mRNA from Homo sapiens (Homo sapiens adlican mRNA, complete cds) (E=2.3e⁻¹²⁷). Public nucleotide databases include all GenBank databases and the GeneSeq patent database.

The disclosed NOV12f polypeptide (SEQ ID NO: 56) encoded by SEQ ID NO: 55 has 2591 amino acid residues and is presented in Table 12M using the one-letter amino acid code. Signal P, Psort and/or Hydropathy results predict that NOV12 has a signal peptide and is likely to be localized extracellularly with a certainty of 0.8200. In other embodiments, NOV12 may also be localized to the lysosome (lumen) with acertainty of 0.1900, the nucleus with a certainty of 0.1080, or to the endoplasmic reticulum (membrane) with a certainty of 0.1000. The most likely cleavage site for NOV12 is between positions 28 and 29: GKA-CP.

TABLE 12M

| | |
|---|---|
| Encoded NOV12f protein sequence. | (SEQ ID NO:56) |

MKVKGRGITCLLVSFAVICLVATPGGKACPRRCACYMPTEVHCTFRYLTSIPDSIPPNVERINLGYNSLVRL
METDFSGLTKLELLMLHSNGIHTIPDKTFSDLQALQVRLMVLKMSYNKVRKLQKDTFYGLRSLTRLHMDHNN
IEFINPEVFYGLNFLRLVHVLEGNQLTKLHPDTFVSLSYLQIFKISFIKFLYSDNFLTSLPQEMVSTMPDLD
SLYLHGNPWTCKCHLKWLSDWIQEKPGIYIVLPDVIKCKKDRSPSSAQQCPLCMNPRTSKGKPLAMVSAAAF
QCAKPTIDSSLKSKSLTILEDSSSAFISPQGFMAPFGSLTLNMTDQSGNEANMVCSIQKPSRTSPAIFTEEN
DYIVLNTSFSTFLVCNIDYGHIQPVWQILALYSDSPLILERSHLLSETPQLYYKYKQVAPKPEDIFTNIEAD
LRADPSWLMQDQISLQLNRTATTFSTLQIQYSSDAQITLPRAEMRPVKHKWTMISRDNNTKLEHTVLVGGTV
GLNCPGQGDPTPHVDWLLADGSKVRAPYVSEDGRILIDKSGKLELQMADSFDTGVYHCISSNYDDADILTYR
ITVVEPLVEAYQENGIHHTVFIGETLDLPCHSTGIPDASISWVIPGNNVLYQSSRDKKVLNNGTLRILQVTP
KDQGYYRCVAANPSGVDFLIFQVSVKMKGQRPLEHDGETEGSGLDESNPIAHLKEPPGAQLRTSALMEAEVG
KHTSSTSKRHNYRELTLQRRGDSTHRRFRENRRHFPPSARRIDPQHWAALLEKAKKNAMPDKRENTTVSPPP
VVTQLPNIPGEEDDSSGMLALHEEFMVPATKALNLPARTVTADSRTISDSPMTNINYGTEFSPVVNSQILPP
EEPTDFKLSTAIKTTAMSKNINPTMSSQIQGTTNQHSSTVFPLLLGATEFQDSDQMGRGREHFQSRPPITVR
TMIKDVNVKMLSSTTNKLLLESVNTTNSHQTSVREVSEPRHNHFYSHTTQILSTSTFPSDPHTAAHSQFPIP
RNSTVNIPLFRRFGRQRKIGGRGRIISPYRTPVLRRHRYSIFRSTTRGSSEKSTTAFSATVLNVTCLSCLPR
ERLTTATAALSFPSAAPITFPKADIARVPSEESTTLVQNPLLLLENKPSVEKTTPTIKYFRTEISQVTPTGA
VMTYAPTSIPMEKTHRVNASYPRVSSTNEAXRDSVITSSLSGAITKPPMTIIAITRFSRRKIPWQQNFVNNH
NPKGRLRNQHKVSLQKSTAVMLPKTSPALPQRQSLPSHHTTTKTHNPGSLPTKKELPFPPLNPMLPSIISKD
SSTKSIISTQTAIPATTPTFPASVITYETQTERSRAOTIQREQEPQKKNRTDPMISPDQSSGFTTPTAMTPP
VLTTAETSVKPSVSAFTHSPPENTTGISSTISFHSRTLNLTDVIEELAQASTQTLKSTIASETTLSSKSHQS
TTTRKAIIRHSTIPPFLSSSATLMPVPTSPPFTQRAVTDNVATPISGLMTNTVVKLHESSRHNAKPQQLVAE
VATSPKVHPNAKFTIGTTHFIYSNLLHSTPMPALTTVKSQNSKLTPSPWAENQFWHKPYSEIAEKGKKPEVS
MLATTGLSEATTLVSDWDGQKNTKKSDFDKKPVQEATTSKLLPFDSLSRYIFEKPRIVGGKAASFTIPANSD
AFLPCEAVGNPLPTIHWTRVPSGMSGLDLSKRKQNSRVOThPNGTLSIQRVEIQDRGQYLCSASNLFGTDHL
HVTLSVVSYPPRILERRTKEITVMSGSTVELKCRAEGRPSPTVTWILANQTVVSESSQGSRQAVVTVDGTLV
LHNLSIYDRGFYKCVASNPGGQDSLLVKIQVIAAPPVILEQRRQVIVGTWGESLKLPCTAKGTPQPSVYWVL
SDGTEVKPLQFTNSKLFLFSNGTLYIRNLASSDRGTYECIATSSTGSERRVVMLTMEERVTSPRIEAASQKR
TEVNFGDKLLLNCSATGEPKPQIMWRLPSKAVVDQQHRVGSWIHVYPNCSLFIGSVTEKDSGVYLCVARNKM
GDDLILMHVSLELKPAKIDHKQYFRKQVLHGKDFQVDCKASGSPVPEISWSLPDGTMINNAMQADDSGHRTR
RYTLFNNGTLYFNKVGVAEEGDYTCYAQNTLGKDEMKVHLTVITAAPRIRQSNKTNKRIKAGDTAVLDCEVI
HANGSLTINKVKLLDSGEYVCVARNPSGDDTKNYKLDVVSKPPLINGLYTNRTVIKATAVRHSKKHFDCRAE
GTPSPEVMWIMPDNIFLTAPYYGSRITVHKNGTLEIRNVRLSDSADFICVARNEGGESVLVVQLEVLEMLRR
PTFRNPFNEKIVAQLGKSTALNCSVDGNPPPEIIWILPNGTRFSNGPQSYQYLIASNGSFIISKTTREDAGK
YRCAARNKVGYIEKLVILEIGQKPVILTYAPGTVKGISGESLSLHCVSDGIPKPNIKWTMPSGYVVDRPQIN
GKYILHDNGTLVIKEATAYDRGNYICKAQNSVGHTLITVPVMIVAYPPRITNRPPRSIVTRTGAAFQLHCVA
LGVPKPEITWEMPDHSLLSTASKERTHGSEQLHLQGTLVIQNPQTSDSGIYKCTAKNPLGSDYAATYIQVI

A search of sequence databases reveals that the NOV12f amino acid sequence has 246 of 522 amino acid residues (47%) identical to, and 348 of 522 amino acid residues (66%) similar to, the 2828 amino acid residue ptnr:SPTREMBL-ACC:Q9NR99 protein from Homo sapiens (Human) (Adlican) (E=0.0). Public amino acid databases include the GenBank databases, SwissProt, PDB and PIR.

NOV12f is expressed in at least the following tissues: mammalian tissue, parotid salivary glands, liver, small intestine, peripheral blood, pituitary gland, mammary gland/breast, testis, lung, lung pleura, skin, heart, tonsil, brain, uterus, cochlea. Expression information was derived from the tissue sources of the sequences that were included in the derivation of the sequence of NOV12f.

The disclosed NOV12a polypeptide has homology to the amino acid sequences shown in the BLASTP data listed in Table 12N.

TABLE 12N
BLAST results for NOV12a

| Gene Index/ | | Length | Identity | Positives | |
|---|---|---|---|---|---|
| Identifier | Protein/Organism | (aa) | (%) | (%) | Expect |
| gi\|9280405\|gb\| | adlican | 2828 | 440/980 | 626/980 | 0.0 |
| AAF86402.1\| | [Homo sapiens] | | (44%) | (62%) | |
| AF245505_1 | | | | | |
| (AF245505) | | | | | |
| gi\|17444262\|ref\|XP_{—} | hemicentrin [Homo sapiens] | 3645 | 259/880 | 390/880 | 1e−84 |
| 053531.2\| | | | (29%) | (43%) | |
| (XM_053531) | | | | | |
| gi\|14575679\|gb\| | hemicentin | 5636 | 259/880 | 390/880 | 1e−84 |
| AAK68690.1\| | [Homo sapiens] | | (29% | (43%) | |
| AF156100_1 | | | | | |
| (AF156100) | | | | | |

The homology between these and other sequences is shown graphically in the ClustalW analysis shown in Table 12O. In the ClustalW alignment of the NOV12 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

[image]

[image]

[image]

[image]

[image]

[image]

[image]

[image]

[image]

[image]

[image]

[image]

[image]

[image]

Tables 12P-12V lists the domain description from DOMAIN analysis results against NOV12. This indicates that the NOV12 sequence has properties similar to those of other proteins known to contain this domain. Domain analysis for NOV12 revealed numerous alignments of four different domains. Representations of each domain are disclosed herein.

TABLE 12P

Domain Analysis of NOV12

gnL|Smart|smart00409, 1G, Immunoglobulin (SEQ ID NO:129)
CD-Length=86 residues, 91.9% aligned
Score=65.9 bits (159), Expect=3e−11

| | | | |
|---|---|---|---|
| Query: | 2148 | KAGDTAVLDCEVTGDPKPKIFWLLPSNDMISFSIDRYTFHANG---SLTINKVKLLDSGE | 2204 |
| | | \| \|++ \| \|\| +\|+\| \| + \| +++ \| \|++ +\| +\|\|\|+ \| \|\|\| | |
| Sbjct: | 7 | KEGESVTLSCEASGNPPPTVTWYKQGGKLLAES-GRFSVSRSGGNSTLTISNVTPEDSGT | 65 |
| Query: | 2205 | YVCVARNPSGDDTKMYKLDV 2224 | |
| | | \| \| \| \| \|\| + \| \| | |
| Sbjct: | 66 | YTCAATNSSGSASSGTTLTV 85 | |

TABLE 12Q

Domain Analysis of NOV12

gnl|Smart|smart00409, 1G. Immunoglobulin (SEQ ID NO:129)
CD-Length=86 residues, 95.3% aligned
Score=65.5 bits (158), Expect=4e−11

| | | | |
|---|---|---|---|
| Query: | 595 | TVFIGETLDLPCHSTGIPDASISWVIPGNNVLYQSSRDK--KVLNNGTLRILQVTPKDQG | 652 |
| | | \|\| \|\|++ \| \| ++\| \| +++\| \| +\| +\| \| + \| \|\| \| \|\|\|+\| \| | |
| Sbjct: | 5 | TVKEGESVTLSCEASGNPPPTVTWYKQGGKLLAESGRFSVSRSGGNSTLTISNVTPEDSG | 64 |
| Query: | 653 | YYRCVAANPSGVDFLIFQVSVK 674 | |
| | | \| \| \| \| \|\| ++\| | |
| Sbjct: | 65 | TYTCAATNSSGSASSGTTLTVL 86 | |

TABLE 12R

Domain Analysis of NOV12

gnl|Smart|smart00408, IGc2, Immunoglobulin C-2 Type (SEQ ID NO:130)
CD-Length 63 residues, 96.8% aligned
Score=60.8 bits (146). Expect=9e−10

| | | | |
|---|---|---|---|
| Query: | 2150 | GDTAVLDCEVTGDPKPKIFWLLPSNDMISFSIDRYTFHANGSLTINKVKLLDSGEYVCVA | 2209 |
| | | \|++ \| \| +\|\|\| \| \| \|\| + \| + +\|\|\| \| \| \|\|\| \| \|\|\| | |
| Sbjct: | 3 | GESVTLTCPASGDPVPNITWLK-DGKPLPES---RVVASGSTLTIKNVSLEDSGLYTCVA | 58 |
| Query: | 2210 | RNPSG 2214 | |
| | | \|\| \| | |
| Sbjct: | 59 | RNSVG 63 | |

TABLE 12S

Domain Analysis of NOV12

gnl|Smart″smart00408, IGc2, Immunoglobulin C-2 Type (SEQ ID NO:130)
CD-Length=63 residues, 100.0% aligned
Score=60.1 bits (144). Expect=2e−09

| | | | |
|---|---|---|---|
| Query: | 1752 | HSGSTVELKCRAEGRPSPTVTWILANQTWSESSQGSRQACCTCDGTLVLHNLSIYDRGF | 1811 |
| | | \| +\| \| \| \| \| \| \| +\|\|+ + + \| \|\| + \|+\|+ \| \| | |
| Sbjct: | 1 | LEGESVTLTCPASGDPVPNITWLKDGKPLPESRVVAS-------GSTLTIKNVSLEDSGL | 53 |
| Query: | 1812 | YKCVASNPGG 1821 | |
| | | \| \|\|\| \| \| | |
| Sbjct: | 54 | YTCVARNSVG 63 | |

TABLE 12T

Domain Analysis of NOV12
gnl|Pfam|pfam01463, LRRCT, Leucine rich repeat C-term-
inal domain. Leucine Rich Repeats pfam00560 are short se-
quence motifs present in a number of proteins with diverse
functions and cellular locations. Leucine Rich Repeats are
often flanked by cysteine rich domains. This domain is
often found at the C-terminus of tandem leucine rich re-
peats. (SEQ ID N0:131)
CD-Length=51 residues, 74.5% aligned
Score=49.7 bits (117), Expect=2e−06

| | | | |
|---|---|---|---|
| Query: | 223 | NPWTCDCHLKWLSDWIQEKPGIYIVLPDVIKCKKDRSPSSAQQ | 265 |
| | | \|\|+ \|\|\| \|+\|\| \|++\| + \|+ ++\| \|\| \| + | |
| Sbjct: | 1 | NPFICDCELRWTLRWLREP--RRLEDPEDLRC---ASPESLRG | 38 |

TABLE 12U
Domain Analysis of NOV12
gnl|Pfam|pfam00047. ig, Immunoglobulin domain. Members of the
immunoglobulin superfamily are found in hundreds of proteins of
different functions. Examples include antibodies, the giant muscle
kinase titin and receptor tyrosine kinases. Immunoglobulin-like
domains may be involved in protein-protein and protein-ligand
interactions. The Pfam alignments do not include the first and last
strand of the immunoglobulin-like domain. (SEQ ID NO:132)
CD-Length = 68 residues, 100.0% aligned
Score = 45.1 bits (105), Expect = 5e−05

| | | | |
|---|---|---|---|
| Query: | 1851 | GESLKLPCTAXGTP-QPSVYWVLSDGTEVKPL-----QFTNSKLFLFSNGTLYIRNLASS | 1904 |
| | | \|\|\|+ \| \|+ \| \| \|+\| \| \| \|\| \|++ \| + ++ \| \|+ +\| \| ++ | |
| Sbjct: | 1 | GESVTLTCSVSGYPPDPTVTW-LRDGKEIELLGSSESRVSSGGRFSISSLSLTISSVTPE | 59 |
| Query: | 1905 | DRGTYECIA 1913 | |
| | | \| \|\|\| \|+ | |
| Sbjct: | 60 | DSGTYTCVV 68 | |

TABLE 12V
Domain Analysis of NOV12
gnl|Pfam|pfam00047, ig, Immunoglobulin domain. Members of the
immunoglobulin superfamily are found in hundreds of proteins of
different functions. Examples include antibodies, the giant muscle
kinase titin and receptor tyrosine kinases. immunoglobulin-like
domains may be involved in protein-protein and protein-ligand
interactions. The Pf am alignments do not include the first and last
strand of the immunoglobulin-like domain. (SEQ ID NO:132)
CD-Length = 68 residues, 100.0% aligned
Score = 42.4 bits (98), Expect = 3e−04

| | | | |
|---|---|---|---|
| Query: | 2150 | GDTAVLDCEVTGDPK-PKIFWLLPSNDMISFSIDRYTFHANG-------SLTINKVKLLD | 2201 |
| | | \|++ \| \| \|+\| \| \| + \|\| ++ + \| \|\|\|\|+ \| \| | |
| Sbjct: | 1 | GESVTLTCSVSGYPPDPTVTWLRDGKEIELLGSSESRVSSGGRFSISSLSLTISSVTPED | 60 |
| Query: | 2202 | SGEYVCVA 2209 | |
| | | \|\| \| \|\| | |
| Sbjct: | 61 | SGTYTCVV 68 | |

Mechanical stress or force is known to be an important modulator of cellular morphology and function in variety of tissues. It has been implicated in stretching the cell membrane and alter receptor or G protein conformation thereby initiating signaling pathways usually used by the growth factors. It has been shown to induce changes in bone, modulate fibrogenic activity of human VSM cells, platelet aggregations and tooth movements (Stoltz et al., 2000, Biorheology vol. 37: 3-14; Nomura S and Takano-YamamotoT 2000, Matrix Biol., vol 19: 91-96; Li C and Xu Q, 2000 Cell Signal vol 12: 435-45). As a response to mechanical stress, expression of many stress related proteins such as HSP 70, glutamate/aspartate transporter, nitric oxide synthetase, prostaglandin G/H synthetase etc. are induced. In case of bone cells the mechanical stress is converted to series of biochemical reactions which activates osteoclasts and oteoblasts to cause bone resorption and formation. Recently, Einat P, Mor O, Skaliter R, Feinstein E, and Faerman A have described a new mechanical stress induced cDNA for protein 608 in rat (Geneseq database) and have implicated its role in osteoporosis. Here we describe a human paralogue of this novel mechanical stress induced protein gene.

The disclosed NOV12 nucleic acid of the invention encoding a Mechanical Stress Induced Protein-like protein includes the nucleic acid whose sequence is provided in Table 12A or a fragment thereof. The invention also includes a mutant or variant nucleic acid any of whose bases may be changed from the corresponding base shown in Table 12A while still encoding a protein that maintains its Mechanical Stress Induced Protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 20% percent of the bases may be so changed.

The disclosed NOV12 protein of the invention includes the Mechanical Stress Induced Protein-like protein whose sequence is provided in Table 12B. The invention also includes a mutant or variant protein any of whose residues may be changed from the corresponding residue shown in Table 12B while still encoding a protein that maintains its Mechanical Stress Induced Protein-like activities and physiological functions, or a functional fragment thereof. In the mutant or variant protein, up to about 57% percent of the residues may be so changed.

The invention further encompasses antibodies and antibody fragments, such as F_{ab} or (F_{ab})₂, that bind immunospecifically to any of the proteins of the invention.

The above defined information for this invention suggests that this Mechanical Stress Induced Protein-like protein (NOV12) may function as a member of a “Mechanical Stress Induced Protein family”. Therefore, the NOV12 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV12 nucleic acids and proteins of the invention are useful in potential therapeutic applications implicated in cancer including but not limited to various pathologies and disorders as indicated below. For example, a cDNA encoding the Mechanical Stress Induced Protein-like protein (NOV12) may be useful in gene therapy, and the Mechanical Stress Induced Protein-like protein (NOV12) may be useful when administered to a subject in need thereof. By way of nonlimiting example, the compositions of the present invention will have efficacy for treatment of patients suffering from osteoporosis, osteoarthritis, cardiac hypertrophy, atherosclerosis, hypertension, restenosis, and other pathologies and conditions. The NOV12 nucleic acid encoding the Mechanical Stress Induced Protein-like protein of the invention, or fragments thereof, may further be useful in diagnostic applications, wherein the presence or amount of the nucleic acid or the protein are to be assessed.

NOV12 nucleic acids and polypeptides are further useful in the generation of antibodies that bind immuno-specifically to the novel NOV12 substances for use in therapeutic or diagnostic methods. These antibodies may be generated according to methods known in the art, using prediction from hydrophobicity charts, as described in the “Anti-NOVX Antibodies” section below. These novel proteins can be used in assay systems for functional analysis of various human disorders, which will help in understanding of pathology of the disease and development of new drug targets for various disorders.

NOV13

A disclosed NOV13a nucleic acid of 840 nucleotides (also referred to as Curagen Accession No. CG55908-01) encoding a novel Integrin-like FG-GAP domain containing novel protein-like protein is shown in Table 13A. An open reading frame was identified beginning with an GCC initiation codon at nucleotides 24 and ending with a TAA codon at nucleotides 836-838. The untranslated regions are underlined and the start and stop codons are in bold letters in Table 13A. The start codon for NOV13 is not a traditional initiation codon. Therefore, NOV13 may be a partial open reading frame extending further into the 5′ region.

TABLE 13A
NOV13a nucleotide sequence.

(SEQ ID NO:57)

GGCCTCCGGGATTTGCTACCTTTTTGGCTCCCTGCTCGTCGAACTGCTCTTCTCACGGGCTGTCGCCTTCAA
TCTGGACGTGATGGGTGCCTTGCGCAAGGAGGGCGAGCCAGGCAGCCTCTTCGGCTTCTCTGTGGCCCTGCA
CCGGCAGTTGCAGCCCCGACCCCAGAGCTGGCTGCTGGTGGGTGCTCCCCAGGCCCTGGCTCTTCCTGGGCA
GCAGGCGAATCGCACTGGAGGCCTCTTCGCTTGCCCGTTGAGCCTGGAGGAGACTGACTGCTACAGAGTGGA
CATCGACCAGGGAGCTGATATGCAAAAGGAAAGCAAGGAGAACCAGTGGTTGGGAGTCAGTGTTCGGAGCCA
GGGGCCTGGGGGCAACATTGTTGACTGCGCCCGGGGCACGGCCAACTGTGTGGTGTTCAGCTGCCCACTCTA
CAGCTTTGACCGCGCGGCTGTGCTGCATGTCTGGGGCCGTCTCTGGAACAGCACCTTTCTGGAGGAGTACTC
AGCTGTGAAGTCCCTGGAAGTGATTGTCCGGGCCAACATCACAGTGAAGTCCTCCATAAAGAACTTGATGCT
CCGAGATGCCTCCACAGTGATCCCAGTGATGGTATACTTGGACCCCATGGCTGTGGTGGCAGAAGGAGTGCC
CTGGTGGGTCATCCTCCTGGCTGTACTGGCTGGGCTGCTGGTGCTAGCACTGCTGGTGCTGCTCCTGTGGAA
GTGTGGCTTCTTCCATCGGAGCAGCCAGAGCTCATCTTTTCCCACCAACTATCACCGGGCCTGTCTGGCTGT
GCAGCCTTCAGCCATGGAAGTTGGGGGTCCAGGGACTGTGGGGTAACT

In a search of public sequence databases, the NOV13a nucleic acid sequence, located on the q13 region of chromosome 12, has 388 of 392 bases (98%) identical to a gb:GENBANK-ID:AF072132|acc:AF072132.1 mRNA from Homo sapiens (Homo sapiens integrin alpha-7 mRNA, complete cds) (E=3.9e⁻⁸¹). Public nucleotide databases include all GenBank databases and the GeneSeq patent database.

The disclosed NOV13a polypeptide (SEQ ID NO 58) encoded by SEQ ID NO: 57 has 278 amino acid residues and is presented in Table 13B using the one-letter amino acid code. Signal P, Psort and/or Hydropathy results predict that NOV13a has no signal peptide and is likely to be localized in the plasma membrane with a certainty of 0.7300. In other embodiments, NOV13a may also be localized to the endoplasmic reticulum (membrane) with acertainty of 0.6400, the microbody (peroxisome) with a certainty of 0.1665, or in the endoplasmic reticulum (lumen) with a certainty of 0.1000. The most likely cleavage site for NOV13 is between positions 22 and 23: AVA-FN.

TABLE 13B
Encoded NOV13a protein sequence.

(SEQ ID NO:58)

ASGICYLFGSLLVELLFSRAVAFNLDVMGALRKEGEPGSLFGFSVALHRQLQPRPQSWLLVGAPQALALPGQ
QANRTGGLFACPLSLEETDCYRVDIDQGADMQKESKENQWLGVSVRSQGPGGKIVDCARGTANCVVFSCPLY
SFDRAAVLHVWGRLWNSTFLEEYSAVKSLEVIVRANITVKSSIKNLMLRDASTVIPVMNYLDPMAVVAEGVP
WWVILLAVLAGLLVLALLVLLLW1CCGFFHRSSQSSSFPTNHRACLAVQPSAMEVGGPGTVG

A search of sequence databases reveals that the NOV13a amino acid sequence has 158 of 225 amino acid residues (70%) identical to, and 170 of 225 amino acid residues (75%) similar to, the 1161 amino acid residue ptnr:SPTREMBL-ACC:O88731 protein from Mus musculus (Mouse) (Integrin Alpha 7 Precursor) (E=3.7e⁻⁷⁵). Public amino acid databases include the GenBank databases, SwissProt, PDB and PIR.

NOV13 is expressed in at least the following tissues: brain, lymph node. This information was derived by determining the tissue sources of the sequences that were included in the invention including but not limited to SeqCalling sources, Public EST sources, Literature sources, and/or RACE sources.

The disclosed NOV13a polypeptide has homology to the amino acid sequences shown in the BLASTP data listed in Table 13C.

TABLE 13C
BLAST results for NOV13a

| Gene Index/ | | Length | Identity | Positives | |
|---|---|---|---|---|---|
| Identifier | Protein/Organism | (aa) | (%) | (%) | Expect |
| gi\|3378243\|emb\| | integrin alpha 7 | 1161 | 128/175 | 133/175 | 7e−67 |
| CAA73024.1\| | [Mus musculus] | | (73%) | (75%) | |
| (Y12380) | | | | | |
| gi\|12643723\|sp\| | Integrin alpha-7 | 1181 | 116/130 | 116/130 | 4e−62 |
| Q13683\| | precurso | | (89%) | (89%) | |
| ITA7_HUMAN | | | | | |
| gi\|3158408\|gb\| | integrin alpha 7 | 1137 | 116/130 | 116/130 | 4e−62 |
| AAC18968.1\| | [Homo sapiens] | | (89%) | (89%) | |
| (AF052050) | | | | | |
| gi\|4504753\|ref\|NP_0 | integrin alpha 7 | 1137 | 116/130 | 116/130 | 4e−62 |
| 02197.1\| | precursor [Homo sapiens] | | (89%) | (89%) | |
| (NM_002206) | | | | | |
| gi\|4699891\|emb\| | integrin alpha 7 | 1141 | 116/130 | 116/130 | 5e−62 |
| CAB41534.1\| | chain [Homo sapiens] | | (89%) | (89%) | |
| (AJ228836) | | | | | |

The homology between these and other sequences is shown graphically in the ClustalW analysis shown in Table 13D. In the ClustalW alignment of the NOV13 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

The integrins are a family of heterodimeric membrane glycoproteins that mediate a wide spectrum of cell-cell and cell-matrix interactions. Their capacity to participate in cellular adhesive processes underlies a wide range of functions. The integrins have preeminent roles in cell migration and morphologic development, differentiation, and metastasis. To a large extent, the diversity and specificity of functions mediated by integrins rest in the structural diversity of the 16 different alpha and 8 beta chains that have been identified and in their ligand-binding and signal transduction capacity. One structural difference in the alpha chains appears to divide them into 2 subgroups. The I-integrin alpha chains have an insertion of about 180 amino acids in the extracellular region, and the non-I-integrins do not. The functional significance of the I-domain is not known. Alternate splicing increases the structural diversity in the cytoplasmic domains of several integrin alpha and beta chains, and this presumably further expands their functional repertoire.

Expression of the alpha-7 integrin gene (ITGA7) is developmentally regulated during the formation of skeletal muscle. Increased levels of expression and production of isoforms containing different cytoplasmic and extracellular domains accompany myogenesis. From examining the rat and human genomes by Southern blot analysis₄and in situ hybridization, Wang et al. (1995) determined that both genomes contain a single alpha-7 gene. In the human, ITGA7 is present on 12q13, as localized by fluorescence in situ hybridization (Wang et al., 1995). Phylogenetic analysis of the integrin alpha-chain sequences suggested that the early integrin genes evolved in 2 pathways to form the I-integrins and the non-I-integrins. The I-integrin alpha chains apparently arose as a result of an early insertion into the non-I-gene. The I-chain subfamily further evolved by duplications within the same chromosome. The non-I-integrin alpha-chain genes are located in clusters on chromosomes 2, 12, and 17, which coincides closely with the localization of the human homeobox gene clusters. Non-I-integrin alpha-chain genes appear to have evolved in parallel and in proximity to the HOX clusters. Thus, the HOX genes that underlie the design of body structure and the integrin genes that underlie informed cell-cell and cell-matrix interactions appear to have evolved in parallel and coordinate fashions.

ITGA7 is a specific cellular receptor for the basement membrane protein laminin-1, as well as for the laminin isoforms-2 and -4. The alpha-7 subunit is expressed mainly in skeletal and cardiac muscle and may be involved in differentiation and migration processes during myogenesis. Three cytoplasmic and 2 extracellular splice variants are developmentally regulated and expressed in different sites in the muscle. In adult muscle, the alpha-7A and alpha-7B subunits are concentrated in myotendinous junctions but can also be detected in neuromuscular junctions and along the sarcolemmal membrane. To study the involvement of alpha-7 integrin during myogenesis and its role in muscle integrity and function, Mayer et al. (1997) generated a null allele of the ITGA7 gene in the germline of mice by homologous recombination in embryonic stem (ES) cells. To their surprise, mice homozygous for the mutation were viable and fertile, indicating that the gene is not essential for myogenesis. However, histologic analysis of skeletal muscle showed typical signs of progressive muscular dystrophy starting soon after birth, but with a distinct variability in different muscle types. The histopathologic changes indicated an impairment of function of the myotendinous junctions. Thus, ITGA7 represents an indispensable linkage between the muscle fiber and extracellular matrix that is independent of the dystrophin-dystroglycan complex-mediated interaction of the cytoskeleton with the muscle basement membrane.

The basal lamina of muscle fibers plays a crucial role in the development and function of skeletal muscle. An important laminin receptor in muscle is integrin alpha-7/beta-1D. Integrin beta-1 (ITGB1; 135630) is expressed throughout the body, while integrin alpha-7 is more muscle-specific. To address the role of integrin alpha-7 in human muscle disease, Hayashi et al. (1998) determined alpha-7 protein expression in muscle biopsies from 117 patients with unclassified congenital myopathy and congenital muscular dystrophy by immunocytochemistry. They found 3 unrelated patients with integrin alpha-7 deficiency and normal laminin alpha-2 chain expression. (Deficiency of LAMA2 (156225) causes congenital muscular dystrophy, and a secondary deficiency of integrin alpha-7 was observed in some cases.) The 3 patients were found to carry mutations in the ITGA7 gene. Hayashi et al. (1998) noted that the finding in these patients accords well with the findings in Itga7 knockout mice (Mayer et al., 1997).

ALLELIC VARIANTS (selected examples)

0.0001 MYOPATHY, CONGENITAL [ITGA7, 21-BP INS]

In a 4-year-old Japanese boy born at term from nonconsanguineous parents, Hayashi et al. (1998) observed compound heterozygosity for 2 splicing mutations: one causing a 21-bp insertion in the conserved cysteine-rich region and the other causing a 98-bp deletion. The child's psychomotor milestones were delayed; he acquired the ability to roll over at 9 months, and walked at 2.5 years. He could not jump or run. Mental retardation was also observed, and verbal abilities were limited to only a few words. Serum creatine kinase (CK) activity was mildly elevated. Brain MRI and EEG were normal. It was unclear whether mental retardation was caused by alpha-7-deficiency, but Hayashi et al. (1998) observed that alpha-7 is also expressed in the developing nervous system. Muscle biopsy at 15 months showed changes consistent with congenital myopathy. Sequence analysis of genomic DNA from this patient showed an A-to-G transition at position −2 of the splice-acceptor site in cDNA nucleotide 1506, and a T-to-C substitution at the splice-donor site at position +2 in cDNA nucleotide 2712, respectively. The second mutation was found in the unaffected father, whereas the first was not detected in either parent, suggesting a new mutation.

0.0002 MYOPATHY, CONGENITAL [ITGA7, 98-BP DEL]

See 600536.0001 and Hayashi et al. (1998). The 98-bp frameshift deletion caused a premature termination codon 12 bp downstream.

0.0003 MYOPATHY, CONGENITAL [ITGA7,]

In an 11-year-old Japanese girl with nonconsanguineous parents and signs of congenital myopathy, Hayashi et al. (1998) found compound heterozygosity for the 98-bp deletion (600536.0002) and a 1-bp frameshift deletion at cDNA nucleotide 1204, which created a premature termination codon at amino acid 505. At 2 months of age, the girl was diagnosed with congenital dislocation of the hip and torticollis, which required surgical intervention. She acquired independent ambulation at 2 years, and Gowers sign and waddling gait were observed. She had never been able to climb stairs without support and could not run. There was no cognitive impairment. Serum CK was mildly elevated. Muscle biopsy showed changes consistent with congenital myopathy, with substantial fatty replacement and fiber size variation.

Another patient with congenital myopathy and marked deficiency of ITGA7 mRNA showed hypotonia and torticollis from birth. No mutation was identified in the ITGA7 cDNA.

### References

1. Hayashi, Y. K.; Chou, F.-L.; Engvall, E.; Ogawa, M.; Matsuda, C.; Hirabayashi, S.; Yokochi, K.; Ziober, B. L.; Kramer, R. H.; Kaufman, S. J.; Ozawa, E.;Goto, Y.; Nonaka, I.; Tsukahara, T.; Wang, J.; Hoffman, E. P.; Arahata, K.: Mutations in the integrin alpha-7 gene cause congenital myopathy. Nature Genet. 19: 94-97, 1998. PubMed ID: 9590299

2. Mayer, U.; Saher, G.; Fassler, R.; Bornemann, A.; Echtermeyer, F.; von der Mark, H.; Miosge, N.; Poschl, E.; von der Mark, K.: Absence of integrin alpha-7 causes a novel form of muscular dystrophy. Nature Genet. 17: 318-323, 1997. PubMed ID: 9354797

3. Wang, W.; Wu, W.; Desai, T.; Ward, D. C.; Kaufman, S. J.: Localization of the alpha-7 integrin gene (ITGA7) on human chromosome 12q13: clustering of integrin and Hox genes implies parallel evolution of these gene families. Genomics 26: 563-570, 1995.

The disclosed NOV13 nucleic acid of the invention encoding a Integrin-like FG-GAP domain containing novel protein-like protein includes the nucleic acid whose sequence is provided in Table 13A or a fragment thereof. The invention also includes a mutant or variant nucleic acid any of whose bases may be changed from the corresponding base shown in Table 13A while still encoding a protein that maintains its Integrin-like FG-GAP domain containing novel protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2% percent of the bases may be so changed.

The disclosed NOV13 protein of the invention includes the Integrin-like FG-GAP domain containing novel protein-like protein whose sequence is provided in Table 13B. The invention also includes a mutant or variant protein any of whose residues may be changed from the corresponding residue shown in Table 13B while still encoding a protein that maintains its Integrin-like FG-GAP domain containing novel protein-like activities and physiological functions, or a functional fragment thereof. In the mutant or variant protein, up to about 30% percent of the residues may be so changed.

The invention further encompasses antibodies and antibody fragments, such as F_{ab} or (F_{ab})₂, that bind immunospecifically to any of the proteins of the invention.

The above defined information for this invention suggests that this Integrin-like FG-GAP domain containing novel protein-like protein (NOV13) may function as a member of a “Integrin-like FG-GAP domain containing novel protein family”. Therefore, the NOV13 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV13 nucleic acids and proteins of the invention are useful in potential therapeutic applications implicated in cancer including but not limited to various pathologies and disorders as indicated below. For example, a cDNA encoding the Integrin-like FG-GAP domain containing novel protein-like protein (NOV13) may be useful in gene therapy, and the Integrin-like FG-GAP domain containing novel protein-like protein (NOV13) may be useful when administered to a subject in need thereof. By way of nonlimiting example, the compositions of the present invention will have efficacy for treatment of patients suffering from Achalasia-addisonianism-alacrimia syndrome; Cataract, polymorphic and lamellar, Cyclic ichthyosis with epidermolytic hyperkeratosis; Diabetes insipidus, nephrogenic, autosomal dominant; Diabetes insipidus, nephrogenic, autosomal recessive; Enuresis, nocturnal, 2; Epidermolysis bullosa simplex, Koebner, Dowling-Meara, and Weber-Cockayne types; Epidermolytic hyperkeratosis; Fundus albipunctatus; Glioma; Ichthyosis bullosa of Siemens; Keratoderma, palmoplantar, nonepidermolytic; Meesmann corneal dystrophy; Monilethrix; Myopathy, congenital; Pachyonychia congenita, Jackson-Lawler type; Pachyonychia congenita, Jadassohn-Lewandowsky type; Palmoplantar keratoderma, Bothnia type; Persistent Mullerian duct syndrome, type II; Spastic paraplegia-10; White sponge nevus; Liver disease, susceptibility to, from hepatotoxins or viruses; Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection; lymphedema, allergies, and other pathologies and conditions. The NOV13 nucleic acid encoding the Integrin-like FG-GAP domain containing novel protein-like protein of the invention, or fragments thereof, may further be useful in diagnostic applications, wherein the presence or amount of the nucleic acid or the protein are to be assessed.

NOV13 nucleic acids and polypeptides are further useful in the generation of antibodies that bind immuno-specifically to the novel NOV13 substances for use in therapeutic or diagnostic methods. These antibodies may be generated according to methods known in the art, using prediction from hydrophobicity charts, as described in the “Anti-NOVX Antibodies” section below. The disclosed NOV13 protein has multiple hydrophilic regions, each of which can be used as an immunogen. In one embodiment, a contemplated NOV13 epitope is from about amino acids 30 to 130. In another embodiment, a NOV13 epitope is from about amino acids 240 to 270. These novel proteins can be used in assay systems for functional analysis of various human disorders, which will help in understanding of pathology of the disease and development of new drug targets for various disorders.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57 as a hybridization probe, NOVX molecules can be isolated using standard hybridization and cloning techniques (e.g., as described in Sambrook, et al., (eds.), Molecular Cloning: A Laboratory Manual 2^{nd} Ed., Cold Spring Harbor Laboratory Press, Cold Spring Harbor, N.Y., 1989; and Ausubel, et al., (eds.), Current Protocols in Molecular Biology, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1,3, 5, 7, 9, 11, 13, 15, 17, 19, 21, 23, 25, 27, 29, 31, 33, 35, 37, 39, 41, 43, 45, 47, 49, 51, 53, 55, and 57, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, or 57 is one that is sufficiently complementary to the nucleotide sequence shown SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, or 57 that it can hydrogen bond with little or no mismatches to the nucleotide sequence shown SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., Current Protocols in Molecular Biology, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, or 57; or an anti-sense strand nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, or 57; or of a naturally occurring mutant of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, or 57, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57 due to degeneracy of the genetic code and thus encode the same NOVX proteins as that encoded by the nucleotide sequences shown in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57. In another embodiment, an isolated nucleic acid molecule of the invention has a nucleotide sequence encoding a protein having an amino acid sequence shown in SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58.

In addition to the human NOVX nucleotide sequences shown in SEQ ID NOS: 1, 3, 5, 7, 9, 11, 13, 15, 17, 19, 21, 23, 25, 27, 29, 31, 33, 35, 37, 39,41, 43, 45, 47, 49, 51, 53, 55, and 57, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, Current Protocols in Molecular Biology, John Wiley & Sons, NY, and Kriegler, 1990; Gene Transfer and Expression, A Laboratory Manual, Stockton Press, NY.

In a third embodiment, a nucleic acid that is hybridizable to the nucleic acid molecule comprising the nucleotide sequences SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, Current Protocols in Molecular Biology, John Wiley & Sons, NY, and Kriegler, 1990, Gene Transfer and Expression, A Laboratory Manual, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58. Preferably, the protein encoded by the nucleic acid molecule is at least about 60% homologous to SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58; more preferably at least about 70% homologous SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58; still more preferably at least about 80% homologous to SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58; even more preferably at least about 90% homologous to SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58; and most preferably at least about 95% homologous to SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58.

An isolated nucleic acid molecule encoding an NOVX protein homologous to the protein of SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58 can be created by introducing one or more nucleotide substitutions, additions or deletions into the nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, such that one or more amino acid substitutions, additions or deletions are introduced into the encoded protein.

Mutations can be introduced into SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g. alanine, valine, leucine, isoleucine, proline, phenylalanine, methionine, tryptophan), beta-branched side chains (e.g. threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, or fragments, analogs or derivatives thereof. An “antisense” nucleic acid comprises a nucleotide sequence that is complementary to a “isense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58, or antisense nucleic acids complementary to an NOVX nucleic acid sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2, 2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (ie., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996. supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g. Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58. The invention also includes a mutant or variant protein any of whose residues may be changed from the corresponding residues shown in SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS: 2, 4, 6, 8, 10, 12, 14, 30 16, 18, 20, 22, 24, 26, 28, 30, 32, 34, 26, 28, 40, 42, 44, 46, 48, 50, 52, 54, 56, or 58) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58. In other embodiments, the NOVX protein is substantially homologous to SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58, and retains the functional activity of the protein of SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58, and retains the functional activity of the NOVX proteins of SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J. Mol Biol 48: 443453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g. A, T, C, G, U, or I, in the case of nucleic acids) occurs in both sequences to yield the number of matched positions, dividing the number of matched positions by the total number of positions in the region of comparison (i e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, or 58, whereas a “non-NOVX polypeptide” refers to a polypeptide having an amino acid sequence corresponding to a protein that is not substantially homologous to the NOVX protein, e.g. a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g. mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) Current Protocols in Molecular Biology, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2}, fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: Monoclonal Antibodies and Cancer Therapy, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: Monoclonal Antibodies and Cancer Therapy, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂, fragments. These fragments are reduced in the presence of the dithiol comHSP90 co-chaperoneg agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoetbylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fe region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidinibiotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g. replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g. in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g. NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57), but more preferably, is a non-human homologue of a human NOVX gene. For example, a mouse homologue of human NOVX gene of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: Teratocarcinomas and Embryonic Stem Cells: A Practical Approach, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used: The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat No. 5,233,409), plasmids (Cull, et al., 1992. Proc. Natl. Acad. Sci USA 89: 1865-1869) or on phage (Scott and Smith, 1990. Science 249: 386-390; Devlin, 1990. Science 249: 404406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²+, diacylglycerol, IP_{3,} etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-1 14, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be, bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g. GAL4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (eg., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, eg., DNA or RNA, to high-density arrays containing hundreds or thousands of oligonucleotides probes. See, e.g. Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pbarmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOYX expression or activity can be identified by, for example, any or a combination of diagnostic or prognostic assays as described herein. Administration of a prophylactic agent can occur prior to the manifestation of symptoms characteristic of the NQVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

Identification of NOVX Clones

The novel NOVX target sequences identified in the present invention were subjected to the exon linking process to confirm the sequence. PCR primers were designed by starting at the most upstream sequence available, for the forward primer, and at the most downstream sequence available for the reverse primer. Table 14A shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. Table 14B shows a list of these bacterial clones. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE 14A
PCR Primers for Exoii Linking

| | | SEQ | | SEQ |
|---|---|---|---|---|
| NOVX | | ID | | ID |
| Clone | Primer 1 (5′-3′) | NO | Primer 2 (5′-3′) | NO |
| NOV3a | AGCACGCACTTGCCCAGAGCTATC | 138 | CCTATGGCTGAAGGCCGAGGT | 139 |
| NOV3b | CTGGGTCTCCCCTCCCAC | 140 | GTTTATTCTGAGCACCGGGAA | 141 |
| NOV4a | AGCACGCACTTGCCCAGAGCTATC | 142 | CATTTGCTGTCTCCTCTGCTCACCAG | 143 |
| NOV4b | ATGACGCCCGATCTGCTCAACTTCA | 144 | GTCCAGCGAGTCCCGGGTGTG | 145 |
| NOV5 | GCTTCTCCGAGCCCGAATACTC | 146 | CTCGAGGCCATTCACGAGTTGT | 147 |
| NOV6a | GTTTGGGGATGTGGTCCTTGCT | 148 | CTCACCTGCGGTCCGGTAG | 149 |
| NOV6b | GTTTGGGGATGTGGTCCTTGCT | 150 | CTCACCTGCGGTGCGGTAG | 151 |
| NOV6c | GTTTGGGGATGTGGTCCTTGCT | 152 | CTCACCTGCGCTGCGGTAG | 153 |
| NOV8 | GCGACTGGAGCTTTCTGGGAAGACT | 154 | CACTAGATGGCCAAGTCCTCCGGTC | 155 |
| NOV12a | ATGAAGGTAAAAGGCAGAGGAATCACC | 156 | GCCACACAGCGATAATAACCTTGGTCT | 157 |
| NOV13 | GGCCTCCGOGATTTGCTACCTTTT | 158 | AGTTACCCCACAGTCCCTGGAC | 159 |

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

TABLE 14B
Physical Clones for PCR products

| NOVX | |
|---|---|
| Clone | Bacterial Clone |
| NOV1a | Physical clone: 147235722 |
| NOV2 | Physical clone: AC008962, S3ft: 1018515 |
| NOV3b | Physical clone: 165390393 |
| NOV3c | Physical clone: gb_AL353113 |
| NOV4b | Physical clone: gb: L24804.1 |
| NOV6b | Physical clone: 151299399, 151299404, 147249435 |
| NOV6c | Physical clone: 151299399, 151299404, 147249435 |
| NOV8 | Physical clone: 146315970, 146315964, 146315967 |
| NOV10 | Physical clone: scdb3: 5055223, AC011492 |
| NOV11a | Physical clone: 147758163 |
| NOV11b | Physical clone: 152231160 152766718 137044002 139952328 |
| NOV11c | Physical clone: gb_BE905283.1 , AC022468.5 |
| NOV13 | Bacterial clone: 87897:: AC073487.698180.M18 |

### Example 2

Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

### Example 2

Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISMS 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58′-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C.for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel_{—v}1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4. ID) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶cells/ml onto Falcon 6 well tissue culture plates that had been coated overnight with 0.5 μg/ml anti-CD28 (Pharmingen) and 3 μg/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5× ⁻⁵M (Gibco), and 10 mM Hepes (Gibco) and IL-2. The expanded CD8 cells were then activated again with plate bound anti-CI)3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10 ⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 n/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0. I mM dbcAMP at 5×10⁵cells/ml for 8 days, changing the media every 3 days and adjusting the cell concentration to 5×10⁵cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD 106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD 1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at -20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 51l DTT, 7 μl RNAsin and 8 μL DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_comprehensive Panel_v1.0

The plates for Al_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the Al_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample.

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 51 panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.0 1 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at -80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus;

the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx Inferior Temporal Cortex

NOV9

Expression of gene NOV9 was assessed using the primer-probe sets Ag2708, Ag299 and Ag 1347, described in Tables 15, 16 and 17. Results of the RTQ-PCR runs are shown in Tables 18, 19, 20, 21, 22, and 23.

TABLE 15
Probe Name Ag2708

| Primers | Sequences | Length | Start Position | SEQ ID NO: |
|---|---|---|---|---|
| Forward | 5′-ggtgctcatcttgcttttaaca-3′ | 22 | 491 | 160 |
| Probe | TET-5′-tctttgtgaggttcctccattgctca-3′-TAMRA | 26 | 542 | 161 |
| Reverse | 5′-gtagtgtcaacacaggcaagct-3′ | 22 | 568 | 162 |

TABLE 16
Probe Name Ag299

| | | | | SEQ ID |
|---|---|---|---|---|
| Primers | Sequences | Length | Start Position | NO: |
| Forward | 5′-gtgatggcatttgaccgctat-3′ | 21 | 360 | 163 |
| Probe | TET-5′-agctgtttgcaggcccctccactac-3′-TAMRA | 25 | 383 | 164 |
| Reverse | 5′-catacagacaagggtgcatgactac-3′ | 25 | 411 | 165 |

TABLE 17
Probe Name Ag1347

| | | | | SEQ ID |
|---|---|---|---|---|
| Primers | Sequences | Length | Start Position | NO: |
| Forward | 5′-acaccatcattacacccatgat-3′ | 22 | 844 | 166 |
| Probe | TET-5′-cccctcatatatacactgaggaacaagga-3′-TAMRA | 29 | 870 | 167 |
| Reverse | 5′-gcaccttcttaagtgctccttt-3′ | 22 | 903 | 168 |

TABLE 18
CNS_neurodegeneration_v1.0

| | Rel. Exp. (%) | Rel. Exp. (%) | | Rel. Exp. (%) | Rel. Exp. (%) |
|---|---|---|---|---|---|
| | Ag2708, Run | Ag299, Run | Tissue | Ag2708, Run | Ag299, Run |
| Tissue Name | 208394380 | 206914240 | Name | 208394380 | 206914240 |
| AD 1 Hippo | 0.0 | 8.4 | Control | 7.4 | 0.0 |
| | | | (Path) 3 | | |
| | | | Temporal | | |
| | | | Ctx | | |
| AD 2 Hippo | 0.0 | 54.3 | Control | 19.5 | 15.0 |
| | | | (Path) 4 | | |
| | | | Temporal | | |
| | | | Ctx | | |
| AD 3 Hippo | 0.0 | 0.0 | AD 1 | 9.0 | 15.5 |
| | | | Occipital | | |
| | | | Ctx | | |
| AD 4 Hippo | 0.0 | 9.3 | AD 2 | 0.0 | 0.0 |
| | | | Occipital | | |
| | | | Ctx | | |
| | | | (Missing) | | |
| AD 5 hippo | 15.3 | 37.1 | AD 3 | 20.7 | 10.3 |
| | | | Occipital | | |
| | | | Ctx | | |
| AD 6 Hippo | 15.4 | 50.3 | AD 4 | 21.2 | 9.7 |
| | | | Occipital | | |
| | | | Ctx | | |
| Control 2 | 25.9 | 15.0 | AD 5 | 0.0 | 5.8 |
| Hippo | | | Occipital | | |
| | | | Ctx | | |
| Control 4 | 75.8 | 80.1 | AD 6 | 21.9 | 5.6 |
| Hippo | | | Occipital | | |
| | | | Ctx | | |
| Control (Path) 3 | 0.0 | 0.0 | Control 1 | 0.0 | 0.0 |
| Hippo | | | Occipital | | |
| | | | Ctx | | |
| AD 1 Temporal | 38.2 | 37.4 | Control 2 | 41.5 | 15.7 |
| Ctx | | | Occipital | | |
| | | | Ctx | | |
| AD 2 Temporal | 46.0 | 32.8 | Control 3 | 7.8 | 8.1 |
| Ctx | | | Occipital | | |
| | | | Ctx | | |
| AD 3 Temporal | 0.0 | 0.0 | Control 4 | 32.1 | 21.3 |
| Ctx | | | Occipital | | |
| | | | Ctx | | |
| AD 4 Temporal | 0.0 | 6.7 | Control | 29.9 | 39.0 |
| Ctx | | | (Path) 1 | | |
| | | | Occipital | | |
| | | | Ctx | | |
| AD 5 Inf | 23.2 | 0.0 | Control | 41.2 | 45.4 |
| Temporal Ctx | | | (Path) 2 | | |
| | | | Occipital | | |
| | | | Ctx | | |
| AD 5 | 23.2 | 15.8 | Control | 0.0 | 0.0 |
| SupTemporal | | | (Path) 3 | | |
| Ctx | | | Occipital | | |
| | | | Ctx | | |
| AD 6 Inf | 39.0 | 14.3 | Control | 11.8 | 9.2 |
| Temporal Ctx | | | (Path) 4 | | |
| | | | Occipital | | |
| | | | Ctx | | |
| AD 6 Sup | 100.0 | 46.0 | Control 1 | 9.4 | 18.0 |
| Temporal Ctx | | | Parietal Ctx | | |
| Control 1 | 0.0 | 0.0 | Control 2 | 23.8 | 31.0 |
| Temporal Ctx | | | Parietal Ctx | | |
| Control 2 | 13.2 | 0.0 | Control 3 | 9.3 | 66.4 |
| Temporal Ctx | | | Parietal Ctx | | |
| Control 3 | 0.0 | 0.0 | Control | 38.4 | 45.1 |
| Temporal Ctx | | | (Path) 1 | | |
| | | | Parietal Ctx | | |
| Control 4 | 37.6 | 34.6 | Control | 9.2 | 20.0 |
| Temporal Ctx | | | (Path) 2 | | |
| | | | Parietal Ctx | | |
| Control (Path) 1 | 31.0 | 43.8 | Control | 0.0 | 0.0 |
| Temporal Ctx | | | (Path) 3 | | |
| | | | Parietal Ctx | | |
| Control (Path) 2 | 41.8 | 100.0 | Control | 73.7 | 33.4 |
| Temporal Ctx | | | (Path) 4 | | |
| | | | Parietal Ctx | | |

TABLE 19
Panel 1

| | Rel. Exp. (%) Ag299, | | Rel. Exp. (%) Ag299, |
|---|---|---|---|
| Tissue Name | Run 117874412 | Tissue Name | Run 117874412 |
| Endothelial cells | 0.0 | Renal ca. 786-0. | 1.3 |
| Endothelial cells | 0.0 | Renal ca. A498 | 1.5 |
| (treated) | | | |
| Pancreas | 3.6 | Renal ca. RXF 393 | 0.4 |
| Pancreatic ca. CAPAN 2 | 0.9 | Renal ca. ACHN | 0.0 |
| Adrenal gland | 4.5 | Renal ca. UO-31 | 1.6 |
| Thyroid | 2.9 | Renal ca. TK-10 | 2.7 |
| Salivary gland | 3.5 | Liver | 1.7 |
| Pituitary gland | 0.0 | Liver (fetal) | 0.0 |
| Brain (fetal) | 2.0 | Liver ca. | 9.0 |
| | | (hepatoblast) HepG2 | |
| Brain (whole) | 1.6 | Lung | 0.1 |
| Brain (amygdala) | 3.6 | Lung (fetal) | 0.7 |
| Brain (cerebellum) | 4.3 | Lung ca. (small cell) | 1.4 |
| | | LX-1 | |
| Brain (hippocampus) | 4.5 | Lung ca. (small cell) | 12.9 |
| | | NCI-H69 | |
| Brain (substantia nigra) | 4.4 | Lung ca. (s.cell var.) | 0.8 |
| | | SHP-77 | |
| Brain (thalamus) | 4.2 | Lung ca. (large | 5.2 |
| | | cell)NCI-H460 | |
| Brain (hypothalamus) | 4.5 | Lung ca. (non-sm. | 2.1 |
| | | cell) A549 | |
| Spinal cord | 0.9 | Lung ca. (non-s.cell) | 2.3 |
| | | NCI-H23 | |
| glio/astro U87-MG | 0.0 | Lung ca. (non-s.cell) | 8.4 |
| | | HOP-62 | |
| glio/astro U-118-MG | 0.6 | Lung ca. (non-s.cl) | 27.4 |
| | | NCI-H522 | |
| astrocytoma SW1783 | 0.5 | Lung ca. (squam.) | 5.5 |
| | | SW 900 | |
| neuro*; met SK-N-AS | 0.0 | Lung ca. (squam.) | 3.8 |
| | | NCI-H596 | |
| astrocytoma SF-539 | 1.0 | Mammary gland | 7.0 |
| astrocytoma SNB-75 | 0.0 | Breast ca.* (pl.ef) | 1.8 |
| | | MCF-7 | |
| glioma SNB-19 | 5.8 | Breast ca.* (pl.ef) | 1.6 |
| | | MDA-MB-231 | |
| glioma U251 | 1.6 | Breast ca.* (pl. ef) | 12.3 |
| | | T47D | |
| glioma SF-295 | 0.3 | Breast ca. BT-549 | 2.1 |
| Heart | 0.9 | Breast ca. MDA-N | 1.7 |
| Skeletal muscle | 0.1 | Ovary | 0.9 |
| Bone marrow | 1.0 | Ovarian ca. OVCAR-3 | 6.5 |
| Thymus | 2.8 | Ovarian ca. OVCAR-4 | 0.2 |
| Spleen | 3.0 | Ovarian ca. OVCAR-5 | 19.8 |
| Lymph node | 0.4 | Ovarian ca. OVCAR-8 | 40.9 |
| Colon (ascending) | 23.8 | Ovarian ca. IGROV-1 | 2.7 |
| Stomach | 0.7 | Ovarian ca. (ascites) | 3.1 |
| | | SK-OV-3 | |
| Small intestine | 0.7 | Uterus | 9.7 |
| Colon ca. SW480 | 0.0 | Placenta | 1.0 |
| Colon ca.* SW620 | 0.7 | Prostate | 4.2 |
| (SW480 met) | | | |
| Colon ca. HT29 | 3.1 | Prostate ca.* (bone | 3.3 |
| | | met) PC-3 | |
| Colon ca. HCT-116 | 4.8 | Testis | 8.7 |
| Colon ca. CaCo-2 | 3.1 | Melanoma | 0.0 |
| | | Hs688(A).T | |
| Colon ca. HCT-15 | 5.3 | Melanoma* (met) | 0.4 |
| | | Hs688(B).T | |
| Colon ca. HCC-2998 | 10.7 | Melanoma UACC-62 | 0.0 |
| Gastric ca.* (liver | 6.9 | Melanoma M14 | 2.7 |
| met) NCI-N87 | | | |
| Bladder | 4.3 | Melanoma LOX | 0.1 |
| | | IMVI | |
| Trachea | 0.7 | Melanoma* (met) | 0.1 |
| | | SK-MEL-5 | |
| Kidney | 8.2 | Melanoma SK-MEL- | 100.0 |
| | | 28 | |
| Kidney (fetal) | 7.7 | | |

TABLE 20
Panel 1.2

| | Rel. Exp. (%) | Rel. Exp. (%) | | Rel. Exp. (%) | Rel. Exp. (%) |
|---|---|---|---|---|---|
| | Ag1347, Run | Ag1347, Run | | Ag1347, Run | Ag1347, Run |
| Tissue Name | 132938391 | 133337395 | Tissue Name | 132938391 | 133337395 |
| Endothelial cells | 2.8 | 0.0 | Renal ca. 786-0 | 2.0 | 1.2 |
| Heart (Fetal) | 1.0 | 2.0 | Renal ca. A498 | 4.9 | 12.9 |
| Pancreas | 4.2 | 8.3 | Renal ca. | 0.8 | 0.0 |
| | | | RXF 393 | | |
| Pancreatic ca. | 0.7 | 1.5 | Renal ca. | 0.7 | 0.8 |
| CAPAN 2 | | | ACHN | | |
| Adrenal Gland | 16.0 | 20.0 | Renal ca. | 3.8 | 0.0 |
| | | | UO-31 | | |
| Thyroid | 11.3 | 16.6 | Renal ca. | 10.3 | 6.6 |
| | | | TK-10 | | |
| Salivary gland | 20.4 | 26.4 | Liver | 3.4 | 12.9 |
| Pituitary gland | 13.3 | 11.7 | Liver (fetal) | 3.4 | 6.7 |
| Brain (fetal) | 10.2 | 6.7 | Liver ca. | 33.4 | 12.2 |
| | | | (hepatoblast) | | |
| | | | HepG2 | | |
| Brain (whole) | 7.6 | 12.9 | Lung | 2.1 | 3.6 |
| Brain | 5.8 | 11.6 | Lung (fetal) | 4.0 | 3.2 |
| (amygdala) | | | | | |
| Brain | 6.6 | 15.3 | Lung ca. | 2.7 | 0.0 |
| (cerebellum) | | | (small cell) | | |
| | | | LX-1 | | |
| Brain | 19.6 | 24.0 | Lung ca. | 27.5 | 25.5 |
| (hippocampus) | | | (small cell) | | |
| | | | NCI-H69 | | |
| Brain (thalamus) | 10.8 | 12.9 | Lung ca. (s.cell | 2.2 | 2.3 |
| | | | var.) SHP-77 | | |
| Cerebral Cortex | 22.1 | 27.7 | Lung ca. (large | 17.6 | 25.0 |
| | | | cell)NCI-H460 | | |
| Spinal cord | 3.0 | 6.7 | Lung ca. (non- | 4.2 | 3.6 |
| | | | sm. cell) A549 | | |
| glio/astro | 1.5 | 0.0 | Lung ca. | 5.1 | 7.0 |
| U87-MG | | | (non-s.cell) | | |
| | | | NCI-H23 | | |
| glio/astro | 2.6 | 4.1 | Lung ca. | 16.7 | 29.5 |
| U-118-MG | | | (non-s.cell) | | |
| | | | HOP-62 | | |
| astrocytoma | 1.3 | 2.4 | Lung ca. | 84.1 | 100.0 |
| SW1783 | | | (non-s.cl) | | |
| | | | NCI-H522 | | |
| neuro*; met | 2.5 | 0.0 | Lung ca. | 1.2 | 6.1 |
| SK-N-AS | | | (squam.) SW | | |
| | | | 900 | | |
| astrocytoma | 4.6 | 2.9 | Lung ca. | 4.8 | 3.3 |
| SF-539 | | | (squam.) NCI | | |
| | | | H596 | | |
| astrocytoma | 0.0 | 0.0 | Mammary | 5.4 | 12.8 |
| SNB-75 | | | gland | | |
| glioma SNB-19 | 9.0 | 8.1 | Breast ca.* | 6.6 | 6.8 |
| | | | (pl.ef) MCF-7 | | |
| glioma U251 | 1.6 | 3.6 | Breast ca.* | 2.9 | 3.0 |
| | | | (pl.ef) | | |
| | | | MDA-MB-231 | | |
| glioma SF-295 | 0.0 | 1.0 | Breast ca.* (pl. | 29.5 | 51.4 |
| | | | ef) T47D | | |
| Heart | 3.4 | 6.0 | Breast ca. | 5.6 | 11.5 |
| | | | BT-549 | | |
| Skeletal Muscle | 4.4 | 0.3 | Breast ca. | 6.4 | 2.1 |
| | | | MDA-N | | |
| Bone marrow | 4.1 | 3.5 | Ovary | 2.9 | 3.4 |
| Thymus | 2.8 | 4.1 | Ovarian ca. | 5.4 | 15.1 |
| | | | OVCAR-3 | | |
| Spleen | 1.8 | 3.5 | Ovarian ca. | 0.5 | 0.8 |
| | | | OVCAR-4 | | |
| Lymph node | 5.6 | 1.6 | Ovarian ca. | 25.9 | 13.5 |
| | | | OVCAR-5 | | |
| Colorectal | 11.0 | 8.6 | Ovarian ca. | 100.0 | 53.2 |
| Tissue | | | OVCAR-8 | | |
| Stomach | 4.7 | 7.2 | Ovarian ca. | 10.7 | 1.0 |
| | | | IGROV-1 | | |
| Small intestine | 0.6 | 2.2 | Ovarian ca. | 5.3 | 4.9 |
| | | | (ascites) | | |
| | | | SK-OV-3 | | |
| Colon ca. | 0.0 | 0.0 | Uterus | 7.5 | 6.7 |
| SW480 | | | | | |
| Colon ca.* | 0.8 | 1.5 | Placenta | 31.0 | 24.1 |
| SW620 | | | | | |
| (SW480met) | | | | | |
| Colon ca. HT29 | 7.4 | 3.0 | Prostate | 18.6 | 20.9 |
| Colon ca. | 0.7 | 9.6 | Prostate ca.* | 7.9 | 5.1 |
| HCT-116 | | | (bone met) | | |
| | | | PC-3 | | |
| Colon ca. CaCo-2 | 3.8 | 9.2 | Testis | 34.4 | 11.8 |
| Colon ca. Tissue | 20.6 | 27.5 | Melanoma | 0.0 | 1.4 |
| (ODO3866) | | | Hs688(A).T | | |
| Colon ca. HCC- | 38.4 | 19.5 | Melanoma* | 5.1 | 0.0 |
| 2998 | | | (met) | | |
| | | | Hs688(B).T | | |
| Gastric ca.* | 6.2 | 1.1 | Melanoma | 0.0 | 0.0 |
| (liver met) | | | UACC-62 | | |
| NCI-N87 | | | | | |
| Bladder | 5.3 | 6.8 | Melanoma | 8.5 | 16.0 |
| | | | M14 | | |
| Trachea | 4.4 | 15.1 | Melanoma | 0.0 | 1.4 |
| | | | LOXIMVI | | |
| Kidney | 12.3 | 12.5 | Melanoma* | 3.5 | 1.7 |
| | | | (met) | | |
| | | | SK-MEL-5 | | |
| Kidney (fetal) | 15.1 | 34.2 | | | |

TABLE 21
Panel 1.3D

| | Rel. Exp. (%) | Rel. Exp. (%) | Rel. Exp. (%) | | Rel. Exp. (%) | Rel. Exp. (%) | Rel. Exp. (%) |
|---|---|---|---|---|---|---|---|
| | Ag299, Run | Ag299, Run | Ag299, Run | | Ag299, Run | Ag299, Run | Ag299, Run |
| Tissue Name | 150863120 | 150981000 | 151719344 | Tissue Name | 150863120 | 150981000 | 151719344 |
| Liver | 9.3 | 0.0 | 0.0 | Kidney | 0.0 | 4.0 | 9.7 |
| adenocarcin | | | | (fetal) | | | |
| oma | | | | | | | |
| Pancreas | 0.0 | 10.2 | 18.9 | Renal ca. | 28.3 | 0.0 | 0.0 |
| | | | | 786-0 | | | |
| Pancreatic | 0.0 | 8.8 | 0.0 | Renal ca. | 9.2 | 31.4 | 9.9 |
| ca. CAPAN 2 | | | | A498 | | | |
| Adrenal | 0.0 | 0.0 | 14.6 | Renal ca. | 0.0 | 0.0 | 0.0 |
| gland | | | | RXF 393 | | | |
| Thyroid | 25.5 | 0.0 | 12.2 | Renal ca. | 0.0 | 0.0 | 24.3 |
| | | | | ACHN | | | |
| Salivary | 12.9 | 14.4 | 3.9 | Renal ca. | 0.0 | 0.0 | 0.0 |
| gland | | | | UO-31 | | | |
| Pituitary | 18.4 | 18.7 | 17.0 | Renal ca. | 0.0 | 6.3 | 13.1 |
| gland | | | | TK-10 | | | |
| Brain (fetal) | 11.6 | 0.0 | 0.0 | Liver | 0.0 | 14.0 | 14.8 |
| Brain | 0.0 | 6.5 | 0.0 | Liver (fetal) | 3.8 | 0.0 | 0.0 |
| (whole) | | | | | | | |
| Brain | 13.8 | 10.9 | 66.0 | Liver ca. | 2.9 | 10.5 | 11.7 |
| (amygdala) | | | | (hepatoblast) | | | |
| | | | | HepG2 | | | |
| Brain | 31.0 | 8.6 | 3.5 | Lung | 0.0 | 10.6 | 0.0 |
| (cerebellum) | | | | | | | |
| Brain | 20.0 | 48.6 | 22.7 | Lung (fetal) | 0.0 | 9.9 | 18.4 |
| (hippocampus) | | | | | | | |
| Brain | 5.2 | 7.7 | 0.0 | Lung ca. | 10.7 | 3.2 | 0.0 |
| (substantia | | | | (small cell) | | | |
| nigra) | | | | LX-1 | | | |
| Brain | 14.4 | 5.0 | 8.8 | Lung ca. | 14.3 | 0.0 | 19.1 |
| (thalamus) | | | | (small cell) | | | |
| | | | | NCI-H69 | | | |
| Cerebral | 27.9 | 12.6 | 36.6 | Lung ca. | 0.0 | 0.0 | 0.0 |
| Cortex | | | | (s.cell var.) | | | |
| | | | | SHP-77 | | | |
| Spinal cord | 19.8 | 0.0 | 35.1 | Lung ca. | 0.0 | 0.0 | 0.0 |
| | | | | (large | | | |
| | | | | cell) | | | |
| | | | | NCI-H460 | | | |
| glio/astro | 0.0 | 0.0 | 0.0 | Lung ca. | 0.0 | 0.0 | 0.0 |
| U87-MG | | | | (non-sm. | | | |
| | | | | cell) A549 | | | |
| glio/astro | 0.0 | 0.0 | 22.8 | Lung ca. | 10.8 | 15.0 | 0.0 |
| U-118-MG | | | | (non-s.cell) | | | |
| | | | | NCI-H23 | | | |
| astrocytoma | 10.7 | 8.0 | 0.0 | Lung ca. | 20.9 | 24.7 | 43.2 |
| SW1783 | | | | (non-s.cell) | | | |
| | | | | HOP-62 | | | |
| neuro*; met | 0.0 | 8.8 | 0.0 | Lung ca. | 47.3 | 57.0 | 72.7 |
| SK-N-AS | | | | (non-s.cl) | | | |
| | | | | NCI-H522 | | | |
| astrocytoma | 19.2 | 7.1 | 20.2 | Lung ca. | 11.1 | 23.3 | 0.0 |
| SF-539 | | | | (squam.) | | | |
| | | | | SW900 | | | |
| astrocytoma | 0.0 | 0.0 | 17.9 | Lung ca. | 0.0 | 0.0 | 0.0 |
| SNB-75 | | | | (squam.) | | | |
| | | | | NCI-H596 | | | |
| glioma | 0.0 | 0.0 | 14.0 | Mammary | 0.0 | 11.3 | 0.0 |
| SNB-19 | | | | gland | | | |
| glioma | 35.8 | 0.0 | 0.0 | Breast ca.* | 25.5 | 9.7 | 9.0 |
| U251 | | | | (pl.ef) | | | |
| | | | | MCF-7 | | | |
| glioma | 0.0 | 0.0 | 12.5 | Breast ca.* | 32.8 | 4.4 | 14.6 |
| SF-295 | | | | (pl.ef) | | | |
| | | | | MDA-MB- | | | |
| | | | | 231 | | | |
| Heart (fetal) | 0.0 | 0.0 | 0.0 | Breast ca.* | 29.7 | 16.6 | 27.9 |
| | | | | (pl.ef) | | | |
| | | | | T47D | | | |
| Heart | 10.8 | 0.0 | 0.0 | Breast ca. | 0.0 | 0.0 | 0.0 |
| | | | | BT-549 | | | |
| Skeletal | 67.4 | 27.5 | 100.0 | Breast ca. | 0.0 | 0.0 | 10.7 |
| muscle | | | | MDA-N | | | |
| (fetal) | | | | | | | |
| Skeletal | 0.0 | 0.0 | 1.7 | Ovary | 0.0 | 0.0 | 0.0 |
| muscle | | | | | | | |
| Bone | 0.0 | 0.0 | 14.7 | Ovarian ca. | 28.7 | 8.5 | 62.0 |
| marrow | | | | OVCAR-3 | | | |
| Thymus | 0.0 | 0.0 | 11.4 | Ovarian ca. | 0.0 | 0.0 | 0.0 |
| | | | | OVCAR-4 | | | |
| Spleen | 19.1 | 14.4 | 0.0 | Ovarian ca. | 7.0 | 9.5 | 23.7 |
| | | | | OVCAR-5 | | | |
| Lymph node | 7.0 | 10.6 | 0.0 | Ovarian ca. | 100.0 | 89.5 | 45.7 |
| | | | | OVCAR-8 | | | |
| Colorectal | 0.0 | 30.4 | 19.3 | Ovarian ca. | 0.0 | 10.9 | 14.5 |
| | | | | IGROV-1 | | | |
| Stomach | 17.0 | 10.4 | 12.2 | Ovarian | 9.4 | 15.2 | 22.8 |
| | | | | ca.* | | | |
| | | | | (ascites) | | | |
| | | | | SK-OV-3 | | | |
| Small | 0.0 | 0.0 | 0.0 | Uterus | 4.0 | 6.8 | 0.0 |
| intestine | | | | | | | |
| Colon ca. | 0.0 | 0.0 | 25.0 | Plancenta | 0.0 | 0.0 | 12.2 |
| SW480 | | | | | | | |
| Colon ca.* | 10.2 | 0.0 | 0.0 | Prostate | 10.2 | 7.0 | 11.3 |
| SW620(SW | | | | | | | |
| 480 met) | | | | | | | |
| Colon ca. | 14.7 | 0.0 | 11.5 | Prostate | 0.0 | 0.0 | 0.0 |
| HT29 | | | | ca.* (bone | | | |
| | | | | met)PC-3 | | | |
| Colon ca. | 9.7 | 29.1 | 42.9 | Testis | 44.8 | 100.0 | 66.9 |
| HCT-116 | | | | | | | |
| Colon ca. | 7.4 | 4.2 | 6.8 | Melanoma | 0.0 | 0.0 | 0.0 |
| CaCo-2 | | | | Hs688(A).T | | | |
| Colon ca. | 0.0 | 0.0 | 0.0 | Melanoma* | 0.0 | 0.0 | 0.0 |
| tissue(ODO | | | | (met) | | | |
| 3866) | | | | Hs688(B).T | | | |
| Colon ca. | 14.3 | 23.8 | 32.5 | Melanoma | 0.0 | 0.0 | 0.0 |
| HCC-2998 | | | | UACC-62 | | | |
| Gastric ca.* | 10.2 | 62.4 | 11.4 | Melanoma | 0.0 | 0.0 | 0.0 |
| (liver met) | | | | M14 | | | |
| NCI-N87 | | | | | | | |
| Bladder | 0.0 | 4.3 | 5.8 | Melanoma | 0.0 | 0.0 | 0.0 |
| | | | | LOX IMVI | | | |
| Trachea | 4.6 | 20.4 | 0.0 | Melanoma* | 0.0 | 0.0 | 0.0 |
| | | | | (met) | | | |
| | | | | SK-MEL-5 | | | |
| Kidney | 0.0 | 0.0 | 0.0 | Adipose | 0.0 | 0.0 | 4.3 |

TABLE 22
Panel 2D

| | Rel. Exp. (%) | Rel. Exp. (%) | | Rel. Exp. (%) | Rel. Exp. (%) |
|---|---|---|---|---|---|
| | Ag2708, Run | Ag299, Run | | Ag2708, Run | Ag299, Run |
| Tissue Name | 153218692 | 150811734 | Tissue Name | 153218692 | 150811734 |
| Normal Colon | 3.0 | 25.2 | Kidney | 1.0 | 0.0 |
| | | | Margin | | |
| | | | 8120608 | | |
| CC Well to Mod | 1.9 | 1.3 | Kidney Cancer | 0.0 | 3.8 |
| Diff (ODO3866) | | | 8120613 | | |
| CC Margin | 0.0 | 3.5 | Kidney | 0.0 | 8.6 |
| (ODO3866) | | | Margin | | |
| | | | 8120614 | | |
| CC Gr.2 | 0.0 | 2.7 | Kidney Cancer | 0.0 | 0.0 |
| rectosigmoid | | | 9010320 | | |
| (ODO3868) | | | | | |
| CC Margin | 1.1 | 0.0 | Kidney | 1.3 | 3.8 |
| (ODO3868) | | | Margin | | |
| | | | 9010321 | | |
| CC Mod Diff | 0.0 | 0.0 | Normal Uterus | 0.0 | 0.0 |
| (ODO3920) | | | | | |
| CC Margin | 0.0 | 6.0 | Uterus Cancer | 10.4 | 22.2 |
| (ODO3920) | | | 064011 | | |
| CC Gr.2 ascend | 0.0 | 2.6 | Normal | 8.5 | 9.6 |
| colon | | | Thyroid | | |
| (ODO3921) | | | | | |
| CC Margin | 0.7 | 1.9 | Thyroid | 0.0 | 0.0 |
| (ODO3921) | | | Cancer 064010 | | |
| CC from Partial | 0.0 | 6.7 | Thyroid | 4.7 | 14.9 |
| Hepatectomy | | | Cancer | | |
| (ODO4309) | | | A302152 | | |
| Mets | | | | | |
| Liver Margin | 1.2 | 1.0 | Thyroid | 3.6 | 14.2 |
| (ODO4309) | | | Margin | | |
| | | | A302153 | | |
| Colon mets to | 1.1 | 3.7 | Normal Breast | 5.4 | 12.4 |
| lung | | | | | |
| (OD04451-01) | | | | | |
| Lung Margin | 1.7 | 2.6 | Breast Cancer | 2.4 | 4.1 |
| (OD04451-02) | | | (OD04566) | | |
| Normal Prostate | 6.1 | 5.3 | Breast Cancer | 3.0 | 20.3 |
| 6546-1 | | | (OD04590-01) | | |
| Prostate Cancer | 1.6 | 2.5 | Breast Cancer | 1.7 | 12.9 |
| (OD04410) | | | Mets | | |
| | | | (OD04590-03) | | |
| Prostate Margin | 0.6 | 11.3 | Breast Cancer | 0.5 | 7.7 |
| (OD04410) | | | Metastasis | | |
| | | | (OD04655-05) | | |
| Prostate Cancer | 20.3 | 100.0 | Breast Cancer | 0.0 | 2.6 |
| (OD04720-01) | | | 064006 | | |
| Prostate Margin | 9.2 | 60.3 | Breast Cancer | 100.0 | 62.0 |
| (OD04720-02) | | | 1024 | | |
| Normal Lung | 19.5 | 24.3 | Breast Cancer | 2.1 | 2.0 |
| 061010 | | | 9100266 | | |
| Lung Met to | 0.0 | 11.7 | Breast Margin | 1.5 | 4.1 |
| Muscle | | | 9100265 | | |
| (ODO4286) | | | | | |
| Muscle Margin | 5.1 | 10.9 | Breast Cancer | 8.0 | 18.4 |
| (ODO4286) | | | A209073 | | |
| Lung Malignant | 0.0 | 8.3 | Breast Margin | 9.3 | 47.3 |
| Cancer | | | A2090734 | | |
| (OD03126) | | | | | |
| Lung Margin | 1.8 | 4.2 | Normal Liver | 4.4 | 4.1 |
| (OD03126) | | | | | |
| Lung Cancer | 1.1 | 5.8 | Liver Cancer | 1.2 | 12.1 |
| (OD04404) | | | 064003 | | |
| Lung Margin | 0.0 | 17.1 | Liver Cancer | 0.0 | 0.0 |
| (OD04404) | | | 1025 | | |
| Lung Cancer | 0.0 | 4.1 | Liver Cancer | 1.2 | 0.0 |
| (OD04565) | | | 1026 | | |
| Lung Margin | 1.2 | 3.7 | Liver Cancer | 2.3 | 0.0 |
| (OD04565) | | | 6004-T | | |
| Lung Cancer | 0.0 | 0.0 | Liver Tissue | 2.3 | 15.7 |
| (OD04237-01) | | | 6004-N | | |
| Lung Margin | 0.5 | 0.0 | Liver Cancer | 0.0 | 0.0 |
| (OD04237-02) | | | 6005-T | | |
| Ocular Mel Met | 1.5 | 0.0 | Liver Tissue | 0.0 | 0.0 |
| to Liver | | | 6005-N | | |
| (ODO4310) | | | | | |
| Liver Margin | 0.4 | 0.0 | Normal | 0.6 | 0.0 |
| (ODO4310) | | | Bladder | | |
| Melanoma Mets | 1.0 | 0.0 | Bladder | 2.5 | 0.0 |
| to Lung | | | Cancer 1023 | | |
| (OD04321) | | | | | |
| Lung Margin | 0.0 | 0.0 | Bladder | 1.0 | 12.3 |
| (OD04321) | | | Cancer | | |
| | | | A302173 | | |
| Normal Kidney | 33.0 | 94.6 | Bladder | 0.0 | 12.1 |
| | | | Cancer | | |
| | | | (OD04718-01) | | |
| Kidney Ca, | 8.0 | 47.0 | Bladder | 0.0 | 8.1 |
| Nuclear grade 2 | | | Normal | | |
| (OD04338) | | | Adjacent | | |
| | | | (OD04718-03) | | |
| Kidney Margin | 2.1 | 10.4 | Normal Ovary | 0.0 | 0.0 |
| (OD04338) | | | | | |
| Kidney Ca | 2.2 | 8.0 | Ovarian | 0.0 | 4.5 |
| Nuclear grade | | | Cancer 064008 | | |
| 1/2 (OD04339) | | | | | |
| Kidney Margin | 7.8 | 42.0 | Ovarian | 2.4 | 3.5 |
| (OD04339) | | | Cancer | | |
| | | | (OD04768-07) | | |
| Kidney Ca, Clear | 0.0 | 0.0 | Ovary Margin | 0.0 | 0.0 |
| cell type | | | (OD04768-08) | | |
| (OD04340) | | | | | |
| Kidney Margin | 8.6 | 24.3 | Normal | 1.5 | 4.2 |
| (OD04340) | | | Stomach | | |
| Kidney Ca, | 0.0 | 3.7 | Gastric Cancer | 0.0 | 0.0 |
| Nuclear grade 3 | | | 9060358 | | |
| (OD04348) | | | | | |
| Kidney Margin | 5.0 | 4.1 | Stomach | 0.0 | 0.0 |
| (OD04348) | | | Margin | | |
| | | | 9060359 | | |
| Kidney Cancer | 45.7 | 0.0 | Gastric Cancer | 1.1 | 7.4 |
| (OD04622-01) | | | 9060395 | | |
| Kidney Margin | 0.0 | 0.0 | Stomach | 4.7 | 8.4 |
| (OD04622-03) | | | Margin | | |
| | | | 9060394 | | |
| Kidney Cancer | 3.0 | 11.7 | Gastric Cancer | 0.0 | 5.7 |
| (OD04450-01) | | | 9060397 | | |
| Kidney Margin | 17.4 | 51.8 | Stomach | 0.0 | 0.0 |
| (OD04450-03) | | | Margin | | |
| | | | 9060396 | | |
| Kidney Cancer | 0.0 | 0.0 | Gastric Cancer | 6.3 | 2.8 |
| 8120607 | | | 064005 | | |

TABLE 23
Panel 4D

| | Rel. Exp. (%) | Rel. Exp. (%) | | Rel. Exp. (%) | Rel. Exp. (%) |
|---|---|---|---|---|---|
| | Ag2708, Run | Ag299, Run | | Ag2708, Run | Ag299, Run |
| Tissue Name | 153218696 | 162425466 | Tissue Name | 153218696 | 162425466 |
| Secondary Th1 act | 0.0 | 0.0 | HUVEC IL-1beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 9.4 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 0.0 | Lung | 0.0 | 0.0 |
| | | | Microvascular EC | | |
| | | | none | | |
| Primary Th1 act | 0.0 | 0.0 | Lung | 2.3 | 14.4 |
| | | | Microvascular EC | | |
| | | | TNFalpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 10.1 |
| | | | Dermal EC | | |
| | | | TNFalpha + IL- | | |
| | | | 1beta | | |
| Primary Th1 rest | 0.0 | 0.0 | Bronchial | 0.0 | 11.7 |
| | | | epithelium | | |
| | | | TNFalpha + | | |
| | | | IL1beta | | |
| Primary Th2 rest | 0.0 | 9.7 | Small airway | 5.8 | 0.0 |
| | | | epithelium none | | |
| Primary Tr1 rest | 9.3 | 0.0 | Small airway | 9.3 | 57.0 |
| | | | epithelium | | |
| | | | TNFalpha + IL- | | |
| | | | 1beta | | |
| CD45RA CD4 | 4.4 | 0.0 | Coronery artery | 0.0 | 0.0 |
| lymphocyte act | | | SMC rest | | |
| CD45RO CD4 | 6.4 | 11.9 | Coronery artery | 0.0 | 0.0 |
| lymphocyte act | | | SMC TNFalpha + | | |
| | | | IL-1beta | | |
| CD8 lymphocyte | 0.0 | 0.0 | Astrocytes rest | 5.5 | 0.0 |
| act | | | | | |
| Secondary CD8 | 0.0 | 0.0 | Astrocytes | 0.0 | 0.0 |
| lymphocyte rest | | | TNFalpha + IL- | | |
| | | | 1beta | | |
| Secondary CD8 | 0.0 | 0.0 | KU-812 | 5.2 | 0.0 |
| lymphocyte act | | | (Basophil) rest | | |
| CD4 lymphocyte | 0.0 | 0.0 | KU-812 | 4.1 | 18.4 |
| none | | | (Basophil) | | |
| | | | PMA/ionomycin | | |
| 2ry | 3.1 | 0.0 | CCD1106 | 0.0 | 0.0 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNFalpha + IL- | | |
| | | | 1beta | | |
| LAK cells IL-2 | 7.4 | 15.8 | Liver cirrhosis | 7.9 | 40.1 |
| LAK cells IL-2 + | 7.2 | 0.0 | Lupus kidney | 8.6 | 0.0 |
| IL-12 | | | | | |
| LAK cells IL-2 + | 17.0 | 0.0 | NCI-H292 none | 4.6 | 19.3 |
| IFN gamma | | | | | |
| LAK cells IL-2 + | 9.4 | 0.0 | NCI-H292 IL-4 | 11.7 | 25.0 |
| IL-18 | | | | | |
| LAK cells | 0.0 | 0.0 | NCI-H292 IL-9 | 0.0 | 0.0 |
| PMA/ionomycin | | | | | |
| NK Cells IL-2 rest | 0.0 | 11.7 | NCI-H292 IL-13 | 0.0 | 0.0 |
| Two Way MLR 3 | 0.0 | 0.0 | NCI-H292 IFN | 0.0 | 9.8 |
| day | | | gamma | | |
| Two Way MLR 5 | 0.0 | 0.0 | HPAEC none | 0.0 | 0.0 |
| day | | | | | |
| Two Way MLR 7 | 14.6 | 5.4 | HPAEC TNF | 3.8 | 0.0 |
| day | | | alpha + IL-1 beta | | |
| PBMC rest | 0.0 | 0.0 | Lung fibroblast | 0.0 | 0.0 |
| | | | none | | |
| PBMC PWM | 4.9 | 0.0 | Lung fibroblast | 15.0 | 0.0 |
| | | | TNF alpha + IL-1 | | |
| | | | beta | | |
| PBMC PHA-L | 0.0 | 8.7 | Lung fibroblast | 0.0 | 17.4 |
| | | | IL-4 | | |
| Ramos (B cell) | 0.0 | 0.0 | Lung fibroblast | 5.4 | 0.0 |
| none | | | IL-9 | | |
| Ramos (B cell) | 2.4 | 0.0 | Lung fibroblast | 0.0 | 0.0 |
| ionomycin | | | IL-13 | | |
| B lymphocytes | 5.4 | 19.9 | Lung fibroblast | 6.5 | 0.0 |
| PWM | | | IFN gamma | | |
| B lymphocytes | 20.4 | 22.2 | Dermal fibroblast | 0.0 | 0.0 |
| CD40L and IL-4 | | | CCD1070 rest | | |
| EOL-1 dbcAMP | 0.0 | 9.6 | Dermal fibroblast | 5.8 | 6.3 |
| | | | CCD1070 TNF | | |
| | | | alpha | | |
| EOL-1 dbcAMP | 0.0 | 0.0 | Dermal fibroblast | 0.0 | 0.0 |
| PMA/ionomycin | | | CCD1070 IL-1 | | |
| | | | beta | | |
| Dendritic cells | 4.7 | 12.0 | Dermal fibroblast | 0.0 | 9.2 |
| none | | | IFN gamma | | |
| Dendritic cells LPS | 0.0 | 0.0 | Dermal fibroblast | 0.0 | 6.3 |
| | | | IL-4 | | |
| Dendritic cells anti- | 0.0 | 6.4 | IBD Colitis 2 | 6.3 | 12.7 |
| CD40 | | | | | |
| Monocytes rest | 0.0 | 0.0 | IBD Crohn's | 0.0 | 0.0 |
| Monocytes LPS | 0.0 | 0.0 | Colon | 0.0 | 0.0 |
| Macrophages rest | 0.0 | 21.0 | Lung | 0.0 | 0.0 |
| Macrophages LPS | 0.0 | 0.0 | Thymus | 100.0 | 100.0 |
| HUVEC none | 0.0 | 0.0 | Kidney | 9.3 | 0.0 |
| HUVEC starved | 0.0 | 11.1 | | | |

CNS_neurodegeneration_v1.0 Summary: Ag299/Ag2708 Two experiments with two different probe and primer sets show that the NOV9 gene is expressed in the brain. However, no difference is detected in the expression of this gene in the postmortem brains of Alzheimer's diseased patients when compared to controls. Please see Panel 1.2 for discussion of utility of this gene in the central nervous system.

Panel 1 Summary: Ag299 The NOV9 gene is expressed at low and in roughly equivalent amounts throughout much of the CNS, including in amygdala, cerebellum, hippocampus, substantia nigra, and thalamus (see Panel 1.2 Summary for potential utility).

In addition, NOV9 gene expression is detected at low levels in pancreas (CT=33), adrenal gland (CT=33), thyroid (CT=33), liver (CT=34), and hypothalamus (CT=33) suggesting that this gene may play a role in neuroendocrine disorders or metabolic diseases, including diabetes and obesity.

Interestingly, the NOV9 gene appears to be overexpressed in a number of liver cancer, lung cancer, and ovarian cancer cell lines relative to their respective normal controls. It is also expressed at high levels in a single melanoma cell line. These observations suggest that the therapeutic inhibition of this gene activity, through the use of small molecule drugs or antibodies, may provide treatment of the cancers listed above. In contrast, NOV9 gene expression appears to be downregulated in colon cancer cell lines and kidney cancer cell lines relative to normal controls. Therefore, therapeutic up-regulation of the activity of the NOV9 gene, through the application of the protein product or agonists might be of use in the treatment of kidney and colon cancers.

Panel 1.2 Summary: Ag1347 Results from two experiments using the same probe/primer set are reasonably concordant and the results are consistent with what was observed in Panel 1. Expression of the NOV9 gene in this panel is skewed by genomic DNA contamination in the adipose sample. Disregarding this sample, low but detectable expression is present in several CNS samples, namely cerebral cortex (CT=33.1), hippocampus (CT=33.3) and thalamus (CT=34.1). Several neurotransmitter receptors are GPCRs, including the dopamine receptor family, the serotonin receptor family, the GABAB receptor, muscarinic acetylcholine receptors, and others; thus the GPCR encoded by the NOV9 gene may represent a novel neurotransmitter receptor. Targeting various neurotransmitter receptors (dopamine, serotonin) has proven to be an effective therapy in psychiatric illnesses such as schizophrenia and depression. Furthermore the cerebral cortex and hippocampus are regions of the brain that are known to play critical roles in Alzheimer's disease, seizure disorders, and in the normal process of memory formation. Therefore, therapeutic modulation of the NOV9 gene or its protein product may be beneficial in one or more of these diseases, as may stimulation and/or blockade of the receptor coded for by the gene.

In addition, NOV9 gene expression is detected in adrenal gland (CT=33.6), thyroid (CT=34), and pituitary gland (CT,=34), suggesting that this gene may play a role in neuroendocrine disorders or metabolic diseases, including diabetes and obesity. The NOV9 transcript is present at very low levels in a number of other normal tissues including colon, kidney, placenta, prostate and testis.

Highest expression of the NOV9 gene is seen in ovarian and lung cancer cell lines. However, this gene is also expressed in a number of other cancer cell lines, including melanoma, breast, colon, and liver. Therefore, the therapeutic inhibition of this gene activity, through the use of small molecule drugs or antibodies, may provide treatment of the cancers listed above.

References:

El Yacoubi M, Ledent C, Parmentier M, Bertorelli R, Ongini E, Costentin J, Vaugeois J M. Adenosine A2A receptor antagonists are potential antidepressants: evidence based on pharmacology and A2A receptor knockout mice. Br J Pharmacol September 2001;134(l):68-77

1. Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’effect. 2. We have designed studies to assess whether adenosine A2A receptor antagonists, or genetic inactivation of the receptor would be effective in established screening procedures, such as tail suspension and forced swim tests, which are predictive of clinical antidepressant activity. 3. Adenosine A2A receptor knockout mice were found to be less sensitive to ‘depressant’ challenges than their wildtype littermates. Consistently, the adenosine A2A receptor blockers SCH 58261 (1-10 mg kg(−1), i.p.) and KW 6002 (0.1-10 mg kg(−1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCH 58261 at 10 mg kg(−1) reduced the immobility time by 61%, while KW 6002 decreased the total immobility time at the doses of 1 and 10 mg kg(-1) by 75 and 79%, respectively. 6. Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

Blier P. Pharmacology of rapid-onset antidepressant treatment strategies. Clin Psychiatry 2001;62 Suppl 15:12-7

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HTIA receptors, to SSRI treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alpha1-adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

Tranquillini M E, Reggiani A. Glycine-site antagonists and stroke. Expert Opin Investig Drugs November 1999;8(11):1837-1848

The excitatory amino acid, (S)-glutamic acid, plays an important role in controlling many neuronal processes. Its action is mediated by two main groups of receptors: the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

Monopoli A, Lozza G, Forlani A, Mattavelli A, Ongini E. Blockade of adenosine A2A receptors by SCH 58261 results in neuroprotective effects in cerebral ischaemia in rats. Neuroreport Dec. 1, 1998;9(17):3955-9

Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p <0.05). Neuroprotective properties of SCH 58261 administered after ischaemia indicate that blockade of A2A adenosine receptors is a potentially useful biological target for the reduction of brain injury.

Panel 1.3D Summary: Ag299 Three experiments with the same probe and primer set show highest expression of the NOV9 gene in the testis, fetal skeletal muscle and an ovarian cancer line (CTs=33-34). Significant expression is also seen in a lung cancer cell line. Thus, the expression of the gene could be used to distinguish these samples from other samples in the panel. In addition, and more specifically, the expression of this gene could also be used to distinguish fetal skeletal muscle from adult skeletal muscle. Moreover, therapeutic modulation of this gene, through the use of small molecule drugs, antibodies or protein therapeutics might be of use in the treatment of lung or ovarian cancer.

In addition, the higher levels of expression in fetal skeletal muscle (CTs=33) when compared to adult skeletal muscle (CTs=40) suggest that this gene product may be involved in the development and homeostasis of this organ. Thus, therapeutic modulation of the expression or function of this gene may be useful in restoring muscle mass or function to weak or dystrophic muscle.

Please note that data from a third experiment with the probe and primer set Ag2708 shows expression that is low/undetectable in all samples on this panel (CTs>35). (Data not shown.)

Panel 2.2 Summary: Ag2708 Expression of the NOV9 gene is low/undetectable (CT values >34.5) in all of the samples on this panel.

Panel 2D Summary: Ag299/Ag2708 Two experiments with two different probe and primer sets low show overall expression of the NOV9 gene on this panel. The highest expression is in a sample derived from prostate cancer tissue (CT=33) and breast cancer tissue (CT=30.7). There are several other cancer tissues (bladder, breast, uterus, kidney and colon cancers) that also express this gene. In addition, the NOV9 transcript is detected in many normal tissues, including stomach, liver, breast, lung, kidney and thyroid. Interestingly, five kidney cancer tissues show overexpression of the NOV9 gene relative to their normal adjacent tissues. Thus, therapeutic up-regulation of the activity of this gene, through the application of the protein product or agonists might be of use in the treatment of kidney cancer. Alternatively, down-regulation of the activity of this gene, through the use of antibodies or small molecule drugs might be of use in the treatment of prostate, bladder, breast, uterus and colon cancers.

Panel 3D Summary: Ag2708 Expression of this gene is low/undetectable (CT values >34.5) in all of the samples on this panel.

Panel 4D Summary: Ag299/Ag2708 The NOV9 transcript is expressed at significant levels only in the thymus (CT=34) in both runs. The putative GPCR encoded for by the NOV9 gene could therefore play an important role in T cell development. Small molecule therapeutics, or antibody therapeutics designed against the GPCR encoded for by this gene could be utilized to modulate immune function (T cell development) and be important for organ transplant, AIDS treatment or post chemotherapy immune reconstitution.

NOV8

Expression of gene NOV8 was assessed using the primer-probe sets Ag2597 and Ag5234, described in Tables 24 and 25. Results of the RTQ-PCR runs are shown in Tables 26 and 27.

TABLE 24
Probe Name Ag2597

| Primers | Sequences | Length | Start Position | SEQ ID NO: |
|---|---|---|---|---|
| Forward | 5′-ggagctttctgggaagactct-3′ | 21 | 11 | 169 |
| Probe | TET-5′-tagaaaatgcacaggagcactccacg-3′-TAMRA | 26 | 32 | 170 |
| Reverse | 5′-caaaatgcggaagatgaaca-3′ | 20 | 86 | 171 |

TABLE 25
Probe Name Ag5234

| | | | | |
|---|---|---|---|---|
| Primers | Sequences | Length | Start Positon | SEQ ID NO: |

| | | | | |
|---|---|---|---|---|
| Forward | 5′-cttcatcatcttcatgctggcg-3′ | 22 | 606 | 172 |
| Probe | TET-5′-cactgctgctcaacatgctggagatata-3′-TAMRA | 28 | 641 | 173 |
| Reverse | 5′ggctggtcacgccctgctt-3′ | 19 | 691 | 174 |

TABLE 26
General_screening_panel_v1.5

| | Rel. Exp. (%) Ag5234, | | Rel. Exp. (%) Ag5234, |
|---|---|---|---|
| Tissue Name | Run 229514466 | Tissue Name | Run 229514466 |
| Adipose | 0.0 | Renal ca. TK-10 | 0.0 |
| Melanoma* | 1.4 | Bladder | 1.8 |
| Hs688(A).T | | | |
| Melanoma* | 0.8 | Gastric ca. (liver met.) | 6.3 |
| Hs688(B).T | | NCI-N87 | |
| Melanoma*M14 | 40.1 | Gastric ca. KATO III | 0.7 |
| Melanoma* | 2.0 | Colon ca. SW-948 | 0.0 |
| LOXIMVI | | | |
| Melanoma* SK- | 32.1 | Colon ca. SW480 | 100.0 |
| MEL-5 | | | |
| Squamous cell | 3.4 | Colon ca.* (SW480 | 0.0 |
| carcinoma SCC-4 | | met) SW620 | |
| Testis Pool | 1.6 | Colon ca. HT29 | 0.0 |
| Prostate ca.* (bone | 3.5 | Colon ca. HCT-116 | 67.8 |
| met) PC-3 | | | |
| Prostate Pool | 0.0 | Colon ca. CaCo-2 | 0.0 |
| Placenta | 1.2 | Colon cancer tissue | 0.0 |
| Uterus Pool | 0.3 | Colon ca. SW1116 | 10.9 |
| Ovarian ca. | 0.8 | Colon ca. Colo-205 | 0.0 |
| OVCAR-3 | | | |
| Ovarian ca. SK-OV-3 | 52.5 | Colon ca. SW-48 | 0.0 |
| Ovarian ca. | 6.5 | Colon Pool | 3.6 |
| OVCAR-4 | | | |
| Ovarian ca. | 15.2 | Small Intestine Pool | 2.1 |
| OVCAR-5 | | | |
| Ovarian ca. IGROV-1 | 4.9 | Stomach Pool | 0.8 |
| Ovarian ca. | 12.9 | Bone Marrow Pool | 0.0 |
| OVCAR-8 | | | |
| Ovary | 0.0 | Fetal Heart | 61.1 |
| Breast ca. MCF-7 | 0.0 | Heart Pool | 5.8 |
| Breast ca. MDA- | 42.9 | Lymph Node Pool | 0.1 |
| MB-231 | | | |
| Breast ca. BT 549 | 0.9 | Fetal Skeletal Muscle | 0.4 |
| Breast ca. T47D | 1.0 | Skeletal Muscle Pool | 0.0 |
| Breast ca. MDA-N | 0.0 | Spleen Pool | 0.0 |
| Breast Pool | 3.8 | Thymus Pool | 0.5 |
| Trachea | 0.7 | CNS cancer (glio/astro) | 0.0 |
| | | U87-MG | |
| Lung | 0.0 | CNS cancer (glio/astro) | 1.3 |
| | | U-118-MG | |
| Fetal Lung | 0.0 | CNS cancer | 0.0 |
| | | (neuro; met) SK-N-AS | |
| Lung ca. NCI-N417 | 0.5 | CNS cancer (astro) SF- | 0.4 |
| | | 539 | |
| Lung ca. LX-1 | 0.6 | CNS cancer (astro) | 0.5 |
| | | SNB-75 | |
| Lung ca. NCI-H146 | 0.0 | CNS cancer (glio) | 3.4 |
| | | SNB-19 | |
| Lung ca. SHP-77 | 0.0 | CNS cancer (glio) SF- | 4.3 |
| | | 295 | |
| Lung ca. A549 | 0.7 | Brain (Amygdala) Pool | 0.3 |
| Lung ca. NCI-H526 | 0.0 | Brain(cerebellum) | 0.0 |
| Lung ca. NCI-H23 | 12.7 | Brain (fetal) | 0.0 |
| Lung ca. NCI-H460 | 1.6 | Brain (Hippocampus) | 0.2 |
| | | Pool | |
| Lung ca. HOP-62 | 3.6 | Cerebral Cortex Pool | 0.0 |
| Lung ca. NCI-H522 | 16.3 | Brain (Substantia nigra) | 0.0 |
| | | Pool | |
| Liver | 0.0 | Brain (Thalamus) Pool | 0.0 |
| Fetal Liver | 0.7 | Brain (whole) | 0.0 |
| Liver ca. HepG2 | 0.6 | Spinal Cord Pool | 0.0 |
| Kidney Pool | 1.5 | Adrenal Gland | 0.0 |
| Fetal Kidney | 6.8 | Pituitary gland Pool | 0.0 |
| Renal ca. 786-0 | 0.6 | Salivary Gland | 0.0 |
| Renal ca. A498 | 0.0 | Thyroid (female) | 0.0 |
| Renal ca. ACHN | 0.0 | Pancreatic ca. CAPAN2 | 26.2 |
| Renal ca. UO-31 | 0.0 | Pancreas Pool | 3.5 |

TABLE 27
Panel 4.1D

| | Rel. Exp. (%) | | Rel. Exp. (%) |
|---|---|---|---|
| | Ag5234, Run | | Ag5234, Run |
| Tissue Name | 229788208 | Tissue Name | 229788208 |
| Secondary Th1 act | 2.6 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microvascular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium | 0.0 |
| | | none | |
| Primary Tr1 rest | 0.0 | Small airway epithelium | 8.4 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 4.8 |
| Secondary CD8 | 1.6 | Astrocytes TNFalpha + | 6.5 |
| lymphocyte rest | | IL-1beta | |
| Secondary CD8 | 0.0 | KU-812 (Basophil) rest | 0.0 |
| lymphocyte act | | | |
| CD4 lymphocyte none | 1.6 | KU-812 (Basophil) | 0.0 |
| | | PMA/ionomycin | |
| 2ry Th1/Th2/Tr1_anti- | 0.0 | CCD1106 (Keratinocytes) | 9.2 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 0.0 |
| LAK cells IL-2 + IL-12 | 1.1 | NCI-H292 none | 0.0 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 2.5 | NCI-H292 IL-13 | 2.7 |
| PMA/ionomycin | | | |
| NK Cells IL-2 rest | 0.0 | NCI-H292 IFN gamma | 0.0 |
| Two Way MLR 3 day | 1.5 | HPAEC none | 0.0 |
| Two Way MLR 5 day | 0.0 | HPAEC TNF alpha + IL-1 | 0.0 |
| | | beta | |
| Two Way MLR 7 day | 0.0 | Lung fibroblast none | 8.7 |
| PBMC rest | 0.0 | Lung fibroblast TNF | 17.0 |
| | | alpha + IL-1 beta | |
| PBMC PWM | 1.2 | Lung fibroblast IL-4 | 2.1 |
| PBMC PHA-L | 8.4 | Lung fibroblast IL-9 | 6.1 |
| Ramos (B cell) none | 0.0 | Lung fibroblast IL-13 | 0.0 |
| Ramos (B cell) | 0.0 | Lung fibroblast IFN | 6.3 |
| ionomycin | | gamma | |
| B lymphocytes PWM | 0.0 | Dermal fibroblast | 0.0 |
| | | CCD1070 rest | |
| B lymphocytes CD40L | 0.0 | Dermal fibroblast | 3.4 |
| and IL-4 | | CCD1070 TNF alpha | |
| EOL-1 dbcAMP | 0.0 | Dermal fibroblast | 3.9 |
| | | CCD1070 IL-1 beta | |
| EOL-1 dbcAMP | 0.0 | Dermal fibroblast IFN | 8.6 |
| PMA/ionomycin | | gamma | |
| Dendritic cells none | 0.0 | Dermal fibroblast IL-4 | 3.2 |
| Dendritic cells LPS | 0.0 | Dermal Fibroblasts rest | 1.8 |
| Dendritic cells anti- | 0.0 | Neutrophils TNFa + LPS | 0.0 |
| CD40 | | | |
| Monocytes rest | 0.0 | Neutrophils rest | 0.0 |
| Monocytes LPS | 100.0 | Colon | 0.0 |
| Macrophages rest | 0.0 | Lung | 0.0 |
| Macrophages LPS | 0.0 | Thymus | 0.0 |
| HUVEC none | 0.0 | Kidney | 2.4 |
| HUVEC starved | 0.0 | | |

CNS_neurodegeneration_v1.0 Summary: Ag2597/Ag5234 Expression of the NOV8 gene is low/undetectable in all samples on this panel (CTs>35). (Data not shown.)

General_screening_panel_v1.5 Summary: Ag5234 The expression of the NOV8 gene appears to be highest in a sample derived from a colon cancer cell line (SW480)(CT=30.4). In addition, there is substantial expression associated with two other colon cancer cell lines, a pancreatic cancer cell line, two lung cancer cell lines, a breast cancer cell line, two melanoma cell lines and a cluster of several ovarian cancer cell lines. Thus, the expression of this gene could be used to distinguish the above samples from the other samples in the panel. Moreover, therapeutic modulation of this gene, through the use of small molecule drugs, antibodies or protein therapeutics might be of benefit in the treatment of ovarian, colon, pancreatic, lung, breast cancers or melanoma.

This gene is also expressed at moderate levels in fetal heart (CT=31.1) and at lower levels in the adult heart (CT=34.5). Thus, expression of this gene may be used to differentiate between fetal and adult heart tissue. Furthermore, the higher levels of expression in fetal heart suggest that the protein encoded by this gene may be important for the pathogenesis, diagnosis, and/or treatment of diseases of the heart.

Panel 1.3D Summary: Ag2597 Expression of the NOV8 gene is low/undetectable in all samples on this panel (CTs>35). (Data not shown.). The amp plot suggests that there is a high probability of a probe failure with this probe and primer set.

Panel 2.2 Summary: Ag2597 Expression of the NOV8 gene is low/undetectable in all samples on this panel (CTs>35). (Data not shown.) The amp plot suggests that there is a high probability of a probe failure with this probe and primer set.

Panel 4.1D Summary: Ag 5234 Highest expression of the NOV8 transcript is in monocytes stimulated with LPS (CT=32.9). Upon activation with pathogens, including bacterial LPS, monocytes contribute to the innate and specific immunity by migrating to the site of tissue injury and releasing inflammatory cytokines. This release contributes to the inflammation process. This transcript encodes for a connexin like protein, a family of proteins that is involved in gap junction and intercellular communication. Thus, the protein encoded by this transcript may play a role in the interaction of activated monocytes with the endothelium.

This is the first step necessary for the migration of these cells to injured tissue. Therefore, modulation of the expression of the protein encoded by this transcript, by antibodies or small molecules can prevent the recruitment of monocytes and the inflammatory process, and lead to improvement of the symptoms of patients suffering from autoimmune and inflammatory diseases such as asthma, allergies, inflammatory bowel disease, lupus erythematosus, or rheumatoid arthritis.

Panel 4D Summary: Ag2597 Expression of the NOV8 gene is low/undetectable in all samples on this panel (CTs>35). (Data not shown.) The amp plot suggests that there is a high probability of a probe failure with this probe and primer set.

NOV7

Expression of gene NOV7 was assessed using the primer-probe sets Ag48l 1, Ag4845 and Ag4944, described in Tables 28, 29 and 30. Results of the RTQ-PCR runs are shown in 30 Tables 31, 32 and 33.

TABLE 28

Probe Name Ag4811

| | | | | |
|---|---|---|---|---|
| Primers | Sequences | Length | Start Positon | SEQ ID NO: |

| | | | | |
|---|---|---|---|---|
| Forward | 5′-catgtctcttttcgggaacata-3′ | 22 | 791 | 175 |
| Probe | TET-5′-cacagtaaacatcaccttcttggaagtg-3′-TAMRA | 28 | 820 | 176 |
| Reverse | 5′gaaggccataatcttccatttt-3′ | 22 | 848 | 177 |

TABLE 29

Probe Name Ag4845

| | | | | |
|---|---|---|---|---|
| Primers | Sequences | Length | Start Positon | SEQ ID NO: |

| | | | | |
|---|---|---|---|---|
| Forward | 5′-catgtctcttttcgggaacata-3′ | 22 | 791 | 178 |
| Probe | TET-5′-cacagtaaacatcaccttcttggaagtg-3′-TAMRA | 28 | 820 | 179 |
| Reverse | 5′gaaggccataatcttccatttt-3′ | 22 | 848 | 180 |

TABLE 30

Probe Name Ag4944

| | | | | |
|---|---|---|---|---|
| Primers | Sequences | Length | Start Positon | SEQ ID NO: |

| | | | | |
|---|---|---|---|---|
| Forward | 5′-cagggaagcagtacgaggttt-3′ | 21 | 537 | 181 |
| Probe | TET-5′-tggaggaagtgtccctaaaatcatttatcc-3′- | 30 | 598 | 182 |
| | TAMRA | | | |
| Reverse | 5′tgactctccagcatgctaga-3′ | 20 | 709 | 183 |

TABLE 31
General_screening_panel_v1.4

| | Rel. Exp. (%) Ag4811, | | Rel. Exp. (%) Ag4811, |
|---|---|---|---|
| Tissue Name | Run 223204488 | Tissue Name | Run 233204488 |
| Adipose | 4.9 | Renal ca. TK-10 | 71.7 |
| Melanoma* | 1.4 | Bladder | 6.5 |
| Hs688(A).T | | | |
| Melanoma* | 2.9 | Gastric ca. (liver met). | 100.0 |
| Hs688(B).T | | NCI-N87 | |
| Melanoma* M14 | 0.0 | Gastric ca. KATO III | 24.1 |
| Melanoma* | 0.0 | Colon ca. SW-948 | 2.4 |
| LOXIMVI | | | |
| Melanoma* SK- | 0.0 | Colon ca. SW480 | 7.1 |
| MEL-5 | | | |
| Squamous cell | 45.4 | Colon ca.* (SW480 | 0.5 |
| carcinoma SCC-4 | | met) SW620 | |
| Testis Pool | 1.7 | Colon ca. HT29 | 16.8 |
| Prostate ca.* (bone | 0.0 | Colon ca. HCT-116 | 0.0 |
| met) PC-3 | | | |
| Prostate Pool | 2.7 | Colon ca. CaCo-2 | 0.5 |
| Placenta | 0.5 | Colon cancer tissue | 12.1 |
| Uterus Pool | 0.4 | Colon ca. SW1116 | 6.9 |
| Ovarian ca. | 1.4 | Colon ca. Colo-205 | 3.7 |
| OVCAR-3 | | | |
| Ovarian ca. SK-OV-3 | 0.3 | Colon ca. SW-48 | 0.0 |
| Ovarian ca. | 45.1 | Colon Pool | 1.6 |
| OVCAR-4 | | | |
| Ovarian ca. | 0.0 | Small Intestine Pool | 1.2 |
| OVCAR-5 | | | |
| Ovarian ca. IGROV-1 | 0.4 | Stomach Pool | 7.0 |
| Ovarian ca. | 25.9 | Bone Marrow Pool | 3.1 |
| OVCAR-8 | | | |
| Ovary | 6.0 | Fetal Heart | 0.4 |
| Breast ca. MCF-7 | 0.0 | Heart Pool | 1.3 |
| Breast ca. MDA- | 32.1 | Lymph Node Pool | 2.8 |
| MB-231 | | | |
| Breast ca. BT 549 | 10.9 | Fetal Skeletal Muscle | 0.6 |
| Breast ca. T47D | 1.2 | Skeletal Muscle Pool | 3.0 |
| Breast ca. MDA-N | 0.0 | Spleen Pool | 4.2 |
| Breast Pool | 6.3 | Thymus Pool | 3.8 |
| Trachea | 8.1 | CNS cancer (glio/astro) | 0.2 |
| | | U87-MG | |
| Lung | 0.1 | CNS cancer (glio/astro) | 0.8 |
| | | U-118-MG | |
| Fetal Lung | 5.3 | CNS cancer | 0.3 |
| | | (neuro; met) SK-N-AS | |
| Lung ca. NCI-N417 | 0.0 | CNS cancer (astro) SF- | 0.4 |
| | | 539 | |
| Lung ca. LX-1 | 3.9 | CNS cancer (astro) | 4.9 |
| | | SNB-75 | |
| Lung ca. NCI-H146 | 0.0 | CNS cancer (glio) | 0.0 |
| | | SNB-19 | |
| Lung ca. SHP-77 | 0.0 | CNS cancer (glio) SF- | 0.0 |
| | | 295 | |
| Lung ca. A549 | 0.1 | Brian (Amygdala) Pool | 4.6 |
| Lung ca. NCI-H526 | 0.0 | Brain (cerebellum) | 0.4 |
| Lung ca. NCI-H23 | 3.8 | Brain (fetal) | 4.7 |
| Lung ca. NCI-H460 | 0.0 | Brain (Hippocampus) | 3.0 |
| | | Pool | |
| Lung ca. HOP-62 | 0.1 | Cerebral Cortex Pool | 6.9 |
| Lung ca. NCI-H522 | 0.0 | Brain (Substantia nigra) | 4.9 |
| | | Pool | |
| Liver | 1.2 | Brain (Thalamus) Pool | 9.2 |
| Fetal Liver | 4.3 | Brain (whole) | 7.0 |
| Liver ca. HepG2 | 0.0 | Spinal Cord Pool | 0.8 |
| Kidney Pool | 3.2 | Adrenal Gland | 12.5 |
| Fetal Kidney | 5.1 | Pituitary gland Pool | 0.1 |
| Renal ca. 786-0 | 0.0 | Salivary Gland | 2.8 |
| Renal ca. A498 | 0.0 | Thyroid (female) | 6.3 |
| Renal ca. ACHN | 0.0 | Pancreatic ca. CAPAN2 | 0.0 |
| Renal ca. UO-31 | 5.3 | Pancreas Pool | 21.0 |

TABLE 32
General_screening_panel_v1.5

| | Rel. Exp. (%) Ag4845, | | Rel. Exp. (%) Ag4845, |
|---|---|---|---|
| Tissue Name | Run 228796340 | Tissue Name | Run 228796340 |
| Adipose | 5.8 | Renal ca. TK-10 | 59.0 |
| Melanoma* | 1.9 | Bladder | 5.5 |
| Hs688(A).T | | | |
| Melanoma* | 1.8 | Gastric ca. (liver met.) | 100.0 |
| Hs688(B).T | | NCI-N87 | |
| Melanoma* M14 | 0.0 | Gastric ca. KATO III | 16.3 |
| Melanoma* | 0.0 | Colon ca. SW-948 | 1.1 |
| LOXIMVI | | | |
| Melanoma* SK- | 0.0 | Colon ca. SW480 | 5.1 |
| MEL-5 | | | |
| Squamous cell | 34.9 | Colon ca.* (SW480 | 0.0 |
| carcinoma SCC-4 | | met) SW620 | |
| Testis Pool | 0.9 | Colon ca. HT29 | 16.7 |
| Prostate ca.* (bone | 0.0 | Colon ca. HCT-116 | 0.0 |
| met) PC-3 | | | |
| Prostate Pool | 1.2 | Colon ca. CaCo-2 | 0.0 |
| Placenta | 0.5 | Colon cancer tissue | 9.2 |
| Uterus Pool | 0.4 | Colon ca. SW1116 | 6.0 |
| Ovarian ca. | 0.6 | Colon ca. Colo-205 | 3.6 |
| OVCAR-3 | | | |
| Ovarian ca. SK-OV-3 | 0.3 | Colon ca. SW-48 | 0.0 |
| Ovarian ca. | 36.3 | Colon Pool | 2.9 |
| OVCAR-4 | | | |
| Ovarian ca. | 0.1 | Small Intestine Pool | 0.8 |
| OVCAR-5 | | | |
| Ovarian ca. IGROV-1 | 0.7 | Stomach Pool | 4.6 |
| Ovarian ca. | 20.2 | Bone Marrow Pool | 1.7 |
| OVCAR-8 | | | |
| Ovary | 3.5 | Fetal Heart | 1.0 |
| Breast ca. MCF-7 | 0.0 | Heart Pool | 1.0 |
| Breast ca. MDA- | 18.2 | Lymph Node Pool | 2.1 |
| MB-231 | | | |
| Breast ca. BT 549 | 6.2 | Fetal Skeletal Muscle | 0.7 |
| Breast ca. T47D | 0.7 | Skeletal Muscle Pool | 3.0 |
| Breast ca. MDA-N | 0.0 | Spleen Pool | 2.6 |
| Breast Pool | 4.6 | Thymus Pool | 3.4 |
| Trachea | 4.5 | CNS cancer (glio/astro) | 0.3 |
| | | U87-MG | |
| Lung | 0.2 | CNS cancer (glio/astro) | 0.9 |
| | | U-118-MG | |
| Fetal Lung | 4.1 | CNS cancer | 0.2 |
| | | (neuro; met) SK-N-AS | |
| Lung ca. NCI-N417 | 0.0 | CNS cancer (astro) SF- | 0.0 |
| | | 539 | |
| Lung ca. LX-1 | 3.1 | CNS cancer (astro) | 3.0 |
| | | SNB-75 | |
| Lung ca. NCI-H146 | 0.0 | CNS cancer (glio) | 0.0 |
| | | SNB-19 | |
| Lung ca. SHP-77 | 0.0 | CNS cancer (glio) SF- | 0.0 |
| | | 295 | |
| Lung ca. A549 | 0.2 | Brain (Amygdala) Pool | 1.3 |
| Lung ca NCI-H526 | 0.1 | Brain (cerebellum) | 1.6 |
| Lung ca. NCI-H23 | 3.6 | Brain (fetal) | 2.8 |
| Lung ca. NCI-H460 | 0.0 | Brain (Hippocampus) | 2.0 |
| | | Pool | |
| Lung ca. HOP-62 | 0.0 | Cerebral Cortex Pool | 7.0 |
| Lung ca. NCI-H522 | 0.0 | Brain (Substantia nigra) | 3.7 |
| | | Pool | |
| Liver | 0.9 | Brain (Thalamus) Pool | 8.2 |
| Fetal Liver | 43.5 | Brain (whole) | 5.4 |
| Liver ca. HepG2 | 0.0 | Spinal Cord Pool | 1.1 |
| Kidney Pool | 2.1 | Adrenal Gland | 7.9 |
| Fetal Kidney | 2.4 | Pituitary gland Pool | 0.2 |
| Renal ca. 786-0 | 0.0 | Salivary Gland | 2.1 |
| Renal ca. A498 | 0.2 | Thyroid (female) | 5.0 |
| Renal ca. ACHN | 0.0 | Pancreatic ca. CAPAN2 | 0.0 |
| Renal ca. UO-31 | 5.2 | Pancreas Pool | 9.7 |

TABLE 33
Panel 4.1D

| | Rel. Exp. (%) | Rel. Exp. (%) | Rel. Exp. (%) | | Rel. Exp. (%) | Rel. Exp. (%) | Rel. Exp. (%) |
|---|---|---|---|---|---|---|---|
| | Ag4811, Run | Ag4845, Run | Ag4944, Run | | Ag4811, Run | Ag4845, Run | Ag4944, Run |
| Tissue Name | 223273603 | 223335760 | 223598842 | Tissue Name | 223273603 | 223335760 | 223598842 |
| Secondary Th1 | 0.0 | 0.0 | 0.0 | HUVEC | 1.7 | 1.9 | 0.0 |
| act | | | | IL-1beta | | | |
| Secondary Th2 | 0.0 | 0.0 | 0.0 | HUVEC IFN | 2.3 | 0.3 | 0.0 |
| act | | | | gamma | | | |
| Secondary Tr1 | 1.5 | 0.0 | 0.0 | HUVEC TNF | 0.0 | 2.5 | 0.0 |
| act | | | | alpha + IFN | | | |
| | | | | gamma | | | |
| Secondary Th1 | 0.0 | 0.0 | 0.0 | HUVEC TNF | 0.0 | 0.0 | 0.0 |
| rest | | | | alpha + IL4 | | | |
| Secondary Th2 | 0.0 | 0.0 | 0.0 | HUVEC IL-11 | 0.0 | 1.7 | 0.0 |
| rest | | | | | | | |
| Secondary Tr1 | 0.0 | 0.0 | 0.0 | Lung | 0.0 | 0.0 | 0.0 |
| rest | | | | Microvascular | | | |
| | | | | EC none | | | |
| Primary Th1 | 0.9 | 2.0 | 0.0 | Lung | 0.0 | 0.5 | 0.0 |
| act | | | | Microvascular | | | |
| | | | | EC | | | |
| | | | | TNFalpha + | | | |
| | | | | IL-1beta | | | |
| Primary Th2 | 5.1 | 3.3 | 0.0 | Microvascular | 0.0 | 0.0 | 0.0 |
| act | | | | Dermal EC | | | |
| | | | | none | | | |
| Primary Tr1 | 2.0 | 1.1 | 0.0 | Microsvascular | 0.0 | 0.0 | 0.0 |
| act | | | | Dermal EC | | | |
| | | | | TNFalpha + | | | |
| | | | | IL-1beta | | | |
| Primary Th1 | 0.0 | 0.0 | 0.0 | Bronchial | 8.5 | 5.0 | 10.7 |
| rest | | | | epithelium | | | |
| | | | | TNFalpha + | | | |
| | | | | IL1beta | | | |
| Primary Th2 | 0.0 | 0.0 | 0.0 | Small airway | 11.1 | 5.9 | 2.7 |
| rest | | | | epithelium | | | |
| | | | | none | | | |
| Primary Tr1 rest | 0.0 | 0.0 | 0.0 | Small airway | 14.9 | 8.7 | 8.1 |
| | | | | epithelium | | | |
| | | | | TNFalpha + | | | |
| | | | | IL-1beta | | | |
| CD45RA CD4 | 0.0 | 0.5 | 0.0 | Coronery | 3.1 | 1.6 | 0.0 |
| lymphocyte act | | | | artery SMC | | | |
| | | | | rest | | | |
| CD45RO CD4 | 3.3 | 1.4 | 0.0 | Coronery | 1.4 | 0.8 | 0.0 |
| lymphocyte act | | | | artery SMC | | | |
| | | | | TNFaplha + | | | |
| | | | | IL-1beta | | | |
| CD8 | 0.7 | 0.4 | 0.0 | Astrocytes rest | 2.2 | 0.6 | 0.0 |
| lymphocyte act | | | | | | | |
| Secondary CD8 | 2.9 | 1.6 | 0.0 | Astrocytes | 0.0 | 0.5 | 0.0 |
| lymphocyte | | | | TNFalpha + | | | |
| rest | | | | IL-1beta | | | |
| Secondary CD8 | 0.7 | 0.0 | 0.0 | KU-812 | 0.0 | 0.0 | 0.0 |
| lymphocyte act | | | | (Basophil) rest | | | |
| CD4 | 0.0 | 0.0 | 0.0 | KU-812 | 0.0 | 0.6 | 0.0 |
| lymphocyte | | | | (Basophil) | | | |
| none | | | | PMA/ionomyc | | | |
| | | | | in | | | |
| 2ry | 0.0 | 0.0 | 0.0 | CCD1106 | 100.0 | 80.1 | 99.3 |
| Th1/Th2/Tr1_an | | | | (Keratinocytes) | | | |
| ti-CD95 CH11 | | | | none | | | |
| LAK cells rest | 3.9 | 2.3 | 0.0 | CCD1106 | 89.5 | 54.7 | 100.0 |
| | | | | (Keratinocytes) | | | |
| | | | | TNFalpha + | | | |
| | | | | IL-1beta | | | |
| LAK cells IL-2 | 0.0 | 0.0 | 0.0 | Liver cirrhosis | 4.6 | 3.0 | 0.0 |
| LAK cells IL-2 + | 0.0 | 1.3 | 0.0 | NCI-H292 | 2.4 | 1.3 | 5.7 |
| IL-12 | | | | none | | | |
| LAK cells IL-2 + | 1.2 | 0.8 | 0.0 | NCI-H292 IL-4 | 5.0 | 0.5 | 0.0 |
| IFN gamma | | | | | | | |
| LAK cells IL-2 + | 0.7 | 0.0 | 0.0 | NCI-H292 IL-9 | 1.9 | 2.2 | 3.1 |
| IL-18 | | | | | | | |
| LAK cells | 11.1 | 11.9 | 7.7 | NCI-H292 | 6.0 | 0.0 | 8.0 |
| PMA/ionomycin | | | | IL-13 | | | |
| NK Cells IL-2 | 0.0 | 0.0 | 0.0 | NCI-H292 | 3.9 | 2.1 | 3.2 |
| rest | | | | IFN gamma | | | |
| Two Way MLR | 0.0 | 1.8 | 0.0 | HPAEC none | 2.4 | 0.5 | 0.0 |
| 3 day | | | | | | | |
| Two Way MLR | 0.7 | 0.0 | 0.0 | HPAEC TNF | 0.0 | 1.3 | 0.0 |
| 5 day | | | | alpha + IL-1 | | | |
| | | | | beta | | | |
| Two Way MLR | 1.6 | 0.0 | 0.0 | Lung | 26.1 | 7.4 | 3.9 |
| 7 day | | | | fibroblast | | | |
| | | | | none | | | |
| PBMC rest | 0.0 | 0.0 | 0.0 | Lung | 17.1 | 11.2 | 10.2 |
| | | | | fibroblast TNF | | | |
| | | | | alpha + IL-1 | | | |
| | | | | beta | | | |
| PBMC PWM | 4.8 | 1.3 | 0.0 | Lung | 33.9 | 21.2 | 19.5 |
| | | | | fibroblast IL-4 | | | |
| PBMC PHA-L | 0.0 | 0.5 | 0.0 | Lung | 27.7 | 17.1 | 31.9 |
| | | | | fibroblast IL-9 | | | |
| Ramos (B cell) | 0.0 | 0.0 | 0.0 | Lung | 25.9 | 14.5 | 23.8 |
| none | | | | fibroblast | | | |
| | | | | IL-13 | | | |
| Ramos (B cell) | 0.0 | 0.0 | 0.0 | Lung | 52.1 | 20.7 | 33.2 |
| ionomycin | | | | fibroblast IFN | | | |
| | | | | gamma | | | |
| B lymphocytes | 10.7 | 4.3 | 10.0 | Dermal | 9.8 | 7.6 | 0.0 |
| PWM | | | | fibroblast | | | |
| | | | | CCD1070 rest | | | |
| B lymphocytes | 0.8 | 0.5 | 0.0 | Dermal | 5.4 | 5.2 | 4.5 |
| CD40L and IL-4 | | | | fibroblast | | | |
| | | | | CCD1070 | | | |
| | | | | TNF alpha | | | |
| EOL-1 dbcAMP | 0.0 | 0.0 | 0.0 | Dermal | 4.3 | 1.3 | 0.0 |
| | | | | fibroblast | | | |
| | | | | CCD1070 | | | |
| | | | | IL-1 beta | | | |
| EOL-1 dbcAMP | 2.7 | 3.1 | 0.0 | Dermal | 5.6 | 6.1 | 0.0 |
| PMA/ionomycin | | | | fibroblast IFN | | | |
| | | | | gamma | | | |
| Dendritic cells | 9.4 | 8.0 | 6.9 | Dermal | 14.9 | 4.4 | 12.2 |
| none | | | | fibroblast IL-4 | | | |
| Dendritic cells | 0.6 | 3.5 | 0.0 | Dermal | 7.5 | 6.0 | 3.8 |
| LPS | | | | Fibroblasts | | | |
| | | | | rest | | | |
| Dendritic cells | 15.5 | 12.7 | 6.2 | Neutrophils | 54.7 | 13.8 | 23.5 |
| anti-CD40 | | | | TNFa + LPS | | | |
| Monocytes rest | 0.0 | 0.0 | 0.0 | Neutrophils | 0.9 | 4.0 | 2.5 |
| | | | | rest | | | |
| Monocytes LPS | 8.8 | 4.4 | 0.0 | Colon | 0.8 | 3.7 | 0.0 |
| Macrophages | 1.6 | 0.0 | 0.0 | Lung | 4.4 | 5.3 | 0.0 |
| rest | | | | | | | |
| Macrophages | 0.0 | 0.6 | 0.0 | Thymus | 4.5 | 17.0 | 0.0 |
| LPS | | | | | | | |
| HUVEC none | 0.0 | 0.5 | 0.0 | Kidney | 29.9 | 100.0 | 15.3 |
| HUVEC starved | 0.0 | 0.5 | 0.0 | | | | |

General_screening_panel_v1.4 Summary/General_screening_panel_v1.5

Summary: Ag4811/Ag4845 Two experiments with the same probe and primer set show highest expression of the NOV7 gene in a gastric cancer cell line (NCI-N87) (CT=29.2). In addition, there appears to be substantial expression associated with other gastric cancer cell lines, colon cancer cell lines, breast cancer cell lines, ovarian cancer cell lines and a renal cancer cell line. Thus, the expression of this gene could be used to distinguish these samples from other samples in the panel. Moreover, therapeutic modulation of this gene, through the use of small molecule drugs, antibodies or protein therapeutics might be of benefit in the treatment of gastric cancer, colon cancer, breast cancer, ovarian cancer or renal cancer.

This gene is also moderately expressed in a number of metabolic tissues including adipose, fetal liver, skeletal muscle, adrenal, thyroid and pancreas. Thus, this gene product may be important for the pathogenesis, diagnosis, and/or treatment of metabolic diseases, including obesity and Types 1 and 2 diabetes.

This gene encodes a protein with homology to an interleukin 1 receptor-like protein. Interleukins are involved in many brain pathologies, due to the proinflammatory action of interleukins. Inflammation is a key pathological process mediating the damage due to stroke, Alzheimer's disease, and spinocerebellar ataxias, among others. IL-1 receptor like protein has been shown to bind IL-18, which is involved in CNS inflammation. Moreover, anti-inflammatory agents that are associated with reduced risk of Alzheimer's disease, such as NSAIDS, have been shown to inhibit IL-18 expression. Therefore, this gene product, which is expressed in the brain, is an ideal target for pharmacological interference with the inflammatory processes underlying these CNS disorders and any other CNS disorder in which inflammation plays a role.

References:

Evert B O, Vogt I R, Kindermann C, Ozimek L, de Vos R A, Brunt E R, Schmitt I, Klockgether T, Wullner U. Inflammatory genes are upregulated in expanded ataxin-3-expressing cell lines and spinocerebellar ataxia type 3 brains. J Neurosci Aug. 1, 2001;21(15):5389-96

Spinocerebellar ataxia type 3 (SCA3) is a polyglutamine disorder caused by a CAG repeat expansion in the coding region of a gene encoding ataxin-3. To study putative alterations of gene expression induced by expanded ataxin-3, we performed PCR-based cDNA subtractive hybridization in a cell culture model of SCA3. In rat mesencephalic CSM14.1 cells stably expressing expanded ataxin-3, we found a significant upregulation of mRNAs encoding the endopeptidase matrix metalloproteinase 2 (MMP-2), the transmembrane protein amyloid precursor protein, the interleukin-1 receptor-related Fos-inducible transcript, and the cytokine stromal cell-derived factor 1 alpha (SDF1alpha). Immunohistochemical, studies of the corresponding or associated proteins in human SCA3 brain tissue confirmed these findings, showing increased expression of MMP-2 and amyloid beta-protein (Abeta) in pontine neurons containing nuclear inclusions. In addition, extracellular Abeta-immunoreactive deposits were detected in human SCA3 pons. Furthermore, pontine neurons of SCA3 brains strongly expressed the _ignaling_mmatory interleukin-1 receptor antagonist, the proinflammatory cytokine interleukin-1beta, and the proinflammatory chemokine SDF1. Finally, increased numbers of reactive astrocytes and activated microglial cells were found in SCA3 pons. These results suggest that inflammatory processes are involved in the pathogenesis of SCA3.

Hoshino K, Tsutsui H, Kawai T, Takeda K, Nakanishi K, Takeda Y, Akira S. Cutting edge: generation of IL-18 receptor-deficient mice: evidence for IL-1 receptor-related protein as an essential IL-18 binding receptor. J Immunol May 1, 1999;162(9):5041-4

IL-18 is a proinflammatory cytokine that plays an important role in NK cell activation and Th1cell response. Recently IL-1R-related protein (IL-1Rrp) has been cloned as the receptor for IL-18. However, the functional role of IL-1Rrp is still controversial due to its low affinity to IL-18 as well as the possibility of the presence of another high-affinity binding receptor. In the present study, we have generated and characterized IL-1Rrp-deficient mice. The binding of murine rIL-18 was not detected in Th1-developing splenic CD4+ T cells isolated from IL-1Rrp-deficient mice. The activation of NF-kappa B or c-Jun N-terminal kinase were also not observed in the Th1cells. NK cells from IL-1Rrp-deficient mice had defects in cytolytic activity and IFN-gamma production in response to IL-18. Th1 cell development was also impaired in IL-1Rrp-deficient mice. These data demonstrate that IL-1Rrp is a ligand-binding receptor that is essential for IL-18-mediated signaling events.

Fiorucci S, Antonelli E, Burgaud J L, Morelli A. Nitric oxide-releasing NSAIDs: a review of their current status. Drug Saf2001;24(11):801-11

Nonsteroidal anti-inflammatory drugs (NSAIDs) are among the most widely prescribed drugs worldwide owing to their anti-inflammatory, antipyretic and analgesic properties. However, their use is hampered by gastrointestinal (GI) toxicity, the most common drug-related serious adverse event in_ignaling_mmat nations. Nitric oxide (NO)-releasing NSAIDs, a recently described class of drugs, are generated by adding a nitroxybutyl or a nitrosothiol moiety to the parent NSAID via a short-chain ester linkage. While efficacy of nitrosothiol-NO-NSAIDs still awaits investigation, nitroxybutyl-NO-NSAIDs have been extensively studied in animals, thus the abbreviation NO-NSAIDs used here refers to the latter group of NSAID derivatives. NO-NSAIDs retain the anti-inflammatory and antipyretic activity of original NSAIDs, although they exhibit markedly reduced gastrointestinal toxicity. NO-NSAIDs are nonselective cyclo-oxygenase (COX) inhibitors, and they also exert COX-independent activities that are NO-dependent. Indeed, NO-NSAIDs suppress production of the cytokines interleukin (IL)-1beta, IL-18 and interferon-gamma by causing the S-nitrosilation/inhibition of caspase-1. In acute and chronic animal models of inflammation, it has been demonstrated that NO-NSAIDs abrogated prostaglandin E2 as well as thromboxane B2 generation. In a murine model, NO-naproxen was approximately 10-fold more potent than naproxen in reducing animal writhing after intraperitoneal injection of acetic acid. Similar data have been obtained in chronic models of pain such as rat adjuvant arthritis. In vivo and in vitro studies suggest that NO-aspirin (acetylsalicylic acid) exerts more potent antithrombotic action than aspirin, probably by coupling the ability to inhibit COX-1 with the anti-adhesive effect of NO. Moreover, in a model of renal injury NO-flurbiprofen not only has been demonstrated to be devoid of nephrotoxicity but also to ameliorate renal function. Finally, in an animal model of chronic neurodegenerative disease, NO-flurbiprofen and NO-aspirin attenuated the brain inflammatory response. The GI toxicity of NO-flurbiprofen and NO-naproxen is currently being investigated in healthy individuals.

Stoll G, Jander S, Schroeter M. Cytokines in CNS disorders: neurotoxicity versus neuroprotection. J Neural Transm Suppl 2000;59:81-9

Cytokines orchestrate T cell-mediated immune responses. In experimental autoimmune encephalomyelitis (EAE) the proinflammatory cytokines interferon (WN)-gamma, tumor necrosis factor (TNF)-alpha, interleukin (IL)-1beta, IL-6, IL-12 and IL-18 are critically involved in the initiation and amplification of the local immune response in the CNS which is counter-balanced by upregulation of antiinflammatory cytokines such as IL-10. The predicted function of individual cytokines during EAE has recently been challenged by transgenic animal studies and neutralization experiments. Cytokine induction is not restricted to autoimmunity in the nervous system. Cytokines are involved in nerve regeneration and induced in focal cerebral ischemia both at the site of infarction and in remote nonischemic brain regions. In cerebral ischemia TNF-alpha and IL-1beta probably have dual functions: In concert with upregulation of inducible NO synthase (iNOS) they exert neurotoxicity while in the absence of iNOS, TNF-alpha and IL-1 beta may contribute to neuroprotection and plasticity. The interplay between glial cells, infiltrating leukocytes and induced cytokines leading to CNS pathology is complex and incompletely understood. Further assessment of the functional contribution of cytokines critically depends on the elucidation of downstream secondary signaling mechanisms.

Masada T, Hua Y, Xi G, Yang G Y, Hoff J T, Keep R F. Attenuation of intracerebral hemorrhage and thrombin-induced brain edema by overexpression of interleukin-1 receptor antagonist. J Neurosurg October 2001;95(4):680-6

OBJECT: Adenovirus-mediated overexpression of interleukin-1 receptor antagonist (IL-1ra) attenuates the inflammatory reaction and brain injury that follows focal cerebral ischemia. Recently, an inflammatory reaction after intracerebral hemorrhage (ICH) was identified. In this study the authors examine the hypothesis that overexpression of IL-1ra reduces brain injury (specifically edema formation) after ICH. METHODS: Adenoviruses expressing IL-1ra (Ad.RSVIL-1ra) or LacZ, a control protein (Ad.RSVlacZ), or saline were injected into the left lateral cerebral ventricle in rats. On the 5^{th} day after virus injection, 100 microl of autologous blood or 5 U thrombin was infused into the right basal ganglia. Rats with ICH were killed 24 or 72 hours later for measurement of brain water and ion content. Thrombin-treated rats were killed 24 hours later for edema measurements and an assessment of polymorphonuclear leukocyte (PMNL) infiltration by myeloperoxidase (MPO) assay, as well as histological evaluation. Compared with saline-treated and Ad.RSVlacZ-transduced controls, Ad.RSVIL-1ra-transduced rats had significantly attenuated edema in the ipsilateral basal ganglia 3 days after ICH (81.5+/−0.3% compared with 83.4+/−0.4% and 83.3+/−0.5% in control animals). Thrombin-induced brain edema was also reduced in Ad.RSVIL-1ra-treated rats (81.3+/−0.4% compared with 83.2+/−0.4% and 82.5+/−0.4% in control rats). The reduction in thrombin-induced edema was associated with a reduction in PMNL infiltration into the basal ganglia, as assessed by MPO assay (49% reduction) and histological examination. CONCLUSIONS: Overexpression of IL-1ra by using an adenovirus vector attenuated brain edema formation and thrombin-induced intracerebral inflammation following ICH. The reduction in ICH-induced edema with IL-1ra may result from reduction of thrombin-induced brain inflammation.

Panel 4.1D Summary: Ag 4811/4845/4944 Three experiments with two-different probes and primers show high expression of the NOV7 transcript in keratinocytes, regardless of their treatment with TNF-a plus IL-1b. It is also found to a lesser extent in neutrophils and lung fibroblasts. This transcript encodes for a novel IL-1 receptor related protein that may have the potential to trigger novel members of the IL-1 family members as described by Debets and al. (Ref 1 and 2). Novel IL1 receptor like molecules have been cloned and reported to lead to activation of NF-kB and IL18 production, a potent inflammatory cytokine associated with lung inflammation, IBD and psoriasis. IL1 R family members have also been shown to mediate inflammatory signals through NF-KB activation, among other ignaling pathways. Therefore, modulation of the function of this receptor by the use of protein therapeutics or antibodies could potentially prevent or reduce the severity of inflammatory processes observed in inflammatory lung and skin diseases such as asthma, bronchitis, emphysema and psoriasis.

References:

Debets R, Timans J C, Homey B, Zurawski S, Sana T R, Lo S, Wagner J, Edwards G, Clifford T, Menon S, Bazan J F, Kastelein R A. Two novel IL-1 family members, IL-1 delta and IL-1 epsilon, function as an antagonist and agonist of NF-kappa B activation through the orphan IL-1 receptor-related protein 2 J Immunol Aug. 1, 2001;167(3):1440-6

IL-1 is of utmost importance in the host response to immunological challenges. We identified and functionally characterized two novel IL-1 ligands termed IL-1delta and IL-1epsilon. Northern blot analyses show that these IL-1s are highly abundant in embryonic tissue and tissues containing epithelial cells (i.e., skin, lung, and stomach). In extension, quantitative real-time PCR revealed that of human skin-derived cells, only keratinocytes but not fibroblasts, endothelial cells, or melanocytes express IL-1delta and epsilon. Levels of keratinocyte IL-1delta are approximately 10-fold higher than those of IL-1epsilon. In vitro stimulation of keratinocytes with IL-1beta/TNF-alpha significantly up-regulates the expression of IL-1epsilon mRNA, and to a lesser extent of IL-1delta mRNA. In NF-kappaB-luciferase reporter assays, we demonstrated that IL-1delta and epsilon proteins do not initiate a functional response via classical IL-1R pairs, which confer responsiveness to IL-1alpha and beta or IL-18. However, IL-1epsilon activates NF-kappaB through the orphan IL-1R-related protein 2 (IL-1Rrp2), whereas IL-1delta, which shows striking homology to IL-1receptor antagonist, specifically and potently inhibits this IL-1epsilon response. In lesional psoriasis skin, characterized by chronic cutaneous inflammation, the mRNA expression of both IL-1 ligands as well as IL-1Rrp2 are increased relative to normal healthy skin. In total, IL-1delta and epsilon and IL-1Rrp2 may constitute an independent signaling system, analogous to IL-1alphabeta/receptor agonist and IL-1R1, that is present in epithelial barriers of our body and takes part in local inflammatory responses.

Parnet P, Garka K E, Bonnert T P, Dower S K, Sims J E. IL-1Rrp is a novel receptor.like molecule similar to the type I interleukin-1 receptor and its homologues T1/ST2 and IL-1R AcP. :2-J Biol Chem Feb. 23, 1996;271(8):3967-70

A novel member of the interleukin-1 receptor family has been cloned by polymerase chain reaction using degenerate oligonucleotide primers derived from regions of sequence conservation, using as template a yeast artificial chromosome known to contain both interleukin-1 (IL-1) receptors and T1/ST2. The new receptor, called IL-1 receptor-related protein or IL-1Rrp, fails to bind any of the known IL-1 ligands. A chimeric receptor, in which the IL-1Rrp cytoplasmic domain is fused to the extracellular and transmembrane regions of the IL-1 receptor, responds to IL-1 following transfection into COS cells by activation of NFkappaB and induction of IL-8 promoter function.

NOV6b and NOV6c

Expression of gene NOV6b and variant NOV6c was assessed using the primer-probe sets Ag4811, Ag4845 and Ag4946, described in Tables 34, 35 and 36. Results of the RTQ-PCR runs are shown in Tables 37, 38 and 39.

TABLE 34

Probe Name Ag4811

| | | | | |
|---|---|---|---|---|
| Primers | Sequences | Length | Start Positon | SEQ ID NO: |

| | | | | |
|---|---|---|---|---|
| Forward | 5′-catgtctcttttcgggaacata-3′ | 22 | 848 | 184 |
| Probe | TET-5′-cacagtaaacatcaccttcttggaagtg-3′-TAMRA | 28 | 877 | 185 |
| Reverse | 5′gaaggccataatcttccatttt-3′ | 22 | 905 | 186 |

TABLE 35

Probe Name Ag4845

| | | | | |
|---|---|---|---|---|
| Primers | Sequences | Length | Start Positon | SEQ ID NO: |

| | | | | |
|---|---|---|---|---|
| Forward | 5′-catgtctcttttcgggaacata-3′ | 22 | 848 | 187 |
| Probe | TET-5′-cacagtaaacatcaccttcttggaagtg-3′-TAMRA | 28 | 877 | 188 |
| Reverse | 5′gaaggccataatcttccatttt-3′ | 22 | 905 | 189 |

TABLE 36

Probe Name Ag4946

| | | | | |
|---|---|---|---|---|
| Primers | Sequences | Length | Start Positon | SEQ ID NO: |

| | | | | |
|---|---|---|---|---|
| Forward | 5′-ggcatcactgtgagcattagta-3′ | 22 | 695 | 190 |
| Probe | TET-5′-ctctgattgtggactgcaatgtaacagac-3′-TAMRA | 29 | 720 | 191 |
| Reverse | 5′tgactctccagcatcgtaga-3′ | 20 | 766 | 192 |

TABLE 37
General_screening_panel_v1.4

| | Rel. Exp. (%) Ag4811, | | Rel. Exp. (%) Ag4811, |
|---|---|---|---|
| Tissue Name | Run 223204488 | Tissue Name | Run 223204488 |
| Adipose | 4.9 | Renal ca. TK-10 | 71.7 |
| Melanoma* | 1.4 | Bladder | 6.5 |
| Hs688(A).T | | | |
| Melanoma* | 2.9 | Gastric ca. (liver met.) | 100.0 |
| Hs688(B).T | | NCI-N87 | |
| Melanoma* M14 | 0.0 | Gastric ca. KATO III | 24.1 |
| Melanoma* | 0.0 | Colon ca. SW-948 | 2.4 |
| LOXIMVI | | | |
| Melanoma* SK- | 0.0 | Colon ca. SW480 | 7.1 |
| MEL-5 | | | |
| Squamous cell | 45.4 | Colon ca.* (SW480 | 0.5 |
| carcinoma SCC-4 | | met) SW620 | |
| Testis Pool | 1.7 | Colon ca. HT29 | 16.8 |
| Prostate ca.* (bone | 0.0 | Colon ca. HCT-116 | 0.0 |
| met) PC-3 | | | |
| Prostate Pool | 2.7 | Colon ca. CaCo-2 | 0.5 |
| Placenta | 0.5 | Colon cancer tissue | 12.1 |
| Uterus Pool | 0.4 | Colon ca. SW1116 | 6.9 |
| Ovarian ca. | 1.4 | Colon ca. Colo-205 | 3.7 |
| OVCAR-3 | | | |
| Ovarian ca. SK-OV-3 | 0.3 | Colon ca. SW-48 | 0.0 |
| Ovarian ca. | 45.1 | Colon Pool | 1.6 |
| OVCAR-4 | | | |
| Ovarian ca. | 0.0 | Small Intestine Pool | 1.2 |
| OVCAR-5 | | | |
| Ovarian ca. IGROV-1 | 0.4 | Stomach Pool | 7.0 |
| Ovarian ca. | 25.9 | Bone Marrow Pool | 3.1 |
| OVCAR-8 | | | |
| Ovary | 6.0 | Fetal Heart | 0.4 |
| Breast ca. MCF-7 | 0.0 | Heart Pool | 1.3 |
| Breast ca. MDA- | 32.1 | Lymph Node Pool | 2.8 |
| MB-231 | | | |
| Breast ca. BT 549 | 10.9 | Fetal Skeletal Muscle | 0.6 |
| Breast ca. T47D | 1.2 | Skeletal Muscle Pool | 3.0 |
| Breast ca. MDA-N | 0.0 | Spleen Pool | 4.2 |
| Breast Pool | 6.3 | Thymus Pool | 3.8 |
| Trachea | 8.1 | CNS cancer (glio/astro) | 0.2 |
| | | U87-MG | |
| Lung | 0.1 | CNS cancer (glio/astro) | 0.8 |
| | | U-118-MG | |
| Fetal Lung | 5.3 | CNS cancer | 0.3 |
| | | (neuro; met) SK-N-AS | |
| Lung ca. NCI-N417 | 0.0 | CNS cancer (astro) SF- | 0.4 |
| | | 539 | |
| Lung ca. LX-1 | 3.9 | CNS cancer (astro) | 4.9 |
| | | SNB-75 | |
| Lung ca. NCI-H146 | 0.0 | CNS cancer (glio) | 0.0 |
| | | SNB-19 | |
| Lung ca. SHP-77 | 0.0 | CNS cancer (glio) SF- | 0.0 |
| | | 295 | |
| Lung ca. A549 | 0.1 | Brain (Amygdala) Pool | 4.6 |
| Lung ca. NCI-H526 | 0.0 | Brain (cerebellum) | 0.4 |
| Lung ca. NCI-H23 | 3.8 | Brain (fetal) | 4.7 |
| Lung ca. NCI-H460 | 0.0 | Brain (Hippocampus) | 3.0 |
| | | Pool | |
| Lung ca. HOP-62 | 0.1 | Cerebral Cortex Pool | 6.9 |
| Lung ca. NCI-H522 | 0.0 | Brain (Substantia nigra) | 4.9 |
| | | Pool | |
| Liver | 1.2 | Brain (Thalamus) Pool | 9.2 |
| Fetal Liver | 4.3 | Brain (whole) | 7.0 |
| Liver ca. HepG2 | 0.0 | Spinal Cord Pool | 0.8 |
| Kidney Pool | 3.2 | Adrenal Gland | 12.5 |
| Fetal Kidney | 5.1 | Pituitary gland Pool | 0.1 |
| Renal ca. 786-0 | 0.0 | Salivary Gland | 2.8 |
| Renal ca. A498 | 0.0 | Thyroid (female) | 6.3 |
| Renal ca. ACHN | 0.0 | Pancreatic ca. CAPAN2 | 0.0 |
| Renal ca. UO-31 | 5.3 | Pancreas Pool | 21.0 |

TABLE 38
General_screening_panel_v1.5

| | Rel. Exp. (%) Ag4845, | | Rel. Exp. (%) Ag4845, |
|---|---|---|---|
| Tissue Name | Run 228796340 | Tissue Name | Run 228796340 |
| Adipose | 5.8 | Renal ca. TK-10 | 59.0 |
| Melanoma* | 1.9 | Bladder | 5.5 |
| Hs688(A).T | | | |
| Melanoma* | 1.8 | Gastric ca. (liver met.) | 100.0 |
| Hs688(B).T | | NCI-N87 | |
| Melanoma* M14 | 0.0 | Gastric ca. KATO III | 16.3 |
| Melanoma* | 0.0 | Colon ca. SW-948 | 1.1 |
| LOXIMVI | | | |
| Melanoma* SK- | 0.0 | Colon ca. SW480 | 5.1 |
| MEL-5 | | | |
| Squamous cell | 34.9 | Colon ca.* (SW480 | 0.0 |
| carcinoma SCC-4 | | met) SW620 | |
| Testis Pool | 0.9 | Colon ca. HT29 | 16.7 |
| Prostate ca.* (bone | 0.0 | Colon ca. HCT-116 | 0.0 |
| met) PC-3 | | | |
| Prostate Pool | 1.2 | Colon ca. CaCo-2 | 0.0 |
| Placenta | 0.5 | Colon cancer tissue | 9.2 |
| Uterus Pool | 0.4 | Colon ca. SW1116 | 6.0 |
| Ovarian ca. | 0.6 | Colon ca. Colo-205 | 3.6 |
| OVCAR-3 | | | |
| Ovarian ca. SK-OV-3 | 0.3 | Colon ca. SW-48 | 0.0 |
| Ovarian ca. | 36.3 | Colon Pool | 2.9 |
| OVCAR-4 | | | |
| Ovarian ca. | 0.1 | Small Intestine Pool | 0.8 |
| OVCAR-5 | | | |
| Ovarian ca. IGROV-1 | 0.7 | Stomach Pool | 4.6 |
| Ovarian ca. | 20.2 | Bone Marrow Pool | 1.7 |
| OVCAR-8 | | | |
| Ovary | 3.5 | Fetal Heart | 1.0 |
| Breast ca. MCF-7 | 0.0 | Heart Pool | 1.0 |
| Breast ca. MDA- | 18.2 | Lymph Node Pool | 2.1 |
| MB-231 | | | |
| Breast ca. BT 549 | 6.2 | Fetal Skeletal Muscle | 0.7 |
| Breast ca. T47D | 0.7 | Skeletal Muscle Pool | 3.0 |
| Breast ca. MDA-N | 0.0 | Spleen Pool | 2.6 |
| Breast Pool | 4.6 | Thymus Pool | 3.4 |
| Trachea | 4.5 | CNS cancer (glio/astro) | 0.3 |
| | | U87-MG | |
| Lung | 0.2 | CNS cancer (glio/astro) | 0.9 |
| | | U-118-MG | |
| Fetal Lung | 4.1 | CNS cancer | 0.2 |
| | | (neuro; met) SK-N-AS | |
| Lung ca. NCI-N417 | 0.0 | CNS cancer (astro) SF- | 0.0 |
| | | 539 | |
| Lung ca. LX-1 | 3.1 | CNS cancer (astro) | 3.0 |
| | | SNB-75 | |
| Lung ca. NCI-H146 | 0.0 | CNS cancer (glio) | 0.0 |
| | | SNB-19 | |
| Lung ca. SHP-77 | 0.0 | CNS cancer (glio) SF- | 0.0 |
| | | 295 | |
| Lung ca. A549 | 0.2 | Brain (Amygdala) Pool | 1.3 |
| Lung ca. NCI-H526 | 0.1 | Brain (cerebellum) | 1.6 |
| Lung ca. NCI-H23 | 3.6 | Brain (fetal) | 2.8 |
| Lung ca. NCI-H460 | 0.0 | Brain (Hippocampus) | 2.0 |
| | | Pool | |
| Lung ca. HOP-62 | 0.0 | Cerebral Cortex Pool | 7.0 |
| Lung ca. NCI-H522 | 0.0 | Brain (Substantia nigra) | 3.7 |
| | | Pool | |
| Liver | 0.9 | Brain (Thalamus) Pool | 8.2 |
| Fetal Liver | 43.5 | Brain (whole) | 5.4 |
| Liver ca. HepG2 | 0.0 | Spinal Cord Pool | 1.1 |
| Kidney Pool | 2.1 | Adrenal Gland | 7.9 |
| Fetal Kidney | 2.4 | Pituitary gland Pool | 0.2 |
| Renal ca. 786-0 | 0.0 | Salivary Gland | 2.1 |
| Renal ca. A498 | 0.2 | Thyroid (female) | 5.0 |
| Renal ca. ACHN | 0.0 | Pancreatic ca. CAPAN2 | 0.0 |
| Renal ca. UO-31 | 5.2 | Pancreas Pool | 9.7 |

TABLE 39
Panel 4.1D

| | Rel. Exp. (%) | Rel. Exp. (%) | Rel. Exp. (%) | | Rel. Exp. (%) | Rel. Exp. (%) | Rel. Exp. (%) |
|---|---|---|---|---|---|---|---|
| | Ag4811, Run | Ag4845, Run | Ag4946, Run | | Ag4811, Run | Ag4845, Run | Ag4946, Run |
| Tissue Name | 223273603 | 223335760 | 223626614 | Tissue Name | 223273603 | 223335760 | 223626614 |
| Secondary Th1 | 0.0 | 0.0 | 0.0 | HUVEC | 1.7 | 1.9 | 0.0 |
| act | | | | IL-1beta | | | |
| Secondary Th2 | 0.0 | 0.0 | 0.0 | HUVEC IFN | 2.3 | 0.3 | 0.0 |
| act | | | | gamma | | | |
| Secondary Tr1 | 1.5 | 0.0 | 0.0 | HUVEC TNF | 0.0 | 2.5 | 0.0 |
| act | | | | alpha + IFN | | | |
| | | | | gamma | | | |
| Secondary Th1 | 0.0 | 0.0 | 0.0 | HUVEC TNF | 0.0 | 0.0 | 0.0 |
| rest | | | | alpha + IL4 | | | |
| Secondary Th2 | 0.0 | 0.0 | 0.0 | HUVEC IL-11 | 0.0 | 1.7 | 0.0 |
| rest | | | | | | | |
| Secondary Tr1 | 0.0 | 0.0 | 0.0 | Lung | 0.0 | 0.0 | 0.0 |
| rest | | | | Microvascular | | | |
| | | | | EC none | | | |
| Primary Th1 act | 0.9 | 2.0 | 0.0 | Lung | 0.0 | 0.5 | 0.0 |
| | | | | Microvascular | | | |
| | | | | EC | | | |
| | | | | TNFalpha + | | | |
| | | | | IL-1beta | | | |
| Primary Th2 act | 5.1 | 3.3 | 0.0 | Microvascular | 0.0 | 0.0 | 0.0 |
| | | | | Dermal EC | | | |
| | | | | none | | | |
| Primary Tr1 act | 2.0 | 1.1 | 0.0 | Microsvasular | 0.0 | 0.0 | 0.0 |
| | | | | Dermal EC | | | |
| | | | | TNFalpha + | | | |
| | | | | IL-1beta | | | |
| Primary Th1 | 0.0 | 0.0 | 0.0 | Bronchial | 8.5 | 5.0 | 12.9 |
| rest | | | | epithelium | | | |
| | | | | TNFalpha + | | | |
| | | | | IL1beta | | | |
| Primary Th2 | 0.0 | 0.0 | 0.0 | Small airway | 11.1 | 5.9 | 8.6 |
| rest | | | | epithelium | | | |
| | | | | none | | | |
| Primary Tr1 rest | 0.0 | 0.0 | 0.0 | Small airway | 14.9 | 8.7 | 2.9 |
| | | | | epithelium | | | |
| | | | | TNFalpha + | | | |
| | | | | IL-1beta | | | |
| CD45RA CD4 | 0.0 | 0.5 | 0.0 | Coronery | 3.1 | 1.6 | 7.3 |
| lymphocyte act | | | | artery SMC | | | |
| | | | | rest | | | |
| CD45RO CD4 | 3.3 | 1.4 | 13.5 | Coronery | 1.4 | 0.8 | 0.0 |
| lymphocyte act | | | | artery SMC | | | |
| | | | | TNFalpha + | | | |
| | | | | IL-1beta | | | |
| CD8 | 0.7 | 0.4 | 0.0 | Astrocytes rest | 2.2 | 0.6 | 0.0 |
| lymphocyte act | | | | | | | |
| Secondary CD8 | 2.9 | 1.6 | 0.0 | Astrocytes | 0.0 | 0.5 | 0.0 |
| lymphocyte rest | | | | TNFalpha + | | | |
| | | | | IL-1beta | | | |
| Secondary CD8 | 0.7 | 0.0 | 0.0 | KU-812 | 0.0 | 0.0 | 0.0 |
| lymphocyte act | | | | (Basophil) rest | | | |
| CD4 | 0.0 | 0.0 | 0.0 | KU-812 | 0.0 | 0.6 | 0.0 |
| lymphocyte | | | | (Basophil) | | | |
| none | | | | PMA/ionomyc | | | |
| | | | | in | | | |
| 2ry | 0.0 | 0.0 | 0.0 | CCD1106 | 100.0 | 80.1 | 57.8 |
| Th1/Th2/Tr1_an | | | | (Keratinocytes) | | | |
| ti-CD95 CH11 | | | | none | | | |
| LAK cells rest | 3.9 | 2.3 | 6.8 | CCD1106 | 89.5 | 54.7 | 100.0 |
| | | | | (Keratinocytes) | | | |
| | | | | TNFalpha + | | | |
| | | | | IL-1beta | | | |
| LAK cells IL-2 | 0.0 | 0.0 | 0.0 | Liver cirrhosis | 4.6 | 3.0 | 5.4 |
| LAK cells IL-2 + | 0.0 | 1.3 | 0.0 | NCI-H292 | 2.4 | 1.3 | 5.2 |
| IL-12 | | | | none | | | |
| LAK cells IL-2 | 1.2 | 0.8 | 0.0 | NCI-H292 IL-4 | 5.0 | 0.5 | 14.5 |
| IFN gamma | | | | | | | |
| LAK cells IL-2 + | 0.7 | 0.0 | 0.0 | NCI-H292 IL-9 | 1.9 | 2.2 | 15.6 |
| IL-18 | | | | | | | |
| LAK cells | 11.1 | 11.9 | 35.8 | NCI-H292 | 6.0 | 0.0 | 0.0 |
| PMA/ionomycin | | | | IL-13 | | | |
| NK Cells IL-2 | 0.0 | 0.0 | 0.0 | NCI-H292 | 3.9 | 2.1 | 15.4 |
| rest | | | | IFN gamma | | | |
| Two Way MLR | 0.0 | 1.8 | 0.0 | HPAEC none | 2.4 | 0.5 | 0.0 |
| 3 day | | | | | | | |
| Two Way MLR | 0.7 | 0.0 | 0.0 | HPAEC TNF | 0.0 | 1.3 | 0.0 |
| 5 day | | | | alpha + IL-1 | | | |
| | | | | beta | | | |
| Two Way MLR | 1.6 | 0.0 | 0.0 | Lung | 26.1 | 7.4 | 10.2 |
| 7 day | | | | fibroblast | | | |
| | | | | none | | | |
| PBMC rest | 0.0 | 0.0 | 0.0 | Lung | 17.1 | 11.2 | 48.6 |
| | | | | fibroblast TNF | | | |
| | | | | alpha + IL-1 | | | |
| | | | | beta | | | |
| PBMC PWM | 4.8 | 1.3 | 0.0 | Lung | 33.9 | 21.2 | 60.3 |
| | | | | fibroblast IL-4 | | | |
| PBMC PHA-L | 0.0 | 3.5 | 0.0 | Lung | 27.7 | 17.1 | 19.6 |
| | | | | fibroblast IL-9 | | | |
| Ramos (B cell) | 0.0 | 0.0 | 0.0 | Lung | 25.9 | 14.5 | 74.2 |
| none | | | | fibroblast | | | |
| | | | | IL-13 | | | |
| Ramos (B cell) | 0.0 | 0.0 | 0.0 | Lung | 52.1 | 20.7 | 28.9 |
| ionomycin | | | | fibroblast IFN | | | |
| | | | | gamma | | | |
| B lymphocytes | 10.7 | 4.3 | 6.7 | Dermal | 9.8 | 7.6 | 9.9 |
| PWM | | | | fibroblast | | | |
| | | | | CCD1070 rest | | | |
| B lymphocytes | 0.8 | 0.5 | 0.0 | Dermal | 5.4 | 5.2 | 0.0 |
| CD40L and IL-4 | | | | fibroblast | | | |
| | | | | CCD1070 | | | |
| | | | | TNF alpha | | | |
| EOL-1 dbcAMP | 0.0 | 0.0 | 0.0 | Dermal | 4.3 | 1.3 | 11.7 |
| | | | | fibroblast | | | |
| | | | | CCD1070 | | | |
| | | | | IL-1 beta | | | |
| EOL-1 dbcAMP | 2.7 | 3.1 | 0.0 | Dermal | 5.6 | 6.1 | 8.3 |
| PMA/ionomycin | | | | fibroblast IFN | | | |
| | | | | gamma | | | |
| Dendritic cells | 9.4 | 8.0 | 0.0 | Dermal | 14.9 | 4.4 | 12.8 |
| none | | | | fibroblast IL-4 | | | |
| Dendritic cells | 0.6 | 3.5 | 15.2 | Dermal | 7.5 | 6.0 | 0.0 |
| LPS | | | | Fibroblasts | | | |
| | | | | rest | | | |
| Dendritic cells | 15.5 | 12.7 | 25.9 | Neutrophils | 54.7 | 13.8 | 43.2 |
| anti-CD40 | | | | TNFa+ LPS | | | |
| Monocytes rest | 0.0 | 0.0 | 0.0 | Neutrophils | 0.9 | 4.0 | 0.0 |
| | | | | rest | | | |
| Monocytes LPS | 8.8 | 4.4 | 0.0 | Colon | 0.8 | 3.7 | 0.0 |
| Macrophages | 1.6 | 0.0 | 0.0 | Lung | 4.4 | 5.3 | 0.0 |
| rest | | | | | | | |
| Macrophages | 0.0 | 0.6 | 0.0 | Thymus | 4.5 | 17.0 | 16.6 |
| LPS | | | | | | | |
| HUVEC none | 0.0 | 0.5 | 0.0 | Kidney | 29.9 | 100.0 | 8.9 |
| HUVEC starved | 0.0 | 0.5 | 0.0 | | | | |

General_screening_panel_v1.4 Summary/General_screening_panel_v1.5

Summary: Ag4811/Ag4845 Two experiments with the same probe and primer set show highest expression of the NOV7 gene appears to be highest in a sample derived from a gastric cancer cell line (NCI-N87)(CT=29.2). In addition there appears to be substantial expression associated with other gastric cancer cell lines, colon cancer cell lines, breast cancer cell lines, ovarian cancer cell lines and a renal cancer cell line. Thus, the expression of this gene could be used to distinguish these samples from other samples in the panel. Moreover, therapeutic modulation of this gene, through the use of small molecule drugs, antibodies or protein therapeutics might be of benefit in the treatment of gastric cancer, colon cancer, breast cancer, ovarian cancer or renal cancer.

This gene is also moderately expressed in a number of metabolic tissues including adipose, fetal liver, skeletal muscle, adrenal, thyroid and pancreas. Thus, this gene product may be important for the pathogenesis, diagnosis, and/or treatment of metabolic diseases, including obesity and Types 1 and 2 diabetes.

This gene encodes a protein with homology to an interleukin 1 receptor-like protein. Interleukins are involved in many brain pathologies, due to the proinflammatory action of interleukins. Inflammation is a key pathological process mediating the damage due to stroke, Alzheimer's disease, and spinocerebellar ataxias, among others. IL-1 receptor like protein has been shown to bind IL-18, which is involved in CNS inflammation. Moreover, anti-inflammatory agents that are associated with reduced risk of Alzheimer's disease, such as NSAIDS, have been shown to inhibit IL-18 expression. Therefore, this gene product, which is expressed in the brain, is an ideal target for pharmacological interference with the inflammatory processes underlying these CNS disorders and any other CNS disorder in which inflammation plays a role.

References:

Evert B O, Vogt I R, Kindermann C, Ozimek L, de Vos R A, Brunt E R, Schmitt I, Klockgether T, Wullner U. Inflammatory genes are upregulated in expanded ataxin-3-expressing cell lines and spinocerebellar ataxia type 3 brains. J Neurosci Aug. 1, 2001;21(15):5389-96

Spinocerebellar ataxia type 3 (SCA3) is a polyglutamine disorder caused by a CAG repeat expansion in the coding region of a gene encoding ataxin-3. To study putative alterations of gene expression induced by expanded ataxin-3, we performed PCR-based cDNA subtractive hybridization in a cell culture model of SCA3. In rat mesencephalic CSM14.1 cells stably expressing expanded ataxin-3, we found a significant upregulation of mRNAs encoding the endopeptidase matrix metalloproteinase 2 (MMP-2), the transmembrane protein amyloid precursor protein, the interleukin-1 receptor-related Fos-inducible transcript, and the cytokine stromal cell-derived factor 1 alpha (SDF 1 alpha). Immunohistochemical studies of the corresponding or associated proteins in human SCA3 brain tissue confirmed these findings, showing increased expression of MMP-2 and amyloid beta-protein (Abeta) in pontine neurons containing nuclear inclusions. In addition, extracellular Abeta-immunoreactive deposits were detected in human SCA3 pons. Furthermore, pontine neurons of SCA3 brains strongly expressed the antiinflammatory interleukin-1 receptor antagonist, the proinflammatory cytokine interleukin-1 beta, and the proinflammatory chemokine SDF1. Finally, increased numbers of reactive astrocytes and activated microglial cells were found in SCA3 pons. These results suggest that inflammatory processes are involved in the pathogenesis of SCA3.

Hoshino K, Tsutsui H, Kawai T, Takeda K, Nakanishi K, Takeda Y, Akira S. Cutting edge: generation of IL-18 receptor-deficient mice: evidence for IL-1 receptor-related protein as an essential IL-18 binding receptor. J Immunol May 1, 1999;162(9):5041-4

IL-18 is a proinflammatory cytokine that plays an important role in NK cell activation and Th1cell response. Recently IL-1R-related protein (IL-1Rrp) has been cloned as the receptor for IL-18. However, the functional role of IL-1Rrp is still controversial due to its low affinity to IL-18 as well as the possibility of the presence of another high-affinity binding receptor. In the present study, we have generated and characterized IL-1Rrp-deficient mice. The binding of murine rIL-18 was not detected in Th1-developing splenic CD4+ T cells isolated from IL-1Rrp-deficient mice. The activation of NF-kappa B or c-Jun N-terminal kinase were also not observed in the Th1cells. NK cells from IL-1Rrp-deficient mice had defects in cytolytic activity and IFN-gamma production in response to IL-18. ml cell development was also impaired in IL-1Rrp-deficient mice. These data demonstrate that IL-1Rrp is a ligand-binding receptor that is essential for IL-18-mediated signaling events.

Fiorucci S, Antonelli E, Burgaud J L, Morelli A. Nitric oxide-releasing NSAIDs: a review of their current status. Drug Saf 200 1;24(11):801-11

Nonsteroidal anti-inflammatory drugs (NSAIDs) are among the most widely prescribed drugs worldwide owing to their anti-inflammatory, antipyretic and analgesic properties. However, their use is hampered by gastrointestinal (GI) toxicity, the most common drug-related serious adverse event in industrialised nations. Nitric oxide (NO)-releasing NSAIDs, a recently described class of drugs, are generated by adding a nitroxybutyl or a nitrosothiol moiety to the parent NSAID via a short-chain ester linkage. While efficacy of nitrosothiol-NO-NSAIDs still awaits investigation, nitroxybutyl-NO-NSAIDs have been extensively studied in animals, thus the abbreviation NO-NSAIDs used here refers to the latter group of NSAID derivatives. NO-NSAIDs retain the anti-inflammatory and antipyretic activity of original NSAIDs, although they exhibit markedly reduced gastrointestinal toxicity. NO-NSAIDs are nonselective cyclo-oxygenase (COX) inhibitors, and they also exert COX-independent activities that are NO-dependent. Indeed, NO-NSAIDs suppress production of the cytokines interleukin (IL)-1beta, IL-18 and interferon-gamma by causing the S-nitrosilation/inhibition of caspase-1. In acute and chronic animal models of inflammation, it has been demonstrated that NO-NSAIDs abrogated prostaglandin E2 as well as thromboxane B2 generation. In a murine model, NO-naproxen was approximately 10-fold more potent than naproxen in reducing animal writhing after intraperitoneal injection of acetic acid. Similar data have been obtained in chronic models of pain such as rat adjuvant arthritis. In vivo and in vitro studies suggest that NO-aspirin (acetylsalicylic acid) exerts more potent antithrombotic action than aspirin, probably by coupling the ability to inhibit COX-1 with the anti-adhesive effect of NO. Moreover, in a model of renal injury NO-flurbiprofen not only has been demonstrated to be devoid of nephrotoxicity but also to ameliorate renal function. Finally, in an animal model of chronic neurodegenerative disease, NO-flurbiprofen and NO-aspirin attenuated the brain inflammatory response. The GI toxicity of NO-flurbiprofen and NO-naproxen is currently being investigated in healthy individuals.

Stoll G, Jander S, Schroeter M. Cytokines in CNS disorders: neurotoxicity versus neuroprotection. J Neural Transm Suppl 2000;59:81-9

Cytokines orchestrate T cell-mediated immune responses. In experimental autoimmune encephalomyelitis (EAE) the proinflammatory cytokines interferon (IFN)-gamma, tumor necrosis factor (TNF)-alpha, interleukin (IL)-1beta, IL-6, IL-12 and IL-18 are critically involved in the initiation and amplification of the local immune response in the CNS which is counter-balanced by upregulation of antiinflammatory cytokines such as IL-10. The predicted function of individual cytokines during EAE has recently been challenged by transgenic animal studies and neutralization experiments. Cytokine induction is not restricted to autoimmunity in the nervous system. Cytokines are involved in nerve regeneration and induced in focal cerebral ischemia both at the site of infarction and in remote nonischemic brain regions. In cerebral ischemia TNF-alpha and IL-1beta probably have dual functions: In concert with upregulation of inducible NO synthase (iNOS) they exert neurotoxicity while in the absence of iNOS, TNF-alpha and IL-1beta may contribute to neuroprotection and plasticity. The interplay between glial cells, infiltrating leukocytes and induced cytokines leading to CNS pathology is complex and incompletely understood. Further assessment of the functional contribution of cytokines critically depends on the elucidation of downstream secondary signaling mechanisms.

Masada T, Hua Y, Xi G, Yang G Y, Hoff J T, Keep R F. Attenuation of intracerebral hemorrhage and thrombin-induced brain edema by overexpression of interleukin-1receptor antagonist. J Neurosurg October 2001;95(4):680-6

OBJECT: Adenovirus-mediated overexpression of interleukin-1 receptor antagonist (IL-1ra) attenuates the inflammatory reaction and brain injury that follows focal cerebral ischemia. Recently, an inflammatory reaction after intracerebral hemorrhage (ICH) was identified. In this study the authors examine the hypothesis that overexpression of IL-1ra reduces brain injury (specifically edema formation) after ICH. METHODS: Adenoviruses expressing IL-1ra (Ad.RSVIL-1ra) or LacZ, a control protein (Ad.RSVlacZ), or saline were injected into the left lateral cerebral ventricle in rats. On the 5th day after virus injection, 100 microl of autologous blood or 5 U thrombin was infused into the right basal ganglia. Rats with ICH were killed 24 or 72 hours later for measurement of brain water and ion content. Thrombin-treated rats were killed 24 hours later for edema measurements and an assessment of polymorphonuclear leukocyte (PMNL) infiltration by myeloperoxidase (MPO) assay, as well as histological evaluation. Compared with saline-treated and Ad.RSVlacZ-transduced controls, Ad.RSVIL-1ra-transduced rats had significantly attenuated edema in the ipsilateral basal ganglia 3 days after ICH (81.5+/−0.3% compared with 83.4+/−0.4% and 83.3+/−0.5% in control animals). Thrombin-induced brain edema was also reduced in Ad.RSVIL-1ra-treated rats (81.3+/31 0.4% compared with 83.2+/31 0.4% and 82.5+/−0.4% in control rats). The reduction in thrombin-induced edema was associated with a reduction in PMNL infiltration into the basal ganglia, as assessed by MPO assay (49% reduction) and histological examination. CONCLUSIONS: Overexpression of IL-1ra by using an adenovirus vector attenuated brain edema formation and thrombin-induced intracerebral inflammation following ICH. The reduction in ICH-induced edema with IL-1ra may result from reduction of thrombin-induced brain inflammation.

Panel 4.1D Summary: Ag 4811/4845/4946 Three experiments with two different probes and primers show high expression of the NOV7 transcript in keratinocytes, regardless of their treatment with TNF-a plus IL-1b. This transcript encodes for a novel IL-1 receptor related protein that may have the potential to trigger novel members of the IL-1 family members as described by Debets and al. (Ref 1 and 2). Novel IL1 receptor like molecules have been cloned and reported to lead to activation of NF-kB and IL18 production, a potent inflammatory cytokine associated with lung inflammation, IBD and psoriasis. IL1 R family members have also been shown to mediate inflammatory signals through NF-kB activation, among other signalling pathways. Therefore, modulation of the function of this receptor by the use of protein therapeutics or antibodies could potentially prevent or reduce the severity of inflammatory processes observed in inflammatory lung and skin diseases such as asthma, bronchitis, emphysema and psoriasis.

Significant levels of expression are also seen in neutrophils stimulated with TNF-a and LPS. The expression of this transcript in neutrophils suggest an important role of this receptor in innate immunity and in inflammation associated with neutrophil accummulation such as observed in inflammatory bowel diseases, psoriasis, asthma, chronic bronchitis and rheumatoid arthritis. Therefore modulation of the function of this receptor by the use of protein therapeutics or antibodies could potentially prevent or reduce these diseases.

References:

Debets R, Timans J C, Homey B, Zurawski S, Sana T R, Lo S, Wagner J, Edwards G, Clifford T, Menon S, Bazan J F, Kastelein R A. Two novel IL-1 family members, IL-1 delta and IL-1 epsilon, function as an antagonist and agonist of NF-kappa B activation through the orphan IL-1 receptor-related protein 2 J Immunol Aug. 1, 2001; 167(3):1440-6

IL-1 is of utmost importance in the host response to immunological challenges. We identified and functionally characterized two novel IL-1 ligands termed IL-1delta and IL-1 epsilon. Northern blot analyses show that these IL-1s are highly abundant in embryonic tissue and tissues containing epithelial cells (i.e., skin, lung, and stomach). In extension, quantitative real-time PCR revealed that of human skin-derived cells, only keratinocytes but not fibroblasts, endothelial cells, or melanocytes express IL-1delta and epsilon. Levels of keratinocyte IL-1delta are approximately 10-fold higher than those of IL-1epsilon. In vitro stimulation of keratinocytes with IL-1beta/TNF-alpha significantly up-regulates the expression of IL-1epsilon mRNA, and to a lesser extent of IL-1delta mRNA. In NF-kappaB-luciferase reporter assays, we demonstrated that IL-1delta and epsilon proteins do not initiate a functional response via classical IL-1R pairs, which confer responsiveness to IL-1alpha and beta or IL-18. However, IL-1epsilon activates NF-kappaB through the orphan IL-1R-related protein 2 (IL-1Rrp2), whereas IL-1delta, which shows striking homology to IL-1 receptor antagonist, specifically and potently inhibits this IL-1epsilon response. In lesional psoriasis skin, characterized by chronic cutaneous inflammation, the mRNA expression of both IL-1 ligands as well as IL-1Rrp2 are increased relative to normal healthy skin. In total, IL-1delta and epsilon and IL-1Rrp2 may constitute an independent signaling system, analogous to IL-1alphabeta/receptor agonist and IL-1R1, that is present in epithelial barriers of our body and takes part in local inflammatory responses.

Parnet P, Garka K E, Bonnert T P, Dower S K, Sims J E. IL-1Rrp is a novel receptor-like molecule similar to the type I interleukin-1 receptor and its homologues T1/ST2 and IL-1R AcP.:2-J Biol Chem Feb. 23, 1996;271(8):3967-70

A novel member of the interleukin-1 receptor family has been cloned by polymerase chain reaction using degenerate oligonucleotide primers derived from regions of sequence conservation, using as template a yeast artificial chromosome known to contain both interleukin-1 (IL-1) receptors and T1/ST2. The new receptor, called IL-1 receptor-related protein or IL-1Rrp, fails to bind any of the known IL-1 ligands. A chimeric receptor, in which the IL-1 Rrp cytoplasmic domain is fused to the extracellular and transmembrane regions of the IL-1 receptor, responds to IL-1 following transfection into COS cells by activation of NFkappaB and induction of IL-8 promoter function.

NOV1a

Expression of gene NOVIA was assessed using the primer-probe sets Ag2451 and Ag1455, described in Tables 40 and 41. Results of the RTQ-PCR runs are shown in Tables 42, 43, 44, 45, and 46.

TABLE 40

Probe Name Ag2451

| | | | | |
|---|---|---|---|---|
| Primers | Sequences | Length | Start Positon | SEQ ID NO: |

| | | | | |
|---|---|---|---|---|
| Forward | 5′-ggccaagtaatgacaatttgaa-3′ | 22 | 362 | 193 |
| Probe | TET-5′-tcagtttattcctaacaatgagaacgca-3′-TAMRA | 28 | 405 | 194 |
| Reverse | 5′tgcaaaatgttatcagcttgtg-3′ | 22 | 440 | 195 |

TABLE 41

Probe Name Ag1455

| | | | | |
|---|---|---|---|---|
| Primers | Sequences | Length | Start Positon | SEQ ID NO: |

| | | | | |
|---|---|---|---|---|
| Forward | 5′-caggatttggatccattgtaga-3′ | 22 | 998 | 196 |
| Probe | TET-5′-caaaatacacttcggcaagccagagt-3′-TAMRA | 26 | 1033 | 197 |
| Reverse | 5′cacatcagtgcttatggtttcc-3′ | 22 | 1059 | 198 |

TABLE 42
AI_comprehensive panel_v1.0

| | Rel. Exp. (%) Ag2451, | | Rel. Exp. (%) Ag2451, |
|---|---|---|---|
| Tissue Name | Run 228157424 | Tissue Name | Run 228157424 |
| 110967 COPD-F | 19.2 | 112427 Match Control | 7.7 |
| | | Psoriasis-F | |
| 110980 COPD-F | 3.0 | 112418 Psoriasis-M | 40.1 |
| 110968 COPD-M | 39.2 | 112723 Match Control | 24.1 |
| | | Psoriasis-M | |
| 110977 COPD-M | 2.7 | 112419 Psoriasis-M | 15.0 |
| 110989 Emphysema-F | 14.6 | 112424 Match Control | 11.4 |
| | | Psoriasis-M | |
| 110992 Emphysema-F | 0.0 | 112420 Psoriasis-M | 100.0 |
| 110993 Emphysema-F | 4.9 | 112425 Match Control | 5.5 |
| | | Psoriasis-M | |
| 110994 Emphysema-F | 5.2 | 104689 (MF) OA | 17.3 |
| | | Bone-Backus | |
| 110995 Emphysema-F | 8.3 | 104690 (MF) Adj | 12.2 |
| | | “Normal⇄ Bone-Backus | |
| 110996 Emphysema-F | 0.0 | 104691 (MF) OA | 8.1 |
| | | Synovium-Backus | |
| 110997 Asthma-M | 21.9 | 104692 (BA) OA | 7.7 |
| | | Cartilage-Backus | |
| 111001 Asthma-F | 0.0 | 104694 (BA) OA | 9.0 |
| | | Bone-Backus | |
| 111002 Asthma-F | 5.3 | 104695 (BA) Adj | 2.9 |
| | | “Normal⇄ Bone-Backus | |
| 111003 Atopic | 5.3 | 104696 (BA) OA | 24.1 |
| Asthma-F | | Synovium-Backus | |
| 111004 Atopic | 22.7 | 104700 (SS) OA Bone- | 0.0 |
| Asthma-F | | Backus | |
| 111005 Atopic | 7.6 | 104701 (SS) Adj | 8.1 |
| Asthma-F | | “Normal⇄ Bone-Backus | |
| 111006 Atopic | 0.0 | 104702 (SS) OA | 6.7 |
| Asthma-F | | Synovium-Backus | |
| 111417 Allergy-M | 6.1 | 117093 OA Cartilage | 55.1 |
| | | Rep7 | |
| 112347 Allergy-M | 0.0 | 112672 OA Bone5 | 25.0 |
| 112349 Normal Lung-F | 0.0 | 112673 OA Synovium5 | 8.0 |
| 112357 Normal Lung-F | 9.4 | 112674 OA Synovial | 2.6 |
| | | Fluid cells5 | |
| 112354 Normal Lung-M | 8.5 | 117100 OA Cartilage | 0.0 |
| | | Rep14 | |
| 112374 Crohns-F | 27.0 | 112756 OA Bone9 | 15.2 |
| 112389 Match | 32.5 | 112757 OA Synovium9 | 4.0 |
| Control Crohns-F | | | |
| 112375 Crohns-F | 11.3 | 112758 OA Synovial | 3.4 |
| | | Fluid Cells9 | |
| 112732 Match | 20.9 | 117125 RA Cartilage | 3.8 |
| Control Crohns-F | | Rep2 | |
| 112725 Crohns-M | 17.8 | 113492 Bone2 RA | 6.0 |
| 112387 Match | 0.0 | 113493 Synovium2 RA | 2.1 |
| Control Crohns-M | | | |
| 112378 Crohns-M | 0.7 | 113494 Syn Fluid Cells | 0.0 |
| | | RA | |
| 112390 Match | 33.7 | 113499 Cartilage4 RA | 2.8 |
| Control Crohns-M | | | |
| 112726 Crohns-M | 12.2 | 113500 Bone4 RA | 0.0 |
| 112731 Match | 0.0 | 113501 Synovium4 RA | 4.8 |
| Control Crohns-M | | | |
| 112380 Ulcer Col-F | 39.8 | 113502 Syn Fluid | 8.1 |
| | | Cells4 RA | |
| 112734 Match | 37.9 | 113495 Cartilage3 RA | 0.0 |
| Control Ulcer Col-F | | | |
| 112384 Ulcer Col-F | 19.5 | 113496 Bone3 RA | 0.0 |
| 112737 Match | 2.7 | 113497 Synovium3 RA | 2.8 |
| Control Ulcer Col-F | | | |
| 112386 Ulcer Col-F | 12.9 | 113498 Syn Fluid | 2.9 |
| | | Cells3 RA | |
| 112738 Match | 0.0 | 117106 Normal | 2.4 |
| Control Ulcer Col-F | | Cartilage Rep20 | |
| 112381 Ulcer Col-M | 2.2 | 113663 Bone3 Normal | 7.5 |
| 112735 Match | 52.5 | 113664 Synovium3 | 0.0 |
| Control Ulcer Col-M | | Normal | |
| 112382 Ulcer Col-M | 88.9 | 113665 Syn Fluid | 5.5 |
| | | Cells3 Normal | |
| 112394 Match | 4.2 | 117107 Normal | 43.5 |
| Control Ulcer Col-M | | Cartilage Rep22 | |
| 112383 Ulcer Col-M | 14.6 | 113667 Bone4 Normal | 2.8 |
| 112736 Match | 3.0 | 113668 Synovium4 | 8.1 |
| Control Ulcer Col-M | | Normal | |
| 112423 Psoriasis-F | 2.3 | 113669 Syn Fluid | 17.0 |
| | | Cells4 Normal | |

TABLE 43
CNS_neurodegeneration_v1.0

| | Rel. Exp. (%) Ag2451, | | Rel. Exp. (%) Ag2451, |
|---|---|---|---|
| Tissue Name | Run 206915548 | Tissue Name | Run 206915548 |
| AD 1 Hippo | 7.1 | Control (Path) 3 | 2.8 |
| | | Temporal Ctx | |
| AD 2 Hippo | 21.8 | Control (Path) 4 | 66.9 |
| | | Temporal Ctx | |
| AD 3 Hippo | 4.4 | AD 1 Occipital Ctx | 15.1 |
| AD 4 Hippo | 18.6 | AD 2 Occipital Ctx | 0.0 |
| | | (Missing) | |
| AD 5 Hippo | 62.0 | AD 3 Occipital Ctx | 1.2 |
| AD 6 Hippo | 31.6 | AD 4 Occipital Ctx | 74.2 |
| Control 2 Hippo | 14.5 | AD 5 Occipital Ctx | 11.1 |
| Control 4 Hippo | 11.7 | AD 6 Occipital Ctx | 17.3 |
| Control (Path) 3 | 1.3 | Control 1 Occipital | 1.4 |
| Hippo | | Ctx | |
| AD 1 Temporal Ctx | 21.0 | Control 2 Occipital | 16.6 |
| | | Ctx | |
| AD 2 Temporal Ctx | 22.4 | Control 3 Occipital | 30.8 |
| | | Ctx | |
| AD 3 Temporal Ctx | 2.9 | Control 4 Occipital | 11.7 |
| | | Ctx | |
| AD 4 Temporal Ctx | 77.9 | Control (Path) 1 | 100.0 |
| | | Occipital Ctx | |
| AD 5 Inf Temporal | 26.4 | Control (Path) 2 | 22.4 |
| Ctx | | Occpital Ctx | |
| AD 5 Sup Temporal | 23.2 | Control (Path) 3 | 0.0 |
| Ctx | | Occipital Ctx | |
| AD 6 Inf Temporal | 56.3 | Control (Path) 4 | 38.7 |
| Ctx | | Occipital Ctx | |
| AD 6 Sub Temporal | 48.3 | Control 1 Parietal | 3.2 |
| Ctx | | Ctx | |
| Control 1 Temporal | 5.4 | Control 2 Parietal | 15.7 |
| Ctx | | Ctx | |
| Control 2 Temporal | 8.1 | Control 3 Parietal | 11.0 |
| Ctx | | Ctx | |
| Control 3 Temporal | 10.7 | Control (Path) 1 | 87.1 |
| Ctx | | Parietal Ctx | |
| Control 3 Temporal | 10.9 | Control (Path) 2 | 15.0 |
| Ctx | | Parietal Ctx | |
| Control (Path) 1 | 59.5 | Control (Path) 3 | 2.6 |
| Temporal Ctx | | Parietal Ctx | |
| Control (Path) 2 | 45.4 | Control (Path) 4 | 80.1 |
| Temporal Ctx | | Parietal Ctx | |

TABLE 44
Panel 1.3D

| | Rel. Exp. (%) | Rel. Exp. (%) | | Rel. Exp. (%) | Rel. Exp. (%) |
|---|---|---|---|---|---|
| | Ag2451, Run | Ag2451, Run | | Ag2451, Run | Ag2451, Run |
| Tissue Name | 159906491 | 165518159 | Tissue Name | 159906491 | 165518159 |

| | | | | | |
|---|---|---|---|---|---|
| Liver | 4.4 | 0.0 | Kidney (fetal) | 5.1 | 0.0 |
| adenocarcinoma | | | | | |
| Pancreas | 0.0 | 0.0 | Renal ca. 786-0 | 0.0 | 0.0 |
| Pancreatic ca. | 4.5 | 0.0 | Renal ca. | 3.3 | 0.0 |
| CAPAN2 | | | A498 | | |
| Adrenal gland | 5.3 | 0.0 | Renal ca. RXF | 0.0 | 0.0 |
| | | | 393 | | |
| Thyroid | 2.3 | 0.0 | Renal ca. | 0.0 | 0.0 |
| | | | ACHN | | |
| Salivary gland | 0.0 | 0.0 | Renal ca. | 0.0 | 0.0 |
| | | | UO-31 | | |
| Pituitary gland | 9.0 | 0.0 | Renal ca. | 0.0 | 0.0 |
| | | | TK-10 | | |
| Brain (fetal) | 13.0 | 12.4 | Liver | 0.0 | 0.0 |
| Brain (whole) | 2.1 | 31.2 | Liver (fetal) | 2.1 | 0.0 |
| Brain (amygdala) | 14.3 | 39.8 | Liver ca. | 13.5 | 0.0 |
| | | | (hepatoblast) | | |
| | | | HepG2 | | |
| Brain | 4.7 | 0.0 | Lung | 7.4 | 0.0 |
| (cerebellum) | | | | | |
| Brain | 18.4 | 16.4 | Lung (fetal) | 0.0 | 0.0 |
| (hippocampus) | | | | | |
| Brain | 0.0 | 0.0 | Lung ca. | 0.0 | 0.0 |
| (substantianigra) | | | (small cell) | | |
| | | | LX-1 | | |
| Brain (thalamus) | 15.4 | 42.6 | Lung ca. | 3.7 | 0.0 |
| | | | (small cell) | | |
| | | | NCI-H69 | | |
| Cerebral Cortex | 68.8 | 66.0 | Lung ca. | 39.2 | 24.7 |
| | | | (s.cell var.) | | |
| | | | SHP-77 | | |
| Spinal cord | 6.5 | 0.0 | Lung ca. (large | 0.0 | 18.8 |
| | | | cell)NCI-H460 | | |
| glio/astro | 10.5 | 0.0 | Lung ca. (non- | 0.0 | 0.0 |
| U87-MG | | | sm. cell) A549 | | |
| glio/astro | 0.0 | 0.0 | Lung ca. (non- | 3.9 | 0.0 |
| U-118-MG | | | s.cell) | | |
| | | | NCI-H23 | | |
| astrocytoma | 0.0 | 22.8 | Lung ca. (non- | 6.6 | 14.9 |
| SW1783 | | | s.cell) HOP-62 | | |
| neuro*; met | 1.6 | 0.0 | Lung ca. (non- | 0.0 | 0.0 |
| SK-N-AS | | | s.cl) NCI- | | |
| | | | H522 | | |
| astrocytoma | 0.0 | 0.0 | Lung ca. | 2.3 | 13.7 |
| SF-539 | | | (squam.) SW | | |
| | | | 900 | | |
| astrocytoma | 6.2 | 0.0 | Lung ca. | 0.0 | 0.0 |
| SNB-75 | | | (squam.) NCI- | | |
| | | | H596 | | |
| glioma SNB-19 | 21.9 | 72.7 | Mammary | 0.0 | 0.0 |
| | | | gland | | |
| glioma U251 | 3.1 | 63.3 | Breast ca.* | 2.4 | 0.0 |
| | | | (pl.ef) MCF-7 | | |
| glioma SF-295 | 0.0 | 0.0 | Breast ca.* | 9.3 | 0.0 |
| | | | (pl.ef) MDA- | | |
| | | | MB-231 | | |
| Heart (fetal) | 0.0 | 0.0 | Breast ca.* | 0.0 | 0.0 |
| | | | (pl.ef) T47D | | |
| Heart | 0.0 | 0.0 | Breast ca. BT- | 17.2 | 0.0 |
| | | | 549 | | |
| Skeletal muscle | 13.8 | 0.0 | Breast ca. | 0.0 | 23.2 |
| (fetal) | | | MDA-N | | |
| Skeletal muscle | 0.0 | 0.0 | Ovary | 3.8 | 0.0 |
| Bone marrow | 4.6 | 0.0 | Ovarian ca. | 5.5 | 0.0 |
| | | | OVCAR-3 | | |
| Thymus | 2.7 | 0.0 | Ovarian ca. | 0.0 | 0.0 |
| | | | OVCAR-4 | | |
| Spleen | 0.0 | 0.0 | Ovarian ca. | 5.2 | 0.0 |
| | | | OVCAR-5 | | |
| Lymph node | 0.0 | 0.0 | Ovarian ca. | 4.8 | 0.0 |
| | | | OVCAR-8 | | |
| Colorectal | 8.6 | 0.0 | Ovarian ca. | 3.8 | 0.0 |
| | | | IGROV-1 | | |
| Stomach | 3.9 | 15.0 | Ovarian ca.* | 3.9 | 4.7 |
| | | | (ascites) SK- | | |
| | | | OV-3 | | |
| Small intestine | 15.1 | 0.0 | Uterus | 0.0 | 15.7 |
| Colon ca. SW480 | 0.0 | 0.0 | Plancenta | 6.8 | 0.0 |
| Colon ca.* | 0.0 | 0.0 | Prostate | 0.0 | 28.1 |
| SW620(SW480 | | | | | |
| met) | | | | | |
| Colon ca. HT29 | 3.0 | 0.0 | Prostate ca.* | 0.0 | 0.0 |
| | | | (bone met) | | |
| | | | PC-3 | | |
| Colon ca. HCT- | 0.0 | 31.4 | Testis | 41.2 | 25.9 |
| 116 | | | | | |
| Colon ca. CaCo-2 | 0.0 | 0.0 | Melanoma | 0.0 | 0.0 |
| | | | Hs688(A).T | | |
| Colon ca. | 0.0 | 14.4 | Melanoma* | 0.0 | 0.0 |
| tissue(ODO3866) | | | (met) | | |
| | | | Hs688(B).T | | |
| Colon ca. HCC- | 0.0 | 0.0 | Melanoma | 0.0 | 0.0 |
| 2998 | | | UACC-62 | | |
| Gastric ca.* (liver | 100.0 | 100.0 | Melanoma | 0.0 | 0.0 |
| met) NCI-N87 | | | M14 | | |
| Bladder | 3.3 | 10.0 | Melanoma | 0.0 | 0.0 |
| | | | LOX IMVI | | |
| Trachea | 9.0 | 0.0 | Melanoma* | 0.0 | 0.0 |
| | | | (met) SK- | | |
| | | | MEL-5 | | |
| Kidney | 4.6 | 0.0 | Adipose | 0.0 | 0.0 |

TABLE 45
Panel 2D

| | Rel. Exp. (%) | Rel. Exp. (%) | | Rel. Exp. (%) | Rel. Exp. (%) |
|---|---|---|---|---|---|
| | Ag2451, Run | Ag2451, Run | | Ag2451, Run | Ag2451, Run |
| Tissue Name | 159906936 | 164977397 | Tissue Name | 159906936 | 164977397 |
| Normal Colon | 0.0 | 14.1 | Kidney | 0.0 | 0.0 |
| | | | Margin | | |
| | | | 8120608 | | |
| CC Well to Mod | 4.0 | 2.9 | Kidney Cancer | 3.8 | 0.0 |
| Diff (ODO3866) | | | 8120613 | | |
| CC Margin | 0.0 | 0.0 | Kidney | 0.0 | 1.2 |
| (ODO3866) | | | Margin | | |
| | | | 8120614 | | |
| CC Gr.2 | 0.0 | 0.0 | Kidney Cancer | 0.0 | 8.2 |
| rectosigmoid | | | 9010320 | | |
| (ODO3868) | | | | | |
| CC Margin | 1.9 | 5.1 | Kidney | 1.6 | 2.6 |
| (ODO3868) | | | Margin | | |
| | | | 9010321 | | |
| CC Mod Diff | 0.0 | 5.9 | Normal Uterus | 0.0 | 6.0 |
| (ODO3920) | | | | | |
| CC Margin | 4.3 | 11.1 | Uterus Cancer | 22.5 | 40.6 |
| (ODO3920) | | | 064011 | | |
| CC Gr.2 ascend | 5.3 | 6.0 | Normal | 0.0 | 0.0 |
| colon | | | Thyroid | | |
| (ODO3921) | | | | | |
| CC Margin | 20.9 | 2.9 | Thyroid | 1.8 | 0.0 |
| (ODO3921) | | | Cancer | | |
| | | | 064010 | | |
| CC from Partial | 3.3 | 1.1 | Thyroid | 6.6 | 7.8 |
| Hepatectomy | | | Cancer | | |
| (ODO4309) | | | A302152 | | |
| Mets | | | | | |
| Liver Margin | 2.2 | 0.0 | Thyroid | 6.4 | 0.0 |
| (ODO4309) | | | Margin | | |
| | | | A302153 | | |
| Colon mets to | 2.3 | 2.9 | Normal Breast | 9.2 | 7.7 |
| lung (OD04451- | | | | | |
| 01) | | | | | |
| Lung Margin | 0.0 | 2.8 | Breast Cancer | 0.0 | 2.7 |
| (OD04451-02) | | | (OD04566) | | |
| Normal Prostate | 0.0 | 12.0 | Breast Cancer | 5.1 | 2.4 |
| 6546-1 | | | (OD04590-01) | | |
| Prostate Cancer | 26.1 | 21.3 | Breast Cancer | 5.8 | 6.2 |
| (OD04410) | | | Mets | | |
| | | | (OD04590-03) | | |
| Prostate Margin | 6.6 | 21.3 | Breast Cancer | 30.4 | 29.3 |
| (OD04410) | | | Metastasis | | |
| | | | (OD04655-05) | | |
| Prostate Cancer | 2.1 | 7.9 | Breast Cancer | 31.6 | 11.0 |
| (OD04720-01) | | | 064006 | | |
| Prostate Margin | 0.0 | 5.7 | Breast Cancer | 20.6 | 32.1 |
| (OD04720-02) | | | 1024 | | |
| Normal Lung | 12.9 | 13.4 | Breast Cancer | 2.2 | 0.0 |
| 061010 | | | 9100266 | | |
| Lung Met to | 5.0 | 1.0 | Breast Margin | 0.0 | 0.0 |
| Muscle | | | 9100265 | | |
| (ODO4286) | | | | | |
| Muscle Margin | 0.9 | 0.0 | Breast Cancer | 10.5 | 17.4 |
| (ODO4286) | | | A209073 | | |
| Lung Malignant | 4.6 | 0.0 | Breast Margin | 12.9 | 7.4 |
| Cancer | | | A2090734 | | |
| (OD03126) | | | | | |
| Lung Margin | 4.7 | 4.3 | Normal Liver | 2.2 | 0.0 |
| (OD03126) | | | | | |
| Lung Cancer | 24.1 | 11.8 | Liver Cancer | 1.6 | 2.9 |
| (OD04404) | | | 064003 | | |
| Lung Margin | 1.4 | 1.4 | Liver Cancer | 0.0 | 0.0 |
| (OD04404) | | | 1025 | | |
| Lung Cancer | 8.5 | 11.1 | Liver Cancer | 0.0 | 0.0 |
| (OD04565) | | | 1026 | | |
| Lung Margin | 0.0 | 3.1 | Liver Cancer | 0.0 | 5.6 |
| (OD04565) | | | 6004-T | | |
| Lung Cancer | 33.4 | 54.7 | Liver Tissue | 4.0 | 0.0 |
| (OD04237-01) | | | 6004-N | | |
| Lung Margin | 6.9 | 2.9 | Liver Cancer | 0.0 | 2.9 |
| (OD04237-02) | | | 6005-T | | |
| Ocular Mel Met | 0.0 | 0.0 | Liver Tissue | 0.0 | 0.0 |
| to Liver | | | 6005-N | | |
| (ODO4310) | | | | | |
| Liver Margin | 3.8 | 0.0 | Normal | 8.4 | 11.7 |
| (ODO4310) | | | Bladder | | |
| Melanoma Mets | 1.9 | 0.0 | Bladder | 0.0 | 0.0 |
| to Lung | | | Cancer 1023 | | |
| (OD04321) | | | | | |
| Lung Margin | 2.7 | 0.0 | Bladder | 69.7 | 95.9 |
| (OD04321) | | | Cancer | | |
| | | | A302173 | | |
| Normal Kidney | 10.2 | 21.9 | Bladder | 2.8 | 1.4 |
| | | | Cancer | | |
| | | | (OD04718-01) | | |
| Kidney Ca, | 26.4 | 70.2 | Bladder | 2.2 | 5.3 |
| Nuclear grade 2 | | | Normal | | |
| (OD04338) | | | Adjacent | | |
| | | | (OD04718-03) | | |
| Kidney Margin | 12.7 | 11.6 | Normal Ovary | 3.6 | 0.0 |
| (OD04338) | | | | | |
| Kidney Ca | 22.4 | 13.7 | Ovarian | 0.0 | 0.0 |
| Nuclear grade | | | Cancer | | |
| 1/2 (OD04339) | | | 064008 | | |
| Kidney Margin | 9.9 | 8.1 | Ovarian | 0.0 | 0.0 |
| (OD04339) | | | Cancer | | |
| | | | (OD04768-07) | | |
| Kidney Ca, Clear | 100.0 | 27.5 | Ovary Margin | 0.0 | 0.0 |
| cell type | | | (OD04768-08) | | |
| (OD04340) | | | | | |
| Kidney Margin | 27.5 | 31.0 | Normal | 3.2 | 12.6 |
| (OD04340) | | | Stomach | | |
| Kidney Ca, | 2.1 | 3.2 | Gastric Cancer | 0.0 | 0.0 |
| Nuclear grade 3 | | | 9060358 | | |
| (OD04348) | | | | | |
| Kidney Margin | 3.1 | 8.7 | Stomach | 2.4 | 0.0 |
| (OD04348) | | | Margin | | |
| | | | 9060359 | | |
| Kidney Cancer | 0.0 | 10.6 | Gastric Cancer | 0.0 | 0.0 |
| (OD04622-01) | | | 9060395 | | |
| Kidney Margin | 1.5 | 2.8 | Stomach | 0.0 | 0.0 |
| (OD04622-03) | | | Margin | | |
| | | | 9060394 | | |
| Kidney Cancer | 76.8 | 100.0 | Gastric Cancer | 0.0 | 0.0 |
| (OD04450-01) | | | 9060397 | | |
| Kidney Margin | 13.7 | 36.1 | Stomach | 0.0 | 0.0 |
| (OD04450-03) | | | Margin | | |
| | | | 9060396 | | |
| Kidney Cancer | 0.0 | 0.0 | Gastric Cancer | 18.2 | 11.5 |
| 8120607 | | | 064005 | | |

TABLE 46
Panel 4D

| | Rel. Exp. (%) | Rel. Exp. (%) | Rel. Exp. (%) | | Rel. Exp. (%) | Rel. Exp. (%) | Rel. Exp. (%) |
|---|---|---|---|---|---|---|---|
| | Ag1455, Run | Ag2451, Run | Ag2451, Run | | Ag1455, Run | Ag2451, Run | Ag2451, Run |
| Tissue Name | 162599079 | 159906953 | 161683394 | Tissue Name | 162599079 | 159906953 | 161683394 |
| Secondary Th1 | 0.0 | 0.0 | 0.0 | HUVEC IL- | 0.0 | 0.0 | 0.0 |
| act | | | | 1beta | | | |
| Secondary Th2 | 0.0 | 6.7 | 9.9 | HUVEC IFN | 0.0 | 0.0 | 0.0 |
| act | | | | gamma | | | |
| Secondary Tr1 | 0.0 | 12.0 | 0.0 | HUVEC TNF | 0.0 | 0.0 | 0.0 |
| act | | | | alpha + IFN | | | |
| | | | | gamma | | | |
| Secondary Th1 | 0.0 | 0.0 | 0.0 | HUVEC TNF | 0.0 | 0.0 | 0.0 |
| rest | | | | alpha + IL4 | | | |
| Secondary Th2 | 0.0 | 0.0 | 0.0 | HUVEC IL-11 | 0.0 | 0.0 | 0.0 |
| rest | | | | | | | |
| Secondary Tr1 | 0.0 | 4.2 | 2.5 | Lung | 0.0 | 7.6 | 13.3 |
| rest | | | | Microvascular | | | |
| | | | | EC none | | | |
| Primary Th1 | 0.0 | 0.0 | 0.0 | Lung | 0.0 | 0.0 | 0.0 |
| act | | | | Microvascular | | | |
| | | | | EC TNFalpha | | | |
| | | | | + IL-1beta | | | |
| Primary Th2 | 0.0 | 0.0 | 0.0 | Microvascular | 0.0 | 0.0 | 0.0 |
| act | | | | Dermal EC | | | |
| | | | | none | | | |
| Primary Tr1 | 0.0 | 0.0 | 0.0 | Microvascular | 0.0 | 0.0 | 0.0 |
| act | | | | Dermal EC | | | |
| | | | | TNFalpha + | | | |
| | | | | IL-1beta | | | |
| Primary Th1 | 0.0 | 4.5 | 4.3 | Bronchial | 1.1 | 0.0 | 0.0 |
| rest | | | | epithelium | | | |
| | | | | TNFalpha + | | | |
| | | | | IL1beta | | | |
| Primary Th2 | 0.0 | 4.2 | 2.5 | Small airway | 13.8 | 17.7 | 3.5 |
| rest | | | | epithelium | | | |
| | | | | none | | | |
| Primary Tr1 | 0.0 | 7.2 | 11.3 | Small airway | 100.0 | 100.0 | 100.0 |
| rest | | | | epithelium | | | |
| | | | | TNFalpha + | | | |
| | | | | IL-1beta | | | |
| CD45RA CD4 | 0.0 | 0.0 | 0.0 | Coronery | 0.0 | 0.0 | 0.0 |
| lymphocyte act | | | | artery SMC | | | |
| | | | | rest | | | |
| CD45RO CD4 | 0.0 | 0.0 | 0.0 | Coronery | 0.0 | 1.6 | 0.0 |
| lymphocyte act | | | | artery SMC | | | |
| | | | | TNFalpha + | | | |
| | | | | IL-1beta | | | |
| CD8 | 0.0 | 0.0 | 0.0 | Astrocytes rest | 0.0 | 0.0 | 3.3 |
| lymphocyte act | | | | | | | |
| Secondary CD8 | 0.0 | 0.0 | 0.0 | Astrocytes | 0.0 | 3.2 | 0.0 |
| lymphocyte rest | | | | TNFalpha + | | | |
| | | | | IL-1beta | | | |
| Secondary CD8 | 0.0 | 0.0 | 0.0 | KU-812 | 7.4 | 8.0 | 15.4 |
| lymphocyte act | | | | (Basophil) rest | | | |
| CD4 | 0.0 | 0.0 | 0.0 | KU-812 | 34.4 | 17.1 | 19.5 |
| lymphocyte | | | | (Basophil) | | | |
| none | | | | PMA/ionomyc | | | |
| | | | | in | | | |
| 2ry | 0.0 | 0.0 | 0.4 | CCD1106 | 1.2 | 0.0 | 0.0 |
| Th1/Th2/Tr1_{—} | | | | (Keratinocytes) | | | |
| anti-CD95 CH11 | | | | none | | | |
| LAK cells rest | 0.0 | 0.0 | 0.0 | CCD1106 | 0.0 | 0.0 | 0.0 |
| | | | | (Keratinocytes) | | | |
| | | | | TNFalpha + | | | |
| | | | | IL-1beta | | | |
| LAK cells IL-2 | 0.0 | 0.0 | 0.0 | Liver cirrhosis | 0.0 | 19.1 | 6.4 |
| LAK cells IL-2 + | 0.0 | 0.0 | 0.0 | Lupus kidney | 0.0 | 17.3 | 8.1 |
| IL-12 | | | | | | | |
| LAK cells IL-2 + | 0.0 | 8.8 | 0.0 | NCI-H292 | 0.0 | 31.2 | 20.3 |
| IFN gamma | | | | none | | | |
| LAK cells IL-2 + | 0.0 | 0.0 | 0.0 | NCI-H292 IL-4 | 1.3 | 26.4 | 11.0 |
| IL-18 | | | | | | | |
| LAK cells | 0.0 | 0.0 | 0.0 | NCI-H292 IL-9 | 0.0 | 28.9 | 17.9 |
| PMA/ionomycin | | | | | | | |
| NK Cells IL-2 | 0.0 | 0.0 | 6.3 | NCI-H292 IL- | 0.0 | 3.8 | 5.1 |
| rest | | | | 13 | | | |
| Two Way MLR | 0.0 | 8.7 | 3.3 | NCI-H292 | 0.0 | 11.6 | 1.6 |
| 3 day | | | | IFN gamma | | | |
| Two Way MLR | 0.0 | 0.0 | 0.0 | HPAEC none | 0.0 | 0.0 | 0.0 |
| 5 day | | | | | | | |
| Two Way MLR | 0.0 | 0.0 | 0.0 | HPAEC TNF | 0.0 | 4.2 | 0.0 |
| 7 day | | | | alpha + IL-1 | | | |
| | | | | beta | | | |
| PBMC rest | 0.0 | 0.0 | 0.0 | Lung | 0.0 | 0.0 | 3.7 |
| | | | | fibroblast | | | |
| | | | | none | | | |
| PBMC PWM | 0.0 | 0.0 | 5.8 | Lung | 0.0 | 0.0 | 3.6 |
| | | | | fibroblast TNF | | | |
| | | | | alpha + IL-1 | | | |
| | | | | beta | | | |
| PBMC PHA-L | 0.0 | 4.0 | 3.5 | Lung | 0.0 | 8.4 | 0.0 |
| | | | | fibroblast IL-4 | | | |
| Ramos (B cell) | 0.0 | 0.0 | 0.0 | Lung | 0.0 | 0.0 | 2.7 |
| none | | | | fibroblast IL-9 | | | |
| Ramos (B cell) | 0.0 | 0.0 | 0.0 | Lung | 0.0 | 4.3 | 0.0 |
| ionomycin | | | | fibroblast IL- | | | |
| | | | | 13 | | | |
| B lymphocytes | 0.0 | 0.0 | 0.0 | Lung | 0.0 | 5.9 | 2.2 |
| PWM | | | | fibroblast IFN | | | |
| | | | | gamma | | | |
| B lymphocytes | 0.0 | 0.0 | 2.5 | Dermal | 0.0 | 0.0 | 0.0 |
| CD40L and IL-4 | | | | fibroblast | | | |
| | | | | CCD1070 rest | | | |
| EOL-1 dbcAMP | 0.0 | 0.0 | 0.0 | Dermal | 0.0 | 2.6 | 6.0 |
| | | | | fibroblast | | | |
| | | | | CCD1070 | | | |
| | | | | TNF alpha | | | |
| EOL-1 dbcAMP | 0.0 | 0.0 | 0.0 | Dermal | 0.0 | 0.0 | 0.0 |
| PMA/ionomycin | | | | fibroblast | | | |
| | | | | CCD1070 IL- | | | |
| | | | | 1 beta | | | |
| Dendritic cells | 0.0 | 0.0 | 0.0 | Dermal | 0.0 | 6.7 | 6.9 |
| none | | | | fibroblast IFN | | | |
| | | | | gamma | | | |
| Dendritic cells | 0.0 | 2.2 | 0.0 | Dermal | 0.0 | 0.0 | 7.6 |
| LPS | | | | fibroblast IL-4 | | | |
| Dentritic cells | 0.0 | 4.4 | 4.2 | IBD Colitis 2 | 0.0 | 12.3 | 3.9 |
| anti-CD40 | | | | | | | |
| Monocytes rest | 0.0 | 0.0 | 7.6 | IBD Crohn's | 0.0 | 0.0 | 2.5 |
| Monocytes LPS | 0.0 | 0.0 | 0.0 | Colon | 0.0 | 4.9 | 0.0 |
| Macrophages | 0.0 | 0.0 | 6.7 | Lung | 0.0 | 8.5 | 7.5 |
| rest | | | | | | | |
| Macrophages | 0.0 | 0.0 | 0.0 | Thymus | 0.0 | 8.5 | 6.4 |
| LPS | | | | | | | |
| HUVEC none | 0.0 | 0.0 | 3.6 | Kidney | 0.6 | 0.9 | 15.1 |
| HUVEC starved | 0.0 | 2.3 | 0.0 | | | | |

AI_comprehensive panel_v1.0 Summary: Ag2451 The NOV1A transcript is expressed in tissue from patients with ulcerative colitis and Crohn's disease in both the disease tissue itself and in the matched tissue which may itself be inflammed. There is also some low expression of this transcript in chronic obstructive pulmonary disorder (COPD) and asthmatic lung tissue. This expression profile is consistent with panel 4D and supports the idea that therapeutics designed to modulate the function of the protein encoded by this gene could block or inhibit inflammation or tissue damage due to lung conditions including asthma, allergies, hypersensitivity reactions, and viral infections. Thus therapeutic modulation of this gene product may reduce or eliminate the symptoms of patients suffering from Crohn's disease and ulcerative colitis. A second experiment with the same probe and primer set is not included because of a problem in one of the sample wells.

CNS_neurodegeneration_v1.0 Summary: Ag2451 Low expression of the NOV1A gene in the brain suggests a potential role for this gene product in CNS processes. However, this panel does not show any association between expression of this gene and Alzheimer's disease.

Panel 1.3D Summary: Ag2451 Two experiments with the same probe and primer set both show expression of the NOVIA gene to be restricted to a sample from the gastric cancer cell line NCI-N87 (CTs=32.8-34.7). Thus, the expression of this gene could be used to distinguish this gastric cancer sample from other samples in the panel. Moreover, therapeutic modulation of this gene, through the use of small molecule drugs, antibodies or protein therapeutics might be of benefit in the treatment of gastric cancer. A third experiment with the probe and primer set Ag1455 shows low/undetectable levels of expression in all the samples on this panel (CTs>35). (Data not shown.)

Panel 2D Summary: Ag2451 Two experiments with the same probe and primer set show highest expression of the NOV IA gene in samples derived from kidney cancer (CTs=31-32). In addition, a number of kidney cancers as well as lung cancer and a bladder cancer show expression of this gene. Thus, the expression of this gene could be used to distinguish those samples listed above from others in the panel. Moreover, therapeutic modulation of this gene, through the use of small molecule drugs, antibodies or protein therapeutics might be of benefit in the treatment of kidney, lung or bladder cancer.

Panel 3D Summary: Ag2451 A single experiment with the NOVIA gene shows low/undetectable levels of expression in all the samples on this panel (CTs>35). (Data not shown.)

Panel 4D Summary: Ag1455/Ag2451 Highest expression of the NOV1A gene is detected in small airway epithelium treated with TNFalpha+IL-1beta. Low but significant expression of this gene is also detected in the basophil cell line KU-812 treated with PMA/ionomycin. Expression in lung derived cells suggests that the protein encoed by this gene may be involved in lung disorders including asthma, allergies, chronic obstructive pulmonary disease, and emphysema. Since basophils play an important role in lung pathology and since this transcript is present in lung derived cells, therapeutics designed to modulate the function of the protein encoded by this gene may block or inhibit inflammation or tissue damage due to lung conditions including asthma, allergies, hypersensitivity reactions, and viral infections. In addition, the KU-812 cell line is a reasonable model for the inflammatory cells that take part in various bowel diseases, such as Crohn's disease, and ulcerative colitis. Therefore, therapeutics that modulate the function of this gene product may reduce or eliminate the symptoms of patients suffering from Crohn's disease and ulcerative colitis.

NOV12

Expression of gene NOV12 was assessed using the primer-probe sets Ag1489, Ag3032, Ag4335, Ag317, and Ag674, described in Tables 47, 48, 49, 50, and 51. Results of the RTQ-PCR runs are shown in Tables 52, 53, 54, 55, 56, 57, 58, 59, 60, and 61.

TABLE 47

Probe Name Ag1489

| | | | | |
|---|---|---|---|---|
| Primers | Sequences | Length | Start Positon | SEQ ID NO: |

| | | | | |
|---|---|---|---|---|
| Forward | 5′-cacagcattgaattgctctgt-3′ | 21 | 7049 | 199 |
| Probe | TET-5′-tctggattttaccaaatggcacacga-3′-TAMRA | 26 | 7096 | 200 |
| Reverse | 5′gataactttgtggtccattgga-3′ | 22 | 7125 | 201 |

TABLE 48

Probe Name Ag3032

| | | | | |
|---|---|---|---|---|
| Primers | Sequences | Length | Start Position | SEQ ID NO: |

| | | | | |
|---|---|---|---|---|
| Forward | 5′-cacctgaagaacccacagatt-3′ | 21 | 2593 | 202 |
| Probe | TET-5′-tgctattaaaactacagccatgtcaaaga-3′-TAMRA | 2627 | 203 | |
| Reverse | 5′-gcttgacatggttgggtttat-3′21 | 2658 | 204 | |

TABLE 49

Probe Name Ag4335

| | | | | |
|---|---|---|---|---|
| Primers | Sequences | Length | Start Position | SEQ ID NO: |

| | | | | |
|---|---|---|---|---|
| Forward | 5′-cacctgaagaacccacagatt-3′ | 21 | 2593 | 202 |
| Probe | TET-5′-tgctattaaaactacagccatgtcaaaga-3′-TAMRA | 2627 | 203 | |
| Reverse | 5′-gcttgacatggttgggtttat-3′21 | 2658 | 204 | |

TABLE 50

Probe Name Ag317

| | | | | |
|---|---|---|---|---|
| Primers | Sequences | Length | Start Position | SEQ ID NO: |

| | | | | |
|---|---|---|---|---|
| Forward | 5′-ggatttcgggctacacatacg-3′ | 21 | 6620 | 208 |
| Probe | TET-5 -actctccagaatcgagcagtttcactttgttg-3′-TAMRA | 32 | 6587 209 | |
| Reverse | 5′-atgccaatgggtctttgacc-3′ | 20 | 6565 | 210 |

TABLE 51

Probe Name Ag674

| | | | | |
|---|---|---|---|---|
| Primers | Sequences | Length | Start Position | SEQ ID NO: |

| | | | | |
|---|---|---|---|---|
| Forward | 5′gtcatccccactgggattt-3′ | 19 | 6635 | 211 |
| Probe | TET-5′-cgtactctccagaatcgagcagtttca-3′-TAMRA | 27 | 6595 | 212 |
| Reverse | 5-caatgggtctttgaccatca-3′ | 20 | 6569 | 213 |

TABLE 52
AI_comprehensive panel_v1.0

| | Rel. Exp. (%) Ag1489, | | Rel. Exp. (%) Ag1489, |
|---|---|---|---|
| Tissue Name | Run 226203351 | Tissue Name | Run 226203351 |
| 110967 COPD-F | 1.8 | 112427 Match Control | 28.5 |
| | | Psoriasis-F | |
| 110980 COPD-F | 12.1 | 112418 Psoriasis-M | 2.1 |
| 110968 COPD-M | 2.7 | 112723 Match Control | 6.9 |
| | | Psoriasis-M | |
| 110977 COPD-M | 0.0 | 112419 Psoriasis-M | 4.2 |
| 110989 Emphysema-F | 4.9 | 112424 Match Control | 4.4 |
| | | Psoriasis-M | |
| 110992 Emphysema-F | 5.7 | 112420 Psoriasis-M | 5.4 |
| 110993 Emphysema-F | 3.3 | 112425 Match Control | 33.2 |
| | | Psoriasis-M | |
| 110994 Emphysema-F | 1.7 | 104689 (MF) OA | 9.0 |
| | | Bone-Backus | |
| 110995 Emphysema-F | 5.2 | 104690 (MF) Adj | 8.1 |
| | | “Normal” Bone-Backus | |
| 110996 Emphysema-F | 3.1 | 104691 (MF) OA | 54.0 |
| | | Synovium-Backus | |
| 110997 Asthma-M | 0.5 | 104692 (BA) OA | 0.3 |
| | | Cartilage-Backus | |
| 111001 Asthma-F | 2.2 | 104694 (BA) OA | 20.3 |
| | | Bone-Backus | |
| 111002 Asthma-F | 2.3 | 104695 (BA) Adj | 3.1 |
| | | “Normal” Bone-Backus | |
| 111003 Atopic | 2.3 | 104696 (BA) OA | 26.4 |
| Asthma-F | | Synovium-Backus | |
| 111004 Atopic | 6.1 | 104700 (SS) OA Bone- | 2.8 |
| Asthma-F | | Backus | |
| 111005 Atopic | 2.6 | 104701 (SS) Adj | 8.6 |
| Asthma-F | | “Normal” Bone-Backus | |
| 111006 Atopic | 0.4 | 104702 (SS) OA | 14.7 |
| Asthma-F | | Synovium-Backus | |
| 111417 Allergy-M | 3.4 | 117093 OA Cartilage | 2.1 |
| | | Rep7 | |
| 112347 Allergy-M | 0.1 | 112672 OA Bone5 | 11.9 |
| 112349 Normal Lung-F | 0.2 | 112673 OA Synovium5 | 5.8 |
| 112357 Normal Lung-F | 100.0 | 112674 OA Synovial | 6.4 |
| | | Fluid cells5 | |
| 112354 Normal Lung-M | 59.0 | 117100 OA Cartilage | 0.7 |
| | | Rep14 | |
| 112374 Crohns-F | 7.3 | 112756 OA Bone9 | 2.8 |
| 112389 Match | 0.5 | 112757 OA Synovium9 | 0.8 |
| Control Crohns-F | | | |
| 112375 Crohns-F | 4.4 | 112758 OA Synovial | 6.1 |
| | | Fluid Cells9 | |
| 112732 Match | 6.1 | 117125 RA Cartilage | 3.4 |
| Control Crohns-M | | Rep2 | |
| 112725 Crohns-M | 0.9 | 113492 Bone2 RA | 40.3 |
| 112387 Match | 2.5 | 113493 Synovium2 RA | 15.8 |
| Control Crohns-M | | | |
| 112378 Crohns-M | 0.4 | 113494 Syn Fluid Cells | 25.2 |
| | | RA | |
| 112390 Match | 7.5 | 113499 Cartilage4 RA | 33.9 |
| Control Crohns-M | | | |
| 112726 Crohns-M | 2.3 | 113500 Bone4 RA | 42.6 |
| 112731 Match | 4.7 | 113501 Synovium4 RA | 26.6 |
| Control Crohns-M | | | |
| 112380 Ulcer Col-F | 2.4 | 113502 Syn Fluid | 20.0 |
| | | Cells4 RA | |
| 112734 Match | 24.1 | 113495 Cartilage3 RA | 27.2 |
| Control Ulcer Col-F | | | |
| 112384 Ulcer Col-F | 4.6 | 113496 Bone3 RA | 32.8 |
| 112737 Match | 1.6 | 113497 Synovium3 RA | 19.3 |
| Control Ulcer Col-F | | | |
| 112386 Ulcer Col-F | 1.7 | 113498 Syn Fluid | 39.5 |
| | | Cells3 RA | |
| 112738 Match | 3.2 | 117106 Normal | 0.0 |
| Control Ulcer Col-F | | Cartilage Rep20 | |
| 112381 Ulcer Col-M | 0.8 | 113663 Bone3 Normal | 0.7 |
| 112735 Match | 3.8 | 113664 Synovium3 | 0.1 |
| Control Ulcer Col-M | | Normal | |
| 112382 Ulcer Col-M | 1.0 | 113665 Syn Fluid | 0.3 |
| | | Cells3 Normal | |
| 112394 Match | 1.9 | 117107 Normal | 2.2 |
| Control Ulcer Col-M | | Cartilage Rep22 | |
| 112383 Ulcer Col-M | 4.0 | 113667 Bone4 Normal | 4.1 |
| 112736 Match | 0.6 | 113668 Synovium4 | 4.8 |
| Control Ulcer Col-M | | Normal | |
| 112423 Psoriasis-F | 8.9 | 113669 Syn Fluid | 5.8 |
| | | Cells4 Normal | |

TABLE 53
CNS_neurodegeneration_v1.0

| | Rel. Exp. (%) Ag4335, | | Rel. Exp. (%) Ag4335, |
|---|---|---|---|
| Tissue Name | Run 224348629 | Tissue Name | Run 224348629 |
| AD 1 Hippo | 38.4 | Control (Path) 3 | 5.9 |
| | | Temporal Ctx | |
| AD 2 Hippo | 87.7 | Control (Path) 4 | 37.4 |
| | | Temporal Ctx | |
| AD 3 Hippo | 26.6 | AD 1 Occipital Ctx | 20.3 |
| AD 4 Hippo | 7.4 | AD 2 Occipital Ctx | 0.0 |
| | | (Missing) | |
| AD 5 Hippo | 24.1 | AD 3 Occipital Ctx | 2.7 |
| AD 6 Hippo | 89.5 | AD 4 Occipital Ctx | 14.1 |
| Control 2 Hippo | 64.6 | AD 5 Occipital Ctx | 38.2 |
| Control 4 Hippo | 26.1 | AD 6 Occipital Ctx | 6.4 |
| Control (Path) 3 | 8.2 | Control 1 Occipital | 2.2 |
| Hippo | | Ctx | |
| AD 1 Temporal Ctx | 39.2 | Control 2 Occipital | 20.2 |
| | | Ctx | |
| AD 2 Temporal Ctx | 42.6 | Control 3 Occipital | 6.8 |
| | | Ctx | |
| AD 3 Temporal Ctx | 13.6 | Control 4 Occipital | 4.6 |
| | | Ctx | |
| AD 4 Temporal Ctx | 7.5 | Control (Path) 1 | 40.9 |
| | | Occipital Ctx | |
| AD 5 Inf Temporal | 27.0 | Control (Path) 2 | 9.2 |
| Ctx | | Occipital Ctx | |
| AD 5 Sup Temporal | 100.0 | Control (Path) 3 | 0.0 |
| Ctx | | Occipital Ctx | |
| AD 6 Inf Temporal | 28.1 | Control (Path) 4 | 9.0 |
| Ctx | | Occipital Ctx | |
| AD 6 Sup Temporal | 8.9 | Control 1 Parietal | 9.9 |
| Ctx | | Ctx | |
| Control 1 Temporal | 11.8 | Control 2 Parietal | 41.2 |
| Ctx | | Ctx | |
| Control 2 Temporal | 38.4 | Control 3 Parietal | 95 |
| Ctx | | Ctx | |
| Control 3 Temporal | 16.5 | Control (Path) 1 | 50.7 |
| Ctx | | Parietal Ctx | |
| Control 3 Temporal | 0.0 | Control (Path) 2 | 30.1 |
| Ctx | | Parietal Ctx | |
| Control (Path) 1 | 57.4 | Control (Path) 3 | 2.2 |
| Temporal Ctx | | Parietal Ctx | |
| Control (Path) 2 | 29.3 | Control (Path) 4 | 27.5 |
| Temporal Ctx | | Parietal Ctx | |

TABLE 54
General_screening_panel_v1.4

| | Rel. Exp. (%) Ag4335, | | Rel. Exp. (%) Ag4335, |
|---|---|---|---|
| Tissue Name | Run 222550589 | Tissue Name | Run 222550589 |
| Adipose | 4.7 | Renal ca. TK-10 | 0.0 |
| Melanoma* | 0.2 | Bladder | 0.5 |
| Hs688(A).T | | | |
| Melanoma* | 0.4 | Gastric ca. (liver met.) | 0.2 |
| Hs688(B).T | | NCI-N87 | |
| Melanoma* M14 | 0.1 | Gastric ca. KATO III | 0.0 |
| Melanoma* | 0.1 | Colon ca. SW-948 | 0.0 |
| LOXIMVI | | | |
| Melanoma* SK- | 0.2 | Colon ca. SW480 | 0.1 |
| MEL-5 | | | |
| Squamous cell | 0.3 | Colon ca.* (SW480 | 0.0 |
| carcinoma SCC-4 | | met) SW620 | |
| Testis Pool | 1.5 | Colon ca. HT29 | 1.8 |
| Prostate ca.* (bone | 0.3 | Colon ca. HCT-116 | 0.1 |
| met) PC-3 | | | |
| Prostate Pool | 0.2 | Colon ca. CaCo-2 | 3.8 |
| Placenta | 0.1 | Colon cancer tissue | 0.3 |
| Uterus Pool | 1.5 | Colon ca. SW1116 | 0.0 |
| Ovarian ca. | 1.1 | Colon ca. Colo-205 | 0.0 |
| OVCAR-3 | | | |
| Ovarian ca. SK-OV-3 | 0.0 | Colon ca. SW-48 | 0.0 |
| Ovarian ca. | 0.0 | Colon Pool | 2.0 |
| OVCAR-4 | | | |
| Ovarian ca. | 0.2 | Small Intestine Pool | 4.2 |
| OVCAR-5 | | | |
| Ovarian ca. IGROV-1 | 0.0 | Stomach Pool | 1.5 |
| Ovarian ca. | 0.1 | Bone Marrow Pool | 1.1 |
| OVCAR-8 | | | |
| Ovary | 3.8 | Fetal Heart | 4.9 |
| Breast ca. MCF-7 | 0.7 | Heart Pool | 2.7 |
| Breast ca. MDA- | 0.1 | Lymph Node Pool | 2.2 |
| MB-231 | | | |
| Breast ca. BT 549 | 0.2 | Fetal Skeletal Muscle | 8.2 |
| Breast ca. T47D | 0.3 | Skeletal Muscle Pool | 0.3 |
| Breast ca. MDA-N | 0.0 | Spleen Pool | 2.2 |
| Breast Pool | 2.1 | Thymus Pool | 2.1 |
| Trachea | 7.0 | CNS cancer (glio/astro) | 0.3 |
| | | U87-MG | |
| Lung | 8.5 | CNS cancer (glio/astro) | 0.4 |
| | | U-118-MG | |
| Fetal Lung | 100.0 | CNS cancer | 0.0 |
| | | (neuro;met) SK-N-AS | |
| Lung ca. NCI-N417 | 0.0 | CNS cancer (astro) SF- | 3.9 |
| | | 539 | |
| Lung ca. LX-1 | 0.0 | CNS cancer (astro) | 10.0 |
| | | SNB-75 | |
| Lung ca. NCI-H146 | 10.7 | CNS cancer (glio) | 0.0 |
| | | SNB-19 | |
| Lung ca. SHP-77 | 0.1 | CNS cancer (glio) SF- | 4.0 |
| | | 295 | |
| Lung ca. A549 | 3.3 | Brain (Amygdala) Pool | 0.5 |
| Lung ca. NCI-H526 | 0.2 | Brain (cerebellum) | 0.3 |
| Lung ca. NCI-H23 | 3.3 | Brain (fetal) | 0.4 |
| Lung ca. NCI-H460 | 0.1 | Brain (Hippocampus) | 1.6 |
| | | Pool | |
| Lung ca. HOP-62 | 2.6 | Cerebral Cortex Pool | 1.1 |
| Lung ca. NCI-H522 | 0.0 | Brain (Substantia nigra) | 0.5 |
| | | Pool | |
| Liver | 0.1 | Brain (Thalamus) Pool | 1.4 |
| Fetal Liver | 0.6 | Brain (whole) | 0.6 |
| Liver ca. HepG2 | 0.0 | Spinal Cord Pool | 0.7 |
| Kidney Pool | 8.0 | Adrenal Gland | 3.8 |
| Fetal Kidney | 1.6 | Pituitary gland Pool | 3.0 |
| Renal ca. 786-0 | 0.3 | Salivary Gland | 1.7 |
| Renal ca. A498 | 0.2 | Thyroid (female) | 1.0 |
| Renal ca. ACHN | 0.0 | Pancreatic ca. CAPAN2 | 0.0 |
| Renal ca. UO-31 | 0.0 | Pancreas Pool | 2.4 |

TABLE 55
Panel 1

| | Rel. Exp. (%) Ag317, | | Rel. Exp. (%) Ag317, |
|---|---|---|---|
| Tissue Name | Run 88164410 | Tissue Name | Run 88164410 |
| Endothelial cells | 0.0 | Renal ca. 786-0 | 0.0 |
| Endothelial cells | 0.0 | Renal ca. A498 | 0.0 |
| (treated) | | | |
| Pancreas | 0.0 | Renal ca. RXF 393 | 0.0 |
| Pancreatic ca. CAPAN 2 | 0.0 | Renal ca. ACHN | 0.0 |
| Adrenal gland | 6.1 | Renal ca. UO-31 | 0.0 |
| Thyroid | 1.0 | Renal ca. TK-10 | 0.0 |
| Salivary gland | 11.0 | Liver | 0.7 |
| Pituitary gland | 1.1 | Liver (fetal) | 0.0 |
| Brain (fetal) | 0.0 | Liver ca. (hepatoblast) | 0.0 |
| | | HepG2 | |
| Brain (whole) | 0.0 | Lung | 0.0 |
| Brain (amygdala) | 0.2 | Lung (fetal) | 37.9 |
| Brain (cerebellum) | 2.3 | Lung ca. (small cell) | 0.0 |
| | | LX-1 | |
| Brain (hippocampus) | 0.1 | Lung ca. (small cell) | 0.0 |
| | | NCI-H69 | |
| Brain (substantia nigra) | 0.1 | Lung ca. (s.cell var.) | 0.0 |
| | | SHP-77 | |
| Brain (thalamus) | 0.0 | Lung ca. (large | 0.0 |
| | | cell)NCI-H460 | |
| Brain (hypothalamus) | 0.1 | Lung ca. (non-sm. | 0.1 |
| | | cell) A549 | |
| Spinal cord | 0.2 | Lung ca. (non-s.cell) | 0.0 |
| | | NCI-H23 | |
| glio/astro U87-MG | 0.0 | Lung ca. (non-s.cell) | 0.0 |
| | | HOP-62 | |
| glio/astro U-118-MG | 0.0 | Lung ca. (non-s.cl) | 0.0 |
| | | NCI-H522 | |
| astrocytoma SW1783 | 0.0 | Lung ca. (squam.) SW | 0.4 |
| | | 900 | |
| neuro*; met SK-N-AS | 0.0 | Lung ca. (squam.) | 0.3 |
| | | NCI-H596 | |
| astrocytoma SF-539 | 0.0 | Mammary gland | 0.0 |
| astrocytoma SNB-75 | 0.0 | Breast ca.* (pl.ef) | 0.0 |
| | | MCF-7 | |
| glioma SNB-19 | 0.0 | Breast ca.* (pl.ef) | 0.0 |
| | | MDA-MB-231 | |
| glioma U251 | 0.0 | Breast ca.* (pl. ef) | 0.0 |
| | | T47D | |
| glioma SF-295 | 0.4 | Breast ca. BT-549 | 0.0 |
| Heart | 7.2 | Breast ca. MDA-N | 0.0 |
| Skeletal muscle | 0.0 | Ovary | 2.0 |
| Bone marrow | 0.0 | Ovarian Ca. OVCAR-3 | 0.0 |
| Thymus | 0.4 | Ovarian ca. OVCAR-4 | 0.0 |
| Spleen | 1.4 | Ovarian ca. OVCAR-5 | 0.0 |
| Lymph node | 13.6 | Ovarian ca. OVCAR-8 | 0.0 |
| Colon (ascending) | 0.7 | Ovarian ca. IGROV-1 | 0.0 |
| Stomach | 2.8 | Ovarian ca. (ascites) | 0.0 |
| | | SK-OV-3 | |
| Small intestine | 15.5 | Uterus | 0.0 |
| Colon ca. SW480 | 0.0 | Placenta | 12.0 |
| Colon ca.* SW620 | 0.0 | Prostate | 0.0 |
| (SW480 met) | | | |
| Colon ca. HT29 | 0.0 | Prostate ca.* (bone | 0.0 |
| | | met) PC-3 | |
| Colon ca. HCT-116 | 0.0 | Testis | 100.0 |
| Colon ca. CaCo-2 | 0.1 | Melanoma | 0.0 |
| | | Hs688(A).T | |
| Colon ca. HCT-15 | 0.0 | Melanoma* (met) | 0.0 |
| | | Hs688(B).T | |
| Colon ca. HCC-2998 | 0.0 | Melanoma UACC-62 | 0.0 |
| Gastric ca. * (liver met) | 0.0 | Melanoma M14 | 0.0 |
| NCI-N87 | | | |
| Bladder | 2.8 | Melanoma LOX | 0.0 |
| | | IMVI | |
| Trachea | 2.3 | Melanoma* (met) SK- | 0.0 |
| | | MEL-5 | |
| Kidney | 0.0 | Melanoma SK-MEL- | 0.0 |
| | | 28 | |
| Kidney (fetal) | 0.0 | | |

TABLE 56
Panel 1.1

| | Rel. Exp. (%) Ag674, | | Rel. Exp. (%) Ag674, |
|---|---|---|---|
| Tissue Name | Run 109485186 | Tissue Name | Run 109485186 |
| Adrenal gland | 29.9 | Renal ca. UO-31 | 0.2 |
| Bladder | 0.7 | Renal ca. RXF 393 | 0.1 |
| Brain (amygdala) | 2.8 | Liver | 6.1 |
| Brain (cerebellum) | 6.8 | Liver (fetal) | 1.8 |
| Brain (hippocampus) | 9.4 | Liver ca. | 0.1 |
| | | (hepatoblast) HepG2 | |
| Brain (substantia | 16.5 | Lung | 49.7 |
| nigra) | | | |
| Brain (thalamus) | 4.8 | Lung (fetal) | 59.9 |
| Cerebal Cortex | 2.9 | Lung ca. (non-s.cell) | 61.6 |
| | | HOP-62 | |
| Brain (fetal) | 3.4 | Lung ca. (large | 2.7 |
| | | cell)NCI-H460 | |
| Brain (whole) | 8.4 | Lung ca. (non-s.cell) | 6.5 |
| | | NCI-H23 | |
| glio/astro U-118-MG | 0.9 | Lung ca. (non-s.cl) | 1.1 |
| | | NCI-H522 | |
| astrocytoma SF-539 | 0.8 | Lung ca. (non-sm. | 15.0 |
| | | cell) A549 | |
| astrocytoma SNB-75 | 3.7 | Lung ca. (s.cell var.) | 0.1 |
| | | SHP-77 | |
| astrocytoma SW1783 | 5.0 | Lung ca. (small cell) | 0.4 |
| | | LX-1 | |
| glioma U251 | 1.7 | Lung ca. (small cell) | 3.1 |
| | | NCI-H69 | |
| glioma SF-295 | 15.8 | Lung ca. (squam.) | 7.2 |
| | | SW 900 | |
| glioma SNB-19 | 1.1 | Lung ca. (squam.) | 22.1 |
| | | NCI-H596 | |
| glio/astro U87-MG | 1.1 | Lymph node | 17.1 |
| neuro*; met SK-N-AS | 0.0 | Spleen | 3.2 |
| Mammary gland | 69.3 | Thymus | 2.7 |
| Breast ca. BT-549 | 0.0 | Ovary | 14.2 |
| Breast ca. MDA-N | 0.1 | Ovarian ca. IGROV-1 | 0.2 |
| Breast ca.* (pl.ef) | 0.9 | Ovarian ca. OVCAR-3 | 12.6 |
| T47D | | | |
| Breast ca.* (pl.ef) | 1.8 | Ovarian ca. OVCAR-4 | 0.0 |
| MCF-7 | | | |
| Breast ca.* (pl.ef) | 0.0 | Ovarian ca. OVCAR-5 | 4.6 |
| MDA-MB-231 | | | |
| Small intestine | 30.6 | Ovarian ca. OVCAR-8 | 0.5 |
| Colorectal | 0.9 | Ovarian ca.* (ascites) | 0.1 |
| | | SK-OV-3 | |
| Colon ca. HT29 | 6.6 | Pancreas | 24.3 |
| Colon ca. CaCo-2 | 4.3 | Pancreatic ca. | 0.0 |
| | | CAPAN 2 | |
| Colon ca. HCT-15 | 0.1 | Pituitary gland | 29.3 |
| Colon ca. HCT-116 | 0.2 | Placenta | 12.8 |
| Colon ca. HCC-2998 | 2.3 | Prostate | 0.8 |
| Colon ca. SW480 | 0.2 | Prostate ca.* (bone | 0.9 |
| | | met) PC-3 | |
| Colon ca.* SW620 | 0.1 | Salivary gland | 65.1 |
| (SW480 met) | | | |
| Stomach | 9.1 | Trachea | 26.6 |
| Gastric ca. (liver met) | 0.5 | Spinal cord | 6.6 |
| NCI-N87 | | | |
| Heart | 100.0 | Testis | 45.7 |
| Skeletal muscle (Fetal) | 5.0 | Thyroid | 13.6 |
| Skeletal muscle | 6.3 | Uterus | 7.9 |
| Endothelial cells | 0.0 | Melanoma M14 | 0.9 |
| Heart (Fetal) | 12.0 | Melanoma LOX | 0.1 |
| | | IMVI | |
| Kidney | 8.3 | Melanoma UACC-62 | 0.1 |
| Kidney (fetal) | 3.7 | Melanoma SK-MEL- | 6.1 |
| | | 28 | |
| Renal ca. 786-0 | 0.2 | Melanoma* (met) | 0.3 |
| | | SK-MEL-5 | |
| Renal ca. A498 | 0.3 | Melanoma | 0.2 |
| | | Hs688(A).T | |
| Renal ca. ACHN | 0.0 | Melanoma* (met) | 0.4 |
| | | Hs688(B).T | |
| Renal ca. TK-10 | 0.2 | | |

TABLE 57
Panel 1.2

| | Rel. Exp. (%) | Rel. Exp. (%) | | Rel. Exp. (%) | Rel. Exp. (%) |
|---|---|---|---|---|---|
| | Ag1489, Run | Ag674, Run | | Ag1489, Run | Ag674, Run |
| Tissue Name | 141889869 | 118424516 | Tissue Name | 141889869 | 118424516 |
| Endothelial cells | 0.0 | 2.2 | Rena1 ca. 786-0 | 0.1 | 0.0 |
| Heart (Fetal) | 12.7 | 3.9 | Renal ca. A498 | 0.1 | 0.3 |
| Pancreas | 0.4 | 17.8 | Renal ca. RXF | 0.0 | 0.1 |
| | | | 393 | | |
| Pancreatic ca. | 0.0 | 0.0 | Renal ca. | 0.2 | 0.0 |
| CAPAN 2 | | | ACHN | | |
| Adrenal Gland | 24.1 | 42.6 | Renal ca. | 0.2 | 0.4 |
| | | | UO-31 | | |
| Thyroid | 0.2 | 29.1 | Renal ca. TK-10 | 0.4 | 0.1 |
| Salivary gland | 93.3 | 54.7 | Liver | 1.8 | 7.0 |
| Pituitary gland | 0.5 | 47.6 | Liver (fetal) | 0.7 | 3.7 |
| Brain (fetal) | 0.0 | 2.0 | Liver ca. | 0.1 | 0.0 |
| | | | (hepatoblast) | | |
| | | | HepG2 | | |
| Brain (whole) | 0.1 | 6.1 | Lung | 1.1 | 75.3 |
| Brain | 1.8 | 9.3 | Lung (fetal) | 1.5 | 74.7 |
| (amygdala) | | | | | |
| Brain | 0.4 | 1.1 | Lung ca. | 0.1 | 0.1 |
| (cerebellum) | | | (small cell) | | |
| | | | LX-1 | | |
| Brain | 2.9 | 9.5 | Lung ca. | 0.7 | 1.1 |
| (hippocampus) | | | (small cell) | | |
| | | | NCI-H69 | | |
| Brain (thalamus) | 0.7 | 2.1 | Lung ca. (s.cell | 0.1 | 0.5 |
| | | | var.) SHP-77 | | |
| Cerebral Cortex | 2.6 | 1.5 | Lung ca. (large | 0.3 | 0.9 |
| | | | cell)NCI-H460 | | |
| Spinal cord | 0.2 | 5.8 | Lung ca. (non- | 2.4 | 8.8 |
| | | | sm. cell) A549 | | |
| glio/astro | 0.8 | 0.5 | Lung ca. (non- | 2.1 | 5.5 |
| U87-MG | | | s.cell) | | |
| | | | NCI-H23 | | |
| glio/astro U- | 0.8 | 0.6 | Lung ca. (non- | 13.2 | 19.6 |
| 118-MG | | | s.cell) HOP-62 | | |
| astrocytoma | 1.5 | 4.6 | Lung ca. (non- | 2.5 | 1.2 |
| SW1783 | | | s.cl) NCI-H522 | | |
| neuro*; met | 0.0 | 0.1 | Lung ca. | 2.8 | 5.9 |
| SK-N-AS | | | (squam.) SW | | |
| | | | 900 | | |
| astrocytoma | 0.3 | 0.9 | Lung ca. | 5.4 | 11.0 |
| SF-539 | | | (squam.) | | |
| | | | NCI-H596 | | |
| astrocytoma | 0.9 | 2.4 | Mammary | 2.4 | 67.4 |
| SNB-75 | | | gland | | |
| glioma SNB-19 | 0.4 | 0.5 | Breast ca.* | 1.2 | 1.3 |
| | | | (pl.ef) MCF-7 | | |
| glioma U251 | 0.5 | 0.6 | Breast ca.* | 0.0 | 0.0 |
| | | | (pl.ef) MDA- | | |
| | | | MB-231 | | |
| glioma SF-295 | 7.2 | 15.4 | Breast ca.* (pl. | 0.3 | 0.6 |
| | | | ef) T47D | | |
| Heart | 100.0 | 82.9 | Breast ca. | 0.2 | 0.0 |
| | | | BT-549 | | |
| Skeletal Muscle | 1.7 | 6.2 | Breast ca. | 0.1 | 0.2 |
| | | | MDA-N | | |
| Bone marrow | 0.7 | 1.7 | Ovary | 25.2 | 7.0 |
| Thymus | 0.3 | 3.9 | Ovarian ca. | 1.4 | 5.0 |
| | | | OVCAR-3 | | |
| Spleen | 0.8 | 4.7 | Ovarian ca. | 0.0 | 0.0 |
| | | | OVCAR-4 | | |
| Lymph node | 0.3 | 19.3 | Ovarian ca. | 1.4 | 1.9 |
| | | | OVCAR-5 | | |
| Colorectal | 0.5 | 0.5 | Ovarian ca. | 0.9 | 0.1 |
| Tissue | | | OVCAR-8 | | |
| Stomach | 0.2 | 9.3 | Ovarian ca. | 0.1 | 0.1 |
| | | | IGROV-1 | | |
| Small intestine | 8.1 | 55.9 | Ovarian ca. | 0.2 | 0.0 |
| | | | (ascites) SK- | | |
| | | | OV-3 | | |
| Colon ca. | 0.2 | 0.0 | Uterus | 2.4 | 14.9 |
| SW480 | | | | | |
| Colon ca.* | 0.1 | 0.0 | Placenta | 2.9 | 17.0 |
| SW620 (SW480 | | | | | |
| met) | | | | | |
| Colon ca. HT29 | 2.3 | 5.3 | Prostate | 0.9 | 1.5 |
| Colon ca. | 0.4 | 0.1 | Prostate ca.* | 0.3 | 0.8 |
| HCT-116 | | | (bone met) PC-3 | | |
| Colon ca. CaCo-2 | 3.7 | 5.0 | Testis | 0.8 | 100.0 |
| Colon ca. Tissue | 0.0 | 0.3 | Melanoma | 0.1 | 0.1 |
| (ODO3866) | | | Hs688(A).T | | |
| Colon ca. HCC- | 1.3 | 1.4 | Melanoma* | 0.2 | 0.9 |
| 2998 | | | (met) | | |
| | | | Hs688(B).T | | |
| Gastric ca.* | 0.2 | 0.3 | Melanoma | 0.4 | 0.0 |
| (liver met) NCI- | | | UACC-62 | | |
| N87 | | | | | |
| Bladder | 2.0 | 0.3 | Melanoma | 0.3 | 0.5 |
| | | | M14 | | |
| Trachea | 9.0 | 33.2 | Melanoma | 0.0 | 0.1 |
| | | | LOX IMVI | | |
| Kidney | 4.7 | 2.8 | Melanoma* | 0.1 | 0.5 |
| | | | (met) SK- | | |
| | | | MEL-5 | | |
| Kidney (fetal) | 0.9 | 2.5 | | | |

TABLE 58
Panel 1.3D

| | Rel. Exp. (%) | Rel. Exp. (%) | | Rel. Exp. (%) | Rel. Exp. (%) |
|---|---|---|---|---|---|
| | Ag1489, Run | Ag1489, Run | | Ag1489, Run | Ag1489, Run |
| Tissue Name | 152615469 | 152615562 | Tissue Name | 152615469 | 152615562 |
| Liver | 0.2 | 0.0 | Kidney (fetal) | 0.7 | 1.5 |
| adenocarcinoma | | | | | |
| Pancreas | 1.9 | 1.7 | Renal ca. 786-0 | 0.6 | 0.7 |
| Pancreatic ca. | 0.2 | 0.0 | Renal ca. | 8.5 | 8.2 |
| CAPAN 2 | | | A498 | | |
| Adrenal gland | 8.2 | 18.3 | Renal ca. RXF | 0.0 | 0.2 |
| | | | 393 | | |
| Thyroid | 7.2 | 6.5 | Renal ca. | 0.0 | 0.0 |
| | | | ACHN | | |
| Salivary gland | 4.2 | 13.9 | Renal ca. | 0.1 | 0.1 |
| | | | UO-31 | | |
| Pituitary gland | 9.0 | 7.7 | Renal ca. | 0.0 | 0.1 |
| | | | TK-10 | | |
| Brain (fetal) | 1.1 | 0.6 | Liver | 1.9 | 1.3 |
| Brain (whole) | 1.7 | 2.0 | Liver (fetal) | 2.5 | 3.6 |
| Brain (amygdala) | 6.3 | 12.7 | Liver ca. | 0.0 | 0.2 |
| | | | (hepatoblast) | | |
| | | | HepG2 | | |
| Brain | 0.3 | 0.7 | Lung | 67.8 | 100.0 |
| (cerebellum) | | | | | |
| Brain | 17.3 | 14.9 | Lung (fetal) | 100.0 | 85.3 |
| (hippocampus) | | | | | |
| Brain (substantia | 0.9 | 1.9 | Lung ca. | 0.0 | 0.0 |
| nigra) | | | (small cell) | | |
| | | | LX-1 | | |
| Brain (thalamus) | 0.9 | 4.2 | Lung ca. | 0.4 | 1.6 |
| | | | (small cell) | | |
| | | | NCI-H69 | | |
| Cerebral Cortex | 3.5 | 5.6 | Lung ca. | 0.4 | 0.4 |
| | | | (s.cell var.) | | |
| | | | SHP-77 | | |
| Spinal cord | 4.6 | 3.9 | Lung ca. (large | 0.1 | 0.0 |
| | | | cell) NCI-H460 | | |
| glio/astro | 0.2 | 0.0 | Lung ca. (non- | 6.3 | 1.9 |
| U87-MG | | | sm. cell) A549 | | |
| glio/astro | 1.5 | 2.9 | Lung ca. (non- | 6.2 | 6.3 |
| U-118-MG | | | s.cell) | | |
| | | | NCI-H23 | | |
| astrocytoma | 4.2 | 6.1 | Lung ca. (non- | 6.0 | 8.5 |
| SW1783 | | | s.cell) HOP-62 | | |
| neuro*; met | 0.0 | 0.0 | Lung ca. (non- | 0.1 | 1.2 |
| SK-N-AS | | | s.cl) | | |
| | | | NCI-H522 | | |
| astrocytoma | 0.9 | 0.5 | Lung ca. | 1.9 | 2.7 |
| SF-539 | | | (squam.) SW | | |
| | | | 900 | | |
| astrocytoma | 16.4 | 15.4 | Lung ca. | 2.5 | 3.2 |
| SNB-75 | | | (squam.) | | |
| | | | NCI-H596 | | |
| glioma SNB-19 | 0.4 | 1.2 | Mammary | 33.4 | 43.5 |
| | | | gland | | |
| glioma U251 | 0.6 | 1.1 | Breast ca.* | 0.6 | 0.6 |
| | | | (pl.ef) MCF-7 | | |
| glioma SF-295 | 4.2 | 8.2 | Breast ca.* | 0.0 | 0.1 |
| | | | (pl.ef) MDA- | | |
| | | | MB-231 | | |
| Heart (fetal) | 9.2 | 10.9 | Breast ca.* | 0.0 | 0.1 |
| | | | (pl.ef) T47D | | |
| Heart | 11.3 | 12.9 | Breast ca. | 0.1 | 0.0 |
| | | | BT-549 | | |
| Skeletal muscle | 10.2 | 24.5 | Breast ca. | 0.0 | 0.3 |
| (fetal) | | | MDA-N | | |
| Skeletal muscle | 0.1 | 0.4 | Ovary | 42.0 | 62.4 |
| Bone marrow | 2.1 | 2.2 | Ovarian ca. | 1.4 | 1.3 |
| | | | OVCAR-3 | | |
| Thymus | 2.4 | 3.4 | Ovarian ca. | 0.0 | 0.0 |
| | | | OVCAR-4 | | |
| Spleen | 8.2 | 9.7 | Ovarian ca. | 0.3 | 0.1 |
| | | | OVCAR-5 | | |
| Lymph node | 17.7 | 27.5 | Ovarian ca. | 0.1 | 0.7 |
| | | | OVCAR-8 | | |
| Colorectal | 0.7 | 1.6 | Ovarian ca. | 0.0 | 0.0 |
| | | | IGROV-1 | | |
| Stomach | 4.8 | 10.9 | Ovarian ca.* | 0.0 | 0.0 |
| | | | (ascites) SK- | | |
| | | | OV-3 | | |
| Small intestine | 21.8 | 32.1 | Uterus | 6.3 | 9.1 |
| Colon ca. SW480 | 0.5 | 0.3 | Plancenta | 3.8 | 7.1 |
| Colon ca.* | 0.0 | 0.0 | Prostate | 0.4 | 1.0 |
| SW620(SW480 | | | | | |
| met) | | | | | |
| Colon ca. | 2.1 | 3.3 | Prostate ca.* | 0.0 | 0.3 |
| HT29 | | | (bone met) | | |
| | | | PC-3 | | |
| Colon ca. | 0.3 | 0.0 | Testis | 43.5 | 56.3 |
| HCT-116 | | | | | |
| Colon ca. CaCo-2 | 3.4 | 4.0 | Melanoma | 0.7 | 0.8 |
| | | | Hs688(A).T | | |
| Colon ca. | 0.4 | 0.6 | Melanoma* | 0.1 | 1.2 |
| tissue(ODO3866) | | | (met) | | |
| | | | Hs688(B).T | | |
| Colon ca. | 0.5 | 1.3 | Melanoma | 0.0 | 0.0 |
| HCC-2998 | | | UACC-62 | | |
| Gastric ca.* (liver | 0.0 | 0.3 | Melanoma | 0.0 | 0.0 |
| met) NCI-N87 | | | M14 | | |
| Bladder | 0.1 | 0.4 | Melanoma | 0.1 | 0.2 |
| | | | LOX IMVI | | |
| Trachea | 33.2 | 43.5 | Melanoma* | 0.3 | 0.0 |
| | | | (met) SK- | | |
| | | | MEL-5 | | |
| Kidney | 0.5 | 0.0 | Adipose | 4.7 | 4.2 |

TABLE 59
Panel 2D

| | Rel. Exp. (%) | Rel. Exp. (%) | | Rel. Exp. (%) | Rel. Exp. (%) |
|---|---|---|---|---|---|
| | Ag1489, Run | Ag1489, Run | | Ag1489, Run | Ag1489, Run |
| Tissue Name | 152799815 | 152799848 | Tissue Name | 152799815 | 152799848 |
| Normal Colon | 24.0 | 17.2 | Kidney | 0.1 | 0.0 |
| | | | Margin | | |
| | | | 8120608 | | |
| CC Well to Mod | 1.4 | 0.4 | Kidney Cancer | 0.0 | 0.0 |
| Diff (ODO3866) | | | 8120613 | | |
| CC Margin | 4.6 | 3.6 | Kidney | 0.1 | 0.1 |
| (ODO3866) | | | Margin | | |
| | | | 8120614 | | |
| CC Gr.2 | 0.4 | 0.7 | Kidney Cancer | 1.1 | 1.0 |
| rectosigmoid | | | 9010320 | | |
| (ODO3868) | | | | | |
| CC Margin | 3.4 | 1.1 | Kidney | 0.1 | 0.2 |
| (ODO3868) | | | Margin | | |
| | | | 9010321 | | |
| CC Mod Diff | 0.0 | 0.1 | Normal Uterus | 3.1 | 2.3 |
| (ODO3920) | | | | | |
| CC Margin | 5.0 | 4.7 | Uterus Cancer | 24.5 | 18.8 |
| (ODO3920) | | | 064011 | | |
| CC Gr.2 ascend | 1.0 | 0.5 | Normal | 10.4 | 9.0 |
| colon | | | Thyroid | | |
| (ODO3921) | | | | | |
| CC Margin | 4.1 | 5.0 | Thyroid | 0.3 | 0.0 |
| (ODO3921) | | | Cancer | | |
| | | | 064010 | | |
| CC from Partial | 2.2 | 0.9 | Thyroid | 0.5 | 0.5 |
| Hepatectomy | | | Cancer | | |
| (ODO4309) | | | A302152 | | |
| Mets | | | | | |
| Liver Margin | 3.2 | 2.0 | Thyroid | 14.5 | 9.2 |
| (ODO4309) | | | Margin | | |
| | | | A302153 | | |
| Colon mets to | 0.4 | 0.7 | Normal Breast | 32.1 | 31.9 |
| lung | | | | | |
| (OD04451-01) | | | | | |
| Lung Margin | 6.0 | 5.2 | Breast Cancer | 0.5 | 0.4 |
| (OD04451-02) | | | (OD04566) | | |
| Normal Prostate | 1.1 | 0.9 | Breast Cancer | 9.3 | 4.4 |
| 6546-1 | | | (OD04590-01) | | |
| Prostate Cancer | 1.4 | 0.6 | Breast Cancer | 9.7 | 5.9 |
| (OD04410) | | | Mets | | |
| | | | (OD04590-03) | | |
| Prostate Margin | 0.2 | 0.7 | Breast Cancer | 8.2 | 5.8 |
| (OD04410) | | | Metastasis | | |
| | | | (OD04655-05) | | |
| Prostate Cancer | 0.9 | 0.6 | Breast Cancer | 8.4 | 5.3 |
| (OD04720-01) | | | 064006 | | |
| Prostate Margin | 1.0 | 1.0 | Breast Cancer | 9.6 | 9.2 |
| (OD04720-02) | | | 1024 | | |
| Normal Lung | 100.0 | 100.0 | Breast Cancer | 1.7 | 2.2 |
| 061010 | | | 9100266 | | |
| Lung Met to | 0.8 | 1.0 | Breast Margin | 10.7 | 9.6 |
| Muscle | | | 9100265 | | |
| (ODO4286) | | | | | |
| Muscle Margin | 2.0 | 1.3 | Breast Cancer | 11.0 | 10.2 |
| (ODO4286) | | | A209073 | | |
| Lung Malignant | 11.1 | 8.4 | Breast Margin | 13.6 | 12.5 |
| Cancer | | | A2090734 | | |
| (OD03126) | | | | | |
| Lung Margin | 64.2 | 51.8 | Normal Liver | 2.2 | 1.9 |
| (OD03126) | | | | | |
| Lung Cancer | 6.6 | 5.1 | Liver Cancer | 0.6 | 0.4 |
| (OD04404) | | | 064003 | | |
| Lung Margin | 37.4 | 31.9 | Liver Cancer | 0.6 | 0.2 |
| (OD04404) | | | 1025 | | |
| Lung Cancer | 1.8 | 1.3 | Liver Cancer | 0.0 | 0.1 |
| (OD04565) | | | 1026 | | |
| Lung Margin | 19.3 | 20.6 | Liver Cancer | 0.9 | 0.3 |
| (OD04565) | | | 6004-T | | |
| Lung Cancer | 2.0 | 2.2 | Liver Tissue | 0.2 | 0.2 |
| (OD04237-01) | | | 6004-N | | |
| Lung Margin | 42.3 | 37.6 | Liver Cancer | 0.0 | 0.2 |
| (OD04237-02) | | | 6005-T | | |
| Ocular Mel Met | 0.0 | 0.2 | Liver Tissue | 0.0 | 0.0 |
| to Liver | | | 6005-N | | |
| (ODO4310) | | | | | |
| Liver Margin | 2.3 | 0.5 | Normal | 2.4 | 0.5 |
| (ODO4310) | | | Bladder | | |
| Melanoma Mets | 1.9 | 1.8 | Bladder | 0.5 | 0.9 |
| to Lung | | | Cancer 1023 | | |
| (OD04321) | | | | | |
| Lung Margin | 49.0 | 37.9 | Bladder | 33.4 | 31.4 |
| (OD04321) | | | Cancer | | |
| | | | A302173 | | |
| Normal Kidney | 5.4 | 4.8 | Bladder | 0.4 | 0.0 |
| | | | Cancer | | |
| | | | (OD04718-01) | | |
| Kidney Ca, | 0.4 | 0.6 | Bladder | 17.4 | 17.6 |
| Nuclear grade 2 | | | Normal | | |
| (OD04338) | | | Adjacent | | |
| | | | (OD04718-03) | | |
| Kidney Margin | 3.2 | 2.1 | Normal Ovary | 22.4 | 14.9 |
| (OD04338) | | | | | |
| Kidney Ca | 0.7 | 0.3 | Ovarian | 7.1 | 4.4 |
| Nuclear grade | | | Cancer | | |
| 1/2 (OD04339) | | | 064008 | | |
| Kidney Margin | 1.2 | 0.2 | Ovarian | 2.1 | 1.8 |
| (OD04339) | | | Cancer | | |
| | | | (OD04768-07) | | |
| Kidney Ca, Clear | 0.4 | 0.4 | Ovary Margin | 1.2 | 1.8 |
| cell type | | | (OD04768-08) | | |
| (OD04340) | | | | | |
| Kidney Margin | 5.1 | 4.2 | Normal | 6.4 | 4.4 |
| (OD04340) | | | Stomach | | |
| Kidney Ca, | 0.3 | 0.1 | Gastric Cancer | 3.1 | 1.4 |
| Nuclear grade 3 | | | 9060358 | | |
| (OD04348) | | | | | |
| Kidney Margin | 2.4 | 1.6 | Stomach | 1.3 | 2.3 |
| (OD04348) | | | Margin | | |
| | | | 9060359 | | |
| Kidney Cancer | 0.1 | 0.2 | Gastric Cancer | 2.4 | 4.5 |
| (OD04622-01) | | | 9060395 | | |
| Kidney Margin | 0.0 | 0.1 | Stomach | 5.3 | 3.4 |
| (OD04622-03) | | | Margin | | |
| | | | 9060394 | | |
| Kidney Cancer | 0.0 | 0.0 | Gastric Cancer | 0.3 | 0.7 |
| (OD04450-01) | | | 9060397 | | |
| Kidney Margin | 1.9 | 1.1 | Stomach | 0.7 | 0.1 |
| (OD04450-03) | | | Margin | | |
| | | | 9060396 | | |
| Kidney Cancer | 0.0 | 0.0 | Gastric Cancer | 6.8 | 4.4 |
| 8120607 | | | 064005 | | |

TABLE 60
Panel 4.1D

| | Rel. Exp. (%) Ag4335, Run | | Rel. Exp. (%) Ag4335, Run |
|---|---|---|---|
| Tissue Name | 184793354 | Tissue Name | 184793354 |
| Secondary Th1 act | 0.0 | HUVEC IL-1beta | 0.0 |
| Secondary Th2 act | 0.0 | HUVEC IFN gamma | 0.9 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.5 |
| Secondary Th2 rest | 0.0 | HUVEC IL-11 | 0.0 |
| Secondary Tr1 rest | 0.0 | Lung Microvasular EC | 1.1 |
| | | none | |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microvascular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium | 0.6 |
| | | none | |
| Primary Tr1 rest | 0.0 | Small airway epithelium | 0.7 |
| | | TNFalpha + IL-1beta | |
| CD45RA CD4 | 0.6 | Coroney artery SMC rest | 1.9 |
| lymphocyte act | | | |
| CD45RO CD4 | 0.0 | Coronery artery SMC | 1.6 |
| lymphocyte act | | TNFalpha + IL-1beta | |
| CD8 lymphocyte act | 0.0 | Astrocytes rest | 0.0 |
| Secondary CD8 | 1.2 | Astrocytes TNFalpha + | 0.0 |
| lymphocyte rest | | IL-1beta | |
| Secondary CD8 | 0.0 | KU-812 (Basophil) rest | 48.6 |
| lymphocyte act | | | |
| CD4 lymphocyte none | 0.0 | KU-812 (Basophil) | 26.1 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 1.5 | CCD1106 (Keratinocytes) | 0.0 |
| | | TNFalpha + IL-1beta | |
| LAK cells IL-2 | 0.0 | Liver cirrhosis | 6.0 |
| LAK cells IL-2 + IL-12 | 0.0 | NCI-H292 none | 0.7 |
| LAK cells IL-2 + IFN | 0.0 | NCI-H292 IL-4 | 0.0 |
| gamma | | | |
| LAK cells IL-2 + IL-18 | 0.9 | NCI-H292 IL-9 | 0.7 |
| LAK cells | 0.0 | NCI-H292 IL-13 | 0.4 |
| PMA/ionomycin | | | |
| NK cells IL-2 rest | 0.3 | NCI-H292 IFN gamma | 1.7 |
| Two Way MLR 3 day | 1.7 | HPAEC none | 0.0 |
| Two Way MLR 5 day | 0.5 | HPAEC TNF alpha + IL-1 | 0.0 |
| | | beta | |
| Two Way MLR 7 day | 0.0 | Lung fibroblast none | 26.8 |
| PBMC rest | 0.0 | Lung fibroblast TNF alpha + | 2.0 |
| | | IL-1 beta | |
| PBMC PWM | 0.5 | Lung fibroblast IL-4 | 20.3 |
| PBMC PHA-L | 0.0 | Lung fibroblast IL-9 | 26.2 |
| Ramos (B cell) none | 0.7 | Lung fibroblast IL-13 | 37.4 |
| Ramos (B cell) | 0.0 | Lung fibroblast IFN | 44.1 |
| ionomycin | | gamma | |
| B lymphocytes PWM | 0.0 | Dermal fibroblast | 3.0 |
| | | CCD1070 rest | |
| B lymphocytes CD40L | 0.8 | Dermal fibroblast | 0.7 |
| and IL-4 | | CCD1070 TNF alpha | |
| EOL-1 dbcAMP | 34.2 | Dermal fibroblast | 2.2 |
| | | CCD1070 IL-1 beta | |
| EOL-1 dbcAMP | 18.6 | Dermal fibroblast IFN | 12.8 |
| PMA/ionomycin | | gamma | |
| Dendritic cells none | 11.9 | Dermal fibroblast IL-4 | 48.3 |
| Dendritic cells LPS | 0.7 | Dermal Fibroblasts rest | 17.9 |
| Dendritic cells anti- | 2.7 | Neutrophils TNFa + LPS | 0.0 |
| CD40 | | | |
| Monocytes rest | 1.4 | Neutrophils rest | 0.7 |
| Monocytes LPS | 0.0 | Colon | 10.7 |
| Macrophages rest | 2.8 | Lung | 100.0 |
| Macrophages LPS | 1.1 | Thymus | 27.9 |
| HUVEC none | 0.0 | Kidney | 10.0 |
| HUVEC starved | 0.0 | | |

TABLE 61
Panel 4D

| | Rel. Exp. (%) | Rel. Exp. (%) | Rel. Exp. (%) | | Rel. Exp. (%) | Rel. Exp. (%) | Rel. Exp. (%) |
|---|---|---|---|---|---|---|---|
| | Ag1489, Run | Ag1489, Run | Ag1489, Run | | Ag1489, Run | Ag1489, Run | Ag1489, Run |
| Tissue Name | 142272577 | 144378640 | 144575346 | Tissue Name | 142272577 | 144378640 | 144575346 |
| Secondary Th1 | 0.0 | 0.5 | 0.0 | HUVEC IL- | 0.0 | 0.3 | 0.0 |
| act | | | | 1beta | | | |
| Secondary Th2 | 0.0 | 0.0 | 0.8 | HUVEC IFN | 0.0 | 0.0 | 0.4 |
| act | | | | gamma | | | |
| Secondary Tr1 | 1.0 | 0.0 | 0.6 | HUVEC TNF | 0.0 | 0.0 | 0.0 |
| act | | | | alpha + IFN | | | |
| | | | | gamma | | | |
| Secondary Th1 | 0.0 | 0.0 | 0.0 | HUVEC TNF | 0.0 | 0.0 | 0.0 |
| rest | | | | alpha + IL4 | | | |
| Secondary Th2 | 0.0 | 0.0 | 0.0 | HUVEC IL-11 | 0.0 | 3.6 | 0.0 |
| rest | | | | | | | |
| Secondary Tr1 | 0.0 | 0.0 | 0.0 | Lung | 0.6 | 0.5 | 0.4 |
| rest | | | | Microvascular | | | |
| | | | | EC none | | | |
| Primary Th1 act | 0.3 | 0.0 | 3.4 | Lung | 0.0 | 0.0 | 0.0 |
| | | | | Microvascular | | | |
| | | | | EC TNFalpha | | | |
| | | | | + IL-1beta | | | |
| Primary Th2 act | 1.3 | 1.1 | 0.0 | Microvascular | 1.7 | 0.0 | 0.0 |
| | | | | Dermal EC | | | |
| | | | | none | | | |
| Primary Tr1 act | 0.4 | 1.9 | 2.2 | Microsvasular | 0.0 | 0.0 | 0.0 |
| | | | | Dermal EC | | | |
| | | | | TNFalpha + | | | |
| | | | | IL-1beta | | | |
| Primary Th1 | 0.6 | 0.4 | 0.0 | Bronchial | 0.0 | 0.7 | 0.0 |
| rest | | | | epithelium | | | |
| | | | | TNFalpha + | | | |
| | | | | IL1beta | | | |
| Primary Th2 | 0.0 | 0.0 | 0.0 | Small airway | 0.0 | 0.0 | 0.0 |
| rest | | | | epithelium | | | |
| | | | | none | | | |
| Primary Tr1 rest | 0.0 | 0.0 | 0.9 | Small airway | 0.0 | 0.0 | 0.0 |
| | | | | epithelium | | | |
| | | | | TNFalpha + | | | |
| | | | | IL-1beta | | | |
| CD45RA CD4 | 0.0 | 0.5 | 0.5 | Coronery | 5.9 | 0.9 | 2.0 |
| lymphocyte act | | | | artery SMC | | | |
| | | | | rest | | | |
| CD45RO CD4 | 0.4 | 0.5 | 0.9 | Coronery | 1.0 | 0.3 | 0.0 |
| lymphocyte act | | | | artery SMC | | | |
| | | | | TNFalpha + | | | |
| | | | | IL-1beta | | | |
| CD8 | 0.6 | 0.0 | 0.0 | Astrocytes rest | 0.6 | 0.0 | 0.4 |
| lymphocyte act | | | | | | | |
| Secondary CD8 | 0.0 | 0.5 | 0.4 | Astrocytes | 0.5 | 0.0 | 0.4 |
| lymphocyte rest | | | | TNFalpha + | | | |
| | | | | IL-1beta | | | |
| Secondary CD8 | 0.0 | 0.5 | 0.0 | KU-812 | 36.6 | 26.4 | 25.9 |
| lymphocyte act | | | | (Basophil) rest | | | |
| CD4 | 1.1 | 0.0 | 0.0 | KU-812 | 33.9 | 16.7 | 24.5 |
| lymphocyte | | | | (Basophil) | | | |
| none | | | | PMA/ | | | |
| | | | | ionomycin | | | |
| 2ry | 0.6 | 1.9 | 0.0 | CCD1106 | 0.0 | 0.0 | 0.5 |
| Th1/Th2/Tr1 | | | | (Keratinocytes) | | | |
| anti-CD95 CH11 | | | | none | | | |
| LAK cells rest | 1.4 | 0.5 | 0.6 | CCD1106 | 0.5 | 0.0 | 0.4 |
| | | | | (Keratinocytes) | | | |
| | | | | TNFalpha + | | | |
| | | | | IL-1beta | | | |
| LAK cells IL-2 | 0.0 | 0.4 | 0.0 | Liver cirrhosis | 5.8 | 2.2 | 3.4 |
| LAK cells IL-2 + | 0.0 | 0.0 | 0.4 | Lupus kidney | 4.2 | 3.3 | 4.0 |
| IL-12 | | | | | | | |
| LAK cells IL-2 + | 0.0 | 0.5 | 0.0 | NCI-H292 | 1.2 | 1.4 | 2.9 |
| IFN gamma | | | | none | | | |
| LAK cells IL-2 + | 1.0 | 0.0 | 0.7 | NCI-H292 IL-4 | 1.5 | 0.4 | 1.3 |
| IL-18 | | | | | | | |
| LAK cells | 0.0 | 0.4 | 4.5 | NCI-H292 IL-9 | 0.5 | 2.0 | 0.4 |
| PMA/ionomycin | | | | | | | |
| NK Cells IL-2 | 0.5 | 0.5 | 0.0 | NCI-H292 IL- | 0.9 | 0.0 | 0.9 |
| rest | | | | 13 | | | |
| Two Way MLR | 1.1 | 1.9 | 1.0 | NCI-H292 | 2.2 | 0.3 | 1.2 |
| 3 day | | | | IFN gamma | | | |
| Two Way MLR | 0.0 | 1.4 | 0.0 | HPAEC none | 0.0 | 0.0 | 0.0 |
| 5 day | | | | | | | |
| Two Way MLR | 0.0 | 0.0 | 0.0 | HPAEC TNF | 0.0 | 0.9 | 0.0 |
| 7 day | | | | alpha +IL-1 | | | |
| | | | | beta | | | |
| PBMC rest | 0.0 | 0.0 | 0.5 | Lung | 22.4 | 9.4 | 7.7 |
| | | | | fibroblast | | | |
| | | | | none | | | |
| PBMC PWM | 3.1 | 4.4 | 1.7 | Lung | 0.5 | 0.0 | 0.5 |
| | | | | fibroblast TNF | | | |
| | | | | alpha + IL-1 | | | |
| | | | | beta | | | |
| PBMC PHA-L | 0.6 | 1.0 | 0.5 | Lung | 33.0 | 31.4 | 29.7 |
| | | | | fibroblast IL-4 | | | |
| Ramos (B cell) | 6.2 | 5.3 | 3.2 | Lung | 6.7 | 8.7 | 19.3 |
| none | | | | fibroblast IL-9 | | | |
| Ramos (B cell) | 8.0 | 10.4 | 11.3 | Lung | 100.0 | 48.6 | 51.8 |
| ionomycin | | | | fibroblast | | | |
| | | | | IL-13 | | | |
| B lymphocytes | 3.4 | 2.4 | 3.0 | Lung | 50.0 | 39.5 | 47.0 |
| PWM | | | | fibroblast IFN | | | |
| | | | | gamma | | | |
| B lymphocytes | 2.4 | 3.4 | 2.4 | Dermal | 2.8 | 2.2 | 1.9 |
| CD40L and IL-4 | | | | fibroblast | | | |
| | | | | CCD1070 rest | | | |
| EOL-1 dbcAMP | 19.2 | 13.0 | 21.2 | Dermal | 1.5 | 2.2 | 2.9 |
| | | | | fibroblast | | | |
| | | | | CCD1070 | | | |
| | | | | TNF alpha | | | |
| EOL-1 dbcAMP | 8.4 | 6.5 | 6.7 | Dermal | 0.0 | 1.7 | 1.5 |
| PMA/ionomycin | | | | fibroblast | | | |
| | | | | CCD1070 IL- | | | |
| | | | | 1 beta | | | |
| Dendritic cells | 3.8 | 2.9 | 5.9 | Dermal | 6.1 | 4.5 | 3.0 |
| none | | | | fibroblast IFN | | | |
| | | | | gamma | | | |
| Dendritic cells | 0.0 | 0.0 | 0.0 | Dermal | 33.2 | 27.5 | 25.5 |
| LPS | | | | fibroblast IL-4 | | | |
| Dendritic cells | 4.2 | 2.0 | 3.9 | IBD Colitis 2 | 0.6 | 1.0 | 1.6 |
| anti-CD40 | | | | | | | |
| Monocytes rest | 1.8 | 0.4 | 0.9 | IBD Crohn's | 11.8 | 10.0 | 6.1 |
| Monocytes LPS | 0.0 | 0.0 | 0.0 | Colon | 25.9 | 27.7 | 24.5 |
| Macrophages | 1.0 | 0.0 | 0.8 | Lung | 92.0 | 100.0 | 100.0 |
| rest | | | | | | | |
| Macrophages | 1.7 | 0.4 | 1.3 | Thymus | 21.9 | 16.7 | 18.3 |
| LPS | | | | | | | |
| HUVEC none | 0.0 | 0.4 | 0.0 | Kidney | 30.8 | 18.9 | 13.1 |
| HUVEC starved | 0.4 | 0.4 | 0.6 | | | | |

AI_comprehensive panel_v1.0 Summary: Ag1489 Expression of the NOV12 transcript is induced in most rheumatoid arthritis (RA) tissues and in synovium and bone tissues in osteoarthritis (OA) patients. It is also highly expressed in normal lung in 2 out of 3 normal lungs in this panel and in panels 1.1, 1.2, 1.3, 4 and 4.1. In addition, the expression in diseased lung is reduced or absent. These data suggest that lung expression of the protein encoded for by this transcript may serve an important function that is lost in disease states, but that OA/RA promotes the expression of this gene in the joint. Therefore, therapies designed with the protein encoded by this transcript may be important for the treament of OA/RA and lung diseases such as chronic pulmonary obstructive disease, emphysema, allergy and asthma.

CNS_neurodegeneration_v1.0 Summary: Ag4335 The NOV12 gene is not expressed differentially in Alzheimer's disease. However, widespread expression of this gene in the brain suggests a role for the gene product in CNS processes. Therapeutic modulation of the expression or function of this protein may be useful in the treatment of neurological disorders and stroke. A second experiment with Ag3032 showed low/undetectable expression in all samples on this panel (CTs>35). (Data not shown.)

General_screening_panel_v1.4 Summary: Ag4335 Highest expression of the NOV12 gene is seen in fetal lung (CT=25.4), with overall expression associated with normal tissues. Furthermore, expression of this gene is higher in fetal lung when compared to expression in adult lung (CT=29) and in fetal skeletal muscle (CT=29) when compared to adult skeletal muscle (CT=33). Thus, this gene product may be involved in the development and homeostasis of these organs. Therapeutic modulation of the expression or function of this gene may maintain or restore function to lung and skeletal muscle affected by disease.

This gene is also expressed in a variety of metabolic tissues including adipose, adult and fetal liver, adult and fetal heart, pituitary, thyroid and pancreas. This expression profile suggests that this gene product could potentially be used to treat metabolic disorders, including obesity and diabetes.

This gene shows widespread moderate expression in the brain. This result is in concordance with CNS_neurodegeneration_V1.0. Please see that panel for discussion of potential utility in the central nervous system.

Panel 1 Summary: Ag317 Highest expression of the NOV12 gene is seen in the testis (CT=26.6). There is also substantial expression seen in fetal lung tissue (CT=28), especially when compared to expression in adult lung (CT=40). This result is in concordance with the results from Panel General_screening_panel_v1.4, as is association of expression with normal tissue. Thus, this gene product may be involved in the development and homeostasis of the lung. Therapeutic modulation of the expression or function of this gene may maintain or restore function to lung affected by disease.

As in General_screening_panel_v1.4, this gene is moderately expressed in adipose, heart, pituitary, thyroid and pancreas. Thus, this gene product may be important for the pathogenesis, diagnosis, and/or treatment of metabolic disease, including Types 1 and 2 diabetes, and obesity. In addition, this gene appears to be differentially expressed in adult (CT value=34) versus fetal liver (CT value=40) and may be useful for the identification of the fetal vs adult source of this tissue.

As in the preceding panels, this gene is also widely expressed in the brain. Please see that panel for discussion of potential utility in the central nervous system.

Panel 1.1 Summary: Ag674 Highest expression of the NOV12 gene is in the heart (CT=27.4). This gene is also moderately expressed in a variety of other metabolic tissues including adult and fetal liver, fetal heart, adult and fetal skeletal muscle, adrenal, pituitary, thyroid and pancreas. Thus, this gene product may be important for the pathogenesis, diagnosis, and/or treatment of metabolic disease, including Types 1 and 2 diabetes, and obesity.

There is also substantial expression in samples derived from mammary gland, salivary gland, testis, and a number of lung derived samples, a preference seen in previous panel results. Thus, the expression of this gene could be used to distinguish these samples from other samples in the panel. Moreover, therapeutic modulation of this gene product, through the use of small molecule drugs, antibodies or protein therapeutics might be of benefit in the treatment of lung cancer.

As in the preceding panels, this gene is also widely expressed in the brain. Please see that panel for discussion of potential utility in the central nervous system.

Panel 1.2 Summary: Ag1489/Ag674 The expression of the NOV12 gene was analysed in two independent runs on panel 1.2 with two different probe/primer pairs. The data is somewhat discordant which may reflect size and/or post-transcriptional processing of this gene. The expression of the gene appeared to be highest in testis tissue in one run and heart tissue in the other run. In addition there is substantial expression associated with salivary gland, adrenal gland, lung tissue, small intestine and mammary gland. Thus, the expression of this gene could be used to distinguish these tissues from other tissues in the panel.

This gene is also moderately expressed in a variety of metabolic tissues including adult and fetal liver, adult and fetal heart, adrenal, pituitary, thyroid and pancreas. Thus, this gene product may be important for the pathogenesis, diagnosis, and/or treatment of metabolic disease, including Types 1 and 2 diabetes, and obesity.

As in the previous panels, this gene is also widely expressed in the brain. Please see that panel for discussion of potential utility in the central nervous system.

Panel 1.3D Summary: Ag1489 Two experiments with the same probe and primer set show excellent concordance. The expression of the NOV12 gene appears to be highest in a sample derived from normal fetal and adult lung tissue. This association with lung is evident in the preceding panels as well. In addition, there is substantial expression in normal ovary, testis, mammary gland and trachea. Thus, the expression of this gene could be used to distinguish these tissues from other tissues in the panel. Overall, the gene shows association with normal tissues, as seen in previous panels.

There is also significant association with tissues of metabolic relevance, including pancreas, adrenal, thyroid, pituitary, adult and fetal heart, adult and fetal liver, and adipose. Thus, this gene product may be important for the pathogenesis, diagnosis, and/or treatment of metabolic disease, including Types 1 and 2 diabetes, and obesity.

This gene product appears to be differentially expressed in fetal (CTs=29-30) vs adult (CTs=35-37) skeletal muscle and may be useful for the identification of the adult vs fetal source of this tissue. Furthermore, the higher levels of expression of this gene in the fetal tissue suggest that this gene product may be involved in the development and homeostasis of the skeletal muscle. Therapeutic modulation of the expression or function of this gene may maintain or restore function to weak or dystrophic muscle.

As in the previous panels, this gene is also widely expressed in the brain. Please see that panel for discussion of potential utility in the central nervous system. A third experiment with Ag3032 showed low/undetectable expression in all samples on this panel (CTs>35). (Data not shown.) The amp plot indicates that there is a high probability of a probe failure.

Panel 2D Summary: Ag1489 The expression of the NOV12 gene was analyzed in two independent runs in panel 2D with excellent concordance between the runs. The expression of this gene appears to be highest in a sample of normal lung tissue, in agreement with previous results. In addition, there is substantial expression in all of the normal lung tissue samples adjacent to samples derived from lung cancer. Expression in the lung cancers appears to be virtually absent. Thus, the expression of this gene could be used to distinguish normal lung tissue from malignant lung tissue. Moreover, therapeutic modulation of this gene product, through the use of small molecule drugs, antibodies or protein therapeutics might be of benefit in the treatment of lung cancer.

Panel 4.1D and Panel 4D Summary: Ag4335/Ag1489 Multiple experiments show that the NOV12 gene is expressed in the lung, lung fibroblasts, EOL-1 cells and KU-812 cells. Expression of the transcript is downregulated by TNFalpha and IL-1 beta in lung fibroblasts. Based on the expression profile in this panel as well as that in the A/I panel, therapeutics designed with this protein could be important in the treatment of chronic obstructive pulmonary disease, emphysema, allergy and asthma. One experiment with Ag3032 showed low/undetectable expression in all samples on this panel (CTs>35). (Data not shown.) The amp plot indicates that there is a high probability of a probe failure.

NOV11a

Expression of gene NOV11a was assessed using the primer-probe sets Ag2313 and Ag401, described in Tables 62 and 63. Results of the RTQ-PCR runs are shown in Tables 64, 65, 66, 67, 68 and 69.

TABLE 62

Probe Name Ag2313

| Primers | Sequences | Length | Start Position | SEQ ID NO: |
|---|---|---|---|---|
| Forward | 5′tatcttgatgatggccattca-3′ | 21 | 2631 | 214 |
| Probe | TET-5′-tccaatacctccaccagaagcaattt-3′-TAMRA | 26 | 2653 | 215 |
| Reverse | 5′-caccagaacactggaacagaat-3′ | 22 | 2696 | 216 |

TABLE 63

Probe Name Ag401

| | | | | |
|---|---|---|---|---|
| Primers | Sequences | Length | Start Position | SEQ ID NO: |

| | | | | |
|---|---|---|---|---|
| Forward | 5′-ttgtgccaaaacatccatcct-3′ | 21 | 2855 | 217 |
| Probe | TET-5′-agcctggagaagctctcactcaacattgc-3′-TAMRA | 29 | 2877 | 218 |
| Reverse | 5′-tatgatgcggacctcccagt-3′ | 20 | 2911 | 219 |

TABLE 64
AI_comprehensive panel_v1.0

| | Rel. Exp. (%) Ag2313, Run | | Rel. Exp. (%) Ag2313, Run |
|---|---|---|---|
| Tissue Name | 225147624 | Tissue Name | 225147624 |
| 110967 COPD-F | 23.5 | 112427 Match Control | 100.0 |
| | | Psoriasis-F | |
| 110980 COPD-F | 0.5 | 112418 Psoriasis-M | 25.0 |
| 110968 COPD-M | 24.5 | 112723 Match Control | 14.7 |
| | | Psoriasis-M | |
| 110977 COPD-M | 63.3 | 112419 Psoriasis-M | 31.0 |
| 110989 Emphysema-F | 46.7 | 112424 Match Control | 20.9 |
| | | Psoriasis-M | |
| 110992 Emphysema-F | 12.1 | 112420 Psoriasis-M | 88.3 |
| 110993 Emphysema-F | 26.6 | 112425 Match Control | 78.5 |
| | | Psoriasis-M | |
| 110994 Emphysema-F | 13.7 | 104689 (MF) OA | 45.1 |
| | | Bone-Backus | |
| 110995 Emphysema-F | 37.6 | 104690 (MF) Adj | 32.8 |
| | | “Normal” Bone-Backus | |
| 110996 Emphysema-F | 6.8 | 104691 (MF) OA | 40.1 |
| | | Synovium-Backus | |
| 110997 Asthma-M | 6.7 | 104692 (BA) OA | 25.7 |
| | | Cartilage-Backus | |
| 111001 Astham-F | 38.2 | 104694 (BA) OA | 42.6 |
| | | Bone-Backus | |
| 111002 Asthma-F | 24.0 | 104695 (BA) Adj | 27.7 |
| | | “Normal” Bone-Backus | |
| 111003 Atopic | 28.5 | 104696 (BA) OA | 25.2 |
| Asthma-F | | Synovium-Backus | |
| 111004 Atopic | 27.7 | 104700 (SS) OA Bone- | 23.5 |
| Asthma-F | | Backus | |
| 111005 Atopic | 24.3 | 104701 (SS) Adj | 23.3 |
| Asthma-F | | “Normal” Bone-Backus | |
| 111006 Atopic | 5.3 | 104702 (SS) OA | 48.6 |
| Asthma-F | | Synovium-Backus | |
| 111417 Allergy-M | 17.7 | 117093 OA Cartilage | 31.0 |
| | | Rep7 | |
| 112347 Allergy-M | 0.0 | 112672 OA Bone5 | 74.7 |
| 112349 Normal Lung-F | 0.0 | 112673 OA Synovium5 | 31.9 |
| 112357 Normal Lung-F | 55.1 | 112674 OA Synovial | 40.1 |
| | | Fluid cells5 | |
| 112354 Normal Lung-M | 37.9 | 117100 OA Cartilage | 9.3 |
| | | Rep14 | |
| 112374 Crohns-F | 32.5 | 112756 OA Bone9 | 11.5 |
| 112389 Match | 22.8 | 112757 OA Synovium9 | 45.1 |
| Control Crohns-F | | | |
| 112375 Crohns-F | 22.8 | 112758 OA Synovial | 28.9 |
| | | Fluid Cells9 | |
| 112732 Match | 26.1 | 117125 RA Cartilage | 26.6 |
| Control Crohns-F | | Rep2 | |
| 112725 Crohns-M | 10.4 | 113492 Bone2 RA | 36.1 |
| 112387 Match | 27.7 | 113493 Synovium2 RA | 18.7 |
| Control Crohns-M | | | |
| 112378 Crohns-M | 0.0 | 113494 Syn Fluid Cells | 33.0 |
| | | RA | |
| 112390 Match | 99.3 | 113499 Cartilage4 RA | 44.4 |
| Control Crohns-M | | | |
| 112726 Crohns-M | 23.8 | 113500 Bone4 RA | 61.6 |
| 112731 Match | 29.3 | 113501 Synovium4 RA | 38.7 |
| Control Crohns-M | | | |
| 112380 Ulcer Col-F | 29.9 | 113502 Syn Fluid | 19.6 |
| | | Cells4 RA | |
| 112734 Match | 84.1 | 113495 Cartilage3 RA | 30.8 |
| Control Ulcer Col-F | | | |
| 112384 Ulcer Col-F | 64.2 | 113496 Bone3 RA | 35.4 |
| 112737 Match | 14.1 | 113497 Synovium3 RA | 20.7 |
| Control Ulcer Col-F | | | |
| 112386 Ulcer Col-F | 23.5 | 113498 Syn Fluid | 34.6 |
| | | Cells3 RA | |
| 112738 Match | 11.0 | 117106 Normal | 10.2 |
| Control Ulcer Col-F | | Cartilage Rep20 | |
| 112381 Ulcer Col-M | 1.0 | 113663 Bone3 Normal | 1.0 |
| 112735 Match | 25.0 | 113664 Synovium3 | 0.0 |
| Control Ulcer Col-M | | Normal | |
| 112382 Ulcer Col-M | 29.1 | 113665 Syn Fluid | 0.6 |
| | | Cells3 Normal | |
| 112394 Match | 12.6 | 117107 Normal | 18.6 |
| Control Ulcer Col-M | | Cartilage Rep22 | |
| 112383 Ulcer Col-M | 35.8 | 113667 Bone4 Normal | 21.0 |
| 112736 Match | 12.4 | 113668 Synovium4 | 23.7 |
| Control Ulcer Col-M | | Normal | |
| 112423 Psoriasis-F | 28.5 | 113669 Syn Fluid | 31.4 |
| | | Cells4 Normal | |

TABLE 65
CNS_neurodegeneration_v1.0

| | Rel. Exp. (%) Ag2313, | | Rel. Exp. (%) Ag2313, |
|---|---|---|---|
| Tissue Name | Run 206262287 | Tissue Name | Run 206262287 |
| AD 1 Hippo | 10.2 | Control (Path) 3 | 4.6 |
| | | Temporal Ctx | |
| AD 2 Hippo | 19.5 | Control (Path) 4 | 28.3 |
| | | Temporal Ctx | |
| AD 3 Hippo | 7.6 | AD 1 Occipital Ctx | 18.4 |
| AD 4 Hippo | 6.3 | AD 2 Occipital Ctx | 0.0 |
| | | (Missing) | |
| AD 5 hippo | 68.8 | AD 3 Occipital Ctx | 9.0 |
| AD 6 Hippo | 46.3 | AD 4 Occipital Ctx | 22.8 |
| Control 2 Hippo | 23.5 | AD 5 Occipital Ctx | 18.9 |
| Control 4 Hippo | 18.2 | AD 6 Occipital Ctx | 25.3 |
| Control (Path) 3 | 11.0 | Control 1 Occipital | 3.5 |
| Hippo | | Ctx | |
| AD 1 Temporal Ctx | 23.7 | Control 2 Occipital | 41.8 |
| | | Ctx | |
| AD 2 Temporal Ctx | 24.1 | Control 3 Occipital | 8.2 |
| | | Ctx | |
| AD 3 Temporal Ctx | 10.2 | Control 4 Occipital | 5.8 |
| | | Ctx | |
| AD 4 Temporal Ctx | 34.9 | Control (Path) 1 | 62.4 |
| | | Occipital Ctx | |
| AD 5 Inf Temporal | 100.0 | Control (Path) 2 | 13.0 |
| Ctx | | Occipital Ctx | |
| AD 5 Sup Temporal | 48.0 | Control (Path) 3 | 4.4 |
| Ctx | | Occipital Ctx | |
| AD 6 Inf Temporal | 66.9 | Control (Path) 4 | 12.8 |
| Ctx | | Occipital Ctx | |
| AD 6 Sup Temporal | 66.4 | Control 1 Parietal | 8.7 |
| Ctx | | Ctx | |
| Control 1 Temporal | 5.7 | Control 2 Parietal | 42.9 |
| Ctx | | Ctx | |
| Control 2 Temporal | 31.9 | Control 3 Parietal | 10.9 |
| Ctx | | Ctx | |
| Control 3 Temporal | 9.5 | Control (Path) 1 | 53.2 |
| Ctx | | Parietal Ctx | |
| Control 4 Temporal | 10.2 | Control (Path) 2 | 27.4 |
| Ctx | | Parietal Ctx | |
| Control (Path) 1 | 39.5 | Control (Path) 3 | 4.6 |
| Temporal Ctx | | Parietal Ctx | |
| Control (Path) 2 | 37.1 | Control (Path) 4 | 37.1 |
| Temporal Ctx | | Parietal Ctx | |

TABLE 66
Panel 1

| | Rel. Exp. (%) Ag401, | | Rel. Exp. (%) Ag401, |
|---|---|---|---|
| Tissue Name | Run 88225002 | Tissue Name | Run 88225002 |
| Endothelial cells | 1.5 | Renal ca. 786-0 | 10.7 |
| Endothelial cells | 2.5 | Renal ca. A498 | 4.2 |
| (treated) | | | |
| Pancreas | 11.0 | Renal ca. RXF 393 | 4.8 |
| Pancreatic ca. CAPAN 2 | 2.6 | Renal ca. ACHN | 4.9 |
| Adrenal gland | 8.5 | Renal ca. UO-31 | 3.8 |
| Thyroid | 13.6 | Renal ca. TK-10 | 5.0 |
| Salivary gland | 12.7 | Liver | 24.0 |
| Pituitary gland | 8.3 | Liver (fetal) | 4.0 |
| Brain (fetal) | 4.2 | Liver ca. (hepatoblast) | 7.1 |
| | | HepG2 | |
| Brain (whole) | 14.7 | Lung | 3.1 |
| Brain (amygdala) | 4.1 | Lung (fetal) | 12.9 |
| Brain (cerebellum) | 74.2 | Lung ca. (small cell) | 9.5 |
| | | LX-1 | |
| Brain (hippocampus) | 7.4 | Lung ca. (small cell) | 0.8 |
| | | NCI-H69 | |
| Brain (substantia nigra) | 13.6 | Lung ca. (s.cell var.) | 13.2 |
| | | SHP-77 | |
| Brain (thalamus) | 7.3 | Lung ca. (large | 8.5 |
| | | cell)NCI-H460 | |
| Brain (hypothalamus) | 12.6 | Lung ca. (non-sm. | 4.3 |
| | | cell) A549 | |
| Spinal cord | 12.9 | Lung ca. (non-s.cell) | 5.7 |
| | | NCI-H23 | |
| glio/astro U87-MG | 3.5 | Lung ca. (non-s.cl) | 3.7 |
| | | HOP-62 | |
| glio/astro U-118-MG | 7.6 | Lung ca. (non-s.cl) | 10.9 |
| | | NCI-H522 | |
| astrocytoma SW1783 | 2.0 | Lung ca. (squam.) SW | 8.4 |
| | | 900 | |
| neuro*; met SK-N-AS | 4.4 | Lung ca. (squam.) | 1.2 |
| | | NCI-H596 | |
| astrocytoma SF-539 | 5.6 | Mammary gland | 32.1 |
| astrocytoma SNB-75 | 5.4 | Breast ca.* (pl.ef) | 5.6 |
| | | MCF-7 | |
| glioma SNB-19 | 7.0 | Breast ca.* (pl.ef) | 3.3 |
| | | MDA-MB-231 | |
| glioma U251 | 5.5 | Breast ca.* (pl.ef) | 12.3 |
| | | T47D | |
| glioma SF-295 | 5.3 | Breast ca. BT-549 | 6.7 |
| Heart | 8.8 | Breast ca. MDA-N | 8.8 |
| Skeletal muscle | 14.6 | Ovary | 10.4 |
| Bone marrow | 9.3 | Ovarian ca. OVCAR-3 | 3.1 |
| Thymus | 27.7 | Ovarian ca. OVCAR-4 | 1.4 |
| Spleen | 14.6 | Ovarian ca. OVCAR-5 | 12.6 |
| Lymph node | 21.6 | Ovarian ca. OVCAR-8 | 3.4 |
| Colon (ascending) | 13.7 | Ovarian ca. IGROV-1 | 3.2 |
| Stomach | 20.4 | Ovarian ca. (ascites) | 8.3 |
| | | SK-OV-3 | |
| Small intestine | 17.4 | Uterus | 16.7 |
| Colon ca. SW480 | 1.5 | Placenta | 11.2 |
| Colon ca.* SW620 | 4.2 | Prostate | 12.4 |
| (SW480 met) | | | |
| Colon ca. HT29 | 1.5 | Prostate ca.* (bone | 13.1 |
| | | met) PC-3 | |
| Colon Ca. HCT-116 | 5.3 | Testis | 100.0 |
| Colon ca. CaCo-2 | 3.5 | Melanoma | 12.4 |
| | | Hs688(A).T | |
| Colon ca. HCT-15 | 7.2 | Melanoma* (met) | 6.4 |
| | | Hs688(B).T | |
| Colon ca. HCC-2998 | 6.4 | Melanoma UACC-62 | 1.6 |
| Gastric ca.* (liver met) | 26.1 | Melanoma M14 | 33.9 |
| NCI-N87 | | | |
| Bladder | 4.3 | Melanoma LOX | 8.0 |
| | | IMVI | |
| Trachea | 20.9 | Melanoma* (met) SK- | 3.0 |
| | | MEL-5 | |
| Kidney | 14.9 | Melanoma SK-MEL- | 15.9 |
| | | 28 | |
| Kidney (fetal) | 17.3 | | |

TABLE 67
Panel 1.3D

| | Rel. Exp. (%) | Rel. Exp. (%) | | Rel. Exp. (%) | Rel. Exp. (%) |
|---|---|---|---|---|---|
| | Ag2313, Run | Ag401, Run | | Ag2313, Run | Ag401, Run |
| Tissue Name | 157982894 | 165518174 | Tissue Name | 157982894 | 165518174 |
| Liver | 4.4 | 2.9 | Kidney (fetal) | 20.2 | 13.8 |
| adenocarcinoma | | | | | |
| Pancreas | 13.8 | 10.1 | Renal ca. 786-0 | 10.7 | 14.8 |
| Pancreatic ca. | 2.1 | 10.2 | Renal ca. | 26.6 | 13.9 |
| CAPAN 2 | | | A498 | | |
| Adrenal gland | 24.7 | 16.2 | Renal ca. RXF | 3.0 | 18.2 |
| | | | 393 | | |
| Thyroid | 32.5 | 18.9 | Renal ca. | 6.7 | 12.7 |
| | | | ACHN | | |
| Salivary gland | 11.0 | 24.7 | Renal ca. UO-31 | 5.9 | 12.2 |
| Pituitary gland | 27.2 | 20.2 | Renal ca. TK-10 | 7.1 | 5.7 |
| Brain (fetal) | 11.9 | 23.5 | Liver | 17.8 | 16.7 |
| Brain (whole) | 26.2 | 52.1 | Liver (fetal) | 20.4 | 9.0 |
| Brain (amygdala) | 20.3 | 16.2 | Liver ca. | 15.2 | 9.6 |
| | | | (hepatoblast) | | |
| | | | HepG2 | | |
| Brain | 17.9 | 47.6 | Lung | 81.2 | 19.5 |
| (cerebellum) | | | | | |
| Brain | 100.0 | 54.3 | Lung (fetal) | 74.7 | 11.2 |
| (hippocampus) | | | | | |
| Brain (substantia | 12.9 | 20.0 | Lung ca. | 17.8 | 18.7 |
| nigra) | | | (small cell) | | |
| | | | LX-1 | | |
| Brain (thalamus) | 17.7 | 41.5 | Lung ca. | 1.0 | 0.0 |
| | | | (small cell) | | |
| | | | NCI-H69 | | |
| Cerebral Cortex | 24.3 | 15.0 | Lung ca. | 12.0 | 10.9 |
| | | | (s.cell var.) | | |
| | | | SHP-77 | | |
| Spinal cord | 23.5 | 38.7 | Lung ca. (large | 5.0 | 19.5 |
| | | | cell)NCI-H460 | | |
| glio/astro | 6.0 | 9.5 | Lung ca. (non- | 5.7 | 1.6 |
| U87-MG | | | sm. cell) A549 | | |
| glio/astro U-118- | 58.2 | 49.7 | Lung ca. (non- | 16.4 | 9.3 |
| MG | | | s.cell) NCI- | | |
| | | | H23 | | |
| astrocytoma | 8.2 | 10.4 | Lung ca. (non- | 7.8 | 17.8 |
| SW1783 | | | s.cell) HOP-62 | | |
| neuro*; met | 49.7 | 20.2 | Lung ca. (non- | 4.3 | 9.0 |
| SK-N-AS | | | s.cl) NCI- | | |
| | | | H522 | | |
| astrocytoma SF- | 13.6 | 25.7 | Lung ca. | 4.2 | 4.5 |
| 539 | | | (squam.) SW | | |
| | | | 900 | | |
| astrocytoma | 18.8 | 22.4 | Lung ca. | 0.4 | 3.1 |
| SNB-75 | | | (squam.) NCI- | | |
| | | | H596 | | |
| glioma SNB-19 | 16.7 | 25.9 | Mammary | 45.7 | 27.0 |
| | | | gland | | |
| glioma U251 | 18.4 | 59.9 | Breast ca.* | 5.0 | 4.3 |
| | | | (pl.ef) MCF-7 | | |
| glioma SF-295 | 12.1 | 15.4 | Breast ca.* | 39.2 | 47.3 |
| | | | (pl.ef) MDA- | | |
| | | | MB-231 | | |
| Heart (fetal) | 4.2 | 0.5 | Breast ca.* | 8.5 | 6.4 |
| | | | (pl.ef) T47D | | |
| Heart | 8.8 | 14.0 | Breast ca. | 17.0 | 12.9 |
| | | | BT-549 | | |
| Skeletal muscle | 77.9 | 7.0 | Breast ca. | 20.4 | 6.2 |
| (fetal) | | | MDA-N | | |
| Skeletal muscle | 19.2 | 100.0 | Ovary | 28.3 | 6.3 |
| Bone marrow | 33.9 | 33.9 | Ovarian ca. | 5.5 | 10.5 |
| | | | OVCAR-3 | | |
| Thymus | 25.2 | 32.1 | Ovarian ca. | 1.1 | 7.2 |
| | | | OVCAR-4 | | |
| Spleen | 44.1 | 27.9 | Ovarian ca. | 20.6 | 13.6 |
| | | | OVCAR-5 | | |
| Lymph node | 35.4 | 76.8 | Ovarian ca. | 10.5 | 4.6 |
| | | | OVCAR-8 | | |
| Colorectal | 40.6 | 38.7 | Ovarian ca. | 8.7 | 2.5 |
| | | | IGROV-1 | | |
| Stomach | 34.6 | 21.0 | Ovarian ca.* | 25.0 | 17.3 |
| | | | (ascites) | | |
| | | | SK-OV-3 | | |
| Small intestine | 48.0 | 60.7 | Uterus | 33.7 | 61.6 |
| Colon ca. SW480 | 8.6 | 7.6 | Plancenta | 16.7 | 2.9 |
| Colon ca.* | 11.4 | 2.9 | Prostate | 11.7 | 17.8 |
| SW620(SW480 | | | | | |
| met) | | | | | |
| Colon ca. HT29 | 4.5 | 0.8 | Prostate ca.* | 17.0 | 19.6 |
| | | | (bone met)PC-3 | | |
| Colon ca. | 6.3 | 3.6 | Testis | 48.6 | 50.7 |
| HCT-116 | | | | | |
| Colon ca. CaCo-2 | 4.5 | 3.1 | Melanoma | 9.7 | 9.5 |
| | | | Hs688(A).T | | |
| Colon ca. | 31.6 | 7.9 | Melanoma* | 5.6 | 15.8 |
| tissue(ODO3866) | | | (met) | | |
| | | | Hs688(B).T | | |
| Colon ca. | 40.6 | 10.3 | Melanoma | 2.4 | 7.6 |
| HCC-2998 | | | UACC-62 | | |
| Gastric ca.* (liver | 85.9 | 55.1 | Melanoma | 10.2 | 89.5 |
| met) NCI-N87 | | | M14 | | |
| Bladder | 19.6 | 22.2 | Melanoma | 2.2 | 2.2 |
| | | | LOX IMVI | | |
| Trachea | 53.2 | 17.4 | Melanoma* | 3.9 | 1.9 |
| | | | (met) | | |
| | | | SK-MEL-5 | | |
| Kidney | 15.5 | 19.5 | Adipose | 18.3 | 15.5 |

TABLE 68
Panel 2D

| | Rel. Exp. (%) Ag2313, Run | | Rel. Exp. (%) Ag2313, Run |
|---|---|---|---|
| Tissue Name | 160021614 | Tissue Name | 160021614 |
| Normal Colon | 56.3 | Kidney Margin | 4.5 |
| | | 8120608 | |
| CC Well to Mod Diff | 6.9 | Kidney Cancer | 2.7 |
| (ODO3866) | | 8120613 | |
| CC Margin (ODO3866) | 7.4 | Kidney Margin | 9.2 |
| | | 8120614 | |
| CC Gr.2 rectosigmoid | 5.4 | Kidney Cancer | 7.5 |
| (ODO3868) | | 9010320 | |
| CC Margin (ODO3868) | 4.3 | Kidney Margin | 16.3 |
| | | 9010321 | |
| CC Mod Diff (ODO3920) | 14.5 | Normal Uterus | 7.6 |
| CC Margin (ODO3920) | 21.0 | Uterus Cancer 064011 | 24.3 |
| CC Gr.2 ascend colon | 29.3 | Normal Thyroid | 7.6 |
| (ODO3921) | | | |
| CC Margin (ODO3921) | 9.0 | Thyroid Cancer | 10.9 |
| | | 064010 | |
| CC from Partial | 20.9 | Thyroid Cancer | 27.7 |
| Hepatectomy (ODO4309) | | A302152 | |
| Mets | | | |
| Liver Margin (ODO4309) | 11.0 | Thyroid Margin | 13.5 |
| | | A302153 | |
| Colon mets to lung | 8.3 | Normal Breast | 24.7 |
| (OD04451-01) | | | |
| Lung Margin (OD04451- | 14.8 | Breast Cancer | 9.1 |
| 02) | | (OD04566) | |
| Normal Prostate 6546-1 | 5.7 | Breast Cancer | 25.0 |
| | | (OD04590-01) | |
| Prostate Cancer | 14.4 | Breast Cancer Mets | 26.4 |
| (OD04410) | | (OD04590-03) | |
| Prostate Margin | 27.7 | Breast Cancer | 23.0 |
| (OD04410) | | Metastasis | |
| | | (OD04655-05) | |
| Prostate Cancer | 20.4 | Breast Cancer 064006 | 13.8 |
| (OD04720-01) | | | |
| Prostate Margin | 30.4 | Breast Cancer 1024 | 29.3 |
| (OD04720-02) | | | |
| Normal Lung 061010 | 94.6 | Breast Cancer | 14.9 |
| | | 9100266 | |
| Lung Met to Muscle | 7.3 | Breast Margin | 10.5 |
| (ODO4286) | | 9100265 | |
| Muscle Margin | 17.6 | Breast Cancer | 14.3 |
| (ODO4286) | | A209073 | |
| Lung Malignant Cancer | 27.2 | Breast Margin | 20.9 |
| (OD03126) | | A2090734 | |
| Lung Margin (OD03126) | 100.0 | Normal Liver | 13.5 |
| Lung Cancer (OD04404) | 18.7 | Liver Cancer 064003 | 48.3 |
| Lung Margin (OD04404) | 21.3 | Liver Cancer 1025 | 17.2 |
| Lung Cancer (OD04565) | 5.8 | Liver Cancer 1026 | 1.2 |
| Lung Margin (OD04565) | 22.2 | Liver Cancer 6004-T | 23.0 |
| Lung Cancer (OD04237- | 29.3 | Liver Tissue 6004-N | 9.1 |
| 01) | | | |
| Lung Margin (OD04237- | 18.9 | Liver Cancer 6005-T | 2.2 |
| 02) | | | |
| Ocular Mel Met to Liver | 23.8 | Liver Tissue 6005-N | 1.7 |
| (ODO4310) | | | |
| Liver Margin (ODO4310) | 9.5 | Normal Bladder | 27.4 |
| Melanoma Mets to Lung | 6.6 | Bladder Cancer 1023 | 5.1 |
| (OD04321) | | | |
| Lung Margin (OD04321) | 46.7 | Bladder Cancer | 12.3 |
| | | A302173 | |
| Normal Kidney | 94.0 | Bladder Cancer | 6.8 |
| | | (OD04718-01) | |
| Kidney Ca, Nuclear grade | 33.4 | Bladder Normal | 21.9 |
| 2 (OD04338) | | Adjacent (OD04718- | |
| | | 03) | |
| Kidney Margin | 31.2 | Normal Ovary | 4.5 |
| (OD04338) | | | |
| Kidney Ca Nuclear grade | 26.6 | Ovarian Cancer | 12.9 |
| 1/2 (OD04339) | | 064008 | |
| Kidney Margin | 53.2 | Ovarian Cancer | 26.1 |
| (OD04339) | | (OD04768-07) | |
| Kidney Ca, Clear cell | 23.7 | Ovary Margin | 7.3 |
| type (OD04340) | | (OD04768-08) | |
| Kidney Margin | 45.7 | Normal Stomach | 26.4 |
| (OD04340) | | | |
| Kidney Ca, Nuclear grade | 8.4 | Gastric Cancer | 3.8 |
| 3 (OD04348) | | 9060358 | |
| Kidney Margin | 28.1 | Stomach Margin | 6.3 |
| (OD04348) | | 9060359 | |
| Kidney Cancer | 6.4 | Gastric Cancer | 11.7 |
| (OD04622-01) | | 9060395 | |
| Kidney Margin | 4.6 | Stomach Margin | 9.1 |
| (OD04622-03) | | 9060394 | |
| Kidney Cancer | 12.2 | Gastric Cancer | 8.8 |
| (OD04450-01) | | 9060397 | |
| Kidney Margin | 22.7 | Stomach Margin | 6.0 |
| (OD04450-03) | | 9060396 | |
| Kidney Cancer 8120607 | 2.9 | Gastric Cancer | 29.9 |
| | | 064005 | |

TABLE 69
Panel 4D

| | Rel. Exp. (%) Ag2313, Run | | Rel. Exp. (%) Ag2313, Run |
|---|---|---|---|
| Tissue Name | 160021630 | Tissue Name | 160021630 |
| Secondary Th1 act | 7.6 | HUVEC IL-1 beta | 4.6 |
| Secondary Th2 act | 9.1 | HUVEC IFN gamma | 14.8 |
| Secondary Tr1 act | 8.2 | HUVEC TNF alpha + IFN | 9.8 |
| | | gamma | |
| Secondary Th1 rest | 8.0 | HUVEC TNF alpha + IL4 | 6.5 |
| Secondary Th2 rest | 8.4 | HUVEC IL-11 | 3.2 |
| Secondary Tr1 rest | 10.7 | Lung Microvascular EC | 7.3 |
| | | none | |
| Primary Th1 act | 0.0 | Lung Microvascular EC | 7.7 |
| | | TNFalpha + IL-1 beta | |
| Primary Th2 act | 4.4 | Microvascular Dermal EC | 13.7 |
| | | none | |
| Primary Tr1 act | 8.2 | Microsvasular Dermal EC | 7.6 |
| | | TNFalpha + IL-1 beta | |
| Primary Th1 rest | 43.8 | Bronchial epithelium | 25.7 |
| | | TNFalpha + IL1 beta | |
| Primary Th2 rest | 31.0 | Small airway epithelium | 5.4 |
| | | none | |
| Primary Tr1 rest | 16.0 | Small airway epithelium | 100.0 |
| | | TNFalpha + IL-1 beta | |
| CD45RA CD4 | 16.4 | Coronery artery SMC rest | 8.1 |
| lymphocyte act | | | |
| CD45RO CD4 | 11.2 | Coronery artery SMC | 3.9 |
| lymphocyte act | | TNFalpha + IL-1 beta | |
| CD8 lymphocyte act | 8.4 | Astrocytes rest | 3.3 |
| Secondary CD8 | 9.0 | Astrocytes TNFalpha + | 2.5 |
| lymphocyte rest | | IL-1 beta | |
| Secondary CD8 | 7.9 | KU-812 (Basophil) rest | 6.9 |
| lymphocyte act | | | |
| CD4 lymphocyte none | 11.7 | KU-812 (Basophil) | 11.4 |
| | | PMA/ionomycin | |
| 2ry Th1/Th2/Tr1_anti- | 13.4 | CCD1106 (Keratinocytes) | 12.3 |
| CD95 CH11 | | none | |
| LAK cells rest | 33.9 | CCD1106 (Keratinocytes) | 13.6 |
| | | TNFalpha + IL-1 beta | |
| LAK cells IL-2 | 20.7 | Liver cirrhosis | 4.7 |
| LAK cells IL-2 + IL-12 | 13.8 | Lupus kidney | 3.4 |
| LAK cells IL-2 + IFN | 31.2 | NCI-H292 none | 41.5 |
| gamma | | | |
| LAK cells IL-2 + IL-18 | 35.1 | NCI-H292 IL-4 | 54.7 |
| LAK cells | 7.9 | NCI-H292 IL-9 | 43.5 |
| PMA/ionomycin | | | |
| NK Cells IL-2 rest | 15.2 | NCI-H292 IL-13 | 19.3 |
| Two Way MLR 3 day | 23.3 | NCI-H292 IFN gamma | 36.9 |
| Two Way MLR 5 day | 6.3 | HPAEC none | 3.6 |
| Two Way MLR 7 day | 6.6 | HPAEC TNF alpha + IL-1 | 7.7 |
| | | beta | |
| PBMC rest | 10.0 | Lung fibroblast none | 8.1 |
| PBMC PWM | 33.0 | Lung fibroblast TNF alpha | 12.7 |
| | | + IL-1 beta | |
| PBMC PHA-L | 13.0 | Lung fibroblast IL-4 | 12.7 |
| Ramos (B cell) none | 3.2 | Lung fibroblast IL-9 | 10.0 |
| Ramos (B cell) | 11.2 | Lung fibroblast IL-13 | 7.6 |
| ionomycin | | | |
| B lymphocytes PWM | 42.6 | Lung fibroblast IFN | 25.7 |
| | | gamma | |
| B lymphocytes CD40L | 33.7 | Dermal fibroblast | 32.5 |
| and IL-4 | | CCD1070 rest | |
| EOL-1 dbcAMP | 22.8 | Dermal fibroblast | 74.2 |
| | | CCD1070 TNF alpha | |
| EOL-1 dbcAMP | 6.0 | Dermal fibroblast | 17.7 |
| PMA/ionomycin | | CCD1070 IL-1 beta | |
| Dendritic cells none | 21.0 | Dermal fibroblast IFN | 19.1 |
| | | gamma | |
| Dendritic cells LPS | 25.3 | Dermal fibroblast IL-4 | 27.9 |
| Dendritic cells anti- | 26.2 | IBD Colitis 2 | 0.6 |
| CD40 | | | |
| Monocytes rest | 39.5 | IBD Crohn's | 1.2 |
| Monocytes LPS | 2.2 | Colon | 21.6 |
| Macrophages rest | 26.1 | Lung | 17.1 |
| Macrophages LPS | 9.4 | Thymus | 49.3 |
| HUVEC none | 10.2 | Kidney | 35.1 |
| HUVEC starved | 17.1 | | |

AI_comprehensive panel_v1.0 Summary: Ag2313 The NOV11a gene is expressed in normal lung, bone, joint tissue, gut and skin. There is no apparent difference in transcript expression in disease tissue as compared to normal tissue. Please see Panel 4D for discussion of utility of this gene in an autoimmune context.

CNS_neurodegeneration_v1.0 Summary: Ag2313 The expression profile of the NOV11a gene shows higher expression in the temporal cortex of Alzheimer's disease victims. This gene encodes an alpha glucosidase homolog. Brain glucose regulation may play a role in Alzheimer's disease. For example, hyperglycemia may exert a deleterious effect by potentiating the neuronal death produced by pathological processes taking place, such as amyloid deposition. The category of agents that interfere with the absorption of glucose and lipids includes alpha-glucosidase inhibitors. Therefore, modulators of this gene product may be useful in the treatment of Alzheimer's disease.

References:

Messier C, Gagnon M. Glucose regulation and cognitive functions: relation to Alzheimer's disease and diabetes. Behav Brain Res February 1996;75(1-2):1-11

Glucose has been found to improve memory in animals and humans. Animal research has revealed that glucose may improve memory through a facilitation of acetylcholine (ACh) synthesis and release in the brain. This glucose-related memory improvement has prompted research in elderly humans. These studies have shown that the memory-improving action of glucose depends on each individuals' blood glucose regulation. Based on these data, researchers have evaluated the effect of glucose on memory in patients with Alzheimer's disease (AD). Results demonstrated that glucose could improve memory in a subset of patients that had abnormalities in their blood glucose regulation. Interestingly, these alterations in blood glucose regulation were believed to depend on the severity of the disease process. Another line of investigation has focused on alterations in brain glucose metabolism. Both animal models and studies with Type II diabetic elderly patients have shown that altered glucose regulation impairs learning and memory processes. It is possible that in AD patients, hyperglycemia exerts a deleterious effect by potentiating the neuronal death produced by other pathological processes taking place such as amyloid deposition. Based on these data, it appears important to find the prevalence of altered glucoregulation at various stages of AD. Secondly, it may be of interest to determine prospectively whether altered glucoregulation is linked to a faster progression of the disease. Finally, if such a relationship is observed, the next logical step would be to determine whether AD patients could benefit from treatments aimed at normalizing blood glucose regulation and improving insulin sensitivity.

Panel 1 Summary: Ag401 The expression of the NOV11a gene appears to be highest in a sample derived from testis tissue (CT=26.1). In addition to testis tissue, there appears to be substantial expression associated with the cerebellar region of the brain and mammary gland. The other samples in the panel show low uniform expression. Thus, the expression of this gene could be used to distinguish testis, mammary gland and cerebellum from the other samples in the panel.

Please see Panel CNS_neurodegeneration_v1.0 for discussion of potential utility of this gene in the central nervous system.

This gene is also moderately expressed in a number of metabolic tissues including pancreas, adrenal, thyroid, pituitary, heart, skeletal muscle, and adult and fetal liver. This small molecule target may be useful for the treatment of metabolic diseases, including obesity and Types 1 and 2 diabetes.

Panel 1.3D Summary: Ag401/Ag2313 Two experiments expression of the NOV11a gene in a wide variety of metabolic tissues, including pancreas, adipose, adrenal, thyroid, pituitary, adult and fetal heart, adult and fetal skeletal muscle, and adult and fetal liver. Alpha-glucosidase inhibitors are currently used in the treatment of Type 2 diabetes to decrease glucose absorption from the gut. Thus, this gene product may be a small molecule target for the treatment of metabolic diseases, including obesity and Types 1 and 2 diabetes.

Significant brain expression of the gene is also seen in this panel. Please see Panel CNS_neurodegeneration_v1.0 for discussion of potential utility of this gene in the central nervous system.

References:

Raptis S A, Dimitriadis G D. Oral hypoglycemic agents: insulin secretagogues, alpha-glucosidase inhibitors and insulin sensitizers. Exp Clin Endocrinol Diabetes. 2001;109 Suppl 2:S265-87.

In this review we present the agents that are in use in the treatment of type 2 diabetes. Sulfonylureas of the 1st and 2nd generation increase insulin secretion but can induce hyperinsulinemia and sometimes prolonged hypoglycemia. Glimepiride is a new 3rd generation sulfonylurea with some advantages over the other members of this group, such as a lower risk of hypoglycemia, no interaction with cardiovascular KATP-channels and a possibility that it may increase insulin sensitivity. There are also newer insulin secretagogues (such as neteglinide and repaglinide) with a rapid onset of action on the beta-cell, therefore inducing a more physiological profile of insulin secretion during meals. The category of insulin sensitizers includes metformin and thiazolidinediones. Metformin effectively reduces hyperglycemia, hyperlipidemia and macroangiopathy in patients with type 2 diabetes. This agent increases the sensitivity of the liver and peripheral tissues to insulin and, therefore, it could be considered as a drug of choice for the prevention of type 2 diabetes. Thiazolidinediones (rosiglitazone and pioglitazone) increase the sensitivity of the tissues to insulin. This mechanism of action makes them powerful therapeutic tools for the treatment of type 2 diabetes (and possibly other insulin resistant states) either alone or in combination with other oral agents. The category of agents that interfere with the absorption of glucose and lipids includes alpha-glucosidase inhibitors (acarbose and miglitol) and lipase inhibitors (or-listat). alpha-Glucocidase inhibitors improve the time relationship between plasma insulin and glucose increases after a meal. Therefore, these agents may be used in the treatment of patients with type 2 diabetes, either alone at a very early stage of this disease (when insulin secretion is still adequate), or in combination with insulin secretagogues. alpha-Glucosidase inhibition may also prove useful as a supplement to insulin therapy in patients with type 1 diabetes mellitus. The inhibitor of gastrointestinal lipase orlistat may prove, a useful adjunct to hypocaloric diets in patients with type 2 diabetes and obesity.

PMID: 11460577

Panel 2D Summary: Ag2313 The expression of the NOV11a gene appears to be highest in a sample derived from normal lung tissue adjacent to malignant lung (CT=27.3). In addition there is substantial expression associated with another sample of normal lung tissue as well a number of normal kidney tissue samples while absent in adjacent malignant kidney.

Thus, the expression of this gene could be used to distinguish these tissues from other tissues in the panel. Moreover, therapeutic modulation of this gene, through the use of small molecule drugs, antibodies or protein therapeutics might be of benefit in the treatment of lung or kidney cancer.

Panel 4D Summary: Ag2313 The NOV11a transcript is expressed at high levels in small airway epithelium activated with TNFalpha and IL-1beta. It is also expressed in normal tissues such as the lung, thymus and kidney, as seen in Panel AI. The protein encoded by this transcript is related to Alpha glucosidase A. This enzyme is important in lung maturation and may contribute to surfactant phospholipid biosynthesis (see reference). Therefore, therapeutics designed with the protein encoded by this transcript or therapeutics that modulate its production could potentially be used to aid lung surfactant production in premature infants, and to treat lung injuries.

References:

Bourbon J R, Doucet E, Rieutort M. Role of alpha-glucosidase in fetal lung maturation.Biochim Biophys Acta Jan. 13, 1987;917(1):203-10

The role of lysosomal enzyme acid alpha-glucosidase in fetal lung development was investigated with the aid of a specific inhibitor, the pseudosaccharide acarbose. The drug was added to a Waymouth culture medium of fetal rat lung explants cultivated for 48 h fromgestational stage 19.5 days, an in vitro system previously shown to allow morphological and biochemical maturation of alveolar epithelium. Glycogenolysis was reduced by 40% as compared with tissue cultivated on control medium, which means thatalpha-glucosidase could account for as much as 40% of fetal lung glycogenolysis, the remaining 60% being presumably achieved by cytosolic phosphorylase and by a microsomal neutral alpha-glucosidase. By the same time, the increase of phospholipids of surfactant fraction extracted from cultivated explants was partially inhibited: total and saturated phosphatidylcholine, phosphatidylethanolamine, phosphatidylglycerol and phosphatidylinositol were about 30-40% lower than in lungs cultivated on control medium. It should beemphasized that DNA concentration and increases in non-surfactant phospholipids were unchanged by the drug. alpha-Glucosidase activity was evidenced in the lysosomal fraction, in the microsomal fraction and, although in lower amounts, in the surfactant fraction extracted from term fetal lung. The results suggest that lysosomal alpha-glucosidase plays a major role in lung maturation and could facilitate glycogenolysis for the specific use of glycogen stores in providing substrates for surfactant phospholipid biosynthesis.PMID: 3539207

NOV11b

Expression of gene NOV11b was assessed using the primer-probe sets Ag3195 and Ag401, described in Tables 70, 71, and 72. Results of the RTQ-PCR runs are shown in Tables 73, 74, 75, 76, 77, 78 and 79.

TABLE 70

Probe Name Ag3195

| | | | | |
|---|---|---|---|---|
| Primers | Sequences | Length | Start Position | SEQ ID NO: |

| | | | | |
|---|---|---|---|---|
| Forward | 5′-aatagttttgctgaccagagg-3′ | 21 | 2730 | 220 |
| Probe | TET-5′-cccagcaagtgtgtggtggag-3′-TAMRA | 21 | 2760 | 221 |
| Reverse | 5′tgatctttaccatcagatgagt-3′ | 22 | 2830 | 222 |

TABLE 71

Probe Name Ag401

| | | | | |
|---|---|---|---|---|
| Primers | Sequences | Length | Start Position | SEQ ID NO: |

| | | | | |
|---|---|---|---|---|
| Forward | 5′-ttgtgccaaaacatccatcct-3′ | 21 | 70 | 223 |
| Probe | TET-5′-agcctggagaagctctcactcaacattgc-3′-TAMRA | 29 | 2892 | 224 |
| Reverse | 5′-tatgatgcggacctcccagt-3′ | 20 | 2926 | 225 |

TABLE 72

Probe Name Ag3154

| | | | | |
|---|---|---|---|---|
| Primers | Sequences | Length | Start Position | SEQ ID NO: |

| | | | | |
|---|---|---|---|---|
| Forward | 5′-ccgtaatgctgtcccacttat-3′ | 21 | 4249 | 226 |
| Probe | TET-5′-tgtgctctacttagcattctcagggatca-3′-TAMRA | 29 | 4273 | 227 |
| Reverse | 5′-aggagaacgcagacatactgaa-3′ | 22 | 4314 | 228 |

TABLE 73
A1_comprehensive panel_v1.0

| | Rel. Exp. (%) Ag3195, Run | | Rel. Exp. (%) Ag3195, Run |
|---|---|---|---|
| Tissue Name | 225147682 | Tissue Name | 225147682 |
| 110967 COPD-F | 17.3 | 112427 Match Control | 66.9 |
| | | Psoriasis-F | |
| 110980 COPD-F | 15.3 | 112418 Psoriasis-M | 14.2 |
| 110968 COPD-M | 14.4 | 112723 Match Control | 4.0 |
| | | Psoriasis-M | |
| 110977 COPD-M | 37.1 | 112419 Psoriasis-M | 19.5 |
| 110989 Emphysema-F | 29.1 | 112424 Match Control | 13.7 |
| | | Psoriasis-M | |
| 110992 Emphysema-F | 11.3 | 112420 Psoriasis-M | 59.0 |
| 110993 Emphysema-F | 9.9 | 112425 Match Control | 57.8 |
| | | Psoriasis-M | |
| 110994 Emphysema-F | 9.3 | 104689 (MF) OA | 33.4 |
| | | Bone-Backus | |
| 110995 Emphysema-F | 25.3 | 104690 (MF) Adj | 22.4 |
| | | “Normal” Bone-Backus | |
| 110996 Emphysema-F | 3.2 | 104691 (MF) OA | 28.5 |
| | | Synovium-Backus | |
| 110997 Asthma-M | 4.8 | 104692 (BA) OA | 14.8 |
| | | Cartilage-Backus | |
| 111001 Asthma-F | 18.8 | 104694 (BA) OA | 22.1 |
| | | Bone-Backus | |
| 111002 Asthma-F | 13.6 | 104695 (BA) Adj | 27.0 |
| | | “Normal” Bone-Backus | |
| 111003 Atopic | 14.6 | 104696 (BA) OA | 15.5 |
| Asthma-F | | Synovium-Backus | |
| 111004 Atopic | 21.5 | 104700 (SS) OA Bone- | 16.6 |
| Asthma-F | | Backus | |
| 111005 Atopic | 14.5 | 104701 (SS) Adj | 16.5 |
| Asthma-F | | “Normal” Bone-Backus | |
| 111006 Atopic | 2.1 | 104702 (SS) OA | 29.1 |
| Asthma-F | | Synovium-Backus | |
| 111417 Allergy-M | 12.2 | 117093 OA Cartilage | 33.0 |
| | | Rep7 | |
| 112347 Allergy-M | 0.0 | 112672 OA Bone5 | 19.2 |
| 112349 Normal Lung-F | 0.0 | 112673 OA Synovium5 | 11.0 |
| 112357 Normal Lung-F | 11.6 | 112674 OA Synovial | 8.1 |
| | | Fluid cells5 | |
| 112354 Normal Lung-M | 14.9 | 117100 OA Cartilage | 5.0 |
| | | Rep14 | |
| 112374 Crohns-F | 18.6 | 112756 OA Bone9 | 5.4 |
| 112389 Match | 15.7 | 112757 OA Synovium9 | 25.7 |
| Control Crohns-F | | | |
| 112375 Crohns-F | 16.8 | 112758 OA Synovial | 15.6 |
| | | Fluid Cells9 | |
| 112732 Match | 20.4 | 117125 RA Cartilage | 19.1 |
| Control Crohns-F | | Rep2 | |
| 112725 Crohns-M | 3.8 | 113492 Bone2 RA | 26.8 |
| 112387 Match | 15.7 | 113493 Synovium2 RA | 8.9 |
| Control Crohns-M | | | |
| 112378 Crohns-M | 0.0 | 113494 Syn Fluid Cells | 19.5 |
| | | RA | |
| 112390 Match | 100.0 | 113499 Cartilage4 RA | 19.8 |
| Control Crohns-M | | | |
| 112726 Crohns-M | 23.0 | 113500 Bone4 RA | 27.9 |
| 112731 Match | 14.1 | 113501 Synovium4 RA | 19.2 |
| Control Crohns-M | | | |
| 112380 Ulcer Col-F | 41.8 | 113502 Syn Fluid | 13.6 |
| | | Cells4 RA | |
| 112734 Match | 46.7 | 113495 Cartilage3 RA | 17.2 |
| Control Ulcer Col-F | | | |
| 112384 Ulcer Col-F | 51.4 | 113496 Bone3 RA | 19.9 |
| 112737 Match | 6.5 | 113497 Synovium3 RA | 9.8 |
| Control Ulcer Col-F | | | |
| 112386 Ulcer Col-F | 10.4 | 113498 Syn Fluid | 25.2 |
| | | Cells3 RA | |
| 112738 Match | 4.5 | 117106 Normal | 6.2 |
| Control Ulcer Col-F | | Cartilage Rep20 | |
| 112381 Ulcer Col-M | 0.0 | 113663 Bone3 Normal | 0.0 |
| 112735 Match | 7.4 | 113664 Synovium3 | 0.0 |
| Control Ulcer Col-M | | Normal | |
| 112382 Ulcer Col-M | 11.3 | 113665 Syn Fluid | 0.0 |
| | | Cells3 Normal | |
| 112394 Match | 6.4 | 117107 Normal | 8.1 |
| Control Ulcer Col-M | | Cartilage Rep22 | |
| 112383 Ulcer Col-M | 21.2 | 113667 Bone4 Normal | 11.0 |
| 112736 Match | 9.6 | 113668 Synovium4 | 15.7 |
| Control Ulcer Col-M | | Normal | |
| 112423 Psoriasis-F | 17.9 | 113669 Syn Fluid | 18.0 |
| | | Cells4 Normal | |

TABLE 74
CNS_neurodegeneration_v1.0

| | Rel. Exp. (%) Ag3195, | | Rel. Exp. (%) Ag3195, |
|---|---|---|---|
| Tissue Name | Run 209859542 | Tissue Name | Run 209859542 |
| AD 1 Hippo | 16.6 | Control (Path) 3 | 6.5 |
| | | Temporal Ctx | |
| AD 2 Hippo | 31.0 | Control (Path) 4 | 34.4 |
| | | Temporal Ctx | |
| AD 3 Hippo | 6.8 | AD 1 Occipital Ctx | 14.0 |
| AD 4 Hippo | 8.7 | AD 2 Occipital Ctx | 0.0 |
| | | (Missing) | |
| AD 5 hippo | 60.7 | AD 3 Occipital Ctx | 13.1 |
| AD 6 Hippo | 63.7 | AD 4 Occipital Ctx | 29.1 |
| Control 2 Hippo | 32.1 | AD 5 Occipital Ctx | 19.6 |
| Control 4 Hippo | 17.6 | AD 6 Occipital Ctx | 26.8 |
| Control (Path) 3 | 12.8 | Control 1 Occipital | 3.3 |
| Hippo | | Ctx | |
| AD 1 Temporal Ctx | 35.6 | Control 2 Occipital | 51.1 |
| | | Ctx | |
| AD 2 Temporal Ctx | 37.1 | Control 3 Occipital | 15.3 |
| | | Ctx | |
| AD 3 Temporal Ctx | 7.9 | Control 4 Occipital | 12.8 |
| | | Ctx | |
| AD 4 Temporal Ctx | 32.8 | Control (Path) 1 | 100.0 |
| | | Occipital Ctx | |
| AD 5 Inf Temporal | 95.9 | Control (Path) 2 | 16.4 |
| Ctx | | Occipital Ctx | |
| AD 5 SupTemporal | 56.3 | Control (Path) 3 | 8.4 |
| Ctx | | Occipital Ctx | |
| AD 6 Inf Temporal | 90.8 | Control (Path) 4 | 17.4 |
| Ctx | | Occipital Ctx | |
| AD 6 Sup Temporal | 68.8 | Control 1 Parietal | 8.4 |
| Ctx | | Ctx | |
| Control 1 Temporal | 4.9 | Control 2 Parietal | 57.8 |
| Ctx | | Ctx | |
| Control 2 Temporal | 33.7 | Control 3 Parietal | 14.4 |
| Ctx | | Ctx | |
| Control 3 Temporal | 14.1 | Control (Path) 1 | 63.7 |
| Ctx | | Parietal Ctx | |
| Control 4 Temporal | 9.4 | Control (Path) 2 | 28.7 |
| Ctx | | Parietal Ctx | |
| Control (Path) 1 | 49.0 | Control (Path) 3 | 3.6 |
| Temporal Ctx | | Parietal Ctx | |
| Control (Path) 2 | 32.1 | Control (Path) 4 | 56.3 |
| Temporal Ctx | | Parietal Ctx | |

TABLE 75
Panel 1

| | Rel. Exp. (%) Ag401, | | Rel. Exp. (%) Ag401, |
|---|---|---|---|
| Tissue Name | Run 88225002 | Tissue Name | Run 88225002 |
| Endothelial cells | 1.5 | Renal ca. 786-0 | 10.7 |
| Endothelial cells | 2.5 | Renal ca. A498 | 4.2 |
| (treated) | | | |
| Pancreas | 11.0 | Renal ca. RXF 393 | 4.8 |
| Pancreatic ca. CAPAN 2 | 2.6 | Renal ca. ACHN | 4.9 |
| Adrenal gland | 8.5 | Renal ca. UO-31 | 3.8 |
| Thyroid | 13.6 | Renal ca. TK-10 | 5.0 |
| Salivary gland | 12.7 | Liver | 24.0 |
| Pituitary gland | 8.3 | Liver (fetal) | 4.0 |
| Brain (fetal) | 4.2 | Liver ca. (hepatoblast) | 7.1 |
| | | HepG2 | |
| Brain (whole) | 14.7 | Lung | 3.1 |
| Brain (amygdala) | 4.1 | Lung (fetal) | 12.9 |
| Brain (cerebellum) | 74.2 | Lung ca. (small cell) | 9.5 |
| | | LX-1 | |
| Brain (hippocampus) | 7.4 | Lung ca. (small cell) | 0.8 |
| | | NCI-H69 | |
| Brain (substantia nigra) | 13.6 | Lung ca. (s.cell var.) | 13.2 |
| | | SHP-77 | |
| Brain (thalamus) | 7.3 | Lung ca. (large | 8.5 |
| | | cell) NCI-H460 | |
| Brain (hypothalamus) | 12.6 | Lung ca. (non-sm. | 4.3 |
| | | cell) A549 | |
| Spinal cord | 12.9 | Lung ca. (non-s.cell) | 5.7 |
| | | NCI-H23 | |
| glio/astro U87-MG | 3.5 | Lung ca. (non-s.cell) | 3.7 |
| | | HOP-62 | |
| glio/astro U-118-MG | 7.6 | Lung ca. (non-s.cl) | 10.9 |
| | | NCI-H522 | |
| astrocytoma SW1783 | 2.0 | Lung ca. (squam.) SW | 8.4 |
| | | 900 | |
| neuro*; met SK-N-AS | 4.4 | Lung ca. (squam.) | 1.2 |
| | | NCI-H596 | |
| astrocytoma SF-539 | 5.6 | Mammary gland | 32.1 |
| astrocytoma SNB-75 | 5.4 | Breast ca.* (pl.ef) | 5.6 |
| | | MCF-7 | |
| glioma SNB-19 | 7.0 | Breast ca.* (pl.ef) | 3.3 |
| | | MDA-MB-231 | |
| glioma U251 | 5.5 | Breast ca.* (pl. ef) | 12.3 |
| | | T47D | |
| glioma SF-295 | 5.3 | Breast ca. BT-549 | 6.7 |
| Heart | 8.8 | Breast ca. MDA-N | 8.8 |
| Skeletal muscle | 14.6 | Ovary | 10.4 |
| Bone marrow | 9.3 | Ovarian ca. OVCAR-3 | 3.1 |
| Thymus | 27.7 | Ovarian ca. OVCAR-4 | 1.4 |
| Spleen | 14.6 | Ovarian ca. OVCAR-5 | 12.6 |
| Lymph node | 21.6 | Ovarian ca. OVCAR-8 | 3.4 |
| Colon (ascending) | 13.7 | Ovarian ca. IGROV-1 | 3.2 |
| Stomach | 20.4 | Ovarian ca. (ascites) | 8.3 |
| | | SK-OV-3 | |
| Small intestine | 17.4 | Uterus | 16.7 |
| Colon ca. SW480 | 1.5 | Placenta | 11.2 |
| Colon ca.* SW620 | 4.2 | Prostate | 12.4 |
| (SW480 met) | | | |
| Colon ca. HT29 | 1.5 | Prostate ca.* (bone | 13.1 |
| | | met) PC-3 | |
| Colon ca. HCT-116 | 5.3 | Testis | 100.0 |
| Colon ca. CaCo-2 | 3.5 | Melanoma | 12.4 |
| | | Hs688(A).T | |
| Colon ca. HCT-15 | 7.2 | Melanoma* (met) | 6.4 |
| | | Hs688(B).T | |
| Colon ca. HCC-2998 | 6.4 | Melanoma UACC-62 | 1.6 |
| Gastric ca. * (liver met) | 26.1 | Melanoma M14 | 33.9 |
| NCI-N87 | | | |
| Bladder | 4.3 | Melanoma LOX | 8.0 |
| | | IMVI | |
| Trachea | 20.9 | Melanoma* (met) SK- | 3.0 |
| | | MEL-5 | |
| Kidney | 14.9 | Melanoma SK-MEL-28 | 15.9 |
| Kidney (fetal) | 17.3 | | |

TABLE 76
Panel 1.3D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag3195, | Ag3195, | Ag401, | | Ag3195, | Ag3195, | Ag401, |
| | Run | Run | Run | | Run | Run | Run |
| | 16552493 | 16567539 | 16551817 | Tissue | 16552493 | 16567539 | 16551817 |
| Tissue Name | 0 | 1 | 4 | Name | 0 | 1 | 4 |
| Liver | 9.0 | 3.3 | 2.9 | Kidney | 13.9 | 7.2 | 13.8 |
| adenocarcinoma | | | | (fetal) | | | |
| Pancreas | 19.5 | 10.0 | 10.1 | Renal ca. | 14.5 | 7.7 | 14.8 |
| | | | | 786-0 | | | |
| Pancreatic ca. | 0.6 | 0.0 | 10.2 | Renal ca. | 9.9 | 13.9 | 13.9 |
| CAPAN 2 | | | | A498 | | | |
| Adrenal gland | 12.9 | 8.2 | 16.2 | Renal ca. | 10.7 | 9.4 | 18.2 |
| | | | | RXF 393 | | | |
| Thyroid | 14.3 | 11.3 | 18.9 | Renal ca. | 3.6 | 4.1 | 12.7 |
| | | | | ACHN | | | |
| Salivary gland | 11.8 | 15.6 | 24.7 | Renal ca. | 1.8 | 3.1 | 12.2 |
| | | | | UO-31 | | | |
| Pituitary gland | 11.0 | 7.0 | 20.2 | Renal ca. | 3.4 | 3.2 | 5.7 |
| | | | | TK-10 | | | |
| Brain (fetal) | 13.3 | 13.9 | 23.5 | Liver | 21.5 | 16.2 | 16.7 |
| Brain (whole) | 51.4 | 36.3 | 52.1 | Liver (fetal) | 8.8 | 13.8 | 9.0 |
| Brain | 39.5 | 29.7 | 16.2 | Liver ca. | 6.6 | 15.4 | 9.6 |
| (amygdala) | | | | (hepatoblast) | | | |
| | | | | HepG2 | | | |
| Brain | 26.4 | 27.2 | 47.6 | Lung | 26.2 | 47.6 | 19.5 |
| (cerebellum) | | | | | | | |
| Brain | 39.8 | 22.7 | 54.3 | Lung (fetal) | 25.3 | 62.0 | 11.2 |
| (hippocampus) | | | | | | | |
| Brain | 26.6 | 22.2 | 20.0 | Lung ca. | 11.1 | 14.1 | 18.7 |
| (substantia | | | | (small cell) | | | |
| nigra) | | | | LX-1 | | | |
| Brain | 46.3 | 27.0 | 41.5 | Lung ca. | 0.4 | 0.0 | 0.0 |
| (thalamus) | | | | (small cell) | | | |
| | | | | NCI-H69 | | | |
| Cerebral Cortex | 5.0 | 8.2 | 15.0 | Lung ca. | 6.3 | 10.7 | 10.9 |
| | | | | (s.cell var.) | | | |
| | | | | SHP-77 | | | |
| Spinal cord | 46.3 | 38.7 | 38.7 | Lung ca. | 13.5 | 18.0 | 19.5 |
| | | | | (large | | | |
| | | | | cell) NCI- | | | |
| | | | | H460 | | | |
| glio/astro U87- | 2.4 | 1.6 | 9.5 | Lung ca. | 0.8 | 1.8 | 1.6 |
| MG | | | | (non-sm. | | | |
| | | | | cell) A549 | | | |
| glio/astro U- | 28.9 | 29.3 | 49.7 | Lung ca. | 5.1 | 3.2 | 9.3 |
| 118-MG | | | | (non-s.cell) | | | |
| | | | | NCI-H23 | | | |
| astrocytoma | 8.2 | 10.9 | 10.4 | Lung ca. | 19.2 | 7.6 | 17.8 |
| SW1783 | | | | (non-s.cell) | | | |
| | | | | HOP-62 | | | |
| neuro*; met SK- | 11.7 | 6.3 | 20.2 | Lung ca. | 2.2 | 2.9 | 9.0 |
| N-AS | | | | (non-s.cl) | | | |
| | | | | NCI-H522 | | | |
| astrocytoma SF- | 16.4 | 9.2 | 25.7 | Lung ca. | 8.1 | 6.5 | 4.5 |
| 539 | | | | (squam.) | | | |
| | | | | SW 900 | | | |
| astrocytoma | 8.3 | 14.5 | 22.4 | Lung ca. | 0.0 | 1.6 | 3.1 |
| SNB-75 | | | | (squam.) | | | |
| | | | | NCI-H596 | | | |
| glioma SNB-19 | 3.2 | 5.5 | 25.9 | Mammary | 44.1 | 22.1 | 27.0 |
| | | | | gland | | | |
| glioma U251 | 26.6 | 20.3 | 59.9 | Breast ca.* | 4.4 | 1.7 | 4.3 |
| | | | | (pl.ef) | | | |
| | | | | MCF-7 | | | |
| glioma SF-295 | 10.1 | 5.5 | 15.4 | Breast ca.* | 21.0 | 15.4 | 47.3 |
| | | | | (pl.ef) | | | |
| | | | | MDA-MB- | | | |
| | | | | 231 | | | |
| Heart (fetal) | 1.9 | 2.5 | 0.5 | Breast ca.* | 3.1 | 4.9 | 6.4 |
| | | | | (pl.ef) | | | |
| | | | | T47D | | | |
| Heart | 8.0 | 15.2 | 14.0 | Breast ca. | 12.5 | 7.3 | 12.9 |
| | | | | BT-549 | | | |
| Skeletal muscle | 2.9 | 1.8 | 7.0 | Breast ca. | 4.4 | 1.9 | 6.2 |
| (fetal) | | | | MDA-N | | | |
| Skeletal muscle | 57.0 | 58.2 | 100.0 | Ovary | 10.9 | 6.5 | 6.3 |
| Bone marrow | 28.1 | 13.0 | 33.9 | Ovarian ca. | 8.4 | 6.3 | 10.5 |
| | | | | OVCAR-3 | | | |
| Thymus | 27.2 | 16.0 | 32.1 | Ovarian ca. | 3.6 | 2.9 | 7.2 |
| | | | | OVCAR-4 | | | |
| Spleen | 39.0 | 27.0 | 27.9 | Ovarian ca. | 11.2 | 9.2 | 13.6 |
| | | | | OVCAR-5 | | | |
| Lymph node | 100.0 | 100.0 | 76.8 | Ovarian ca. | 3.9 | 4.5 | 4.6 |
| | | | | OVCAR-8 | | | |
| Colorectal | 25.0 | 23.2 | 38.7 | Ovarian ca. | 16.3 | 1.6 | 2.5 |
| | | | | IGROV-1 | | | |
| Stomach | 23.3 | 25.3 | 21.0 | Ovarian | 10.1 | 11.6 | 17.3 |
| | | | | ca.* | | | |
| | | | | (ascites) | | | |
| | | | | SK-OV-3 | | | |
| Small intestine | 55.9 | 41.8 | 60.7 | Uterus | 65.5 | 61.1 | 61.6 |
| Colon ca. | 5.6 | 2.9 | 7.6 | Placenta | 6.6 | 10.2 | 2.9 |
| SW480 | | | | | | | |
| Colon ca.* | 6.7 | 4.9 | 2.9 | Prostate | 14.8 | 8.5 | 17.8 |
| SW620(SW480 | | | | | | | |
| met) | | | | | | | |
| Colon ca. HT29 | 2.8 | 1.1 | 0.8 | Prostate | 3.5 | 6.9 | 19.6 |
| | | | | ca.* (bone | | | |
| | | | | met)PC-3 | | | |
| Colon ca. HCT- | 4.5 | 3.2 | 3.6 | Testis | 45.1 | 23.8 | 50.7 |
| 116 | | | | | | | |
| Colon ca. | 2.0 | 2.0 | 3.1 | Melanoma | 7.2 | 3.7 | 9.5 |
| CaCo-2 | | | | Hs688(A).T | | | |
| Colon ca. | 14.5 | 5.8 | 7.9 | Melanoma* | 10.0 | 6.7 | 15.8 |
| tissue(ODO3866) | | | | (met) | | | |
| | | | | Hs688(B).T | | | |
| Colon ca. HCC- | 6.4 | 4.6 | 10.3 | Melanoma | 16.5 | 12.5 | 7.6 |
| 2998 | | | | UACC-62 | | | |
| Gastric ca.* | 48.0 | 34.9 | 55.1 | Melanoma | 73.7 | 51.8 | 89.5 |
| (liver met) NCI- | | | | M14 | | | |
| N87 | | | | | | | |
| Bladder | 21.3 | 23.3 | 22.2 | Melanoma | 1.1 | 0.6 | 2.2 |
| | | | | LOX IMVI | | | |
| Trachea | 21.5 | 26.1 | 17.4 | Melanoma* | 0.4 | 1.7 | 1.9 |
| | | | | (met) SK- | | | |
| | | | | MEL-5 | | | |
| Kidney | 44.8 | 35.8 | 19.5 | Adipose | 22.1 | 9.9 | 15.5 |

TABLE 77
Panel 2D

| | Rel. Exp. (%) Ag3195, Run | | Rel. Exp. (%) Ag3195, Run |
|---|---|---|---|
| Tissue Name | 165003127 | Tissue Name | 165003127 |
| Normal Colon | 51.1 | Kidney Margin | 1.4 |
| | | 8120608 | |
| CC Well to Mod Diff | 6.9 | Kidney Cancer | 2.5 |
| (ODO3866) | | 8120613 | |
| CC Margin (ODO3866) | 5.9 | Kidney Margin | 6.7 |
| | | 8120614 | |
| CC Gr.2 rectosigmoid | 4.3 | Kidney Cancer | 9.0 |
| (ODO3868) | | 9010320 | |
| CC Margin (ODO3868) | 2.4 | Kidney Margin | 15.5 |
| | | 9010321 | |
| CC Mod Diff (ODO3920) | 6.4 | Normal Uterus | 5.9 |
| CC Margin (ODO3920) | 12.6 | Uterus Cancer 064011 | 31.9 |
| CC Gr.2 ascend colon | 24.3 | Normal Thyroid | 10.2 |
| (ODO3921) | | | |
| CC Margin (ODO3921) | 9.2 | Thyroid Cancer | 12.5 |
| | | 064010 | |
| CC from Partial | 22.8 | Thyroid Cancer | 16.2 |
| Hepatectomy (ODO4309) | | A302152 | |
| Mets | | | |
| Liver Margin (ODO4309) | 14.7 | Thyroid Margin | 18.9 |
| | | A302153 | |
| Colon mets to lung | 3.5 | Normal Breast | 24.8 |
| (OD04451-01) | | | |
| Lung Margin (OD04451- | 7.1 | Breast Cancer | 5.8 |
| 02) | | (OD04566) | |
| Normal Prostate 6546-1 | 25.5 | Breast Cancer | 16.0 |
| | | (OD04590-01) | |
| Prostate Cancer | 10.6 | Breast Cancer Mets | 28.9 |
| (OD04410) | | (OD04590-03) | |
| Prostate Margin | 9.4 | Breast Cancer | 7.9 |
| (OD04410) | | Metastasis | |
| | | (OD04655-05) | |
| Prostate Cancer | 15.2 | Breast Cancer 064006 | 10.4 |
| (OD04720-01) | | | |
| Prostate Margin | 24.0 | Breast Cancer 1024 | 10.7 |
| (OD04720-02) | | | |
| Normal Lung 061010 | 100.0 | Breast Cancer | 7.2 |
| | | 9100266 | |
| Lung Met to Muscle | 5.3 | Breast Margin | 7.2 |
| (ODO4286) | | 9100265 | |
| Muscle Margin | 13.7 | Breast Cancer | 17.2 |
| (ODO4286) | | A209073 | |
| Lung Malignant Cancer | 24.0 | Breast Margin | 12.2 |
| (OD03126) | | A2090734 | |
| Lung Margin (OD03126) | 74.7 | Normal Liver | 27.0 |
| Lung Cancer (OD04404) | 14.0 | Liver Cancer 064003 | 45.7 |
| Lung Margin (OD04404) | 18.9 | Liver Cancer 1025 | 8.6 |
| Lung Cancer (OD04565) | 8.0 | Liver Cancer 1026 | 1.8 |
| Lung Margin (OD04565) | 16.7 | Liver Cancer 6004-T | 0.7 |
| Lung Cancer (OD04237- | 23.8 | Liver Tissue 6004-N | 6.0 |
| 01) | | | |
| Lung Margin (OD04237- | 17.3 | Liver Cancer 6005-T | 1.7 |
| 02) | | | |
| Ocular Mel Met to Liver | 19.3 | Liver Tissue 6005-N | 1.5 |
| (ODO4310) | | | |
| Liver Margin (ODO4310) | 14.3 | Normal Bladder | 35.6 |
| Melanoma Mets to Lung | 7.6 | Bladder Cancer 1023 | 3.2 |
| (OD04321) | | | |
| Lung Margin (OD04321) | 40.3 | Bladder Cancer | 15.7 |
| | | A302173 | |
| Normal Kidney | 87.7 | Bladder Cancer | 6.7 |
| | | (OD04718-01) | |
| Kidney Ca, Nuclear grade | 28.9 | Bladder Normal | 21.6 |
| 2 (OD04338) | | Adjacent (OD04718- | |
| | | 03) | |
| Kidney Margin | 25.2 | Normal Ovary | 3.7 |
| (OD04338) | | | |
| Kidney Ca Nuclear grade | 34.4 | Ovarian Cancer | 11.3 |
| 1/2 (OD04339) | | 064008 | |
| Kidney Margin | 35.6 | Ovarian Cancer | 25.0 |
| (OD04339) | | (OD04768-07) | |
| Kidney Ca, Clear cell | 20.9 | Ovary Margin | 3.7 |
| type (OD04340) | | (OD04768-08) | |
| Kidney Margin | 34.2 | Normal Stomach | 18.4 |
| (OD04340) | | | |
| Kidney Ca, Nuclear grade | 7.7 | Gastric Cancer | 4.0 |
| 3 (OD04348) | | 9060358 | |
| Kidney Margin | 28.5 | Stomach Margin | 8.2 |
| (OD04348) | | 9060359 | |
| Kidney Cancer | 8.0 | Gastric Cancer | 11.8 |
| (OD04622-01) | | 9060395 | |
| Kidney Margin | 2.9 | Stomach Margin | 8.7 |
| (OD04622-03) | | 9060394 | |
| Kidney Cancer | 13.0 | Gastric Cancer | 7.0 |
| (OD04450-01) | | 9060397 | |
| Kidney Margin | 17.9 | Stomach Margin | 3.7 |
| (OD04450-03) | | 9060396 | |
| Kidney Cancer 8120607 | 1.9 | Gastric Cancer | 38.7 |
| | | 064005 | |

TABLE 78
Panel 3D

| | Rel. Exp. (%) Ag3195, Run | | Rel. Exp. (%) Ag3195, Run |
|---|---|---|---|
| Tissue Name | 165301766 | Tissue Name | 165301766 |
| Daoy- Medulloblastoma | 15.4 | Ca Ski- Cervical epidermoid | 40.1 |
| | | carcinoma (metastasis) | |
| TE671- Medulloblastoma | 19.5 | ES-2- Ovarian clear cell | 6.7 |
| | | carcinoma | |
| D283 Med- | 62.4 | Ramos- Stimulated with | 0.0 |
| Medulloblastoma | | PMA/ionomycin 6 h | |
| PFSK-1- Primitive | 14.9 | Ramos- Stimulated with | 1.4 |
| Neuroectodermal | | PMA/ionomycin 14 h | |
| XF-498- CNS | 42.6 | MEG-01- Chronic | 25.5 |
| | | myelogenous leukemia | |
| | | (megokaryoblast) | |
| SNB-78- Glioma | 17.3 | Raji- Burkitt's lymphoma | 3.9 |
| SF-268- Glioblastoma | 29.1 | Daudi- Burkitt's lymphoma | 7.0 |
| T98G- Glioblastoma | 16.4 | U266- B-cell plasmacytoma | 76.8 |
| SK-N-SH- | 65.1 | CA46- Burkitt's lymphoma | 9.9 |
| Neuroblastoma | | | |
| (metastasis) | | | |
| SF-295- Glioblastoma | 10.9 | RL- non-Hodgkin's B-cell | 1.3 |
| | | lymphoma | |
| Cerebellum | 83.5 | JM1- pre-B-cell lymphoma | 39.5 |
| Cerebellum | 4.7 | Jurkat- T cell leukemia | 13.3 |
| NCI-H292- | 100.0 | TF-1- Erythroleukemia | 16.6 |
| Mucoepidermoid lung | | | |
| carcinoma | | | |
| DMS-114- Small cell | 10.4 | HUT 78- T-cell lymphoma | 25.2 |
| lung cancer | | | |
| DMS-79- Small cell lung | 87.7 | U937- Histiocytic lymphoma | 43.5 |
| cancer | | | |
| NCI-H146- Small cell | 16.2 | KU-812- Myelogenous | 31.9 |
| lung cancer | | leukemia | |
| NCI-H526- Small cell | 66.4 | 769-P- Clear cell renal | 21.5 |
| lung cancer | | carcinoma | |
| NCI-N417- Small cell | 14.3 | Caki-2- Clear cell renal | 14.9 |
| lung cancer | | carcinoma | |
| NCI-H82- Small cell | 7.1 | SW 839- Clear cell renal | 18.4 |
| lung cancer | | carcinoma | |
| NCI-H157- Squamous | 0.2 | G401- Wilms' tumor | 6.9 |
| cell lung cancer | | | |
| (metastasis) | | | |
| NCI-H1155- Large cell | 34.6 | Hs766T- Pancreatic | 19.1 |
| lung cancer | | carcinoma (LN metastasis) | |
| NCI-H1299- Large cell | 16.4 | CAPAN-1- Pancreatic | 13.6 |
| lung cancer | | adenocarcinoma (liver | |
| | | metastasis) | |
| NCI-H727- Lung | 0.0 | SU86.86- Pancreatic | 7.3 |
| carcinoid | | carcinoma (liver metastasis) | |
| NCI-UMC-11- Lung | 61.6 | BxPC-3- Pancreatic | 22.7 |
| carcinoid | | adenocarcinoma | |
| LX-1- Small cell lung | 30.4 | HPAC- Pancreatic | 0.7 |
| cancer | | adenocarcinoma | |
| Colo-205- Colon cancer | 60.3 | MIA PaCa-2- Pancreatic | 0.7 |
| | | carcinoma | |
| KM12- Colon cancer | 38.2 | CFPAC-1- Pancreatic ductal | 92.0 |
| | | adenocarcinoma | |
| KM20L2- Colon cancer | 2.1 | PANC-1- Pancreatic | 20.7 |
| | | epithelioid ductal carcinoma | |
| NCI-H716- Colon cancer | 43.5 | T24- Bladder carcinma | 8.4 |
| | | (transitional cell) | |
| SW-48- Colon | 29.5 | 5637- Bladder carcinoma | 26.1 |
| adenocarcinoma | | | |
| SW1116- Colon | 6.6 | HT-1197- Bladder carcinoma | 7.0 |
| adenocarcinoma | | | |
| LS 174T- Colon | 12.1 | UM-UC-3- Bladder carcinma | 1.4 |
| adenocarcinoma | | (transitional cell) | |
| SW-948- Colon | 3.0 | A204- Rhabdomyosarcoma | 10.4 |
| adenocarcinoma | | | |
| SW-480- Colon | 8.4 | HT-1080- Fibrosarcoma | 30.1 |
| adenocarcinoma | | | |
| NCI-SNU-5- Gastric | 9.0 | MG-63- Osteosarcoma | 7.6 |
| carcinoma | | | |
| KATO III- Gastric | 46.7 | SK-LMS-1- Leiomyosarcoma | 39.2 |
| carcinoma | | (vulva) | |
| NCI-SNU-16- Gastric | 26.4 | SJRH30- Rhabdomyosarcoma | 24.5 |
| carcinoma | | (met to bone marrow) | |
| NCI-SNU-1- Gastric | 84.7 | A431- Epidermoid carcinoma | 47.0 |
| carcinoma | | | |
| RF-1- Gastric | 30.4 | WM266-4- Melanoma | 25.5 |
| adenocarcinoma | | | |
| RF-48- Gastric | 15.8 | DU 145- Prostate carcinoma | 0.0 |
| adenocarcinoma | | (brain metastasis) | |
| MKN-45- Gastric | 8.8 | MDA-MB-468- Breast | 0.9 |
| carcinoma | | adenocarcinoma | |
| NCI-N87- Gastric | 12.4 | SCC-4- Squamous cell | 0.0 |
| carcinoma | | carcinoma of tongue | |
| OVCAR-5- Ovarian | 4.5 | SCC-9- Squamous cell | 0.0 |
| carcinoma | | carcinoma of tongue | |
| RL95-2- Uterine | 2.3 | SCC-15- Squamous cell | 0.6 |
| carcinoma | | carcinoma of tongue | |
| HelaS3- Cervical | 19.8 | CAL 27- Squamous cell | 25.9 |
| adenocarcinoma | | carcinoma of tongue | |

TABLE 79
Panel 4D

| | Rel. Exp. (%) Ag3195, Run | | Rel. Exp. (%) Ag3195, Run |
|---|---|---|---|
| Tissue Name | 164530142 | Tissue Name | 164530142 |
| Secondary Th1 act | 5.7 | HUVEC IL-1beta | 1.7 |
| Secondary Th2 act | 4.6 | HUVEC IFN gamma | 4.5 |
| Secondary Tr1 act | 5.1 | HUVEC TNF alpha + IFN | 9.0 |
| | | gamma | |
| Secondary Th1 rest | 1.7 | HUVEC TNF alpha + IL4 | 3.3 |
| Secondary Th2 rest | 2.9 | HUVEC IL-11 | 1.6 |
| Secondary Tr1 rest | 4.2 | Lung Microvascular EC | 7.4 |
| | | none | |
| Primary Th1 act | 0.8 | Lung Microvascular EC | 6.9 |
| | | TNFalpha + IL-1beta | |
| Primary Th2 act | 1.5 | Microvascular Dermal EC | 11.2 |
| | | none | |
| Primary Tr1 act | 2.5 | Microsvasular Dermal EC | 6.0 |
| | | TNFalpha + IL-1beta | |
| Primary Th1 rest | 24.5 | Bronchial epithelium | 18.2 |
| | | TNFalpha + IL1beta | |
| Primary Th2 rest | 14.1 | Small airway epithelium | 6.8 |
| | | none | |
| Primary Tr1 rest | 5.2 | Small airway epithelium | 100.0 |
| | | TNFalpha + IL-1beta | |
| CD45RA CD4 | 11.6 | Coronery artery SMC rest | 2.8 |
| lymphocyte act | | | |
| CD45RO CD4 | 6.4 | Coronery artery SMC | 1.5 |
| lymphocyte act | | TNFalpha + IL-1beta | |
| CD8 lymphocyte act | 6.3 | Astrocytes rest | 2.5 |
| Secondary CD8 | 6.7 | Astrocytes TNFalpha + | 1.8 |
| lymphocyte rest | | IL-1beta | |
| Secondary CD8 | 4.9 | KU-812 (Basophil) rest | 4.6 |
| lymphocyte act | | | |
| CD4 lymphocyte none | 10.1 | KU-812 (Basophil) | 11.0 |
| | | PMA/ionomycin | |
| 2ry Th1/Th2/Tr1_anti- | 7.7 | CCD1106 (Keratinocytes) | 9.5 |
| CD95 CH11 | | none | |
| LAK cells rest | 19.2 | CCD1106 (Keratinocytes) | 10.3 |
| | | TNFalpha + IL-1beta | |
| LAK cells IL-2 | 14.2 | Liver cirrhosis | 1.6 |
| LAK cells IL-2 + IL-12 | 12.7 | Lupus kidney | 3.3 |
| LAK cells IL-2 + IFN | 20.3 | NCI-H292 none | 30.8 |
| gamma | | | |
| LAK cells IL-2 + IL-18 | 17.7 | NCI-H292 IL-4 | 26.2 |
| LAK cells | 3.9 | NCI-H292 IL-9 | 27.4 |
| PMA/ionomycin | | | |
| NK Cells IL-2 rest | 7.6 | NCI-H292 IL-13 | 14.7 |
| Two Way MLR 3 day | 17.1 | NCI-H292 IFN gamma | 22.8 |
| Two Way MLR 5 day | 5.9 | HPAEC none | 2.8 |
| Two Way MLR 7 day | 4.3 | HPAEC TNF alpha + IL-1 | 4.7 |
| | | beta | |
| PBMC rest | 3.3 | Lung fibroblast none | 3.2 |
| PBMC PWM | 27.2 | Lung fibroblast TNF alpha | 3.1 |
| | | + IL-1 beta | |
| PBMC PHA-L | 7.1 | Lung fibroblast IL-4 | 4.4 |
| Ramos (B cell) none | 1.0 | Lung fibroblast IL-9 | 4.2 |
| Ramos (B cell) | 3.7 | Lung fibroblast IL-13 | 4.7 |
| ionomycin | | | |
| B lymphocytes PWM | 23.7 | Lung fibroblast IFN | 7.5 |
| | | gamma | |
| B lymphocytes CD40L | 14.5 | Dermal fibroblast | 23.3 |
| and IL-4 | | CCD1070 rest | |
| EOL-1 dbcAMP | 14.8 | Dermal fibroblast | 38.4 |
| | | CCD1070 TNF alpha | |
| EOL-1 dbcAMP | 6.0 | Dermal fibroblast | 12.2 |
| PMA/ionomycin | | CCD1070 IL-1 beta | |
| Dendritic cells none | 13.5 | Dermal fibroblast IFN | 14.5 |
| | | gamma | |
| Dendritic cells LPS | 13.7 | Dermal fibroblast IL-4 | 19.9 |
| Dendritic cells anti- | 16.3 | IBD Colitis 2 | 1.2 |
| CD40 | | | |
| Monocytes rest | 29.9 | IBD Crohn's | 2.1 |
| Monocytes LPS | 5.3 | Colon | 11.6 |
| Macrophages rest | 11.7 | Lung | 5.8 |
| Macrophages LPS | 9.7 | Thymus | 39.8 |
| HUVEC none | 3.3 | Kidney | 31.0 |
| HUVEC starved | 9.3 | | |

AI_comprehensive panel_v1.0 Summary: Ag3195 The NOV11b gene is expressed in normal lung, bone, joint tissue, gut and skin. There is no apparent difference in transcript expression in disease tissue as compared to normal tissue. Please see Panel 4D for discussion of utility of this gene in an autoimmune context.

CNS_neurodegeneration_v1.0 Summary: Ag3195 The expression profile of the NOV11b gene shows higher expression in the temporal cortex of Alzheimer's disease victims. This gene encodes an alpha glucosidase homolog. Brain glucose regulation may play a role in Alzheimer's disease. For example, hyperglycemia may exert a deleterious effect by potentiating the neuronal death produced by pathological processes taking place, such as amyloid deposition. The category of agents that interfere with the absorption of glucose and lipids includes alpha-glucosidase inhibitors. Therefore, modulators of this gene product may be useful in the treatment of Alzheimer's disease. Data from a second experiment with probe/primer set Ag3154 is not included due to the high probability of a probe failure.

References:

Messier C, Gagnon M. Glucose regulation and cognitive functions: relation to Alzheimer's disease and diabetes. Behav Brain Res February 1996;75(1-2):1-11

Glucose has been found to improve memory in animals and humans. Animal research has revealed that glucose may improve memory through a facilitation of acetylcholine (ACh) synthesis and release in the brain. This glucose-related memory improvement has prompted research in elderly humans. These studies have shown that the memory-improving action of glucose depends on each individuals' blood glucose regulation. Based on these data, researchers have evaluated the effect of glucose on memory in patients with Alzheimer's disease (AD). Results demonstrated that glucose could improve memory in a subset of patients that had abnormalities in their blood glucose regulation. Interestingly, these alterations in blood glucose regulation were believed to depend on the severity of the disease process. Another line of investigation has focused on alterations in brain glucose metabolism. Both animal models and studies with Type II diabetic elderly patients have shown that altered glucose regulation impairs learning and memory processes. It is possible that in AD patients, hyperglycemia exerts a deleterious effect by potentiating the neuronal death produced by other pathological processes taking place such as amyloid deposition. Based on these data, it appears important to find the prevalence of altered glucoregulation at various stages of AD. Secondly, it may be of interest to determine prospectively whether altered glucoregulation is linked to a faster progression of the disease. Finally, if such a relationship is observed, the next logical step would be to determine whether AD patients could benefit from treatments aimed at normalizing blood glucose regulation and improving insulin sensitivity.

Panel 1 Summary: Ag401 The expression of the NOV11b gene appears to be highest in a sample derived from testis tissue (CT=26. 1). In addition to testis tissue, there appears to be substantial expression associated with the cerebellar region of the brain and mammary gland. The other samples in the panel show low uniform expression. Thus, the expression of this gene could be used to distinguish testis, mammary gland and cerebellum from the other samples in the panel.

Please see Panel CNS_neurodegeneration_v1.0 for discussion of potential utility of this gene in the central nervous system.

This gene is also moderately expressed in a number of metabolic tissues including pancreas, adrenal, thyroid, pituitary, heart, skeletal muscle, and adult and fetal liver. This small molecule target may be useful for the treatment of metabolic diseases, including obesity and Types 1 and 2 diabetes.

Panel 1.3D Summary: Ag401/Ag3195 Three experiments expression of the NOV11b gene in a wide variety of metabolic tissues, including pancreas, adipose, adrenal, thyroid, pituitary, adult and fetal heart, adult and fetal skeletal muscle, and adult and fetal liver. Alpha-glucosidase inhibitors are currently used in the treatment of Type 2 diabetes to decrease glucose absorption from the gut. Thus, this gene product may be a small molecule target for the treatment of metabolic diseases, including obesity and Types 1 and 2 diabetes.

Significant brain expression of the gene is also seen in this panel. Please see Panel CNS_neurodegeneration_v1.0 for discussion of potential utility of this gene in the central nervous system. Data from a second experiment with probe/primer set Ag3154 is not included due to the high probability of a probe failure.

References:

Raptis S A, Dimitriadis G D. Oral hypoglycemic agents: insulin secretagogues, alpha-glucosidase inhibitors and insulin sensitizers. Exp Clin Endocrinol Diabetes. 2001;109 Suppl 2:S265-87.

In this review we present the agents that are in use in the treatment of type 2 diabetes. Sulfonylureas of the 1st and 2nd generation increase insulin secretion but can induce hyperinsulinemia and sometimes prolonged hypoglycemia. Glimepiride is a new 3rd generation sulfonylurea with some advantages over the other members of this group, such as a lower risk of hypoglycemia, no interaction with cardiovascular KATP-channels and a possibility that it may increase insulin sensitivity. There are also newer insulin secretagogues (such as neteglinide and repaglinide) with a rapid onset of action on the beta-cell, therefore inducing a more physiological profile of insulin secretion during meals. The category of insulin sensitizers includes metformin and thiazolidinediones. Metformin effectively reduces hyperglycemia, hyperlipidemia and macroangiopathy in patients with type 2 diabetes. This agent increases the sensitivity of the liver and peripheral tissues to insulin and, therefore, it could be considered as a drug of choice for the prevention of type 2 diabetes. Thiazolidinediones (rosiglitazone and pioglitazone) increase the sensitivity of the tissues to insulin. This mechanism of action makes them powerful therapeutic tools for the treatment of type 2 diabetes (and possibly other insulin resistant states) either alone or in combination with other oral agents. The category of agents that interfere with the absorption of glucose and lipids includes alpha-glucosidase inhibitors (acarbose and miglitol) and lipase inhibitors (or-listat). alpha-Glucocidase inhibitors improve the time relationship between plasma insulin and glucose increases after a meal. Therefore, these agents may be used in the treatment of patients with type 2 diabetes, either alone at a very early stage of this disease (when insulin secretion is still adequate), or in combination with insulin secretagogues. alpha-Glucosidase inhibition may also prove useful as a supplement to insulin therapy in patients with type 1 diabetes mellitus. The inhibitor of gastrointestinal lipase orlistat may prove a useful adjunct to hypocaloric diets in patients with type 2 diabetes and obesity.

PMID: 11460577

Panel 2D Summary: Ag3195 The expression of the NOV11b gene appears to be highest in a sample of normal lung tissue adjacent to malignant lung (CT=27.4). In addition, there appears to be substantial expression in other samples of normal lung tissue, normal kidney tissue and normal colon tissue. Thus, the expresion of this gene could be used to distinguish normal lung, kidney and colon form other samples in the panel. Moreover, therapeutic modulation of this gene, through the use of small molecule drugs, antibodies or protein therapeutics might be of benefit in the treatment of lung, kidney or colon cancer.

Panel 3D Summary: Ag3195 The expression of the NOV11b gene appears to be highest in a sample derived from a lung cancer cell line(CT=29.9). In addition, there appears to be substantial expression in a number of lung cnacer cell lines' in addition to a number of other samples in this panel. Thus, the expression of this gene could be used to distinguish this lung cancer cell line derived sample from other samples in the panel.

Panel 4D Summary: Ag3191 The NOV11b transcript is expressed at high levels in small airway epithelium activated with TNFalpha and IL-1beta. It is also expressed in normal tissues such as the lung, thymus and kidney, as seen in Panel AI. The protein encoded by this transcript is related to Alpha glucosidase A. This enzyme is important in lung maturation and may contribute to surfactant phospholipid biosynthesis (see reference). Therefore, therapeutics designed with the protein encoded by this transcript or therapeutics that modulate its production could potentially be used to aid lung surfactant production in premature infants, and to treat lung injuries. Data from a second experiment with probe/primer set Ag3154 is not included due to the high probability of a probe failure.

References:

Bourbon J R, Doucet E, Rieutort M. Role of alpha-glucosidase in fetal lung maturation.Biochim Biophys Acta Jan. 13, 1987;917(1):203-10

The role of lysosomal enzyme acid alpha-glucosidase in fetal lung development was investigated with the aid of a specific inhibitor, the pseudosaccharide acarbose. The drug was added to a Waymouth culture medium of fetal rat lung explants cultivated for 48 h fromgestational stage 19.5 days, an in vitro system previously shown to allow morphological and biochemical maturation of alveolar epithelium. Glycogenolysis was reduced by 40% as compared with tissue cultivated on control medium, which means thatalpha-glucosidase could account for as much as 40% of fetal lung glycogenolysis, the remaining 60% being presumably achieved by cytosolic phosphorylase and by a microsomal neutral alpha-glucosidase. By the same time, the increase of phospholipids of surfactant fraction extracted from cultivated explants was partially inhibited: total and saturated phosphatidylcholine, phosphatidylethanolamine, phosphatidylglycerol and phosphatidylinositol were about 30-40% lower than in lungs cultivated on control medium. It should beemphasized that DNA concentration and increases in non-surfactant phospholipids were unchanged by the drug. alpha-Glucosidase activity was evidenced in the lysosomal fraction, in the microsomal fraction and, although in lower amounts, in the surfactant fraction extracted from term fetal lung. The results suggest that lysosomal alpha-glucosidase plays a major role in lung maturation and could facilitate glycogenolysis for the specific use of glycogen stores in providing substrates for surfactant phospholipid biosynthesis.

PMID: 3539207

NOV11c

Expression of gene NOV11c was assessed using the primer-probe set Ag401, described in Table 80. Results of the RTQ-PCR runs are shown in Tables 81 and 82.

TABLE 80

Probe Name Ag401

| | | | | |
|---|---|---|---|---|
| Primers | Sequences | Length | Start Position | SEQ ID NO: |

| | | | | |
|---|---|---|---|---|
| Forward | 5′-ttgtgccaaaacatccatcct-3′ | 21 | 2870 | 229 |
| Probe | TET-5′-agcctggagaagctctcactcaacattgc-3′-TAMRA | 29 | 2892 | 230 |
| Reverse | 5′-tatgatgcggacctcccagt-3′ | 20 | 2926 | 231 |

TABLE 81
Panel 1

| | Rel. Exp. (%) Ag401, | | Rel. Exp. (%) Ag401, |
|---|---|---|---|
| Tissue Name | Run 88225002 | Tissue Name | Run 88225002 |
| Endothelial cells | 1.5 | Renal ca. 786-0 | 10.7 |
| Endothelial cells | 2.5 | Renal ca. A498 | 4.2 |
| (treated) | | | |
| Pancreas | 11.0 | Renal ca. RXF 393 | 4.8 |
| Pancreatic ca. CAPAN 2 | 2.6 | Renal ca. ACHN | 4.9 |
| Adrenal gland | 8.5 | Renal ca. UO-31 | 3.8 |
| Thyroid | 13.6 | Renal ca. TK-10 | 5.0 |
| Salivary gland | 12.7 | Liver | 24.0 |
| Pituitary gland | 8.3 | Liver (fetal) | 4.0 |
| Brain (fetal) | 4.2 | Liver ca. (hepatoblast) | 7.1 |
| | | HepG2 | |
| Brain (whole) | 14.7 | Lung | 3.1 |
| Brain (amygdala) | 4.1 | Lung (fetal) | 12.9 |
| Brain (cerebellum) | 74.2 | Lung ca. (small cell) | 9.5 |
| | | LX-1 | |
| Brain (hippocampus) | 7.4 | Lung ca. (small cell) | 0.8 |
| | | NCI-H69 | |
| Brain (substantia nigra) | 13.6 | Lung ca. (s.cell var.) | 13.2 |
| | | SHP-77 | |
| Brain (thalamus) | 7.3 | Lung ca. (large | 8.5 |
| | | cell)NCI-H460 | |
| Brain (hypothalamus) | 12.6 | Lung ca. (non-sm. | 4.3 |
| | | cell) A549 | |
| Spinal cord | 12.9 | Lung ca. (non-s.cell) | 5.7 |
| | | NCI-H23 | |
| glio/astro U87-MG | 3.5 | Lung ca. (non-s.cell) | 3.7 |
| | | HOP-62 | |
| glio/astro U-118-MG | 7.6 | Lung ca. (non-s.cl) | 10.9 |
| | | NCI-H522 | |
| astrocytoma SW1783 | 2.0 | Lung ca. (squam.) SW | 8.4 |
| | | 900 | |
| neuro*; met SK-N-AS | 4.4 | Lung ca. (squam.) | 1.2 |
| | | NCI-H596 | |
| astrocytoma SF-539 | 5.6 | Mammary gland | 32.1 |
| astrocytoma SNB-75 | 5.4 | Breast ca.* (pl.ef) | 5.6 |
| | | MCF-7 | |
| glioma SNB-19 | 7.0 | Breast ca.* (pl.ef) | 3.3 |
| | | MDA-MB-231 | |
| glioma U251 | 5.5 | Breast ca.* (pl. ef) | 12.3 |
| | | T47D | |
| glioma SF-295 | 5.3 | Breast ca. BT-549 | 6.7 |
| Heart | 8.8 | Breast ca. MDA-N | 8.8 |
| Skeletal muscle | 14.6 | Ovary | 10.4 |
| Bone marrow | 9.3 | Ovarian ca. OVCAR-3 | 3.1 |
| Thymus | 27.7 | Ovarian ca. OVCAR-4 | 1.4 |
| Spleen | 14.6 | Ovarian ca. OVCAR-5 | 12.6 |
| Lymph node | 21.6 | Ovarian ca. OVCAR-8 | 3.4 |
| Colon (ascending) | 13.7 | Ovarian ca. IGROV-1 | 3.2 |
| Stomach | 20.4 | Ovarian ca. (ascites) | 8.3 |
| | | SK-OV-3 | |
| Small intestine | 17.4 | Uterus | 16.7 |
| Colon ca. SW480 | 1.5 | Placenta | 11.2 |
| Colon ca.* SW620 | 4.2 | Prostate | 12.4 |
| (SW480 met) | | | |
| Colon ca. HT29 | 1.5 | Prostate ca.* (bone | 13.1 |
| | | met) PC-3 | |
| Colon ca. HCT-116 | 5.3 | Testis | 100.0 |
| Colon ca. CaCo-2 | 3.5 | Melanoma | 12.4 |
| | | Hs688(A).T | |
| Colon ca. HCT-15 | 7.2 | Melanoma* (met) | 6.4 |
| | | Hs688(B).T | |
| Colon ca. HCC-2998 | 6.4 | Melanoma UACC-62 | 1.6 |
| Gastric ca. * (liver met) | 26.1 | Melanoma M14 | 33.9 |
| NCI-N87 | | | |
| Bladder | 4.3 | Melanoma LOX | 8.0 |
| | | IMVI | |
| Trachea | 20.9 | Melanoma* (met) SK- | 3.0 |
| | | MEL-5 | |
| Kidney | 14.9 | Melanoma SK-MEL- | 15.9 |
| | | 28 | |
| Kidney (fetal) | 17.3 | | |

TABLE 82
Panel 1.3D

| | Rel. Exp. (%) Ag401, | | Rel. Exp. (%) Ag401, |
|---|---|---|---|
| Tissue Name | Run 165518174 | Tissue Name | Run 165518174 |
| Liver adenocarcinoma | 2.9 | Kidney (fetal) | 13.8 |
| Pancreas | 10.1 | Renal ca. 786-0 | 14.8 |
| Pancreatic ca. CAPAN 2 | 10.2 | Renal ca. A498 | 13.9 |
| Adrenal gland | 16.2 | Renal ca. RXF 393 | 18.2 |
| Thyroid | 18.9 | Renal ca. ACHN | 12.7 |
| Salivary gland | 24.7 | Renal ca. UO-31 | 12.2 |
| Pituitary gland | 20.2 | Renal ca. TK-10 | 5.7 |
| Brain (fetal) | 23.5 | Liver | 16.7 |
| Brain (whole) | 52.1 | Liver (fetal) | 9.0 |
| Brain (amygdala) | 16.2 | Liver ca. | 9.6 |
| | | (hepatoblast) HepG2 | |
| Brain (cerebellum) | 47.6 | Lung | 19.5 |
| Brain (hippocampus) | 54.3 | Lung (fetal) | 11.2 |
| Brain (substantia nigra) | 20.0 | Lung ca. (small cell) | 18.7 |
| | | LX-1 | |
| Brain (thalamus) | 41.5 | Lung ca. (small cell) | 0.0 |
| | | NCI-H69 | |
| Cerebral Cortex | 15.0 | Lung ca. (s.cell var.) | 10.9 |
| | | SHP-77 | |
| Spinal cord | 38.7 | Lung ca. (large | 19.5 |
| | | cell)NCI-H460 | |
| glio/astro U87-MG | 9.5 | Lung ca. (non-sm. | 1.6 |
| | | cell) A549 | |
| glio/astro U-118-MG | 49.7 | Lung ca. (non-s.cell) | 9.3 |
| | | NCI-H23 | |
| astrocytoma SW1783 | 10.4 | Lung ca. (non-s.cell) | 17.8 |
| | | HOP-62 | |
| neuro*; met SK-N-AS | 20.2 | Lung ca. (non-s.cl) | 9.0 |
| | | NCI-H522 | |
| astrocytoma SF-539 | 25.7 | Lung ca. (squam.) | 4.5 |
| | | SW 900 | |
| astrocytoma SNB-75 | 22.4 | Lung ca. (squam.) | 3.1 |
| | | NCI-H596 | |
| glioma SNB-19 | 25.9 | Mammary gland | 27.0 |
| glioma U251 | 59.9 | Breast ca.* (pl.ef) | 4.3 |
| | | MCF-7 | |
| glioma SF-295 | 15.4 | Breast ca.* (pl.ef) | 47.3 |
| | | MDA-MB-231 | |
| Heart (fetal) | 0.5 | Breast ca.* (pl.ef) | 6.4 |
| | | T47D | |
| Heart | 14.0 | Breast ca. BT-549 | 12.9 |
| Skeletal muscle (fetal) | 7.0 | Breast ca. MDA-N | 6.2 |
| Skeletal muscle | 100.0 | Ovary | 6.3 |
| Bone marrow | 33.9 | Ovarian ca. OVCAR-3 | 10.5 |
| Thymus | 32.1 | Ovarian ca. OVCAR-4 | 7.2 |
| Spleen | 27.9 | Ovarian ca. OVCAR-5 | 13.6 |
| Lymph node | 76.8 | Ovarian ca. OVCAR-8 | 4.6 |
| Colorectal | 38.7 | Ovarian ca. IGROV-1 | 2.5 |
| Stomach | 21.0 | Ovarian ca.* (ascites) | 17.3 |
| | | SK-OV-3 | |
| Small intestine | 60.7 | Uterus | 61.6 |
| Colon ca. SW480 | 7.6 | Plancenta | 2.9 |
| Colon ca.* | 2.9 | Prostate | 17.8 |
| SW620(SW480 met) | | | |
| Colon ca. HT29 | 0.8 | Prostate ca.* (bone | 19.6 |
| | | met)PC-3 | |
| Colon ca. HCT-116 | 3.6 | Testis | 50.7 |
| Colon ca. CaCo-2 | 3.1 | Melanoma | 9.5 |
| | | Hs688(A).T | |
| Colon ca. | 7.9 | Melanoma* (met) | 15.8 |
| tissue(ODO3866) | | Hs688(B).T | |
| Colon ca. HCC-2998 | 10.3 | Melanoma UACC-62 | 7.6 |
| Gastric ca.* (liver met) | 55.1 | Melanoma M14 | 89.5 |
| NCI-N87 | | | |
| Bladder | 22.2 | Melanoma LOX | 2.2 |
| | | IMVI | |
| Trachea | 17.4 | Melanoma* (met) | 1.9 |
| | | SK-MEL-5 | |
| Kidney | 19.5 | Adipose | 15.5 |

Panel 1 Summary: Ag401 The expression of the NOV11c gene appears to be highest in a sample derived from testis tissue (CT=26.1). In addition to testis tissue, there appears to be substantial expression associated with the cerebellar region of the brain and mammary gland. The other samples in the panel show low uniform expression. Thus, the expression of this gene could be used to distinguish testis, mammary gland and cerebellum from the other samples in the panel.

This gene is also moderately expressed in a number of metabolic tissues including pancreas, adrenal, thyroid, pituitary, heart, skeletal muscle, and adult and fetal liver. This small molecule target may be useful for the treatment of metabolic diseases, including obesity and Types 1 and 2 diabetes.

Panel 1.3D Summary: Ag401 The NOV11c gene is expressed in a wide variety of metabolic tissues, including pancreas, adipose, adrenal, thyroid, pituitary, adult and fetal heart, adult and fetal skeletal muscle, and adult and fetal liver. Highest expression is seen in skeletal muscle (CT=29.7). In addition, this gene appears to be more highly expressed in adult skeletal muscle and heart (CTs=29-32) than in fetal skeletal muscle and heart (CTs=33-37). Thus, expression of this gene could be used to differentiate between the two sources of these tissues. Furthermore, alpha-glucosidase inhibitors are currently used in the treatment of Type 2 diabetes to decrease glucose absorption from the gut. Thus, this gene product may be a small molecule target for the treatment of metabolic diseases, including obesity and Types 1 and 2 diabetes.

Significant brain expression of the gene is also seen in this panel. Please see Panel CNS_neurodegeneration_v1.0 for discussion of potential utility of this gene in the central nervous system.

Data from a second experiment with probe/primer set Ag3154 is not included due to the high probability of a probe failure.

References:

Raptis S A, Dimitriadis G D. Oral hypoglycemic agents: insulin secretagogues, alpha-glucosidase inhibitors and insulin sensitizers. Exp Clin Endocrinol Diabetes. 2001;109 Suppl 2:S265-87.

In this review we present the agents that are in use in the treatment of type 2 diabetes. Sulfonylureas of the 1st and 2nd generation increase insulin secretion but can induce hyperinsulinemia and sometimes prolonged hypoglycemia. Glimepiride is a new 3rd generation sulfonylurea with some advantages over the other members of this group, such as a lower risk of hypoglycemia, no interaction with cardiovascular KATP-channels and a possibility that it may increase insulin sensitivity. There are also newer insulin secretagogues (such as neteglinide and repaglinide) with a rapid onset of action on the beta-cell, therefore inducing a more physiological profile of insulin secretion during meals. The category of insulin sensitizers includes metformin and thiazolidinediones. Metformin effectively reduces hyperglycemia, hyperlipidemia and macroangiopathy in patients with type 2 diabetes. This agent increases the sensitivity of the liver and peripheral tissues to insulin and, therefore, it could be considered as a drug of choice for the prevention of type 2 diabetes. Thiazolidinediones (rosiglitazone and pioglitazone) increase the sensitivity of the tissues to insulin. This mechanism of action makes them powerful therapeutic tools for the treatment of type 2 diabetes (and possibly other insulin resistant states) either alone or in combination with other oral agents. The category of agents that interfere with the absorption of glucose and lipids includes alpha-glucosidase inhibitors (acarbose and miglitol) and lipase inhibitors (or-listat). alpha-Glucocidase inhibitors improve the time relationship between plasma insulin and glucose increases after a meal. Therefore, these agents may be used in the treatment of patients with type 2 diabetes, either alone at a very early stage of this disease (when insulin secretion is still adequate), or in combination with insulin secretagogues. alpha-Glucosidase inhibition may also prove useful as a supplement to insulin therapy in patients with type 1 diabetes mellitus. The inhibitor of gastrointestinal lipase orlistat may prove a useful adjunct to hypocaloric diets in patients with type 2 diabetes and obesity.

PMID: 11460577

NOV11d

Expression of gene NOV11d was assessed using the primer-probe sets Ag3195 and Ag401, described in Tables 83 and 84. Results of the RTQ-PCR runs are shown in Tables 85, 86, 87, 88, 89, 90 and 91.

TABLE 83
Probe Name Ag3195

| Primers | Sequences | Length | Start Position | SEQ ID NO: |
|---|---|---|---|---|
| Forward | 5′aatagttttgctgaccagagg-3′ | 21 | 2623 | 232 |
| Probe | TET-5′cccagcaagtgtgtggtggag-3′-TAMRA | 21 | 2653 | 233 |
| Reverse | 5′tgatctttaccatcagatgagt-3′ | 22 | 2723 | 234 |

TABLE 84
Probe Name Ag401

| Primers | Sequences | Length | Start Position | SEQ ID NO: |
|---|---|---|---|---|
| Forward | 5′ttgtgccaaaacatccatcct-3′ | 21 | 2763 | 235 |
| Probe | TET-5′agcctggagaagctctcactcaacattgc-3′-TAMRA | 29 | 2785 | 236 |
| Reverse | 5′-tatgatgcggacctcccagt-3′ | 20 | 2819 | 237 |

TABLE 85
A1_comprehensive panel_v1.0

| | Rel. Exp. (%) Ag3195, Run | | Rel. Exp. (%) Ag3195, Run |
|---|---|---|---|
| Tissue Name | 225147682 | Tissue Name | 225147682 |
| 110967 COPD-F | 17.3 | 112427 Match Control | 66.9 |
| | | Psoriasis-F | |
| 110980 COPD-F | 15.3 | 112418 Psoriasis-M | 14.2 |
| 110968 COPD-M | 14.4 | 112723 Match Control | 4.0 |
| | | Psoriasis-M | |
| 110977 COPD-M | 37.1 | 112419 Psoriasis-M | 19.5 |
| 110989 Emphysema-F | 29.1 | 112424 Match Control | 13.7 |
| | | Psoriasis-M | |
| 110992 Emphysema-F | 11.3 | 112420 Psoriasis-M | 59.0 |
| 110993 Emphysema-F | 9.9 | 112425 Match Control | 57.8 |
| | | Psoriasis-M | |
| 110994 Emphysema-F | 9.3 | 104689 (MF) OA | 33.4 |
| | | Bone-Backus | |
| 110995 Emphysema-F | 25.3 | 104690 (MF) Adj | 22.4 |
| | | “Normal” Bone-Backus | |
| 110996 Emphysema-F | 3.2 | 104691 (MF) OA | 28.5 |
| | | Synovium-Backus | |
| 110997 Asthma-M | 4.8 | 104692 (BA) OA | 14.8 |
| | | Cartilage-Backus | |
| 111001 Asthma-F | 18.8 | 104694 (BA) OA | 22.1 |
| | | Bone-Backus | |
| 111002 Asthma-F | 13.6 | 104695 (BA) Adj | 27.0 |
| | | “Normal” Bone-Backus | |
| 111003 Atopic | 14.6 | 104696 (BA) OA | 15.5 |
| Asthma-F | | Synovium-Backus | |
| 111004 Atopic | 21.5 | 104700 (SS) OA Bone- | 16.6 |
| Asthma-F | | Backus | |
| 111005 Atopic | 14.5 | 104701 (SS) Adj | 16.5 |
| Asthma-F | | “Normal” Bone-Backus | |
| 111006 Atopic | 2.1 | 104702 (SS) OA | 29.1 |
| Asthma-F | | Synovium-Backus | |
| 111417 Allergy-M | 12.2 | 117093 OA Cartilage | 33.0 |
| | | Rep7 | |
| 112347 Allergy-M | 0.0 | 112672 OA Bone5 | 19.2 |
| 112349 Normal Lung-F | 0.0 | 112673 OA Synovium5 | 11.0 |
| 112357 Normal Lung-F | 11.6 | 112674 OA Synovial | 8.1 |
| | | Fluid cells5 | |
| 112354 Normal Lung-M | 14.9 | 117100 OA Cartilage | 5.0 |
| | | Rep14 | |
| 112374 Crohns-F | 18.6 | 112756 OA Bone9 | 5.4 |
| 112389 Match | 15.7 | 112757 OA Synovium9 | 25.7 |
| Control Crohns-F | | | |
| 112375 Crohns-F | 16.8 | 112758 OA Synovial | 15.6 |
| | | Fluid Cells9 | |
| 112732 Match | 20.4 | 117125 RA Cartilage | 19.1 |
| Control Crohns-F | | Rep2 | |
| 112725 Crohns-M | 3.8 | 113492 Bone2 RA | 26.8 |
| 112387 Match | 15.7 | 113493 Synovium2 RA | 8.9 |
| Control Crohns-M | | | |
| 112378 Crohns-M | 0.0 | 113494 Syn Fluid Cells | 19.5 |
| | | RA | |
| 112390 Match | 100.0 | 113499 Cartilage4 RA | 19.8 |
| Control Crohns-M | | | |
| 112726 Crohns-M | 23.0 | 113500 Bone4 RA | 27.9 |
| 112731 Match | 14.1 | 113501 Synovium4 RA | 19.2 |
| Control Crohns-M | | | |
| 112380 Ulcer Col-F | 41.8 | 113502 Syn Fluid | 13.6 |
| | | Cells4 RA | |
| 112734 Match | 46.7 | 113495 Cartilage3 RA | 17.2 |
| Control Ulcer Col-F | | | |
| 112384 Ulcer Col-F | 51.4 | 113496 Bone3 RA | 19.9 |
| 112737 Match | 6.5 | 113497 Synovium3 RA | 9.8 |
| Control Ulcer Col-F | | | |
| 112386 Ulcer Col-F | 10.4 | 113498 Syn Fluid | 25.2 |
| | | Cells3 RA | |
| 112738 Match | 4.5 | 117106 Normal | 6.2 |
| Control Ulcer Col-F | | Cartilage Rep20 | |
| 112381 Ulcer Col-M | 0.0 | 113663 Bone3 Normal | 0.0 |
| 112735 Match | 7.4 | 113664 Synovium3 | 0.0 |
| Control Ulcer Col-M | | Normal | |
| 112382 Ulcer Col-M | 11.3 | 113665 Syn Fluid | 0.0 |
| | | Cells3 Normal | |
| 112394 Match | 6.4 | 117107 Normal | 8.1 |
| Control Ulcer Col-M | | Cartilage Rep22 | |
| 112383 Ulcer Col-M | 21.2 | 113667 Bone4 Normal | 11.0 |
| 112736 Match | 9.6 | 113668 Synovium4 | 15.7 |
| Control Ulcer Col-M | | Normal | |
| 112423 Psoriasis-F | 17.9 | 113669 Syn Fluid | 18.0 |
| | | Cells4 Normal | |

TABLE 86
CNS_neurodegeneration_v1.0

| | Rel. Exp. (%) Ag3195, | | Rel. Exp. (%) Ag3195, |
|---|---|---|---|
| Tissue Name | Run 209859542 | Tissue Name | Run 209859542 |
| AD 1 Hippo | 16.6 | Control (Path) 3 | 6.5 |
| | | Temporal Ctx | |
| AD 2 Hippo | 31.0 | Control (Path) 4 | 34.4 |
| | | Temporal Ctx | |
| AD 3 Hippo | 6.8 | AD 1 Occipital Ctx | 14.0 |
| AD 4 Hippo | 8.7 | AD 2 Occipital Ctx | 0.0 |
| | | (Missing) | |
| AD 5 hippo | 60.7 | AD 3 Occipital Ctx | 13.1 |
| AD 6 Hippo | 63.7 | AD 4 Occipital Ctx | 29.1 |
| Control 2 Hippo | 32.1 | AD 5 Occipital Ctx | 19.6 |
| Control 4 Hippo | 17.6 | AD 6 Occipital Ctx | 26.8 |
| Control (Path) 3 | 12.8 | Control 1 Occipital | 3.3 |
| Hippo | | Ctx | |
| AD 1 Temporal Ctx | 35.6 | Control 2 Occipital | 51.1 |
| | | Ctx | |
| AD 2 Temporal Ctx | 37.1 | Control 3 Occipital | 15.3 |
| | | Ctx | |
| AD 3 Temporal Ctx | 7.9 | Control 4 Occipital | 12.8 |
| | | Ctx | |
| AD 4 Temporal Ctx | 32.8 | Control (Path) 1 | 100.0 |
| | | Occipital Ctx | |
| AD 5 Inf Temporal | 95.9 | Control (Path) 2 | 16.4 |
| Ctx | | Occipital Ctx | |
| AD 5 SupTemporal | 56.3 | Control (Path) 3 | 8.4 |
| Ctx | | Occipital Ctx | |
| AD 6 Inf Temporal | 90.8 | Control (Path) 4 | 17.4 |
| Ctx | | Occipital Ctx | |
| AD 6 Sup Temporal | 68.8 | Control 1 Parietal | 8.4 |
| Ctx | | Ctx | |
| Control 1 Temporal | 4.9 | Control 2 Parietal | 57.8 |
| Ctx | | Ctx | |
| Control 2 Temporal | 33.7 | Control 3 Parietal | 14.4 |
| Ctx | | Ctx | |
| Control 3 Temporal | 14.1 | Control (Path) 1 | 63.7 |
| Ctx | | Parietal Ctx | |
| Control 4 Temporal | 9.4 | Control (Path) 2 | 28.7 |
| Ctx | | Parietal Ctx | |
| Control (Path) 1 | 49.0 | Control (Path) 3 | 3.6 |
| Temporal Ctx | | Parietal Ctx | |
| Control (Path) 2 | 32.1 | Control (Path) 4 | 56.3 |
| Temporal Ctx | | Parietal Ctx | |

TABLE 87
Panel 1

| | Rel. Exp. (%) Ag401, | | Rel. Exp. (%) Ag401, |
|---|---|---|---|
| Tissue Name | Run 88225002 | Tissue Name | Run 88225002 |
| Endothelial cells | 1.5 | Renal ca. 786-0 | 10.7 |
| Endothelial cells | 2.5 | Renal ca. A498 | 4.2 |
| (treated) | | | |
| Pancreas | 11.0 | Renal ca. RXF 393 | 4.8 |
| Pancreatic ca. CAPAN | 2.6 | Renal ca. ACHN | 4.9 |
| 2 | | | |
| Adrenal gland | 8.5 | Renal ca. UO-31 | 3.8 |
| Thyroid | 13.6 | Renal ca. TK-10 | 5.0 |
| Salivary gland | 12.7 | Liver | 24.0 |
| Pituitary gland | 8.3 | Liver (fetal) | 4.0 |
| Brain (fetal) | 4.2 | Liver ca. (hepatoblast) | 7.1 |
| | | HepG2 | |
| Brain (whole) | 14.7 | Lung | 3.1 |
| Brain (amygdala) | 4.1 | Lung (fetal) | 12.9 |
| Brain (cerebellum) | 74.2 | Lung ca. (small cell) | 9.5 |
| | | LX-1 | |
| Brain (hippocampus) | 7.4 | Lung ca. (small cell) | 0.8 |
| | | NCI-H69 | |
| Brain (substantia nigra) | 13.6 | Lung ca. (s.cell var.) | 13.2 |
| | | SHP-77 | |
| Brain (thalamus) | 7.3 | Lung ca. (large | 8.5 |
| | | cell)NCI-H460 | |
| Brain (hypothalamus) | 12.6 | Lung ca. (non-sm. | 4.3 |
| | | cell) A549 | |
| Spinal cord | 12.9 | Lung ca. (non-s.cell) | 5.7 |
| | | NCI-H23 | |
| glio/astro U87-MG | 3.5 | Lung ca. (non-s.cell) | 3.7 |
| | | HOP-62 | |
| glio/astro U-118-MG | 7.6 | Lung ca. (non-s.cl) | 10.9 |
| | | NCI-H522 | |
| astrocytoma SW1783 | 2.0 | Lung ca. (squam.) SW | 8.4 |
| | | 900 | |
| neuro*; met SK-N-AS | 4.4 | Lung ca. (squam.) | 1.2 |
| | | NCI-H596 | |
| astrocytoma SF-539 | 5.6 | Mammary gland | 32.1 |
| astrocytoma SNB-75 | 5.4 | Breast ca.* (pl.ef) | 5.6 |
| | | MCF-7 | |
| glioma SNB-19 | 7.0 | Breast ca.* (pl. ef) | 3.3 |
| | | MDA-MB-231 | |
| glioma U251 | 5.5 | Breast ca.* (pl. ef) | 12.3 |
| | | T47D | |
| glioma SF-295 | 5.3 | Breast ca. BT-549 | 6.7 |
| Heart | 8.8 | Breast ca. MDA-N | 8.8 |
| Skeletal muscle | 14.6 | Ovary | 10.4 |
| Bone marrow | 9.3 | Ovarian ca. OVCAR-3 | 3.1 |
| Thymus | 27.7 | Ovarian ca. OVCAR-4 | 1.4 |
| Spleen | 14.6 | Ovarian ca. OVCAR-5 | 12.6 |
| Lymph node | 21.6 | Ovarian ca. OVCAR-8 | 3.4 |
| Colon (ascending) | 13.7 | Ovarian ca. IGROV-1 | 3.2 |
| Stomach | 20.4 | Ovarian ca. (ascites) | 8.3 |
| | | SK-OV-3 | |
| Small intestine | 17.4 | Uterus | 16.7 |
| Colon ca. SW480 | 1.5 | Placenta | 11.2 |
| Colon ca.* SW620 | 4.2 | Prostate | 12.4 |
| (SW480 met) | | | |
| Colon ca. HT29 | 1.5 | Prostate ca.* (bone | 13.1 |
| | | met) PC-3 | |
| Colon ca. HCT-116 | 5.3 | Testis | 100.0 |
| Colon ca. CaCo-2 | 3.5 | Melanoma | 12.4 |
| | | Hs688(A).T | |
| Colon ca. HCT-15 | 7.2 | Melanoma* (met) | 6.4 |
| | | Hs688(B).T | |
| Colon ca. HCC-2998 | 6.4 | Melanoma UACC-62 | 1.6 |
| Gastric ca. * (liver met) | 26.1 | Melanoma M14 | 33.9 |
| NCI-N87 | | | |
| Bladder | 4.3 | Melanoma LOX | 8.0 |
| | | IMVI | |
| Trachea | 20.9 | Melanoma* (met) SK- | 3.0 |
| | | MEL-5 | |
| Kidney | 14.9 | Melanoma SK-MEL- | 15.9 |
| | | 28 | |
| Kidney (fetal) | 17.3 | | |

TABLE 88
Panel 1.3D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag3195, | Ag3195, | Ag401, | | Ag3195, | Ag3195, | Ag401, |
| | Run | Run | Run | | Run | Run | Run |
| | 16552493 | 16567539 | 16551817 | Tissue | 16552493 | 16567539 | 16551817 |
| Tissue Name | 0 | 1 | 4 | Name | 0 | 1 | 4 |
| Liver | 9.0 | 3.3 | 2.9 | Kidney | 13.9 | 7.2 | 13.8 |
| adenocarcinoma | | | | (fetal) | | | |
| Pancreas | 19.5 | 10.0 | 10.1 | Renal ca. | 14.5 | 7.7 | 14.8 |
| | | | | 786-0 | | | |
| Pancreatic ca. | 0.6 | 0.0 | 10.2 | Renal ca. | 9.9 | 13.9 | 13.9 |
| CAPAN 2 | | | | A498 | | | |
| Adrenal gland | 12.9 | 8.2 | 16.2 | Renal ca. | 10.7 | 9.4 | 18.2 |
| | | | | RXF 393 | | | |
| Thyroid | 14.3 | 11.3 | 18.9 | Renal ca. | 3.6 | 4.1 | 12.7 |
| | | | | ACHN | | | |
| Salivary gland | 11.8 | 15.6 | 24.7 | Renal ca. | 1.8 | 3.1 | 12.2 |
| | | | | UO-31 | | | |
| Pituitary gland | 11.0 | 7.0 | 20.2 | Renal ca. | 3.4 | 3.2 | 5.7 |
| | | | | TK-10 | | | |
| Brain (fetal) | 13.3 | 13.9 | 23.5 | Liver | 21.5 | 16.2 | 16.7 |
| Brain (whole) | 51.4 | 36.3 | 52.1 | Liver (fetal) | 8.8 | 13.8 | 9.0 |
| Brain | 39.5 | 29.7 | 16.2 | Liver ca. | 6.6 | 15.4 | 9.6 |
| (amygdala) | | | | (hepatoblast) | | | |
| | | | | HepG2 | | | |
| Brain | 26.4 | 27.2 | 47.6 | Lung | 26.2 | 47.6 | 19.5 |
| (cerebellum) | | | | | | | |
| Brain | 39.8 | 22.7 | 54.3 | Lung (fetal) | 25.3 | 62.0 | 11.2 |
| (hippocampus) | | | | | | | |
| Brain | 26.6 | 22.2 | 20.0 | Lung ca. | 11.1 | 14.1 | 18.7 |
| (substantia | | | | (small cell) | | | |
| nigra) | | | | LX-1 | | | |
| Brain | 46.3 | 27.0 | 41.5 | Lung ca. | 0.4 | 0.0 | 0.0 |
| (thalamus) | | | | (small cell) | | | |
| | | | | NCI-H69 | | | |
| Cerebral Cortex | 5.0 | 8.2 | 15.0 | Lung ca. | 6.3 | 10.7 | 10.9 |
| | | | | (s.cell var.) | | | |
| | | | | SHP-77 | | | |
| Spinal cord | 46.3 | 38.7 | 38.7 | Lung ca. | 13.5 | 18.0 | 19.5 |
| | | | | (large | | | |
| | | | | cell) NCI- | | | |
| | | | | H460 | | | |
| glio/astro U87- | 2.4 | 1.6 | 9.5 | Lung ca. | 0.8 | 1.8 | 1.6 |
| MG | | | | (non-sm. | | | |
| | | | | cell) A549 | | | |
| glio/astro U- | 28.9 | 29.3 | 49.7 | Lung ca. | 5.1 | 3.2 | 9.3 |
| 118-MG | | | | (non-s.cell) | | | |
| | | | | NCI-H23 | | | |
| astrocytoma | 8.2 | 10.9 | 10.4 | Lung ca. | 19.2 | 7.6 | 17.8 |
| SW1783 | | | | (non-s.cell) | | | |
| | | | | HOP-62 | | | |
| neuro*; met SK- | 11.7 | 6.3 | 20.2 | Lung ca. | 2.2 | 2.9 | 9.0 |
| N-AS | | | | (non-s.cl) | | | |
| | | | | NCI-H522 | | | |
| astrocytoma SF- | 16.4 | 9.2 | 25.7 | Lung ca. | 8.1 | 6.5 | 4.5 |
| 539 | | | | (squam.) | | | |
| | | | | SW 900 | | | |
| astrocytoma | 8.3 | 14.5 | 22.4 | Lung ca. | 0.0 | 1.6 | 3.1 |
| SNB-75 | | | | (squam.) | | | |
| | | | | NCI-H596 | | | |
| glioma SNB-19 | 3.2 | 5.5 | 25.9 | Mammary | 44.1 | 22.1 | 27.0 |
| | | | | gland | | | |
| glioma U251 | 26.6 | 20.3 | 59.9 | Breast ca.* | 4.4 | 1.7 | 4.3 |
| | | | | (pl.ef) | | | |
| | | | | NCF-7 | | | |
| glioma SF-295 | 10.1 | 5.5 | 15.4 | Breast ca.* | 21.0 | 15.4 | 47.3 |
| | | | | (pl.ef) | | | |
| | | | | MDA-MB- | | | |
| | | | | 231 | | | |
| Heart (fetal) | 1.9 | 2.5 | 0.5 | Breast ca.* | 3.1 | 4.9 | 6.4 |
| | | | | (pl.ef) | | | |
| | | | | T47D | | | |
| Heart | 8.0 | 15.2 | 14.0 | Breast ca. | 12.5 | 7.3 | 12.9 |
| | | | | BT-549 | | | |
| Skeletal muscle | 2.9 | 1.8 | 7.0 | Breast ca. | 4.4 | 1.9 | 6.2 |
| (fetal) | | | | MDA-N | | | |
| Skeletal muscle | 57.0 | 58.2 | 100.00 | Ovary | 10.9 | 6.5 | 6.3 |
| Bone marrow | 28.1 | 13.0 | 33.9 | Ovarian ca. | 8.4 | 6.3 | 10.5 |
| | | | | OVCAR-3 | | | |
| Thymus | 27.2 | 16.0 | 32.1 | Ovarian ca. | 3.6 | 2.9 | 7.2 |
| | | | | OVCAR-4 | | | |
| Spleen | 39.0 | 27.0 | 27.9 | Ovarian ca. | 11.2 | 9.2 | 13.6 |
| | | | | OVCAR-5 | | | |
| Lymph node | 100.0 | 100.0 | 76.8 | Ovarian ca. | 3.9 | 4.5 | 4.6 |
| | | | | OVCAR-8 | | | |
| Colorectal | 25.0 | 23.2 | 38.7 | Ovarian ca. | 16.3 | 1.6 | 2.5 |
| | | | | IRGOV-1 | | | |
| Stomach | 23.3 | 25.3 | 21.0 | Ovarian | 10.1 | 11.6 | 17.3 |
| | | | | ca.* | | | |
| | | | | (ascites) | | | |
| | | | | SK-OV-3 | | | |
| Small intestine | 55.9 | 41.8 | 60.7 | Uterus | 65.5 | 61.1 | 61.6 |
| Colon ca. | 5.6 | 2.9 | 7.6 | Plancenta | 6.6 | 10.2 | 2.9 |
| SW480 | | | | | | | |
| Colon ca.* | 6.7 | 4.9 | 2.9 | Prostate | 14.8 | 8.5 | 17.8 |
| SW620(SW480 | | | | | | | |
| met) | | | | | | | |
| Colon ca. HT29 | 2.8 | 1.1 | 0.8 | Prostate | 3.5 | 6.9 | 19.6 |
| | | | | ca.* (bone | | | |
| | | | | met) PC-3 | | | |
| Colon ca. HCT- | 4.5 | 3.2 | 3.6 | Testis | 45.1 | 23.8 | 50.7 |
| 116 | | | | | | | |
| Colon ca. | 2.0 | 2.0 | 3.1 | Melanoma | 7.2 | 3.7 | 9.5 |
| CaCo-2 | | | | Hs688(A).T | | | |
| Colon ca. | 14.5 | 5.8 | 7.9 | Melanoma* | 10.0 | 6.7 | 15.8 |
| tissue(ODO3866) | | | | (met) | | | |
| | | | | Hs688(B).T | | | |
| Colon ca. HCC- | 6.4 | 4.6 | 10.3 | Melanoma | 16.5 | 12.5 | 7.6 |
| 2998 | | | | UACC-62 | | | |
| Gastric ca.* | 48.0 | 34.9 | 55.1 | Melanoma | 73.7 | 51.8 | 89.5 |
| (liver met) NCI- | | | | M14 | | | |
| N87 | | | | | | | |
| Bladder | 21.3 | 23.3 | 22.2 | Melanoma | 1.1 | 0.6 | 2.2 |
| | | | | LOX IMVI | | | |
| Trachea | 21.5 | 26.1 | 17.4 | Melanoma* | 0.4 | 1.7 | 1.9 |
| | | | | (met) SK- | | | |
| | | | | MEL-5 | | | |
| Kidney | 44.8 | 35.8 | 19.5 | Adipose | 22.1 | 9.9 | 15.5 |

TABLE 89
Panel 2D

| | Rel. Exp. (%) Ag3195, Run | | Rel. Exp. (%) Ag3195, Run |
|---|---|---|---|
| Tissue Name | 165003127 | Tissue Name | 165003127 |
| Normal Colon | 51.1 | Kidney Margin | 1.4 |
| | | 8120608 | |
| CC Well to Mod Diff | 6.9 | Kidney Cancer | 2.5 |
| (ODO3866) | | 8120613 | |
| CC Margin (ODO3866) | 5.9 | Kidney Margin | 6.7 |
| | | 8120614 | |
| CC Gr.2 rectosigmoid | 4.3 | Kidney Cancer | 9.0 |
| (ODO3868) | | 9010320 | |
| CC Margin (ODO3868) | 2.4 | Kidney Margin | 15.5 |
| | | 9010321 | |
| CC Mod Diff (ODO3920) | 6.4 | Normal Uterus | 5.9 |
| CC Margin (ODO3920) | 12.6 | Uterus Cancer 064011 | 31.9 |
| CC Gr.2 ascend colon | 24.3 | Normal Thyroid | 10.2 |
| (ODO3921) | | | |
| CC Margin (ODO3921) | 9.2 | Thyroid Cancer | 12.5 |
| | | 064010 | |
| CC from Partial | 22.8 | Thyroid Cancer | 16.2 |
| Hepatectomy (ODO4309) | | A302152 | |
| Mets | | | |
| Liver Margin (ODO4309) | 14.7 | Thyroid Margin | 18.9 |
| | | A302153 | |
| Colon mets to lung | 3.5 | Normal Breast | 24.8 |
| (OD04451-01) | | | |
| Lung Margin (OD04451- | 7.1 | Breast Cancer | 5.8 |
| 02) | | (OD04566) | |
| Normal Prostate 6546-1 | 25.5 | Breast Cancer | 16.0 |
| | | (OD04590-01) | |
| Prostate Cancer | 10.6 | Breast Cancer Mets | 28.9 |
| (OD04410) | | (OD04590-03) | |
| Prostate Margin | 9.4 | Breast Cancer | 7.9 |
| (OD04410) | | Metastasis | |
| | | (OD04655-05) | |
| Prostate Cancer | 15.2 | Breast Cancer 064006 | 10.4 |
| (OD04720-01) | | | |
| Prostate Margin | 24.0 | Breast Cancer 1024 | 10.7 |
| (OD04720-02) | | | |
| Normal Lung 061010 | 100.0 | Breast Cancer | 7.2 |
| | | 9100266 | |
| Lung Met to Muscle | 5.3 | Breast Margin | 7.2 |
| (ODO4286) | | 9100265 | |
| Muscle Margin | 13.7 | Breast Cancer | 17.2 |
| (ODO4286) | | A209073 | |
| Lung Malignant Cancer | 24.0 | Breast Margin | 12.2 |
| (OD03126) | | A2090734 | |
| Lung Margin (OD03126) | 74.7 | Normal Liver | 27.0 |
| Lung Cancer (OD04404) | 14.0 | Liver Cancer 064003 | 45.7 |
| Lung Margin (OD04404) | 18.9 | Liver Cancer 1025 | 8.6 |
| Lung Cancer (OD04565) | 8.0 | Liver Cancer 1026 | 1.8 |
| Lung Margin (OD04565) | 16.7 | Liver Cancer 6004-T | 0.7 |
| Lung Cancer (OD04237- | 23.8 | Liver Tissue 6004-N | 6.0 |
| 01) | | | |
| Lung Margin (OD04237- | 17.3 | Liver Cancer 6005-T | 1.7 |
| 02) | | | |
| Ocular Mel Met to Liver | 19.3 | Liver Tissue 6005-N | 1.5 |
| (ODO4310) | | | |
| Liver Margin (ODO4310) | 14.3 | Normal Bladder | 35.6 |
| Melanoma Mets to Lung | 7.6 | Bladder Cancer 1023 | 3.2 |
| (OD04321) | | | |
| Lung Margin (OD04321) | 40.3 | Bladder Cancer | 15.7 |
| | | A302173 | |
| Normal Kidney | 87.7 | Bladder Cancer | 6.7 |
| | | (OD04718-01) | |
| Kidney Ca, Nuclear grade | 28.9 | Bladder Normal | 21.6 |
| 2 (OD04338) | | Adjacent (OD04718- | |
| | | 03) | |
| Kidney Margin | 25.2 | Normal Ovary | 3.7 |
| (OD04338) | | | |
| Kidney Ca Nuclear grade | 34.4 | Ovarian Cancer | 11.3 |
| 1/2 (OD04339) | | 064008 | |
| Kidney Margin | 35.6 | Ovarian Cancer | 25.0 |
| (OD04339) | | (OD04768-07) | |
| Kidney Ca, Clear cell | 20.9 | Ovary Margin | 3.7 |
| type (OD04340) | | (OD04768-08) | |
| Kidney Margin | 34.2 | Normal Stomach | 18.4 |
| (OD04340) | | | |
| Kidney Ca, Nuclear grade | 7.7 | Gastric Cancer | 4.0 |
| 3 (OD04348) | | 9060358 | |
| Kidney Margin | 28.5 | Stomach Margin | 8.2 |
| (OD04348) | | 9060359 | |
| Kidney Cancer | 8.0 | Gastric Cancer | 11.8 |
| (OD04622-01) | | 9060395 | |
| Kidney Margin | 2.9 | Stomach Margin | 8.7 |
| (OD04622-03) | | 9060394 | |
| Kidney Cancer | 13.0 | Gastric Cancer | 7.0 |
| (OD04450-01) | | 9060397 | |
| Kidney Margin | 17.9 | Stomach Margin | 3.7 |
| (OD04450-03) | | 9060396 | |
| Kidney Cancer 8120607 | 1.9 | Gastric Cancer | 38.7 |
| | | 064005 | |

TABLE 90
Panel 3D

| | Rel. Exp. (%) Ag3195, Run | | Rel. Exp. (%) Ag3195, Run |
|---|---|---|---|
| Tissue Name | 165301766 | Tissue Name | 165301766 |
| Daoy- Medulloblastoma | 15.4 | Ca Ski- Cervical epidermoid | 40.1 |
| | | carcinoma (metastasis) | |
| TE671- Medulloblastoma | 19.5 | ES-2- Ovarian clear cell | 6.7 |
| | | carcinoma | |
| D283 Med- | 62.4 | Ramos- Stimulated with | 0.0 |
| Medulloblastoma | | PMA/ionomycin 6 h | |
| PFSK-1- Primitive | 14.9 | Ramos- Stimulated with | 1.4 |
| Neuroectodermal | | PMA/ionomycin 14 h | |
| XF-498- CNS | 42.6 | MEG-01- Chronic | 25.5 |
| | | myelogenous leukemia | |
| | | (megokaryoblast) | |
| SNB-78- Glioma | 17.3 | Raji- Burkitt's lymphoma | 3.9 |
| SF-268- Glioblastoma | 29.1 | Daudi- Burkitt's lymphoma | 7.0 |
| T98G- Glioblastoma | 16.4 | U266- B-cell plasmacytoma | 76.8 |
| SK-N-SH- | 65.1 | CA46- Burkitt's lymphoma | 9.9 |
| Neuroblastoma | | | |
| (metastasis) | | | |
| SF-295- Glioblastoma | 10.9 | RL- non-Hodgkin's B-cell | 1.3 |
| | | lymphoma | |
| Cerebellum | 83.5 | JM1- pre-B-cell lymphoma | 39.5 |
| Cerebellum | 4.7 | Jurkat- T cell leukemia | 13.3 |
| NCI-H292- | 100.0 | TF-1- Erythroleukemia | 16.6 |
| Mucoepidermoid lung | | | |
| carcinoma | | | |
| DMS-114- Small cell | 10.4 | HUT 78- T-cell lymphoma | 25.2 |
| lung cancer | | | |
| DMS-79- Small cell lung | 87.7 | U937- Histiocytic lymphoma | 43.5 |
| cancer | | | |
| NCI-H146- Small cell | 16.2 | KU-812- Myelogenous | 31.9 |
| lung cancer | | leukemia | |
| NCI-H526- Small cell | 66.4 | 769-P- Clear cell renal | 21.5 |
| lung cancer | | carcinoma | |
| NCI-N417- Small cell | 14.3 | Caki-2- Clear cell renal | 14.9 |
| lung cancer | | carcinoma | |
| NCI-H82- Small cell | 7.1 | SW 839- Clear cell renal | 18.4 |
| lung cancer | | carcinoma | |
| NCI-H157- Squamous | 0.2 | G401- Wilms' tumor | 6.9 |
| cell lung cancer | | | |
| (metastasis) | | | |
| NCI-H1155- Large cell | 34.6 | Hs766T- Pancreatic | 19.1 |
| lung cancer | | carcinoma (LN metastasis) | |
| NCI-H1299- Large cell | 16.4 | CAPAN-1- Pancreatic | 13.6 |
| lung cancer | | adenocarcinoma (liver | |
| | | metastasis) | |
| NCI-H727- Lung | 0.0 | SU86.86- Pancreatic | 7.3 |
| carcinoid | | carcinoma (liver metastasis) | |
| NCI-UMC-11- Lung | 61.6 | BxPc-3- Pancreatic | 22.7 |
| carcinoid | | adenocarcinoma | |
| LX-1- Small cell lung | 30.4 | HPAC- Pancreatic | 0.7 |
| cancer | | adenocarcinoma | |
| Colo-205- Colon cancer | 60.3 | MIA PaCa-2- Pancreatic | 0.7 |
| | | carcinoma | |
| KM12- Colon cancer | 38.2 | CFPAC-1- Pancreatic ductal | 92.0 |
| | | adenocarcinoma | |
| KM20L2- Colon cancer | 2.1 | PANC-1- Pancreatic | 20.7 |
| | | epithelioid ductal carcinoma | |
| NCI-H716- Colon cancer | 43.5 | T24- Bladder carcinoma | 8.4 |
| | | (transitional cell) | |
| SW-48- Colon | 29.5 | 5637- Bladder carcinoma | 26.1 |
| adenocarcinoma | | | |
| SW1116- Colon | 6.6 | HT-1197- Bladder carcinoma | 7.0 |
| adenocarcinoma | | | |
| LS 174T- Colon | 12.1 | UM-UC-3- Bladder carcinma | 1.4 |
| adenocarcinoma | | (transitional cell) | |
| SW-948- Colon | 3.0 | A204- Rhabdomyosarcoma | 10.4 |
| adenocarcinoma | | | |
| SW-480- Colon | 8.4 | HT-1080- Fibrosarcoma | 30.1 |
| adenocarcinoma | | | |
| NCI-SNU-5- Gastric | 9.0 | MG-63- Osteosarcoma | 7.6 |
| carcinoma | | | |
| KATO III- Gastric | 46.7 | SK-LMS-1- Leiomyosarcoma | 39.2 |
| carcinoma | | (vulva) | |
| NCI-SNU-16- Gastric | 26.4 | SJRH30- Rhabdomyosarcoma | 24.5 |
| carcinoma | | (met to bone marrow) | |
| NCI-SNU-1- Gastric | 84.7 | A431- Epidermoid carcinoma | 47.0 |
| carcinoma | | | |
| RF-1- Gastric | 30.4 | WM266-4- Melanoma | 25.5 |
| adenocarcinoma | | | |
| RF-48- Gastric | 15.8 | DU 145- Prostate carcinoma | 0.0 |
| adenocarcinoma | | (brain metastasis) | |
| MKN-45- Gastric | 8.8 | MDA-MB-468- Breast | 0.9 |
| carcinoma | | adenocarcinoma | |
| NCI-N87- Gastric | 12.4 | SCC-4- Squamous cell | 0.0 |
| carcinoma | | carcinoma of tongue | |
| OVCAR-5- Ovarian | 4.5 | SCC-9- Squamous cell | 0.0 |
| carcinoma | | carcinoma of tongue | |
| RL95-2- Uterine | 2.3 | SCC-15- Squamous cell | 0.6 |
| carcinoma | | carcinoma of tongue | |
| HelaS3- Cervical | 19.8 | CAL 27- Squamous cell | 25.9 |
| adenocarcinoma | | carcinoma of tongue | |

TABLE 91
Panel 4D

| | Rel. Exp. (%) Ag3195, Run | | Rel. Exp. (%) Ag3195, Run |
|---|---|---|---|
| Tissue Name | 164530142 | Tissue Name | 164530142 |
| Secondary Th1 act | 5.7 | HUVEC IL-1beta | 1.7 |
| Secondary Th2 act | 4.6 | HUVEC IFN gamma | 4.5 |
| Secondary Tr1 act | 5.1 | HUVEC TNF alpha + IFN | 9.0 |
| | | gamma | |
| Secondary Th1 rest | 1.7 | HUVEC TNF alpha + IL4 | 3.3 |
| Secondary Th2 rest | 2.9 | HUVEC IL-11 | 1.6 |
| Secondary Tr1 rest | 4.2 | Lung Microvascular EC | 7.4 |
| | | none | |
| Primary Th1 act | 0.8 | Lung Microvascular EC | 6.9 |
| | | TNFalpha + IL-1beta | |
| Primary Th2 act | 1.5 | Microvascular Dermal EC | 11.2 |
| | | none | |
| Primary Tr1 act | 2.5 | Microsvasular Dermal EC | 6.0 |
| | | TNFalpha + IL-1beta | |
| Primary Th1 rest | 24.5 | Bronchial epithelium | 18.2 |
| | | TNFalpha + IL1beta | |
| Primary Th2 rest | 14.1 | Small airway epithelium | 6.8 |
| | | none | |
| Primary Tr1 rest | 5.2 | Small airway epithelium | 100.0 |
| | | TNFalpha + IL-1beta | |
| CD45RA CD4 | 11.6 | Coronery artery SMC rest | 2.8 |
| lymphocyte act | | | |
| CD45RO CD4 | 6.4 | Coronery artery SMC | 1.5 |
| lymphocyte act | | TNFalpha + IL-1beta | |
| CD8 lymphocyte act | 6.3 | Astrocytes rest | 2.5 |
| Secondary CD8 | 6.7 | Astrocytes TNFalpha + | 1.8 |
| lymphocyte rest | | IL-1beta | |
| Secondary CD8 | 4.9 | KU-812 (Basophil) rest | 4.6 |
| lymphocyte act | | | |
| CD4 lymphocyte none | 10.1 | KU-812 (Basophil) | 11.0 |
| | | PMA/ionomycin | |
| 2ry Th1/Th2/Tr1_anti- | 7.7 | CCD1106 (Keratinocytes) | 9.5 |
| CD95 CH11 | | none | |
| LAK cells rest | 19.2 | CCD1106 (Keratinocytes) | 10.3 |
| | | TNFalpha + IL-1beta | |
| LAK cells IL-2 | 14.2 | Liver cirrhosis | 1.6 |
| LAK cells IL-2 + IL-12 | 12.7 | Lupus kidney | 3.3 |
| LAK cells IL-2 + IFN | 20.3 | NCI-H292 none | 30.8 |
| gamma | | | |
| LAK cells IL-2 + IL-18 | 17.7 | NCI-H292 IL-4 | 26.2 |
| LAK cells | 3.9 | NCI-H292 IL-9 | 27.4 |
| PMA/ionomycin | | | |
| NK Cells IL-2 rest | 7.6 | NCI-H292 IL-13 | 14.7 |
| Two Way MLR 3 day | 17.1 | NCI-H292 IFN gamma | 22.8 |
| Two Way MLR 5 day | 5.9 | HPAEC none | 2.8 |
| Two Way MLR 7 day | 4.3 | HPAEC TNF alpha + IL-1 | 4.7 |
| | | beta | |
| PBMC rest | 3.3 | Lung fibroblast none | 3.2 |
| PBMC PWM | 27.2 | Lung fibroblast TNF alpha + | 3.1 |
| | | IL-1 beta | |
| PBMC PHA-L | 7.1 | Lung fibroblast IL-4 | 4.4 |
| Ramos (B cell) none | 1.0 | Lung fibroblast IL-9 | 4.2 |
| Ramos (B cell) | 3.7 | Lung fibroblast IL-13 | 4.7 |
| ionomycin | | | |
| B lymphocytes PWM | 23.7 | Lung fibroblast IFN | 7.5 |
| | | gamma | |
| B lymphocytes CD40L | 14.5 | Dermal fibroblast | 23.3 |
| and IL-4 | | CCD1070 rest | |
| EOL-1 dbcAMP | 14.8 | Dermal fibroblast | 38.4 |
| | | CCD1070 TNF alpha | |
| EOL-1 dbcAMP | 6.0 | Dermal fibroblast | 12.2 |
| PMA/ionomycin | | CCD1070 IL-1 beta | |
| Dendritic cells none | 13.5 | Dermal fibroblast IFN | 14.5 |
| | | gamma | |
| Dendritic cells LPS | 13.7 | Dermal fibroblast IL-4 | 19.9 |
| Dendritic cells anti- | 16.3 | IBD Colitis 2 | 1.2 |
| CD40 | | | |
| Monocytes rest | 29.9 | IBD Crohn's | 2.1 |
| Monocytes LPS | 5.3 | Colon | 11.6 |
| Macrophages rest | 11.7 | Lung | 5.8 |
| Macrophages LPS | 9.7 | Thymus | 39.8 |
| HUVEC none | 3.3 | Kidney | 31.0 |
| HUVEC starved | 9.3 | | |

AI_comprehensive panel_v1.0 Summary: Ag3195 The NOV11D gene is expressed in normal lung, bone, joint tissue, gut and skin. There is no apparent difference in transcript expression in disease tissue as compared to normal tissue. Please see Panel 4D for discussion of utility of this gene in an autoimmune context.

CNS_neurodegeneration_v1.0 Summary: Ag3195 The expression profile of the NOV11D gene shows higher expression in the temporal cortex of Alzheimers disease victims. This gene encodes an alpha glucosidase homolog. Brain glucose regulation may play a role in Alzheimer's disease. For example, hyperglycemia may exert a deleterious effect by potentiating the neuronal death produced by pathological processes taking place, such as amyloid deposition. The category of agents that interfere with the absorption of glucose and lipids includes alpha-glucosidase inhibitors. Therefore, modulators of this gene product may be useful in the treatment of Alzheimer's disease.

References:

Messier C, Gagnon M. Glucose regulation and cognitive functions: relation to Alzheimer's disease and diabetes. Behav Brain Res February 1996;75(1-2):1-11

Glucose has been found to improve memory in animals and humans. Animal research has revealed that glucose may improve memory through a facilitation of acetylcholine (ACh) synthesis and release in the brain. This glucose-related memory improvement has prompted research in elderly humans. These studies have shown that the memory-improving action of glucose depends on each individuals' blood glucose regulation. Based on these data, researchers have evaluated the effect of glucose on memory in patients with Alzheimer's disease (AD). Results demonstrated that glucose could improve memory in a subset of patients that had abnormalities in their blood glucose regulation. Interestingly, these alterations in blood glucose regulation were believed to depend on the severity of the disease process. Another line of investigation has focused on alterations in brain glucose metabolism. Both animal models and studies with Type II diabetic elderly patients have shown that altered glucose regulation impairs learning and memory processes. It is possible that in AD patients, hyperglycemia exerts a deleterious effect by potentiating the neuronal death produced by other pathological processes taking place such as amyloid deposition. Based on these data, it appears important to find the prevalence of altered glucoregulation at various stages of AD. Secondly, it may be of interest to determine prospectively whether altered glucoregulation is linked to a faster progression of the disease. Finally, if such a relationship is observed, the next logical step would be to determine whether AD patients could benefit from treatments aimed at normalizing blood glucose regulation and improving insulin sensitivity.

Panel 1 Summary: Ag401 The expression of the NOV11d gene appears to be highest in a sample derived from testis tissue (CT=26.1). In addition to testis tissue, there appears to be substantial expression associated with the cerebellar region of the brain and mammary gland. The other samples in the panel show low uniform expression. Thus, the expression of this gene could be used to distinguish testis, mammary gland and cerebellum from the other samples in the panel.

Please see Panel CNS_neurodegeneration_v1.0 for discussion of potential utility of this gene in the central nervous system.

This gene is also moderately expressed in a number of metabolic tissues including pancreas, adrenal, thyroid, pituitary, heart, skeletal muscle, and adult and fetal liver. This small molecule target may be useful for the treatment of metabolic diseases, including obesity and Types 1 and 2 diabetes.

Panel 1.3D Summary: Ag401/Ag3195 Three experiments expression of the NOV11d gene in a wide variety of metabolic tissues, including pancreas, adipose, adrenal, thyroid, pituitary, adult and fetal heart, adult and fetal skeletal muscle, and adult and fetal liver. Alpha-glucosidase inhibitors are currently used in the treatment of Type 2 diabetes to decrease glucose absorption from the gut. Thus, this gene product may be a small molecule target for the treatment of metabolic diseases, including obesity and Types 1 and 2 diabetes.

Significant brain expression of the gene is also seen in this panel. Please see Panel CNS_neurodegeneration_v1.0 for discussion of potential utility of this gene in the central nervous system.

References:

Raptis S A, Dimitriadis G D. Oral hypoglycemic agents: insulin secretagogues, alpha-glucosidase inhibitors and insulin sensitizers. Exp Clin Endocrinol Diabetes. 2001;109 Suppl 2:S265-87.

In this review we present the agents that are in use in the treatment of type 2 diabetes. Sulfonylureas of the 1st and 2nd generation increase insulin secretion but can induce hyperinsulinemia and sometimes prolonged hypoglycemia. Glimepiride is a new 3rd generation sulfonylurea with some advantages over the other members of this group, such as a lower risk of hypoglycemia, no interaction with cardiovascular KATP-channels and a possibility that it may increase insulin sensitivity. There are also newer insulin secretagogues (such as neteglinide and repaglinide) with a rapid onset of action on the beta-cell, therefore inducing a more physiological profile of insulin secretion during meals. The category of insulin sensitizers includes metformin and thiazolidinediones. Metformin effectively reduces hyperglycemia, hyperlipidemia and macroangiopathy in patients with type 2 diabetes. This agent increases the sensitivity of the liver and peripheral tissues to insulin and, therefore, it could be considered as a drug of choice for the prevention of type 2 diabetes. Thiazolidinediones (rosiglitazone and pioglitazone) increase the sensitivity of the tissues to insulin. This mechanism of action makes them powerful therapeutic tools for the treatment of type 2 diabetes (and possibly other insulin resistant states) either alone or in combination with other oral agents. The category of agents that interfere with the absorption of glucose and lipids includes alpha-glucosidase inhibitors (acarbose and miglitol) and lipase inhibitors (or-listat). alpha-Glucocidase inhibitors improve the time relationship between plasma insulin and glucose increases after a meal. Therefore, these agents may be used in the treatment of patients with type 2 diabetes, either alone at a very early stage of this disease (when insulin secretion is still adequate), or in combination with insulin secretagogues. alpha-Glucosidase inhibition may also prove useful as a supplement to insulin therapy in patients with type 1 diabetes mellitus. The inhibitor of gastrointestinal lipase orlistat may prove a useful adjunct to hypocaloric diets in patients with type 2 diabetes and obesity.

PMID: 11460577

Panel 2D Summary: Ag3195 The expression of the NOV11d gene appears to be highest in a sample of normal lung tissue adjacent to malignant lung (CT=27.4). In addition, there appears to be substantial expression in other samples of normal lung tissue, normal kidney tissue and normal colon tissue. Thus, the expresion of this gene could be used to distinguish normal lung, kidney and colon form other samples in the panel. Moreover, therapeutic modulation of this gene, through the use of small molecule drugs, antibodies or protein therapeutics might be of benefit in the treatment of lung, kidney or colon cancer.

Panel 3D Summary: Ag3195 The expression of the NOV11d gene appears to be highest in a sample derived from a lung cancer cell line(CT=29.9). In addition, there appears to be substantial expression in a number of lung cnacer cell lines in addition to a number of other samples in this panel. Thus, the expression of this gene could be used to distinguish this lung cancer cell line derived sample from other samples in the panel.

Panel 4D Summary: Ag3195 The NOV11d transcript is expressed at high levels in small airway epithelium activated with TNFalpha and IL-1beta. It is also expressed in normal tissues such as the lung, thymus and kidney, as seen in Panel AI. The protein encoded by this transcript is related to Alpha glucosidase A. This enzyme is important in lung maturation and may contribute to surfactant phospholipid biosynthesis (see reference). Therefore, therapeutics designed with the protein encoded by this transcript or therapeutics that modulate its production could potentially be used to aid lung surfactant production in premature infants, and to treat lung injuries.

References:

Bourbon J R, Doucet E, Rieutort M. Role of alpha-glucosidase in fetal lung maturation.Biochim Biophys Acta Jan. 13, 1987;917(1):203-10

The role of lysosomal enzyme acid alpha-glucosidase in fetal lung development was investigated with the aid of a specific inhibitor, the pseudosaccharide acarbose. The drug was added to a Waymouth culture medium of fetal rat lung explants cultivated for 48 h fromgestational stage 19.5 days, an in vitro system previously shown to allow morphological and biochemical maturation of alveolar epithelium. Glycogenolysis was reduced by 40% as compared with tissue cultivated on control medium, which means thatalpha-glucosidase could account for as much as 40% of fetal lung glycogenolysis, the remaining 60% being presumably achieved by cytosolic phosphorylase and by a microsomal neutral alpha-glucosidase. By the same time, the increase of phospholipids of surfactant fraction extracted from cultivated explants was partially inhibited: total and saturated phosphatidylcholine, phosphatidylethanolamine, phosphatidylglycerol and phosphatidylinositol were about 30-40% lower than in lungs cultivated on control medium. It should beemphasized that DNA concentration and increases in non-surfactant phospholipids were unchanged by the drug. alpha-Glucosidase activity was evidenced in the lysosomal fraction, in the microsomal fraction and, although in lower amounts, in the surfactant fraction extracted from term fetal lung. The results suggest that lysosomal alpha-glucosidase plays a major role in lung maturation and could facilitate glycogenolysis for the specific use of glycogen stores in providing substrates for surfactant phospholipid biosynthesis. PMID: 3539207

### Example 3

SNP Analysis of NOVX Clones

SeqCallingTM Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (Pyrosequencing, Westborough, Mass.). Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265. In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each DNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels. The DNA and protein sequences for the novel single nucleotide polymorphic variants are reported. Variants are reported individually but any combination of all or a select subset of variants are also included. In addition, the positions of the variant bases and the variant amino acid residues are underlined.

### Results

Variants are reported individually but any combination of all or a select subset of variants are also included as contemplated NOVX embodiments of the invention.

NOV1d SNP Data:

NOV1d has two SNP variants, whose variant positions for its nucleotide and amino acid sequences is numbered according to SEQ ID NOs: 7 and 8, respectively. The nucleotide sequence of the NOV1d variant differs as shown in Table 92.

TABLE 92
cSNP and Coding Variants for NOV3a

| NT Position | Wild Type | | Amino Acid | Amino Acid |
|---|---|---|---|---|
| of cSNP | NT | Variant NT | position | Change |
| 85 | T | C | 29 | C->R |
| 92 | C | T | 31 | A->V |
| 370 | C | T | 124 | F->L |

NOV5 SNP data:

NOV5 has four SNP variants, whose variant positions for its nucleotide and amino acid sequences is numbered according to SEQ ID NOs: 21 and 22, respectively. The nucleotide sequences of the NOV5 variant differs as shown in Table 93.

TABLE 93
cSNP and Coding Variants for NOV5

| NT Position | Wild Type | | Amino Acid | Amino Acid |
|---|---|---|---|---|
| of cSNP | NT | Variant NT | position | Change |
| 200 | C | T | 18 | A->V |
| 285 | C | T | 46 | No change |
| 310 | C | T | 55 | R->C |
| 393 | G | A | 82 | W->End |
| 478 | T | C | 111 | F->S |

NOV7 SNP data:

NOV7 has one SNP variant, whose variant position for its nucleotide and amino acid sequences is numbered according to SEQ ID NOs: 19 and 20, respectively. The nucleotide sequences of the NOV7 variant differs as shown in Table 94.

TABLE 94
cSNP and Coding Variants for NOV7

| NT Position | Wild Type | | Amino Acid | Amino Acid |
|---|---|---|---|---|
| of cSNP | NT | Variant NT | position | Change |
| 530 | A | G | | No change |
| 1580 | C | T | | No change |

NOV8 SNP data:

NOV8 has one SNP variant, whose variant position for its nucleotide and amino acid sequences is numbered according to SEQ ID NOs: 19 and 20, respectively. The nucleotide sequence of the NOV8 variant differs as shown in Table 95.

TABLE 95
cSNP and Coding Variants for NOV8

| NT Position | Wild Type | | | Putative |
|---|---|---|---|---|
| of cSNP | NT | Variant NT | Depth | Allele Freq. |
| 536 | C | T | 23 | 0.130 |
| NT Position | Wild Type | | Amino Acid | Amino Acid |
| of cSNP | NT | Variant NT | position | Change |
| 162 | C | T | 54 | No change |

NOV9 SNP data:

NOV9 has one SNP variant, whose variant position for its nucleotide and amino acid sequences is numbered according to SEQ ID NOs: 19 and 20, respectively. The nucleotide sequence of he NOV9 variant differs as shown in Table 96.

TABLE 96
cSNP and Coding Variants for NOV9

| NT Position | Wild Type | | Amino Acid | Amino Acid |
|---|---|---|---|---|
| of cSNP | NT | Variant NT | position | Change |
| 123 | A | G | 38 | T->A |
| 181 | A | C | 57 | N->T |
| 234 | A | G | 75 | T->A |
| 367 | C | T | 119 | A->V |
| 687 | A | G | 126 | M->V |
| 773 | A | G | 254 | No change |

NOV12a SNP data:

NOV12a has one SNP variant, whose variant position for its nucleotide and amino acid sequences is numbered according to SEQ ID NOs: 19 and 20, respectively. The nucleotide sequence of the NOV12a variant differs as shown in Table 97.

TABLE 97
cSNP and Coding Variants for NOV12a

| NT Position | Wild Type | | Amino Acid | Amino Acid |
|---|---|---|---|---|
| of cSNP | NT | Variant NT | position | Change |
| 1890 | T | C | 629 | S->P |

NOV13 SNP data:

NOV13 has one SNP variant, whose variant position for its nucleotide and amino acid sequences is numbered according to SEQ ID NOs: 19 and 20, respectively. The nucleotide sequence of the NOV13 variant differs as shown in Table 98.

TABLE 98
cSNP and Coding Variants for NOV13

| NT Position | Wild Type | | Amino Acid | Amino Acid |
|---|---|---|---|---|
| of cSNP | NT | Variant NT | position | Change |
| 783 | T | C | 234 | C->R |

### Example 4

In-frame Cloning

NOV1b-1d

For NOV1b-1d, the cDNA coding for the DOMAIN of NOV1a from residues 189 to 420 was targeted for “in-frame” cloning by PCR. The PCR template was based on the previously identified plasmid, when available, or on human cDNA(s).

TABLE 99
Oligonucleotide primers used to clone the target cDNA sequence:

| Primers | Sequences | |
|---|---|---|
| F1 | 5′-AGATCT GTCCAAGGAAGGGAAACAGCTATGGAAGGGGAA-3′ | (SEQ ID NO:238) |
| R1 | 5′-CTCGAG CATACCAGTCTTTGAGGCAATCCAATCTCG-3′ | (SEQ ID NO:239) |

NOV12b-12e

For NOV12b-12e, the cDNA coding for the DOMAIN of NOV12b-12e from residues 29 to 291 was targeted for “in-frame” cloning by PCR. The PCR template was based on the previously identified plasmid, when available, or on human cDNA(s).

TABLE 100
Oligonucleotide primers used to clone the target
cDNA sequence:

| Primers | Sequences | |
|---|---|---|
| SF1 | 5′-AAAGTCCGAAAACTTCAGAAAGATAC -3′ | (SEQ ID NO:240) |
| SF2 | 5′- CTTGTCTGATAACTTCCTGACCTCCC -3′ | (SEQ ID NO:241) |
| SR1 | 5′- TTATAGCTCATTTTTAAGACCATCAGT -3′ | (SEQ ID NO:242) |
| SR2 | 5′-CAGGAAGTTATCAGACAAGTATAGGAAC-3′ | (SEQ ID NO:243) |

For downstream cloning purposes, the forward primer includes an in-frame Hind HI restriction site and the reverse primer contains an in-frame Xho I restriction site.

Two parallel PCR reactions were set up using a total of 0.5-1.0 ng human pooled cDNAs as template for each reaction. The pool is composed of 5 micrograms of each of the following human tissue cDNAs: adrenal gland, whole brain, amygdala, cerebellum, thalamus, bone marrow, fetal brain, fetal kidney, fetal liver, fetal lung, heart, kidney, liver, lymphoma, Burkitt's Raji cell line, mammary gland, pancreas, pituitary gland, placenta, prostate, salivary gland, skeletal muscle, small Intestine, spleen, stomach, thyroid, trachea, uterus.

When the tissue of expression is known and available, the second PCR was performed using the above primers and 0.5 ng-1.0 ng of one of the following human tissue cDNAs:

skeleton muscle, testis, mammary gland, adrenal gland, ovary, colon, normal cerebellum, normal adipose, normal skin, bone marrow, brain amygdala, brain hippocampus, brain substantia nigra, brain thalamus, thyroid, fetal lung, fetal liver, fetal brain, kidney, heart, spleen, uterus, pituitary gland, lymph node, salivary gland, small intestine, prostate, placenta, spinal cord, peripheral blood, trachea, stomach, pancreas, hypothalamus.

The reaction mixtures contained 2 microliters of each of the primers (original concentration: 5 pmol/ul), 1 microliter of 10 mM DNTP (Clontech Laboratories, Palo Alto Calif.) and 1 microliter of 50×Advantage-HF 2 polymerase (Clontech Laboratories) in 50 microliter-reaction volume. The following reaction conditions were used:

PCR condition 1:

| | | |
|---|---|---|
| a) | 96° C. | 3 minutes |
| b) | 96° C. | 30 seconds denaturation |
| c) | 60° C. | 30 seconds, primer annealing |
| d) | 72° C. | 6 minutes extension |

Repeat steps b-d 15 times

| | | |
|---|---|---|
| e) | 96° C. | 15 seconds denaturation |
| f) | 60° C. | 30 seconds, primer annealing |
| g) | 72° C. | 6 minutes extension |

Repeat steps e-g 29 times

| | | |
|---|---|---|
| e) | 72° C. | 10 minutes final extension |

PCR condition 2:

| | | |
|---|---|---|
| a) | 96° C. | 3 minutes |
| b) | 96° C. | 15 seconds denaturation |
| c) | 76° C. | 30 seconds, primer annealing, reducing the temperature by |
| | | 1° C. per cycle |
| d) | 72° C. | 4 minutes extension |

Repeat steps b-d 34 times

| | | |
|---|---|---|
| e) | 72° C. | 10 minutes final extension |

An amplified product was detected by agarose gel electrophoresis. The fragment was gel-purified and ligated into the pCR2. 1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the gene-specific primers in Tables 88 and 89.

TABLE 88
Gene-specific Primers

| NOV | Primers | Sequences | |
|---|---|---|---|
| NOV11c | SF1 | GCCCTCCCGGTCCAGGTC | (SEQ ID NO:200) |
| | SF2 | GGCGACGGCACCAGCATGT | (SEQ ID NO:201) |
| | SR1 | GCCTGGCCTGCCGGGTTCT | (SEQ ID NO:202) |
| | SR2 | CATGAGCACGTGGTAAGCG | (SEQ ID NO:203) |

TABLE 89
Gene-specific Primers

| NOV | Primers | Sequences | |
|---|---|---|---|
| NOV1b | SF1 | GTGCTGGCATTGGAGTGTTTAGTG | (SEQ ID NO:204) |
| | SF2 | ATCAAGCACGTTGACACAGAATGAG | (SEQ ID NO:205) |
| | SF3 | GCATTCACTAACCTAACACCATTTACA | (SEQ ID NO:206) |
| | SF4 | GTTCAGCAGAGATGTCGTCTGACCTTC | (SEQ ID NO:207) |
| | SF5 | GGGATCCTCCACATCCTGTATTTTT | (SEQ ID NO:208) |
| | SF6 | TGAAGAACACATCAACAACAGACATAA | (SEQ ID NO:209) |
| | SR1 | ACTGTTTTCAGCAGCTACCTTAATTTC | (SEQ ID NO:210) |
| | SR2 | CTTGATGAATGTGTGGTACGCGAT | (SEQ ID NO:211) |
| | SR3 | GTGAATGCAAACTTGAGGTCTTTTGT | (SEQ ID NO:212) |
| | SR4 | CCTCATATAATCCTACCATTGGCTGTACT | (SEQ ID NO:213) |
| | SR5 | GAGGATCCCAGTGTAAAAATACTTCTG | (SEQ ID NO:214) |
| | SR6 | TAGCACTTCATAAGCAATAATGATCCC | (SEQ ID NO:215) |
| | SR7 | TGAGTGTACTAGCAGACACCTCAATGAT | (SEQ ID NO:216) |

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58;
(b) a variant of a mature form of an amino acid sequence selected from the group consisting of SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58; and
(d) a variant of an amino acid sequence selected from the group consisting of SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58;
(b) a variant of a mature form of an amino acid sequence selected from the group consisting of SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58;
(d) a variant of an amino acid sequence selected from the group consisting SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57;
(b) a nucleotide sequence differing by one or more nucleotides from a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, and 57, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule, thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to a cancer.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS: 2, 4, 6, 8, 10, 12, 14, 16, 18, 20, 22, 24, 26, 28, 30, 32, 34, 26, 28, 40, 42, 44, 46, 48, 50, 52, 54, 56, and 58, or a biologically active fragment thereof.

49. A method of treating a pathological state in a mammal, the method comprising administering to the mammal the antibody of claim 15 in an amount sufficient to alleviate the pathological state.
